(19) United States
(12) Patent Application Publication
(10) Pub. No.: US 2024/0252454 A1
(43) Pub. Date: Aug. 1, 2024

(54) COMPOSITION COMPRISING AN INHIBITOR OF MITOCHONDRIAL TRANSCRIPTION

(71) Applicants: LEAD DISCOVERY CENTER GMBH, Dortmund (DE); MAX-PLANCK-GESELLSCHAFT ZUR FÖRDERUNG DER WISSENSCHAFTEN E.V., Munchen (DE)

(72) Inventors: Tim BERGBREDE, Dortmund (DE); Raffaella DI LUCREZIA, Wuppertal (DE); Anke UNGER, Dortmund (DE); Axel CHOIDAS, Herdecke (DE); Bert KLEBL, Dortmund (DE); Peter NUSSBAUMER, Dortmund (DE); Sascha MENNINGER, Witten (DE); Peter HABENBERGER, Dortmund (DE); Gunther ZISCHINSKY, Dortmund (DE); Uwe KOCH, Dortmund (DE); Peter SCHRÖDER, Dortmund (DE); Pavla JESTRABOVÁ, Prague 6 (CZ); Lenka PALOVÁ-JELÍNKOVÁ, Klecany (CZ); Klára DÁNOVÁ, Praha 4 (CZ); Maria FALKENBERG-GUSTAFSSON, Mölndal (SE); Laleh ARABANIAN, Sävedalen (SE); Claes GUSTAFSSON, Mölndal (SE); Nils-Göran LARSSON, Sollentuna (SE); Lars PALMQVIST, Goteborg (SE)

(21) Appl. No.: 18/289,283

(22) PCT Filed: May 2, 2022
(86) PCT No.: PCT/EP2022/061705

§ 371 Date: Nov. 2, 2023

(30) Foreign Application Priority Data
May 3, 2021 (EP) 21171865.5

(51) Int. Cl. A61K 31/166; A61K 31/4184; A61K 31/453; A61K 31/47; A61K 31/496; A61K 31/5377; A61K 31/711; A61P 35/00

(52) U.S. Cl. CPC A61K 31/166; A61K 31/4184; A61K 31/453; A61K 31/47; A61K 31/496; A61K 31/5377; A61K 31/711; A61P 35/00

(57) ABSTRACT

The present invention relates to a composition comprising at least one inhibitor of mitochondrial transcription (IMT) and at least one anti-cancer drug. Furthermore, the present invention is directed to compositions for use as a medicament and to compositions for use in the treatment and/or prevention of cancer.

### FIELD OF THE INVENTION

The present invention relates to a composition comprising at least one inhibitor of mitochondrial transcription (IMT) and at least one anti-cancer drug.

### BACKGROUND OF THE INVENTION

Despite enormous research efforts during the last decades and advanced cancer treatments, cancer remains a major public health problem worldwide and is the second leading cause of death in the United States. In the US population, incidence and death rates are even increasing for several cancer types, including liver and pancreas—two of the most fatal cancers (Siegel et al., 2016). Thus, there is still an urgent need to obtain additional and improved treatment options for fighting cancer besides the established chemotherapies, radiation and upcoming immunotherapies. Combination therapies are an important concept for new and effective cancer treatments.

Interfering with the cancer metabolism is another principle to tackle tumor growth. In contrast to normal differentiated cells, which rely primarily on mitochondrial oxidative phosphorylation to generate energy, most cancer cells instead rely on aerobic glycolysis, a phenomenon termed “the Warburg effect” (Vander Heiden et al., 2009). Aerobic glycolysis in the cytoplasm leads to pyruvate generated from glucose, which is not transported into mitochondria for total oxidation for yielding more energy but is converted to lactate, originally described by Warburg (Hsu and Sabatini, 2008). Lactate is transferred to the liver, where the carbon skeleton is used to synthesize glucose known as the “neoplastic or pathological Cori cycle” contributing to the clinical metabolic state of Cachexia, a condition existing in neoplastic patients who suffer massive loss of normal body mass as the neoplasm continues its growth (Tisdale, 2002). Consequently, inhibiting aerobic glycolysis (Warburg effect) and/or neoplastic anabolism (pathological Cori cycle) may be another effective way to interfere with cancer metabolism and effectively treat cancer patients. The inhibition of glycolysis in connection with the Warburg effect for cancer treatment has been described by Pelicano, H. et al. (2006) and Scatena et al. (2008).

However, the relevance of mitochondrial respiration in tumors is varied depending on tumor type. An oxidative class of tumors and tumors with dual capacity for glycolytic and oxidative metabolism is evident and the importance of mitochondria in tumor cell survival and proliferation, including utilization of alternative oxidizable substrates such as glutamine and fatty acids, has been increasingly appreciated. The diversity of carbon substrate utilization pathways in tumors is indicative of metabolic heterogeneity that may not only be relevant across different types of cancer but also manifest within a group of tumors that otherwise share a common diagnosis (Caro et al., 2012). Accordingly, tumors show heterogeneity in fuel utilization even within the same disease entity with some having a significant mitochondrial component, marked by elevated oxidative phosphorylation (OXPHOS), increased contribution of mitochondria to total cellular energy budget, greater incorporation of fatty acid- and glucose-derived carbons into the TCA cycle, and increased lipogenesis from these carbon substrates (Caro et al., 2012).

Indeed, recent evidence supports the hypothesis that acquired resistance to therapy is accompanied by a metabolic shift from aerobic glycolysis toward respiratory metabolism, suggesting that metabolic plasticity can have a role in survival of cells responsible for tumor relapse, suggesting that metabolic plasticity can have a role in survival of cells responsible for tumor relapse. For example, it has been observed that several drug-resistant tumor cells show a higher respiratory activity than parental cells. The metabolic adaptation allows OXPHOS-addicted cancer cells to easily survive drug treatments but leaves cells susceptible to inhibitors of OXPHOS (Denise et al., 2015).

Cancer cell mitochondria are structurally and functionally different from their normal counterparts. Moreover, tumor cells exhibit an extensive metabolic reprogramming that renders them more susceptible to mitochondrial perturbations than non-immortalized cells. Based on these premises, mitochondrially-targeted agents emerge as a means to selectively target tumors. The correction of cancer-associated mitochondrial dysfunctions and the (re)activation of cell death programs by pharmacological agents that induce or facilitate mitochondrial membrane permeabilization represent attractive strategies for cancer therapy. Further, autophagy in the tumor stroma and oxidative mitochondrial metabolism (OXPHOS) in cancer cells can both dramatically promote tumor growth, independently of tumor angiogenesis (Salem et al., 2012) and that cancer-associated fibroblasts undergo aerobic glycolysis, thereby producing lactate, which is utilized as a metabolic substrate by adjacent cancer cells. In this model, “energy transfer” or “metabolic-coupling” between the tumor stroma and epithelial cancer cells “fuels” tumor growth and metastasis, via oxidative mitochondrial metabolism in anabolic cancer cells, the “reverse Warburg effect” (Whitaker-Menezes et al., 2011).

Accordingly, these findings provide a rationale for novel strategies for anti-cancer therapies by employing inhibitors of OXPHOS and mitochondrial functions. Mitochondrial targeted anti-cancer drugs are reviewed by Fulda et al. (2010) and Weinberg and Chandel (2015) including inhibitors of mitochondrial complex 1, inhibitors of the electron transfer chain (ETC) complex, inhibitors of mitochondrial ribosomal machinery, inhibitors of the translation of ETC subunits, inhibitors of mitochondrial chaperone proteins, inhibitors of glutaminases, aminotransferases or glutamate dehydrogenases, short term inhibition of autophagy, mitochondrial-targeted antioxidants.

Recently, mitochondrial RNA polymerase (POLRMT, also known as h-mtRNAP) has been proposed as a new target in acute myeloid leukemia (Bralha et al., 2015). POLRMT is responsible for the transcription of the 13 subunits of the OXPHOS complexes, two rRNAs and 22 tRNAs required for mitochondrial translation and acts as the RNA primase for mitochondrial DNA replication (Wanrooij and Falkenberg, 2010, Scarpulla, 2008). Therefore, this enzyme is of fundamental importance for both expression and replication of the human mitochondrial genome (Arnold et al., 2012).

A number of nucleoside analogues used as antiviral agents to target viral RNA polymerases demonstrate off-target inhibition of POLRMT (Arnold et al., 2012); POLRMT is distantly related to bacteriophage T7 class of single-subunit RNAPs. The finding that treatment with 2-C-methyladenosine, identified as an inhibitor of the RNA-dependent RNA polymerase of hepatitis C virus (Carroll et al., 2003), triggers the death of AML cells allegedly through rather unspecific inhibition of mitochondrial transcription confirms this rational (Bralha et al., 2015).

The invention described relates to the use of various anti-cancer drugs with a new class of inhibitors of mitochondrial RNA polymerase (IMTs) (WO2019/057821, WO2020/188049 and EP3598972), which are based on a novel molecular mechanism of action (Bonekamp et al; 2020).

Similar approaches for combining inhibitors, for example of the MEK, Bcl-2 (WO2020/068979) or PARP signal cascade path (EP02930238), or of the cellular metabolism (GLUT, WO2020/086830), have always been based on a combination with very non-specific polymerase inhibitors (nucleotide analogues) or non-specific inhibitors of the electron transport chain (VLX600).

In the case of the mentioned nucleotide analogues, strand breaks are generated in the polymerase reaction (2-C-methyladenosine; Bralha et al doi: 1018632/oncotarget6129), polymerase arrest and induction of DNA damage repair mechanisms (clofarabine-5′-monophosphate), modification of nucleobases, such as alkylation (melphalan) or phosphorylation (cytarabine), as well as strand intercalation (doxorubicin) is caused. All of these substances affect a broad spectrum of different tissues and in particular have an unfavourable, broad spectrum of potential molecular targets, since in principle all or very many enzymes containing nucleotide-binding domains can be affected; e.g. DNA polymerases, RNA polymerases, (topo-) isomerases, helicases, GPCRs, kinases.

In contrast to this, IMTs are allosteric inhibitors of the human POLRMT, with a proven high specificity for this cellular target molecule alone. Their effect on mitochondrial metabolism, oxidative phosphorylation (OXPHOS) and mitochondrial replication is also based on this and, unlike in the case of e.g. VLX600, is fully understood mechanistically.

All in all, this results in a very advantageous spectrum of activity and side effect profile for the combinations with inhibitors of other oncologically relevant signalling pathways. The observed synergies of the combinations used, allow in some cases significant reductions in the doses of the respective established standard inhibitors, with the same or better effect, and should translate, together with the high specificity and selectivity of IMTs, into a significantly improved side effect profile.

Thus, there is a need for compositions, which specifically inhibit POLRMT and are suitable for use as a medicament. In particular, a need exists for compositions that can be used in the treatment and/or prevention of cancer.

Accordingly, the present invention provides compositions for the treatment of cancer.

### SUMMARY OF THE INVENTION

The present invention, in one aspect, relates to a composition comprising at least one inhibitor of mitochondrial transcription (IMT) and at least one anti-cancer drug.

In one embodiment, the invention relates to a composition, as defined above, wherein the at least one IMT is a Mitochondrial RNA Polymerase inhibitor as determined using an assay as described herein (see Assays 1 and 2).

In a further embodiment, the invention relates to a composition, as defined above, wherein the at least one IMT is a compound of the general formula (I)

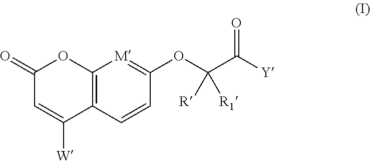

wherein
R′ is —C₁-C₄-alkyl, preferably -methyl or -ethyl, in particular -methyl;
R₁′ is —H, or -methyl, preferably —H;
M′ is CH or N;
   W′ is with

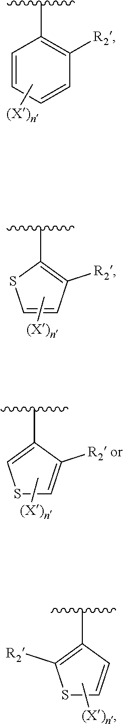

R₂′ is C₁-C₄-alkyl, -halogen, —CN, preferably -methyl, -ethyl, —C₁, or —Br;
X′ is -halogen, or —CN, preferably —Cl, —Br, or —F, in particular —F, with n′=1 or 2;
n′=0, 1, or 2, preferably 0 or 1;
Y′ is —NR₃′R₄′ with
   R₃′ is —H, or —C₁-C₄-alkyl, preferably —H or -methyl, and
   R₄′ is —C₁-C₄-alkyl or —C₃-C₆-cycloalkyl, preferably -methyl, -ethyl, -isopropyl, or -cyclopropyl; or an unsubstituted or substituted pyridine residue; or an unsubstituted or substituted phenyl residue, preferably substituted at the para position;

Y′ is —NR₃′R₄′ with N, R₃′ and R₄′ forming an unsubstituted or substituted 5- or 6-membered saturated heterocycle; or
Y′ is —OR₁₁′, with R₁₁′ is —H or —C₁-C₄-alkyl, preferably —H, -methyl, -ethyl, or -isopropyl, or a pharmaceutically or veterinary acceptable salt, hydrate or solvate thereof;
   or
wherein the at least one IMT is a compound of the general formula (II)

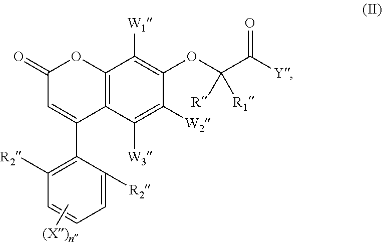

wherein
R′″ is —H or —C₁-C₄-alkyl;
R₁′″ is —H or -methyl;
R₂′ is —H;
n′″=0, 1 or 2;
X′″ is -halogen, —C₁-C₄-alkyl, —OMe or —CN, with n′″=1 or 2;
Y″ is —NR₃′″R₄′″ with
   R₃′″ is —H, or —C₁-C₄-alkyl, and
   R₄′″ is —H, —C₁-C₄-alkyl or —C₃-C₈-cycloalkyl;
   an unsubstituted or substituted pyridine residue;
   an unsubstituted or substituted pyridinylmethyl residue;
   an unsubstituted or substituted morpholinylethyl residue;
   an unsubstituted or substituted furanylmethyl residue;
   an unsubstituted or substituted phenyl residue;
   an unsubstituted or substituted benzyl residue;
   an unsubstituted or substituted phenethyl residue;
   the group

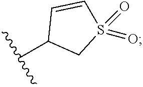 [SMILES: *C1C=CS(=O)(=O)C1]

or
the group

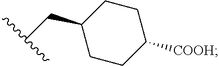 [SMILES: *C[C@H]1CC[C@H](C(=O)O)CC1]

or
Y″ is —NR₃′″R₄′″ with N, R₃′″ and R₄′″ forming an unsubstituted or substituted 5- or 6-membered saturated heterocycle,

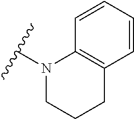 [SMILES: *N1CCCc2ccccc21]

or
Y″ is —OR₁₁′″, with R₁₁′″ is —H or —C₁-C₄-alkyl, phenyl, benzyl or 2-ethoxyethyl; and
W₁′″, W₂′″, and W₃′″ are identical or different, and are —H, -halogen, or —C₁-C₄-alkyl; or a pharmaceutically or veterinary acceptable salt, hydrate or solvate thereof, or
wherein the at least one IMT is a compound of the general formula (III)

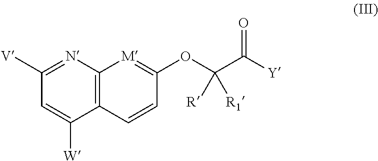

wherein
R′″ is —H, or —C₁-C₄-alkyl, preferably —H, -methyl or -ethyl; in particular methyl;
R₁′″ is —H, or -methyl, preferably —H;
M′″ is CH or N; preferably CH;
V′″ is —H, —OH, —Cl, —F, or —C₁-C₄-alkyl, preferably —H, —Cl, —F, or -methyl;
   W′″ is

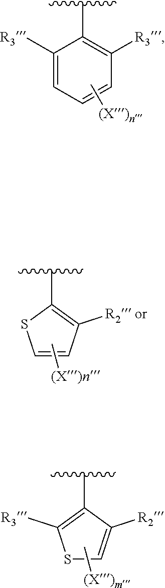

R₂′″ and R₃′″ are identical or different and are
—H, —C₁-C₄-alkyl, halogen-C₁-C₄-alkyl, —C₁-C₄-alkoxy, —C₁-C₄-dialkylamino, —C₂-C₆-alkenyl, —C_{2-C6} alkynyl, -halogen, —CN or —CO—NH₂;
preferably —H, —C₁-C₄-alkyl, —CF₃, —OCH₃, —NHCH₃, —N(CH₃)₂, —F, or —Cl;
X+″ is -halogen, or —CN, preferably —F, with n′″=1 or 2 or with m′″=1;
n′″=0, 1, or 2, preferably 0 or 1;
m′″=0 or 1, preferably 0;
Y′″ is —NR₄′″R₅′″ with
R₄′″ is —H, or —C₁-C₄-alkyl, preferably —H or -methyl, and
R₅′″ is —H, —C₁-C₄-alkyl, an unsubstituted or substituted —C₃-C₆-cycloalkyl, preferably -methyl, -ethyl, -isopropyl, or -cyclopropyl; or an unsubstituted or substituted pyridine residue; or an unsubstituted or substituted phenyl residue, preferably substituted at the para position; or
Y′″ is —NR₄′″R₅′″ with N, R₄′″ and R₅′″ forming an unsubstituted or substituted 4-, 5- or 6-membered saturated heterocycle, preferably an unsubstituted or substituted azetidine residue, an unsubstituted or substituted piperidine residue, an unsubstituted or substituted pyrrolidine residue, an unsubstituted or substituted piperazine residue, an unsubstituted or substituted morpholine residue, or an unsubstituted or substituted tetrahydropyridine residue; or
Y′″ is —OR₆′″, with R₆′″ is —H or —C₁-C₄-alkyl, preferably —H, -methyl, -ethyl, -isopropyl, or -tert-butyl;
or a pharmaceutically or veterinary acceptable salt, hydrate or solvate thereof.

In a further embodiment, the invention relates to a composition, as defined above, wherein the IMT is selected from the group consisting of:
tert-butyl 2-(2-oxo-4-phenyl-chromen-7-yl)oxy-propanoate
N,N-dimethyl-2-(2-oxo-4-phenyl-chromen-7-yl)oxy-propanamide
ethyl 2-[4-(4-chlorophenyl)-2-oxo-chromen-7-yl]oxypropanoate
ethyl 2-[4-(3-chlorophenyl)-2-oxo-chromen-7-yl]oxypropanoate
N-[4-(3-hydroxypropyl)phenyl]-2-(2-oxo-4-phenyl-chromen-7-yl)oxy-propanamide
2-(2-oxo-4-phenyl-chromen-7-yl)oxy-N-(2-pyridyl)propanamide
ethyl 2-[4-(3-fluorophenyl)-2-oxo-chromen-7-yl]oxypropanoate
ethyl 2-[4-(4-fluorophenyl)-2-oxo-chromen-7-yl]oxypropanoate
ethyl 2-[2-oxo-4-(p-tolyl)chromen-7-yl]oxypropanoate
methyl 1-[2-(6-chloro-2-oxo-4-phenyl-chromen-7-yl)oxypropanoyl]piperidine-3-carboxylate
2-(6-chloro-2-oxo-4-phenyl-chromen-7-yl)oxy-N-(2-pyridyl)propanamide
N-isopropyl-2-(2-oxo-4-phenyl-chromen-7-yl)oxy-propanamide
1-[2-(6-chloro-2-oxo-4-phenyl-chromen-7-yl)oxypropanoyl]piperidine-3-carboxylic acid
1-[2-(2-oxo-4-phenyl-chromen-7-yl)oxypropanoyl]piperidine-3-carboxylic acid
methyl 1-[2-(2-oxo-4-phenyl-chromen-7-yl)oxypropanoyl]piperidine-3-carboxylate
N-[4-(2-hydroxyethyl)phenyl]-2-(2-oxo-4-phenyl-chromen-7-yl)oxy-propanamide
2-(6-chloro-2-oxo-4-phenyl-chromen-7-yl)oxy-N-[4-(2-hydroxyethyl)phenyl]propanamide
N-[4-(2-hydroxyethyl)phenyl]-N-methyl-2-(2-oxo-4-phenyl-chromen-7-yl)oxy-propanamide
1-[2-(2-oxo-4-phenyl-chromen-7-yl)oxypropanoyl]piperidine-3-carbonitrile
7-[1-methyl-2-oxo-2-[3-(2H-tetrazol-5-yl)-1-piperidyl]ethoxy]-4-phenyl-chromen-2-one
(3S)-1-[(2R)-2-(2-oxo-4-phenyl-chromen-7-yl)oxypropanoyl]piperidine-3-carboxylic acid
2-(2-oxo-4-phenyl-chromen-7-yl)oxy-N-(2-pyridyl)acetamide
methyl 1-[2-(2-oxo-4-phenyl-chromen-7-yl)oxyacetyl]piperidine-3-carboxylate
phenyl 2-(2-oxo-4-phenyl-chromen-7-yl)oxypropanoate
2-(2-oxo-4-phenyl-chromen-7-yl)oxy-N-phenyl-propanamide
N-methyl-2-(2-oxo-4-phenyl-chromen-7-yl)oxy-propanamide
2-(6-chloro-2-oxo-4-phenyl-chromen-7-yl)oxy-N-(2-hydroxy-2-phenyl-ethyl)propanamide
2-(6-chloro-2-oxo-4-phenyl-chromen-7-yl)oxy-N-[4-(3-hydroxypropyl)phenyl]propanamide
N-(2-hydroxy-2-phenyl-ethyl)-2-(2-oxo-4-phenyl-chromen-7-yl)oxy-propanamide
N-ethyl-2-(2-oxo-4-phenyl-chromen-7-yl)oxy-propanamide
7-(1-methyl-2-oxo-2-pyrrolidin-1-yl-ethoxy)-4-phenyl-chromen-2-one
ethyl 2-methyl-2-(2-oxo-4-phenyl-chromen-7-yl)oxy-propanoate
ethyl (2R)-2-(2-oxo-4-phenyl-chromen-7-yl)oxypropanoate
(2R)—N,N-dimethyl-2-(2-oxo-4-phenyl-chromen-7-yl)oxy-propanamide
ethyl (3S)-1-[(2R)-2-(2-oxo-4-phenyl-chromen-7-yl)oxypropanoyl]piperidine-3-carboxylate
(3S)-1-[2-(2-oxo-4-phenyl-chromen-7-yl)oxypropanoyl]piperidine-3-carboxylic acid
(3R)-1-[2-(2-oxo-4-phenyl-chromen-7-yl)oxypropanoyl]piperidine-3-carboxylic acid
methyl (3R)-1-[2-(2-oxo-4-phenyl-chromen-7-yl)oxypropanoyl]piperidine-3-carboxylate
7-[1-methyl-2-oxo-2-(1-piperidyl)ethoxy]-4-phenyl-chromen-2-one
N-[4-(2-hydroxyethyl)phenyl]-N-methyl-2-(2-oxo-4-phenyl-chromen-7-yl)oxy-acetamide
ethyl 2-[4-(4-bromophenyl)-2-oxo-chromen-7-yl]oxypropanoate
2-(6-chloro-2-oxo-4-phenyl-chromen-7-yl)oxy-N-(2-furylmethyl)propanamide
ethyl 1-[2-(2-oxo-4-phenyl-chromen-7-yl)oxyacetyl]piperidine-3-carboxylate
tert-butyl 2-(2-oxo-4-phenyl-chromen-7-yl)oxyacetate
6-chloro-7-(2-morpholino-2-oxo-ethoxy)-4-phenyl-chromen-2-one
2-(6-chloro-2-oxo-4-phenyl-chromen-7-yl)oxy-N-cyclopropyl-acetamide
2-(6-chloro-2-oxo-4-phenyl-chromen-7-yl)oxy-N,N-diethyl-acetamide
N,N-diethyl-2-(2-oxo-4-phenyl-chromen-7-yl)oxy-acetamide
ethyl 2-(6-chloro-2-oxo-4-phenyl-chromen-7-yl)oxyacetate
ethyl 2-(6-chloro-2-oxo-4-phenyl-chromen-7-yl)oxypropanoate
2-(2-oxo-4-phenyl-chromen-7-yl)oxypropanoic acid
ethyl 2-(2-oxo-4-phenyl-chromen-7-yl)oxypropanoate
isopropyl 2-(2-oxo-4-phenyl-chromen-7-yl)oxypropanoate
1-[2-(2-oxo-4-phenyl-chromen-7-yl)oxyacetyl]piperidine-3-carboxylic acid
2-(2-oxo-4-phenyl-chromen-7-yl)oxy-N-(3-pyridyl)propanamide
methyl 2-(2-oxo-4-phenyl-chromen-7-yl)oxypropanoate
2-(6-chloro-2-oxo-4-phenyl-chromen-7-yl)oxy-N-[4-(2-hydroxyethyl)phenyl]acetamide
methyl 2-(6-chloro-2-oxo-4-phenyl-chromen-7-yl)oxypropanoate
2-(6-chloro-2-oxo-4-phenyl-chromen-7-yl)oxy-N-(3-pyridylmethyl)acetamide
1-[2-(2-oxo-4-phenyl-chromen-7-yl)oxypropanoyl]piperidine-3-carboxamide
2-(2-oxo-4-phenyl-chromen-7-yl)oxy-N-(4-pyridyl)propanamide
N-methyl-1-[2-(2-oxo-4-phenyl-chromen-7-yl)oxypropanoyl]piperidine-3-carboxamide
methyl 1-[2-(2-oxo-4-phenyl-chromen-7-yl)oxypropanoyl]piperidine-4-carboxylate
ethyl rac-(3S)-1-[2-(2-oxo-4-phenyl-chromen-7-yl)oxypropanoyl]piperidine-3-carboxylate
ethyl 2-[4-(4-methoxyphenyl)-2-oxo-chromen-7-yl]oxypropanoate
(2R)-2-(2-oxo-4-phenyl-chromen-7-yl)oxypropanoic acid
2-(2-oxo-4-phenyl-chromen-7-yl)oxypropanamide
rac-(3S)—N,N-dimethyl-1-[2-(2-oxo-4-phenyl-chromen-7-yl)oxypropanoyl]piperidine-3-carboxamide
1-[2-(2-oxo-4-phenyl-chromen-7-yl)oxypropanoyl]piperidine-4-carboxylic acid
N-(2-morpholinoethyl)-2-(2-oxo-4-phenyl-chromen-7-yl)oxy-propanamide
ethyl 2-(2-oxo-4-phenyl-chromen-7-yl)oxybutanoate
N-(4-methoxyphenyl)-2-(2-oxo-4-phenyl-chromen-7-yl)oxy-propanamide
2-ethoxyethyl 2-(2-oxo-4-phenyl-chromen-7-yl)oxyacetate
propyl 2-(2-oxo-4-phenyl-chromen-7-yl)oxyacetate
2-(2-oxo-4-phenyl-chromen-7-yl)oxyacetamide
butyl 2-(2-oxo-4-phenyl-chromen-7-yl)oxyacetate
isobutyl 2-(2-oxo-4-phenyl-chromen-7-yl)oxyacetate
6-chloro-7-[2-(3,4-dihydro-2H-quinolin-1-yl)-2-oxo-ethoxy]-4-phenyl-chromen-2-one
2-(6-chloro-2-oxo-4-phenyl-chromen-7-yl)oxy-N-(2-morpholinoethyl)propanamide
2-(5-fluoro-2-oxo-4-phenyl-chromen-7-yl)oxy-N,N-dimethyl-propanamide
ethyl 2-(2-oxo-4-phenyl-chromen-7-yl)oxyacetate
tert-butyl 2-(6-chloro-2-oxo-4-phenyl-chromen-7-yl)oxyacetate
2-(6-chloro-2-oxo-4-phenyl-chromen-7-yl)oxy-N-(2-morpholinoethyl)acetamide
2-(6-chloro-2-oxo-4-phenyl-chromen-7-yl)oxyacetamide
1-[2-(6-chloro-2-oxo-4-phenyl-chromen-7-yl)oxyacetyl]piperidine-3-carboxylic acid
2-(6-chloro-2-oxo-4-phenyl-chromen-7-yl)oxy-N-cyclooctyl-acetamide
ethyl 2-(5-fluoro-2-oxo-4-phenyl-chromen-7-yl)oxypropanoate
2-(6-chloro-2-oxo-4-phenyl-chromen-7-yl)oxyacetic acid
4-[[2-(6-chloro-2-oxo-4-phenyl-chromen-7-yl)oxypropanoylamino]methyl]cyclohexanecarboxylic acid
2-(6-chloro-2-oxo-4-phenyl-chromen-7-yl)oxy-N-(2-hydroxy-2-phenyl-ethyl)acetamide
methyl 2-(8-methyl-2-oxo-4-phenyl-chromen-7-yl)oxyacetate
propyl 2-(6-chloro-2-oxo-4-phenyl-chromen-7-yl)oxyacetate
benzyl 2-(2-oxo-4-phenyl-chromen-7-yl)oxypropanoate
tert-butyl 2-(8-methyl-2-oxo-4-phenyl-chromen-7-yl)oxyacetate
2-(6-chloro-2-oxo-4-phenyl-chromen-7-yl)oxy-N-(1,1-dioxo-2,3-dihydrothiophen-3-yl)acetamide
2-[2-(2-oxo-4-phenyl-chromen-7-yl)oxypropanoylamino]-2-phenyl-acetic acid
2-(2-oxo-4-phenyl-6-propyl-chromen-7-yl)oxypropanoic acid
4-[[[2-(6-chloro-2-oxo-4-phenyl-chromen-7-yl)oxyacetyl]amino]methyl]cyclohexanecarboxylic acid
2-(6-chloro-2-oxo-4-phenyl-chromen-7-yl)oxy-N-(2-pyridylmethyl)acetamide,
7-[1-methyl-2-oxo-2-(1-piperidyl)ethoxy]-4-(o-tolyl)chromen-2-one, 4-(2-chlorophenyl)-7-[1-methyl-2-oxo-2-(1-piperidyl)ethoxy]chromen-2-one,
(3S)-1-[2-[4-(o-tolyl)-2-oxo-chromen-7-yl]oxypropanoyl]piperidine-3-carboxylic acid,
2-[4-(o-tolyl)-2-oxo-chromen-7-yl]oxy-N-(2-pyridyl)propanamide,
7-(1-methyl-2-oxo-2-pyrrolidin-1-yl-ethoxy)-4-(o-tolyl)chromen-2-one,
methyl 1-[2-[4-(o-tolyl)-2-oxo-chromen-7-yl]oxypropanoyl]piperidine-3-carboxylate,
methyl 1-[2-[4-(2-chlorophenyl)-2-oxo-chromen-7-yl]oxypropanoyl]piperidine-3-carboxylate,
1-[2-[4-(o-tolyl)-2-oxo-chromen-7-yl]oxypropanoyl]piperidine-3-carboxylic acid,
1-[2-[4-(2-chlorophenyl)-2-oxo-chromen-7-yl]oxypropanoyl]piperidine-3-carboxylic acid,
1-[2-[4-(o-tolyl)-2-oxo-chromen-7-yl]oxypropanoyl]piperidine-3-sulfonic acid,
1-[2-[4-(2-chlorophenyl)-2-oxo-chromen-7-yl]oxypropanoyl]piperidine-3-sulfonic acid,
1-[2-[4-(o-tolyl)-2-oxo-chromen-7-yl]oxypropanoyl]piperidine-3-sulfonamide,
4-(2-chlorophenyl)-7-(1-methyl-2-oxo-2-pyrrolidin-1-yl-ethoxy)chromen-2-one,
(3S)-1-[(2R)-2-[4-(2-chloro-4-fluoro-phenyl)-2-oxo-chromen-7-yl]oxypropanoyl]piperidine-3-carboxylic acid,
1-[2-[4-(2-chlorophenyl)-2-oxo-chromen-7-yl]oxypropanoyl]-N-methyl-piperidine-3-carboxamide,
ethyl (3S)-1-[2-[4-(2-chlorophenyl)-2-oxo-chromen-7-yl]oxypropanoyl]piperidine-3-carboxylate,
ethyl (3S)-1-[2-[4-(o-tolyl)-2-oxo-chromen-7-yl]oxypropanoyl]piperidine-3-carboxylate,
N-methyl-1-[2-[4-(o-tolyl)-2-oxo-chromen-7-yl]oxypropanoyl]piperidine-3-sulfonamide,
N-[4-(2-hydroxyethyl)phenyl]-2-[4-(o-tolyl)-2-oxo-chromen-7-yl]oxy-propanamide,
1-[2-[4-(2-chlorophenyl)-2-oxo-chromen-7-yl]oxypropanoyl]-N-methyl-piperidine-3-sulfonamide,
N-methyl-1-[2-[4-(o-tolyl)-2-oxo-chromen-7-yl]oxypropanoyl]piperidine-3-carboxamide,
(3S)-1-[2-[4-(o-tolyl)-2-oxo-chromen-7-yl]oxypropanoyl]pyrrolidine-3-carboxylic acid,
(3S)-1-[(2R)-2-[4-(2-chlorophenyl)-2-oxo-chromen-7-yl]oxypropanoyl]piperidine-3-carboxylic acid,
1-[2-[4-(2-chlorophenyl)-2-oxo-chromen-7-yl]oxypropanoyl]piperidine-3-sulfonamide,
N-cyclopropyl-2-[4-(o-tolyl)-2-oxo-chromen-7-yl]oxy-propanamide,
2-[4-(2-chlorophenyl)-2-oxo-chromen-7-yl]oxy-N-cyclopropyl-propanamide,
(3S)-1-[(2R)-2-[4-(o-tolyl)-2-oxo-chromen-7-yl]oxypropanoyl]piperidine-3-carboxylic acid,
(3S)-1-[(2R)-2-[4-(2-chlorophenyl)-2-oxo-chromen-7-yl]oxypropanoyl]piperidine-3-carbonitrile,
(3S)-1-[2-[4-(2-chlorophenyl)-2-oxo-chromen-7-yl]oxypropanoyl]pyrrolidine-3-carboxylic acid,
7-[(1R)-1-methyl-2-oxo-2-(1-piperidyl)ethoxy]-4-(o-tolyl)chromen-2-one,
2-[4-(2-chlorophenyl)-2-oxo-chromen-7-yl]oxy-N-(2-pyridyl)propanamide,
(3S)-1-[(2R)-2-[4-(o-tolyl)-2-oxo-chromen-7-yl]oxypropanoyl]piperidine-3-carbonitrile,
(3S)-1-[2-[4-(2-chlorophenyl)-2-oxo-chromen-7-yl]oxypropanoyl]piperidine-3-carboxylic acid,
4-(2-chlorophenyl)-7-[(1R)-1-methyl-2-oxo-2-(1-piperidyl)ethoxy]chromen-2-one,
2-[4-(2-chlorophenyl)-2-oxo-chromen-7-yl]oxy-N-[4-(2-hydroxyethyl)phenyl]propanamide,
(2R)—N-isopropyl-2-[4-(o-tolyl)-2-oxo-chromen-7-yl]oxy-propanamide,
1-[2-[4-(2-chloro-4-fluoro-phenyl)-2-oxo-chromen-7-yl]oxypropanoyl]-3-methyl-piperidine-3-carboxylic acid,
N,N-dimethyl-2-[4-(o-tolyl)-2-oxo-chromen-7-yl]oxy-propanamide, 4-(2-chlorophenyl)-7-[1-methyl-2-oxo-2-[3-(2H-tetrazol-5-yl)-1-piperidyl]ethoxy]chromen-2-one,
ethyl 2-[4-(2-chlorophenyl)-2-oxo-chromen-7-yl]oxypropanoate,
1-[2-[4-(o-tolyl)-2-oxo-chromen-7-yl]oxypropanoyl]piperidine-3-carbonitrile,
7-[1-methyl-2-oxo-2-[3-(2H-tetrazol-5-yl)-1-piperidyl]ethoxy]-4-(o-tolyl)chromen-2-one,
3-methyl-1-[2-[4-(o-tolyl)-2-oxo-chromen-7-yl]oxypropanoyl]piperidine-3-carboxylic acid,
(2R)—N,N-dimethyl-2-[4-(o-tolyl)-2-oxo-chromen-7-yl]oxy-propanamide,
(2R)-2-[4-(2-chlorophenyl)-2-oxo-chromen-7-yl]oxy-N,N-dimethyl-propanamide,
(3R)-1-[2-[4-(2-chlorophenyl)-2-oxo-chromen-7-yl]oxypropanoyl]piperidine-3-carboxylic acid,
(3S)-1-[2-[4-(2-chloro-4-fluoro-phenyl)-2-oxo-chromen-7-yl]oxypropanoyl]piperidine-3-carboxylic acid,
1-[2-[4-(2-chlorophenyl)-2-oxo-chromen-7-yl]oxypropanoyl]piperidine-3-carbonitrile,
2-[4-(2-chloro-3-fluoro-phenyl)-2-oxo-chromen-7-yl]oxy-N-isopropyl-propanamide,
(3S)-1-[2-[4-(2-chloro-3-fluoro-phenyl)-2-oxo-chromen-7-yl]oxypropanoyl]piperidine-3-carboxylic acid,
2-[4-(2-chlorophenyl)-2-oxo-chromen-7-yl]oxy-N-methyl-propanamide,
1-[2-[4-(2-chloro-3-fluoro-phenyl)-2-oxo-chromen-7-yl]oxypropanoyl]piperidine-3-sulfonamide,
(3R)-1-[2-[4-(o-tolyl)-2-oxo-chromen-7-yl]oxypropanoyl]piperidine-3-carboxylic acid,
1-[2-[4-(2-chlorophenyl)-2-oxo-chromen-7-yl]oxypropanoyl]-3-methyl-piperidine-3-carboxylic acid,
isopropyl (2R)-2-[4-(o-tolyl)-2-oxo-chromen-7-yl]oxypropanoate,
(2R)-2-[4-(2-chlorophenyl)-2-oxo-chromen-7-yl]oxy-N-isopropyl-propanamide,
ethyl 2-[4-(2-chloro-3-fluoro-phenyl)-2-oxo-chromen-7-yl]oxypropanoate,
ethyl 2-[4-(o-tolyl)-2-oxo-chromen-7-yl]oxypropanoate,
2-[1-[2-[4-(2-chlorophenyl)-2-oxo-chromen-7-yl]oxypropanoyl]-4-piperidyl]acetic acid,
2-[1-[2-[4-(o-tolyl)-2-oxo-chromen-7-yl]oxypropanoyl]-4-piperidyl]acetic acid,
2-[4-(2-chloro-3-fluoro-phenyl)-2-oxo-chromen-7-yl]oxy-N-cyclopropyl-propanamide,
N-isopropyl-2-[4-(o-tolyl)-2-oxo-chromen-7-yl]oxy-propanamide,
1-[2-[4-(o-tolyl)-2-oxo-chromen-7-yl]oxypropanoyl]pyrrolidine-3-sulfonamide,
isopropyl (2R)-2-[4-(2-chlorophenyl)-2-oxo-chromen-7-yl]oxypropanoate,
2-[4-(2-chloro-4-fluoro-phenyl)-2-oxo-chromen-7-yl]oxy-N-isopropyl-propanamide,
2-[1-[2-[4-(2-chlorophenyl)-2-oxo-chromen-7-yl]oxypropanoyl]-3-piperidyl]acetic acid,
(3R)-1-[2-[4-(2-chlorophenyl)-2-oxo-chromen-7-yl]oxypropanoyl]-N,N-dimethyl-piperidine-3-carboxamide,
2-[4-(2-chloro-3-fluoro-phenyl)-2-oxo-chromen-7-yl]oxy-N-ethyl-propanamide,
1-[2-[4-(2-chloro-4-fluoro-phenyl)-2-oxo-chromen-7-yl]oxypropanoyl]piperidine-3-sulfonamide,
2-[4-(2-chlorophenyl)-2-oxo-chromen-7-yl]oxy-N-isopropyl-propanamide,
7-[2-(4,4-difluoro-1-piperidyl)-1-methyl-2-oxo-ethoxy]-4-(o-tolyl)chromen-2-one,
2-[1-[2-[4-(o-tolyl)-2-oxo-chromen-7-yl]oxypropanoyl]-3-piperidyl]acetic acid,
7-[1-methyl-2-[3-(methylsulfonimidoyl)-1-piperidyl]-2-oxo-ethoxy]-4-(o-tolyl)chromen-2-one,
4-(2-chlorophenyl)-7-[2-(4,4-difluoro-1-piperidyl)-1-methyl-2-oxo-ethoxy]chromen-2-one, ethyl 1-[2-[4-(2-chlorophenyl)-2-oxo-chromen-7-yl]oxypropanoyl]-3-methyl-piperidine-3-carboxylate,
(3S)-1-[2-[4-(2-chlorophenyl)-2-oxo-chromen-7-yl]oxypropanoyl]-N,N-dimethyl-piperidine-3-carboxamide,
1-[2-[4-(2-chlorophenyl)-2-oxo-chromen-7-yl]oxypropanoyl]pyrrolidine-3-sulfonamide,
(3R)—N,N-dimethyl-1-[2-[4-(o-tolyl)-2-oxo-chromen-7-yl]oxypropanoyl]piperidine-3-carboxamide,
4-(2-chlorophenyl)-7-[1-methyl-2-[3-(methylsulfonimidoyl)-1-piperidyl]-2-oxo-ethoxy]chromen-2-one,
methyl 2-[1-[2-[4-(2-chlorophenyl)-2-oxo-chromen-7-yl]oxypropanoyl]-4-piperidyl]acetate,
ethyl 2-[4-(2-bromophenyl)-2-oxo-chromen-7-yl]oxypropanoate,
ethyl 2-[4-(2-chloro-4-fluoro-phenyl)-2-oxo-chromen-7-yl]oxypropanoate,
methyl 2-[1-[2-[4-(o-tolyl)-2-oxo-chromen-7-yl]oxypropanoyl]-4-piperidyl]acetate,
(3S)—N,N-dimethyl-1-[2-[4-(o-tolyl)-2-oxo-chromen-7-yl]oxypropanoyl]piperidine-3-carboxamide,
N-ethyl-2-[4-(o-tolyl)-2-oxo-chromen-7-yl]oxy-propanamide, ethyl 2-[1-[2-[4-(2-chlorophenyl)-2-oxo-chromen-7-yl]oxypropanoyl]-3-piperidyl]acetate,
ethyl 2-[1-[2-[4-(o-tolyl)-2-oxo-chromen-7-yl]oxypropanoyl]-3-piperidyl]acetate,
ethyl 3-methyl-1-[2-[4-(o-tolyl)-2-oxo-chromen-7-yl]oxypropanoyl]piperidine-3-carboxylate,
2-[4-(2-bromophenyl)-2-oxo-chromen-7-yl]oxy-N,N-dimethyl-propanamide,
2-[4-(2-chloro-4-fluoro-phenyl)-2-oxo-chromen-7-yl]oxy-N-cyclopropyl-propanamide,
2-[4-(2-chloro-4-fluoro-phenyl)-2-oxo-chromen-7-yl]oxy-N-ethyl-propanamide,
isopropyl 2-[4-(2-bromophenyl)-2-oxo-chromen-7-yl]oxypropanoate,
2-[4-(2-chlorophenyl)-2-oxo-chromen-7-yl]oxy-N,N-dimethyl-propanamide,
2-[4-(2-chloro-3-fluoro-phenyl)-2-oxo-chromen-7-yl]oxypropanoic acid,
ethyl 1-[2-[4-(2-chloro-4-fluoro-phenyl)-2-oxo-chromen-7-yl]oxypropanoyl]-3-methyl-piperidine-3-carboxylate,
2-[4-(2-chlorophenyl)-2-oxo-chromen-7-yl]oxypropanoic acid,
2-[4-(o-tolyl)-2-oxo-chromen-7-yl]oxypropanoic acid,
2-[4-(2-chlorophenyl)-2-oxo-chromen-7-yl]oxy-N-ethyl-propanamide,
2-[4-(2-chloro-4-fluoro-phenyl)-2-oxo-chromen-7-yl]oxypropanoic acid,
methyl 2-[(3S)-1-[(2R)-2-[4-(2-chlorophenyl)-2-oxo-chromen-7-yl]oxypropanoyl]-3-piperidyl]acetate,
methyl 2-[(3R)-1-[(2R)-2-[4-(2-chlorophenyl)-2-oxo-chromen-7-yl]oxypropanoyl]-3-piperidyl]acetate,
2-[(3S)-1-[(2R)-2-[4-(2-chlorophenyl)-2-oxo-chromen-7-yl]oxypropanoyl]-3-piperidyl]acetic acid,
2-[(3R)-1-[(2R)-2-[4-(2-chlorophenyl)-2-oxo-chromen-7-yl]oxypropanoyl]-3-piperidyl]acetic acid,
methyl 2-[(3R)-1-[(2R)-2-[4-(2-chloro-4-fluoro-phenyl)-2-oxo-chromen-7-yl]oxypropanoyl]-3-piperidyl]acetate,
methyl 2-[(3S)-1-[(2R)-2-[4-(2-chloro-4-fluoro-phenyl)-2-oxo-chromen-7-yl]oxypropanoyl]-3-piperidyl]acetate,
2-[(3R)-1-[(2R)-2-[4-(2-chloro-4-fluoro-phenyl)-2-oxo-chromen-7-yl]oxypropanoyl]-3-piperidyl]acetic acid,
2-[(3S)-1-[(2R)-2-[4-(2-chloro-4-fluoro-phenyl)-2-oxo-chromen-7-yl]oxypropanoyl]-3-piperidyl]acetic acid,
(3R)-1-[(2R)-2-[4-(2-chloro-4-fluoro-phenyl)-2-oxo-chromen-7-yl]oxypropanoyl]piperidine-3-carboxylic acid,
ethyl (3S)-1-[2-[4-(2-chlorophenyl)-2-oxo-chromen-7-yl]oxybutanoyl]piperidine-3-carboxylate,
(3S)-1-[2-[4-(2-chlorophenyl)-2-oxo-chromen-7-yl]oxybutanoyl]piperidine-3-carboxylic acid,
ethyl (3S)-1-[(2R)-2-[4-(2-chloro-4-fluoro-phenyl)-2-oxo-chromen-7-yl]oxypropanoyl]piperidine-3-carboxylate,
2-[4-(2-chlorophenyl)-2-oxo-chromen-7-yl]oxy-2-methyl-propanoic acid,
ethyl 2-[4-(2-chlorophenyl)-2-oxo-chromen-7-yl]oxy-2-methyl-propanoate,
2-[4-(2-chlorophenyl)-2-oxo-chromen-7-yl]oxy-N-isopropyl-2-methyl-propanamide,
N-isopropyl-2-methyl-2-[4-(o-tolyl)-2-oxo-chromen-7-yl]oxy-propanamide,
(3S)-1-[2-[4-(2-chlorophenyl)-2-oxo-chromen-7-yl]oxy-2-methyl-propanoyl]piperidine-3-carboxylic acid,
isopropyl (3S)-1-[(2R)-2-[4-(2-chloro-4-fluoro-phenyl)-2-oxo-chromen-7-yl]oxypropanoyl]piperidine-3-carboxylate,
tert-butyl (3S)-1-[(2R)-2-[4-(2-chloro-4-fluoro-phenyl)-2-oxo-chromen-7-yl]oxypropanoyl]piperidine-3-carboxylate,
2-morpholinoethyl (3S)-1-[(2R)-2-[4-(2-chloro-4-fluoro-phenyl)-2-oxo-chromen-7-yl]oxypropanoyl]piperidine-3-carboxylate,
heptyl (3S)-1-[(2R)-2-[4-(2-chloro-4-fluoro-phenyl)-2-oxo-chromen-7-yl]oxypropanoyl]piperidine-3-carboxylate,
isopropoxycarbonyloxymethyl (3S)-1-[(2R)-2-[4-(2-chloro-4-fluoro-phenyl)-2-oxo-chromen-7-yl]oxypropanoyl]piperidine-3-carboxylate,
(3S)—N-methyl-1-[(2R)-2-[4-(2-chloro-4-fluoro-phenyl)-2-oxo-chromen-7-yl]oxypropanoyl piperidine-3-carboxamide,
isopropyl 2-[(3R)-1-[(2R)-2-[4-(2-chloro-4-fluoro-phenyl)-2-oxo-chromen-7-yl]oxypropanoyl]-3-piperidyl]acetate,
tert-butyl 2-[(3R)-1-[(2R)-2-[4-(2-chloro-4-fluoro-phenyl)-2-oxo-chromen-7-yl]oxypropanoyl]-3-piperidyl]acetate,
2-morpholinoethyl 2-[(3R)-1-[(2R)-2-[4-(2-chloro-4-fluoro-phenyl)-2-oxo-chromen-7-yl]oxypropanoyl]-3-piperidyl]acetate,
heptyl 2-[(3R)-1-[(2R)-2-[4-(2-chloro-4-fluoro-phenyl)-2-oxo-chromen-7-yl]oxypropanoyl]-3-piperidyl]acetate,
isopropoxycarbonyloxymethyl 2-[(3R)-1-[(2R)-2-[4-(2-chloro-4-fluoro-phenyl)-2-oxo-chromen-7-yl]oxypropanoyl]-3-piperidyl]acetate, and
2-[(3R)-1-[(2R)-2-[4-(2-chloro-4-fluoro-phenyl)-2-oxo-chromen-7-yl]oxypropanoyl]-3-piperidyl]-N-methyl-acetamide,
(3S)-1-[(2R)-2-[[4-(o-tolyl)-7-quinolyl]oxy]propanoyl]piperidine-3-carboxylic acid,
(3S)-1-[(2R)-2-[[4-(2,6-dimethylphenyl)-7-quinolyl]oxy]propanoyl]piperidine-3-carboxylic acid,
2-[(3R)-1-[(2R)-2-[[4-(2,6-dimethylphenyl)-7-quinolyl]oxy]propanoyl]-3-piperidyl]acetic acid,
ethyl (3S)-1-[(2R)-2-[[4-(2,6-dimethylphenyl)-7-quinolyl]oxy]propanoyl]piperidine-3-carboxylate,
2-[(3S)-1-[(2R)-2-[[4-(2,6-dimethylphenyl)-7-quinolyl]oxy]propanoyl]-3-piperidyl]acetic acid,
ethyl 2-[(3R)-1-[(2R)-2-[[4-(2,6-dimethylphenyl)-7-quinolyl]oxy]propanoyl]-3-piperidyl]acetate,
(3R)-1-[(2R)-2-[[4-(2-chlorophenyl)-7-quinolyl]oxy]propanoyl]piperidine-3-carboxylic acid,
ethyl 2-[(3R)-1-[(2R)-2-[[4-(o-tolyl)-7-quinolyl]oxy]propanoyl]-3-piperidyl]acetate,
(3S)-1-[(2R)-2-[[4-(2-chlorophenyl)-7-quinolyl]oxy]propanoyl]piperidine-3-carboxylic acid,
(3S)-1-[(2R)-2-[[4-(2-chloro-4-fluoro-phenyl)-7-quinolyl]oxy]propanoyl]piperidine-3-carboxylic acid,
(2R)-2-[[4-(2-chloro-4-fluoro-phenyl)-7-quinolyl]oxy]-1-[(3S)-3-(2H-tetrazol-5-yl)-1-piperidyl]propan-1-one,
(3S)-1-[(2R)-2-[[4-(2-chlorophenyl)-2-fluoro-7-quinolyl]oxy]propanoyl]piperidine-3-carboxylic acid,
2-[(3R)-1-[(2R)-2-[[4-(o-tolyl)-7-quinolyl]oxy]propanoyl]-3-piperidyl]acetic acid,
ethyl (3S)-1-[(2R)-2-[[4-(o-tolyl)-7-quinolyl]oxy]propanoyl]piperidine-3-carboxylate,
ethyl 2-[(3R)-1-[(2R)-2-[[4-(2-chloro-4-fluoro-phenyl)-7-quinolyl]oxy]propanoyl]-3-piperidyl]acetate,
2-[(3R)-1-[(2R)-2-[[4-(2-chlorophenyl)-7-quinolyl]oxy]propanoyl]-3-piperidyl]acetic acid
(3R)-1-[(2R)-2-[[4-(2-chloro-4-fluoro-phenyl)-7-quinolyl]oxy]propanoyl]piperidine-3-carboxylic acid,
2-[(3R)-1-[(2R)-2-[[4-(2-chloro-4-fluoro-phenyl)-7-quinolyl]oxy]propanoyl]-3-piperidyl]acetic acid,
(3S)-1-[(2R)-2-[[4-(2-chloro-4-fluoro-phenyl)-7-quinolyl]oxy]propanoyl]pyrrolidine-3-carboxylic acid,
(2R)-2-[[4-(2-chloro-4-fluoro-phenyl)-7-quinolyl]oxy]-1-(1-piperidyl)propan-1-one,
2-[(3R)-1-[(2R)-2-[[2-chloro-4-(o-tolyl)-7-quinolyl]oxy]propanoyl]-3-piperidyl]acetic acid,
2-[(3S)-1-[(2R)-2-[[4-(2-chloro-4-fluoro-phenyl)-7-quinolyl]oxy]propanoyl]-3-piperidyl]acetic acid,
rac-(3S)-1-[2-[[4-(2-chlorophenyl)-7-quinolyl]oxy]propanoyl]piperidine-3-carboxylic acid,
1-[(2R)-2-[[4-(2-chloro-4-fluoro-phenyl)-7-quinolyl]oxy]propanoyl]piperidine-4-carboxylic acid,
(3S)-1-[rac-(2R)-2-[[2-chloro-4-(o-tolyl)-7-quinolyl]oxy]propanoyl]piperidine-3-carboxylic acid,
3-[[(2R)-2-[[4-(2-chloro-4-fluoro-phenyl)-7-quinolyl]oxy]propanoyl]amino]benzoic acid
ethyl (3S)-1-[(2R)-2-[[4-(2-chloro-4-fluoro-phenyl)-7-quinolyl]oxy]propanoyl]piperidine-3-carboxylate,
(2R)-2-[[4-(2-chloro-4-fluoro-phenyl)-7-quinolyl]oxy]-1-(4-propanoylpiperazin-1-yl)propan-1-one,
tert-butyl (2R)-2-[[4-(2-chlorophenyl)-7-quinolyl]oxy]propanoate,
(3S)-1-[(2R)-2-[[4-(2-chloro-4-fluoro-phenyl)-7-quinolyl]oxy]propanoyl]piperidine-3-carbonitrile,
(3S)-1-[(2R)-2-[[2-methyl-4-(o-tolyl)-7-quinolyl]oxy]propanoyl]piperidine-3-carboxylic acid,
(2R)-2-[[4-(2-chloro-4-fluoro-phenyl)-7-quinolyl]oxy]-N-isopropyl-N-methyl-propanamide,
1-[rac-(2R)-2-[[4-(2-chloro-4-fluoro-phenyl)-7-quinolyl]oxy]propanoyl]piperidine-3-sulfonamide,
(3S)-1-[(2R)-2-[[2-chloro-4-(2-chlorophenyl)-7-quinolyl]oxy]propanoyl]piperidine-3-carboxylic acid,
(3S)-1-[(2R)-2-[[2-chloro-4-(4-fluoro-2-methyl-phenyl)-7-quinolyl]oxy]propanoyl]piperidine-3-carboxylic acid,
isopropyl (2R)-2-[[4-(2-chlorophenyl)-7-quinolyl]oxy]propanoate,
methyl 2-[(3R)-1-[(2R)-2-[[4-(2-chloro-4-fluoro-phenyl)-7-quinolyl]oxy]propanoyl]pyrrolidin-3-yl]acetate,
(3S)-1-[(2R)-2-[[4-(2-chloro-4-fluoro-phenyl)-7-quinolyl]oxy]propanoyl]-N-methyl-piperidine-3-carboxamide,
2-[(3S)-1-[(2R)-2-[[4-(2-chloro-4-fluoro-phenyl)-7-quinolyl]oxy]propanoyl]pyrrolidin-3-yl]acetic acid,
2-[(3R)-1-[(2R)-2-[[4-(2-chloro-4-fluoro-phenyl)-7-quinolyl]oxy]propanoyl]pyrrolidin-3-yl]acetic acid,
ethyl (3S)-1-[(2R)-2-[[4-(2-chlorophenyl)-2-fluoro-7-quinolyl]oxy]propanoyl]piperidine-3-carboxylate,
(2R)-2-[[4-(2-chloro-4-fluoro-phenyl)-7-quinolyl]oxy]-1-pyrrolidin-1-yl-propan-1-one,
(2R)-2-[[2-chloro-4-(o-tolyl)-7-quinolyl]oxy]propanoic acid,
(2R)-2-[[4-(2-chloro-4-fluoro-phenyl)-7-quinolyl]oxy]propanoic acid,
(2R)-2-[[4-(2-chloro-4-fluoro-phenyl)-7-quinolyl]oxy]-N,N-dimethyl-propanamide,
rac-(3S)-1-[2-[[4-(2-chlorophenyl)-7-quinolyl]oxy]acetyl]piperidine-3-carboxylic acid,
(2R)—N-tert-butyl-2-[[4-(2-chloro-4-fluoro-phenyl)-7-quinolyl]oxy]propanamide,
(2R)-2-[[4-(2-chlorophenyl)-7-quinolyl]oxy]-N-isopropyl-propanamide,
   ethyl 2-[(3R)-1-[(2R)-2-[[2-methyl-4-(o-tolyl)-7-quinolyl]oxy]propanoyl]-3-piperidyl]acetate,
ethyl (2R)-2-[[4-(2-chlorophenyl)-7-quinolyl]oxy]propanoate,
ethyl 4-[[(2R)-2-[[4-(2-chloro-4-fluoro-phenyl)-7-quinolyl]oxy]propanoyl]amino]benzoate,
(3S)-1-[(2R)-2-[[2-chloro-4-(o-tolyl)-7-quinolyl]oxy]propanoyl]piperidine-3-carboxamide,
(2R)-2-[[4-(2-chlorophenyl)-7-quinolyl]oxy]-1-(1-piperidyl)propan-1-one,
methyl 3-[[(2R)-2-[[4-(2-chloro-4-fluoro-phenyl)-7-quinolyl]oxy]propanoyl]amino]cyclobutanecarboxylate,
(2R)-2-[[4-(2-chloro-4-fluoro-phenyl)-7-quinolyl]oxy]-1-piperazin-1-yl-propan-1-one,
2-[(3R)-1-[(2R)-2-[[2-chloro-4-(2-chloro-4-fluoro-phenyl)-7-quinolyl]oxy]propanoyl]-3-piperidyl]acetic acid,
2-[(3R)-1-[(2R)-2-[[2-chloro-4-(4-fluoro-2-methyl-phenyl)-7-quinolyl]oxy]propanoyl]-3-piperidyl]acetic acid,
ethyl 1-[(2R)-2-[[4-(2-chloro-4-fluoro-phenyl)-7-quinolyl]oxy]propanoyl]piperidine-4-carboxylate,
(3S)-1-[(2R)-2-[[4-(2-chloro-4-fluoro-phenyl)-2-methyl-7-quinolyl]oxy]propanoyl]piperidine-3-carboxylic acid,
4-[[(2R)-2-[[4-(2-chloro-4-fluoro-phenyl)-7-quinolyl]oxy]propanoyl]amino]benzoic acid,
(2R)-2-[[4-(2-chloro-4-fluoro-phenyl)-7-quinolyl]oxy]-N-isopropyl-propanamide,
(3S)-1-[2-[[5-(2-chloro-4-fluoro-phenyl)-1,8-naphthyridin-2-yl]oxy]propanoyl]piperidine-3-carboxylic acid,
4-[(2R)-2-[[4-(2-chloro-4-fluoro-phenyl)-7-quinolyl]oxy]propanoyl]piperazin-2-one,
(3S)-1-[(2R)-2-[[2-chloro-4-(2-chloro-4-fluoro-phenyl)-7-quinolyl]oxy]propanoyl]piperidine-3-carboxylic acid,
2-[(3R)-1-[(2R)-2-[[4-(2-chloro-3-fluoro-phenyl)-7-quinolyl]oxy]propanoyl]-3-piperidyl]acetic acid,
(3S)-1-[(2R)-2-[(4-phenyl-7-quinolyl)oxy]propanoyl]piperidine-3-carboxylic acid,
methyl 2-[(3S)-1-[(2R)-2-[[4-(2-chloro-4-fluoro-phenyl)-7-quinolyl]oxy]propanoyl]pyrrolidin-3-yl]acetate,
2-[(3R)-1-[(2R)-2-[[2-chloro-4-(2-chlorophenyl)-7-quinolyl]oxy]propanoyl]-3-piperidyl]-N-methyl-acetamide,
2-[(3R)-1-[(2R)-2-[[2-methyl-4-(o-tolyl)-7-quinolyl]oxy]propanoyl]-3-piperidyl]acetic acid,
(2R)-2-[[2-chloro-4-(2-chlorophenyl)-7-quinolyl]oxy]-1-[4-(cyclopropanecarbonyl)piperazin-1-yl]propan-1-one,
methyl (3R)-1-[(2R)-2-[[4-(2-chloro-4-fluoro-phenyl)-7-quinolyl]oxy]propanoyl]piperidine-3-carboxylate,
(2R)-2-[[2-chloro-4-(2-chlorophenyl)-7-quinolyl]oxy]-1-(4-propanoylpiperazin-1-yl)propan-1-one,
tert-butyl (2R)-2-[[2-chloro-4-(o-tolyl)-7-quinolyl]oxy]propanoate,
ethyl 2-[(3R)-1-[(2R)-2-[[4-(2-chloro-4-fluoro-phenyl)-2-methyl-7-quinolyl]oxy]propanoyl]-3-piperidyl]acetate,
ethyl (3S)-1-[(2R)-2-[[4-(2-chloro-4-fluoro-phenyl)-7-quinolyl]oxy]propanoyl]pyrrolidine-3-carboxylate,
(3S)-1-[(2R)-2-[[2-chloro-4-(2-chlorophenyl)-7-quinolyl]oxy]propanoyl]piperidine-3-carboxamide,
(2R)-2-[[4-(2-chloro-4-fluoro-phenyl)-7-quinolyl]oxy]-N-cyclopropyl-propanamide,
(3S)-1-[(2S)-2-[[4-(2-chlorophenyl)-7-quinolyl]oxy]propanoyl]piperidine-3-carboxylic acid,
2-[(3R)-1-[(2R)-2-[[4-(2-methyl-3-thienyl)-7-quinolyl]oxy]propanoyl]-3-piperidyl]acetic acid,
2-[rac-(3R)-1-[2-[[5-(2-chloro-4-fluoro-phenyl)-1,8-naphthyridin-2-yl]oxy]propanoyl]-3-piperidyl]acetic acid,
(3S)-1-[(2R)-2-[[2-chloro-4-(2-chloro-4-fluoro-phenyl)-7-quinolyl]oxy]propanoyl]piperidine-3-carboxamide,
isopropyl 2-[[4-(2-chlorophenyl)-7-quinolyl]oxy]acetate,
(3S)-1-[(2R)-2-[[4-(4-fluoro-2,6-dimethyl-phenyl)-7-quinolyl]oxy]propanoyl]piperidine-3-carboxylic acid,
(2R)-1-[4-(2-aminoacetyl)piperazin-1-yl]-2-[[2-chloro-4-(2-chlorophenyl)-7-quinolyl]oxy]propan-1-one,
2-[[4-(2-chlorophenyl)-7-quinolyl]oxy]-N,N-dimethyl-propanamide,
ethyl (3S)-1-[2-[[4-(2-chlorophenyl)-7-quinolyl]oxy]acetyl]piperidine-3-carboxylate,
(2R)-2-[[4-(2-chlorophenyl)-7-quinolyl]oxy]-N,N-dimethyl-propanamide,
ethyl 2-[(3S)-1-[(2R)-2-[[4-(2-chloro-4-fluoro-phenyl)-7-quinolyl]oxy]propanoyl]-3-piperidyl]acetate,
(2R)-2-[[4-(2-chlorophenyl)-2-fluoro-7-quinolyl]oxy]propanoic acid,
(2R)-2-[[2-chloro-4-(2-chloro-4-fluoro-phenyl)-7-quinolyl]oxy]propanoic acid,
ethyl 2-[(3R)-1-[(2R)-2-[[2-chloro-4-(2-chlorophenyl)-7-quinolyl]oxy]propanoyl]-3-piperidyl]acetate,
ethyl 2-[(3R)-1-[(2R)-2-[[2-chloro-4-(2-chloro-4-fluoro-phenyl)-7-quinolyl]oxy]propanoyl]-3-piperidyl]acetate,
ethyl (3S)-1-[(2R)-2-[[2-chloro-4-(4-fluoro-2-methyl-phenyl)-7-quinolyl]oxy]propanoyl]piperidine-3-carboxylate,
(2R)-2-[[4-(2-chloro-4-fluoro-phenyl)-7-quinolyl]oxy]-N-(4-pyridyl)propanamide,
(3S)-1-[(2R)-2-[[2-chloro-4-(2-chlorophenyl)-7-quinolyl]oxy]propanoyl]-N-methyl-piperidine-3-carboxamide,
methyl 3-[[(2R)-2-[[2-chloro-4-(2-chlorophenyl)-7-quinolyl]oxy]propanoyl]amino]cyclobutanecarboxylate,
2-[(3R)-1-[(2R)-2-[[2-chloro-4-(2-chloro-4-fluoro-phenyl)-7-quinolyl]oxy]propanoyl]-3-piperidyl]-N-methyl-acetamide,
(2R)-2-[[2-chloro-4-(2-chlorophenyl)-7-quinolyl]oxy]-1-(1-piperidyl)propan-1-one,
(3S)-1-[(2R)-2-[[2-chloro-4-(2-chlorophenyl)-7-quinolyl]oxy]propanoyl]-N,N-dimethyl-piperidine-3-carboxamide,
2-[(3R)-1-[(2R)-2-[[4-(2-chloro-4-fluoro-phenyl)-2-methyl-7-quinolyl]oxy]propanoyl]-3-piperidyl]acetic acid,
(3R)-1-[(2S)-2-[[4-(2-chlorophenyl)-7-quinolyl]oxy]propanoyl]piperidine-3-carboxylic acid,
(2R)-2-[[4-(2-chlorophenyl)-7-quinolyl]oxy]propanoic acid,
(2R)-2-[[2-chloro-4-(2-chlorophenyl)-7-quinolyl]oxy]-1-piperazin-1-yl-propan-1-one,
(3S)—N-methyl-1-[(2R)-2-[[2-chloro-4-(2-chloro-4-fluoro-phenyl)-7-quinolyl]oxy]propanoyl]piperidine-3-carboxamide,
2-[[4-(o-tolyl)-7-quinolyl]oxy]acetamide,
(2R)-2-[[2-chloro-4-(2-chloro-4-fluoro-phenyl)-7-quinolyl]oxy]-1-(1-piperidyl)propan-1-one,
ethyl 3-[[(2R)-2-[[4-(2-chloro-4-fluoro-phenyl)-7-quinolyl]oxy]propanoyl]amino]benzoate,
2-[[5-(2-chloro-4-fluoro-phenyl)-1,8-naphthyridin-2-yl]oxy]-N-isopropyl-propanamide,
2-[(3R)-1-[(2R)-2-[[4-(2-fluorophenyl)-7-quinolyl]oxy]propanoyl]-3-piperidyl]acetic acid,
tert-butyl (3S)-1-[(2R)-2-[[2-methyl-4-(o-tolyl)-7-quinolyl]oxy]propanoyl]piperidine-3-carboxylate,
2-[1-[(2R)-2-[[4-(2-chlorophenyl)-7-quinolyl]oxy]propanoyl]-4-piperidyl]acetic acid,
(3S)-1-[rac-(2R)-2-[[4-(2,6-dimethylphenyl)-2-methyl-7-quinolyl]oxy]propanoyl]piperidine-3-sulfonamide,
(3R)-1-[rac-(2R)-2-[[4-(2,6-dimethylphenyl)-2-methyl-7-quinolyl]oxy]propanoyl]piperidine-3-sulfonamide,
(3R)-1-[rac-(2R)-2-[[4-(2,6-dimethylphenyl)-2-methyl-7-quinolyl]oxy]propanoyl]piperidine-3-sulfonamide,
(3R)-1-[(2R)-2-[[4-(2-chloro-4-fluoro-phenyl)-2-methyl-7-quinolyl]oxy]propanoyl]piperidine-3-sulfonamide,
(3S)-1-[(2R)-2-[[4-(2-chloro-4-fluoro-phenyl)-7-quinolyl]oxy]propanoyl]piperidine-3-sulfonamide,
(3R)-1-[(2R)-2-[[4-(2-chloro-4-fluoro-phenyl)-7-quinolyl]oxy]propanoyl]piperidine-3-sulfonamide,
3-[1-[(2R)-2-[[4-(2-chlorophenyl)-7-quinolyl]oxy]propanoyl]-4-piperidyl]propanoic acid,
1-[rac-(2R)-2-[[4-(2,6-dimethylphenyl)-7-quinolyl]oxy]propanoyl]piperidine-3-sulfonamide,
2-[(3R)-1-[(2R)-2-[[4-(2,6-dichlorophenyl)-7-quinolyl]oxy]propanoyl]-3-piperidyl]acetic acid,
2-[(3R)-1-[(2R)-2-[[4-(2-ethylphenyl)-7-quinolyl]oxy]propanoyl]-3-piperidyl]acetic acid,
2-[(3R)-1-[(2R)-2-[[4-(2-isopropylphenyl)-7-quinolyl]oxy]propanoyl]-3-piperidyl]acetic acid,
[1-[rac-(2R)-2-[[4-(2-chloro-4-fluoro-phenyl)-7-quinolyl]oxy]propanoyl]-3-piperidyl]methanesulfonamide,
2-[(3R)-1-[(2R)-2-[[4-(2,6-dimethylphenyl)-2-methyl-7-quinolyl]oxy]propanoyl]-3-piperidyl]acetic acid,
(2R)-2-[[4-(2-chloro-4-fluoro-phenyl)-7-quinolyl]oxy]-N-[4-(2-hydroxyethyl)phenyl]propanamide,
(3S)-1-[(2R)-2-[[5-(2-chloro-4-fluoro-phenyl)-1,8-naphthyridin-2-yl]oxy]propanoyl]piperidine-3-carboxylic acid,
(3S)-1-[(2S)-2-[[5-(2-chloro-4-fluoro-phenyl)-1,8-naphthyridin-2-yl]oxy]propanoyl]piperidine-3-carboxylic acid,
(3S)-1-[(2S)-2-[[5-(2-chloro-4-fluoro-phenyl)-1,8-naphthyridin-2-yl]oxy]propanoyl]piperidine-3-carboxylic acid,
2-[(3R)-1-[(2R)-2-[[5-(2-chloro-4-fluoro-phenyl)-1,8-naphthyridin-2-yl]oxy]propanoyl]-3-piperidyl]acetic acid,
2-[(3R)-1-[(2R)-2-[[5-(2-chloro-4-fluoro-phenyl)-1,8-naphthyridin-2-yl]oxy]propanoyl]-3-piperidyl]acetic acid,
(3S)-1-[(2R)-2-[[4-(2,6-dichlorophenyl)-7-quinolyl]oxy]propanoyl]piperidine-3-carboxylic acid,
(2R)-2-[[4-(2-chloro-4-fluoro-phenyl)-7-quinolyl]oxy]-N-(2-pyridyl)propanamide,
(2R)-2-[[4-(2-chloro-4-fluoro-phenyl)-7-quinolyl]oxy]-N-ethyl-propanamide,
(3S)-1-[(2R)-2-[[4-(2,6-dichloro-4-fluoro-phenyl)-7-quinolyl]oxy]propanoyl]piperidine-3-carboxylic acid,
2-[(3R)-1-[(2R)-2-[[4-(4-methyl-3-thienyl)-7-quinolyl]oxy]propanoyl]-3-piperidyl]acetic acid,
2-[(3R)-1-[(2R)-2-[[4-(3-methyl-2-thienyl)-7-quinolyl]oxy]propanoyl]-3-piperidyl]acetic acid,
2-[(3R)-1-[(2R)-2-[[4-(2-methoxyphenyl)-7-quinolyl]oxy]propanoyl]-3-piperidyl]acetic acid,
2-[(3R)-1-[(2R)-2-[[4-[2-(trifluoromethyl)phenyl]-7-quinolyl]oxy]propanoyl]-3-piperidyl]acetic acid,
2-[(3R)-1-[(2R)-2-[[4-[2-(trifluoromethoxy)phenyl]-7-quinolyl]oxy]propanoyl]-3-piperidyl]acetic acid,
(3S)-1-[(2R)-2-[[4-(2,6-dimethylphenyl)-7-quinolyl]oxy]propanoyl]piperidine-3-sulfonamide,
2-[(3R)-1-[2-[[4-(2-chlorophenyl)-7-quinolyl]oxy]-2-methyl-propanoyl]-3-piperidyl]acetic acid,
2-[rac-(3R)-1-[2-[[4-(2-chlorophenyl)-7-quinolyl]oxy]butanoyl]-3-piperidyl]acetic acid,
2-[(3R)-1-[(2R)-2-[[4-(2-chloro-6-methyl-phenyl)-7-quinolyl]oxy]propanoyl]-3-piperidyl]acetic acid,
2-[(3R)-1-[(2R)-2-[[4-(2-bromophenyl)-7-quinolyl]oxy]propanoyl]-3-piperidyl]acetic acid,
2-[(3R)-1-[(2R)-2-[[4-(2-cyanophenyl)-7-quinolyl]oxy]propanoyl]-3-piperidyl]acetic acid,
2-[(3R)-1-[(2R)-2-[[4-(2-ethynylphenyl)-7-quinolyl]oxy]propanoyl]-3-piperidyl]acetic acid,
2-[(3R)-1-[(2R)-2-[[4-[2-(dimethylamino)phenyl]-7-quinolyl]oxy]propanoyl]-3-piperidyl]acetic acid,
2-[(3R)-1-[(2R)-2-[[4-(2-carbamoylphenyl)-7-quinolyl]oxy]propanoyl]-3-piperidyl]acetic acid,
2-[(3R)-1-[(2R)-2-[[4-(2,6-difluorophenyl)-7-quinolyl]oxy]propanoyl]-3-piperidyl]acetic acid,
2-[(3R)-1-[(2R)-2-[[4-(2,4-dimethyl-3-thienyl)-7-quinolyl]oxy]propanoyl]-3-piperidyl]acetic acid,
2-[(3R)-1-[(2R)-2-[[4-[2-chloro-6-(trifluoromethyl)phenyl]-7-quinolyl]oxy]propanoyl]-3-piperidyl]acetic acid,
2-[(3R)-1-[(2R)-2-[[4-(2-bromo-6-chloro-phenyl)-7-quinolyl]oxy]propanoyl]-3-piperidyl]acetic acid,
2-[(3R)-1-[(2R)-2-[[4-[2,6-bis(trifluoromethyl)phenyl]-7-quinolyl]oxy]propanoyl]-3-piperidyl]acetic acid,
2-[(3R)-1-[(2R)-2-[[4-(2-chloro-6-methoxy-phenyl)-7-quinolyl]oxy]propanoyl]-3-piperidyl]acetic acid,
2-[(3R)-1-[(2R)-2-[[4-(2,6-diisopropylphenyl)-7-quinolyl]oxy]propanoyl]-3-piperidyl]acetic acid,
(2R)-1-[(3R)-3-amino-1-piperidyl]-2-[[4-(2,6-dimethylphenyl)-7-quinolyl]oxy]propan-1-one,
(2R)-1-[(3S)-3-amino-1-piperidyl]-2-[[4-(2,6-dimethylphenyl)-7-quinolyl]oxy]propan-1-one,
N-[(3S)-1-[(2R)-2-[[4-(2,6-dimethylphenyl)-7-quinolyl]oxy]propanoyl]-3-piperidyl]propanamide,
N-tert-butyl-4-[(2R)-2-[[4-(2,6-dimethylphenyl)-7-quinolyl]oxy]propanoyl]piperazine-1-carboxamide,
(2R)-2-[[4-(2,6-dimethylphenyl)-7-quinolyl]oxy]-1-[3-(1-hydroxycyclopropyl)-1-piperidyl]propan-1-one,
8-[(2R)-2-[[4-(2,6-dimethylphenyl)-7-quinolyl]oxy]propanoyl]-2,8-diazaspiro[4.5]decan-1-one,
(2R)-2-[[4-(2,6-dimethylphenyl)-7-quinolyl]oxy]-1-(3,5-dimethylpiperazin-1-yl)propan-1-one,
N-[(3S)-1-[(2R)-2-[[4-(2,6-dimethylphenyl)-7-quinolyl]oxy]propanoyl]-3-piperidyl]-N-hydroxy-acetamide,
1-[4-[(2R)-2-[[4-(2,6-dimethylphenyl)-7-quinolyl]oxy]propanoyl]piperazin-1-yl]-2,2-dimethyl-propan-1-one,
N-[(3S)-1-[(2R)-2-[[4-(2,6-dimethylphenyl)-7-quinolyl]oxy]propanoyl]-3-piperidyl]methanesulfonamide,
N-[(3S)-1-[(2R)-2-[[4-(2,6-dimethylphenyl)-7-quinolyl]oxy]propanoyl]-3-piperidyl]benzamide,
(3S)—N-cyano-1-[(2R)-2-[[4-(2,6-dimethylphenyl)-7-quinolyl]oxy]propanoyl]piperidine-3-carboxamide,
(3S)-1-[(2R)-2-[[4-(2,6-dimethylphenyl)-7-quinolyl]oxy]propanoyl]piperidine-3-carbohydroxamic acid,
2-[(3R)-1-[(2R)-2-[[4-(2,6-dimethylphenyl)-7-quinolyl]oxy]propanoyl]-3-piperidyl]ethanehydroxamic acid,
(2R)-1-(3-aminoazetidin-1-yl)-2-[[4-(2,6-dimethylphenyl)-7-quinolyl]oxy]propan-1-one,
(2R)-2-[[4-(2,6-dimethylphenyl)-7-quinolyl]oxy]-1-[3-(1H-tetrazol-5-yl)azetidin-1-yl]propan-1-one,
3-hydroxy-1-[(2R)-2-[[4-(2,6-dimethylphenyl)-7-quinolyl]oxy]propanoyl]piperidine-3-carboxylic acid,
5-[(2R)-2-[[4-(2,6-dimethylphenyl)-7-quinolyl]oxy]propanoyl]-5-azaspiro[2.5]octane-2-carboxylic acid,
(3R)-1-[(2R)-2-[[4-(2,6-dimethylphenyl)-7-quinolyl]oxy]propanoyl]piperidine-3-sulfonamide,
(3S)-1-[(2R)-2-[[4-(2,6-dichloro-4-fluoro-phenyl)-7-quinolyl]oxy]propanoyl]-3-methyl-piperidine-3-carboxylic acid,
5-[rac-(2R)-2-[[4-(4-fluoro-2,6-dimethyl-phenyl)-7-quinolyl]oxy]propanoyl]-5-azaspiro[2.5]octane-2-carboxylic acid,
(3R)-1-[(2R)-2-[[4-(4-fluoro-2,6-dimethyl-phenyl)-7-quinolyl]oxy]propanoyl]-3-methyl-piperidine-3-carboxylic acid,
(2R)-2-[[4-(4-fluoro-2,6-dimethyl-phenyl)-7-quinolyl]oxy]-1-[(2S)-2-methyl-1-piperidyl]propan-1-one,
5-[rac-(2R)-2-[[4-(2,6-dichloro-4-fluoro-phenyl)-7-quinolyl]oxy]propanoyl]-5-azaspiro[2.5]octane-2-carboxylic acid,
(2R)-2-[[4-(4-fluoro-2,6-dimethyl-phenyl)-7-quinolyl]oxy]-1-morpholino-propan-1-one,
(2R)-2-[[4-(4-fluoro-2,6-dimethyl-phenyl)-7-quinolyl]oxy]-1-(2-oxa-7-azaspiro[3.5]nonan-7-yl)propan-1-one,
rac-(2R)-2-[[4-(4-fluoro-2,6-dimethyl-phenyl)-7-quinolyl]oxy]-1-[3-(1-hydroxycyclopropyl)-1-piperidyl]propan-1-one,
8-[(2R)-2-[[4-(4-fluoro-2,6-dimethyl-phenyl)-7-quinolyl]oxy]propanoyl]-2,8-diazaspiro[4.5]decan-1-one,
1-[1-[rac-(2R)-2-[[4-(4-fluoro-2,6-dimethyl-phenyl)-7-quinolyl]oxy]propanoyl]-3-piperidyl]cyclopropanecarboxylic acid,
2-[(3R)-1-[(2R)-2-[[4-(4-fluoro-2,6-dimethyl-phenyl)-7-quinolyl]oxy]propanoyl]-3-piperidyl]acetic acid,
(3S)-1-[(2R)-2-[[4-(4-fluoro-2,6-dimethyl-phenyl)-7-quinolyl]oxy]propanoyl]-3-methyl-piperidine-3-carboxylic acid,
N-[(3S)-1-[(2R)-2-[[4-(4-fluoro-2,6-dimethyl-phenyl)-7-quinolyl]oxy]propanoyl]-3-piperidyl]-N-hydroxy-acetamide,
2-methyl-2-[1-[rac-(2R)-2-[[4-(4-fluoro-2,6-dimethyl-phenyl)-7-quinolyl]oxy]propanoyl]-3-piperidyl]propanoic acid,
(2R)-2-[[4-(4-fluoro-2,6-dimethyl-phenyl)-7-quinolyl]oxy]-1-[(2R)-2-methyl-1-piperidyl]propan-1-one,
(3R)-1-[(2R)-2-[[4-(2,6-dichloro-4-fluoro-phenyl)-7-quinolyl]oxy]propanoyl]-3-methyl-piperidine-3-carboxylic acid,
(3S)-1-[(2R)-2-[[4-(2-chloro-4-fluoro-phenyl)-7-quinolyl]oxy]propanoyl]-3-methyl-piperidine-3-carboxylic acid,
[1-[rac-(2R)-2-[[4-(2,6-dimethylphenyl)-7-quinolyl]oxy]propanoyl]-3-piperidyl]methanesulfonamide,
rac-(2R)-1-(2,6-dimethyl-1-piperidyl)-2-[[4-(4-fluoro-2,6-dimethyl-phenyl)-7-quinolyl]oxy]propan-1-one,
2-[(3R)-1-[(2R)-2-[[4-(2,6-dichloro-4-fluoro-phenyl)-7-quinolyl]oxy]propanoyl]-3-piperidyl]acetic acid,
5-[rac-(2R)-2-[[4-(2-chloro-4-fluoro-phenyl)-7-quinolyl]oxy]propanoyl]-5-azaspiro[2.5]octane-2-carboxylic acid,
1-[1-[rac-(2R)-2-[[4-(2-chloro-4-fluoro-phenyl)-7-quinolyl]oxy]propanoyl]-3-piperidyl]cyclopropanecarboxylic acid,
2-methyl-2-[1-[rac-(2R)-2-[[4-(2,6-dichloro-4-fluoro-phenyl)-7-quinolyl]oxy]propanoyl]-3-piperidyl]propanoic acid,
1-[1-[rac-(2R)-2-[[4-(2,6-dichloro-4-fluoro-phenyl)-7-quinolyl]oxy]propanoyl]-3-piperidyl]cyclopropanecarboxylic acid,
2-methyl-2-[1-[rac-(2R)-2-[[4-(2-chloro-4-fluoro-phenyl)-7-quinolyl]oxy]propanoyl]-3-piperidyl]propanoic acid,
(3R)-1-[(2R)-2-[[4-(2-chloro-4-fluoro-phenyl)-7-quinolyl]oxy]propanoyl]-3-methyl-piperidine-3-carboxylic acid,
(2R)-2-[[4-(2,6-dichloro-4-fluoro-phenyl)-7-quinolyl]oxy]-1-[(2S)-2-methyl-1-piperidyl]propan-1-one,
rac-(2R)-2-[[4-(2-chloro-4-fluoro-phenyl)-7-quinolyl]oxy]-1-(2-methyl-1-piperidyl)propan-1-one,
(2R)-2-[[4-(2,6-dichloro-4-fluoro-phenyl)-7-quinolyl]oxy]-1-morpholino-propan-1-one,
8-[(2R)-2-[[4-(2,6-dichloro-4-fluoro-phenyl)-7-quinolyl]oxy]propanoyl]-2,8-diazaspiro[4.5]decan-1-one,
rac-(2R)-2-[[4-(2,6-dichloro-4-fluoro-phenyl)-7-quinolyl]oxy]-1-[3-(1-hydroxycyclopropyl)-1-piperidyl]propan-1-one,
(2R)-2-[[4-(2,6-dichloro-4-fluoro-phenyl)-7-quinolyl]oxy]-1-[(2R)-2-methyl-1-piperidyl]propan-1-one,
8-[(2R)-2-[[4-(2-chloro-4-fluoro-phenyl)-7-quinolyl]oxy]propanoyl]-2,8-diazaspiro[4.5]decan-1-one,
(2R)-2-[[4-(2-chloro-4-fluoro-phenyl)-7-quinolyl]oxy]-1-morpholino-propan-1-one,
(2R)-2-[[4-(2-chloro-4-fluoro-phenyl)-7-quinolyl]oxy]-1-[3-(1-hydroxycyclopropyl)-1-piperidyl]propan-1-one,
(2R)-2-[[4-(2,6-dichloro-4-fluoro-phenyl)-7-quinolyl]oxy]-1-(2-oxa-7-azaspiro[3.5]nonan-7-yl)propan-1-one,
N-hydroxy-N-[rac-(3S)-1-[rac-(2R)-2-[[4-(2-chloro-4-fluoro-phenyl)-7-quinolyl]oxy]propanoyl]-3-piperidyl]acetamide,
(2R)-2-[[4-(2-chloro-4-fluoro-phenyl)-7-quinolyl]oxy]-1-(2-oxa-7-azaspiro[3.5]nonan-7-yl)propan-1-one,
(2R)-2-[[4-(2-chloro-4-fluoro-phenyl)-7-quinolyl]oxy]-1-(2,6-dimethyl-1-piperidyl)propan-1-one,
(2R)-2-[[4-(2-chloro-4-fluoro-phenyl)-7-quinolyl]oxy]-1-(2-oxa-8-azaspiro[3.5]nonan-8-yl)propan-1-one,
(2R)-2-[[4-(2-chloro-4-fluoro-phenyl)-7-quinolyl]oxy]-1-(2-oxa-7-azaspiro[3.4]octan-7-yl)propan-1-one,
1-tert-butyl-3-[(3R)-1-[(2R)-2-[[4-(2-chloro-4-fluoro-phenyl)-7-quinolyl]oxy]propanoyl]pyrrolidin-3-yl]urea,
(2R)-2-[[4-(2-chloro-4-fluoro-phenyl)-7-quinolyl]oxy]-1-(3,3,5,5-tetramethylpiperazin-1-yl)propan-1-one,
(2R)-2-[[4-(2-chloro-4-fluoro-phenyl)-7-quinolyl]oxy]-1-(3,5-dimethylpiperazin-1-yl)propan-1-one,
(2R)-2-[[4-(2-chloro-4-fluoro-phenyl)-7-quinolyl]oxy]-1-[(1R)-2,5-diazabicyclo[2.2.1]heptan-2-yl]propan-1-one,
(2R)-2-[[4-(2-chloro-4-fluoro-phenyl)-7-quinolyl]oxy]-1-(2-oxa-6-azaspiro[3.3]heptan-6-yl)propan-1-one,
1-[(2R)-2-[[4-(2-chloro-4-fluoro-phenyl)-7-quinolyl]oxy]propanoyl]-3,6-dihydro-2H-pyridine-5-carboxylic acid,
2-[(3R)-1-[(2R)-2-[[5-(2,6-dichloro-4-fluoro-phenyl)-1,8-naphthyridin-2-yl]oxy]propanoyl]-3-piperidyl]acetic acid, and
2-[(3R)-1-[(2R)-2-[[5-(4-fluoro-2,6-dimethyl-phenyl)-1,8-naphthyridin-2-yl]oxy]propanoyl]-3-piperidyl]acetic acid,
   or a pharmaceutically or veterinary acceptable salt, hydrate or solvate thereof.

In a further embodiment, the invention relates to a composition, as defined above, wherein the IMT is selected from the group consisting of:
N,N-dimethyl-2-[4-(o-tolyl)-2-oxo-chromen-7-yl]oxy-propanamide, 1-[2-[4-(2-chlorophenyl)-2-oxo-chromen-7-yl]oxypropanoyl]piperidine-3-sulfonamide,
(3S)-1-[(2R)-2-[4-(2-chloro-4-fluoro-phenyl)-2-oxo-chromen-7-yl]oxypropanoyl]piperidine-3-carboxylic acid,
(3S)-1-[2-[4-(2-chloro-4-fluoro-phenyl)-2-oxo-chromen-7-yl]oxypropanoyl]piperidine-3-carboxylic acid, and
2-[(3R)-1-[(2R)-2-[4-(2-chloro-4-fluoro-phenyl)-2-oxo-chromen-7-yl]oxypropanoyl]-3-piperidyl]acetic acid,
   or a pharmaceutically or veterinary acceptable salt, hydrate or solvate thereof.

In a particular preferred embodiment, the invention relates to a composition, as defined above, wherein the IMT is (3S)-1-[(2R)-2-[4-(2-chloro-4-fluoro-phenyl)-2-oxo-chromen-7-yl]oxypropanoyl]piperidine-3-carboxylic acid, or a pharmaceutically or veterinary acceptable salt, hydrate or solvate thereof.

In a further embodiment, the invention relates to a composition, as defined above, wherein, the at least one anti-cancer drug is selected from the group of (i) a B-cell lymphocyte-2 anti-apoptotic protein (Bcl-2) inhibitor, (ii) an inhibitor of the MEK/ERK pathway, including but not limited to a mitogen-activated protein (MAP) kinase inhibitor, MEK inhibitor or ERK inhibitor, (iii) an inhibitor of poly-ADP Ribose-Polymerase (PARPi), (iv) a Glucose consumption/uptake inhibitor, including but not limited to 2-deoxy glucose and derivatives and inhibitors of glucose transporters (GLUT), (v) a dihydroorotate-dehydrogenase (DHODH) inhibitor, (vi) a phosphatidylinositol-4,5-bisphosphate 3-kinase PIK3Cα (p110α) inhibitor, and (vii) an immunotherapeutic agent.

An anti-cancer drug as used herein is any compound which may be used as the sole drug, i.e. the only active ingredient, in anti-cancer therapy, or may be a substance which may be used in combination with further compounds in anti-cancer therapy.

In a further embodiment, the invention relates to a composition, as defined above, wherein the B-cell lymphocyte-2 anti-apoptotic protein (Bcl-2) inhibitor is selected from the group consisting of Venetoclax (ABT-199), Navitoclax (ABT-263) and Oblimersen (G3139).

In a further embodiment, the invention relates to a composition, as defined above, wherein the inhibitor of the MEK/ERK pathway is selected from the group consisting of Vemurafenib, Dabrafenib, Ulixertinib, Encorafenib (LGX818, (S)-methyl (1-((4-(3-(5-chloro-2-fluoro-3-(methylsulfonamido)phenyl)-1-isopropyl-1H-pyrazol-4-yl)pyrimidin-2-yl)amino)propan-2-yl)carbamate), Trametinib (GSK1120212), Binimetinib (MEK162), Cobimetinib (XL518, GDC0973), Selumetinib (AZD6244), N-[(2R)-2,3-Dihydroxypropoxy]-3,4-difluoro-2-[(2-fluoro-4-iodophenyl)amino]-benzamid (PD-325901), 2-(2-chloro-4-iodophenylamino)-N-cyclopropylmethoxy-3,4-difluorobenzamide (PD-184352/CI-1040), 3-[(2R)-2,3-Dihydroxypropyl]-6-fluor-5-[(2-fluor-4-iodphenyl)amino]-8-methylpyrido[2,3-d]pyrimidin-4,7(3H,8H)-dion (TAK-733), 2-((2-Fluoro-4-iodophenyl)amino)-N-(2-hydroxyethoxy)-1,5-dimethyl-6-oxo-1,6-dihydropyridine-3-carboxamide (AZD8330), and 5-Brom-N-(2,3-dihydroxypropoxy)-3,4-difluor-2-[(2-fluor-4-iodphenyl)amino]benzamid (PD-318088).

In a further embodiment, the invention relates to a composition, as defined above, wherein the inhibitor of poly-ADP Ribose-Polymerase (PARPi) is selected from the group consisting of Olaparib, Rucaparib, Niraparib, Talazoparib, Veliparib, Pamiparib, CEP9722 (11-methoxy-2-((4-methylpiperazin-1-yl)methyl)-4,5,6,7-tetrahydro-1H-cyclopenta[a]pyrrolo[3,4-c]carbazole-1,3(2H)-dione), E7016 (10-((4-Hydroxypiperidin-1-yl)methyl)chromeno[4,3,2-de]phthalazin-3(2H)-one), Iniparib, and 3-aminobenzamide.

In a further embodiment, the invention relates to a composition, as defined above, wherein the Glucose consumption/uptake inhibitor is selected from 2-deoxy glucose and derivatives, and the GLUT inhibitor is BAY-876.

In a further embodiment, the invention relates to a composition, as defined above, wherein the dihydroorotate-dehydrogenase (DHODH) inhibitor is selected from the group consisting of Brequinar, Leflunomide/Teriflunomide, Enliuracil, Vidofludimus, GNF-Pf-4706 (Ethyl 4-(4-ethoxybenzyl)-3,5-dimethyl-1H-pyrrole-2-carboxylate), (E)-2-((2-(4-(2-chlorophenyl)thiazol-2-yl)hydrazono)methyl)benzoic acid (S312) and (E)-2-((2-(4-(2-chlorophenyl)thiazol-2-yl)-2-methylhydrazono)methyl)benzoic acid (S416).

In a further embodiment, the invention relates to a composition, as defined above, wherein the phosphatidylinositol-4,5-bisphosphate 3-kinase PIK3Cα (p110α) inhibitor is selected from the group consisting of Duvelisib, Wortmannin, LY294002 (2-(4-Morpholinyl)-8-phenyl-4H-chromen-4-one), Copanlisib (BAY80-6946; 2-Amino-N-{7-methoxy-8-[3-(4-morpholinyl)propoxy]-2,3-dihydroimidazo[1,2-c]quinazolin-5-yl}-5-pyrimidinecarboxamide), AZD6482 (2-({(1R)-1-[7-Methyl-2-(4-morpholinyl)-4-oxo-4H-pyrido[1,2-a]pyrimidin-9-yl]ethyl}amino)benzoic acid), Bimiralisib (5-(4,6-dimorpholin-4-yl-1,3,5-triazin-2-yl)-4-(trifluoromethyl)pyridin-2-amine), Pictilisib (GDC0941; 2-(1H-Indazol-4-yl)-6-[[4-(methylsulfonyl)-1-piperazinyl]methyl]-4-(4-morpholinyl)thieno[3,2-d]pyrimidine), ZSTK474 (2-(Difluoromethyl)-1-[4,6-di(4-morpholinyl)-1,3,5-triazin-2-yl]-1H-benzimidazole), Omipalisib (GSK2126458; 2,4-Difluoro-N-[2-methoxy-5-[4-(4-pyridazinyl)-6-quinolinyl]-3-pyridinyl]benzenesulfonamide) and Buparlisib (BKM120; 5-[2,6-Di(4-morpholinyl)-4-pyrimidinyl]-4-(trifluoromethyl)-2-pyridinamine).

In a further embodiment, the invention relates to a composition, as defined above, wherein the immunotherapeutic agent is selected from the group consisting of immune-stimulating agents interferone gamma, axitinib (N-Methyl-2-[[3-[(E)-2-pyridin-2-ylethenyl]-1H-indazol-6-yl]sulfanyl]benzamide), lenalidomide ((3RS)-3-(4-Amino-1-oxo-1,3-dihydro-2H-isoindol-2-yl)piperidine-2,6-dione), immune check-point inhibitors pembrolizumab, cemiplimab, durvalumab, ipilimumab, nivolumab, PD-1 ligand inhibitors atezolizumab, avelumab, anti-angiogenic agents ramucirumab, bevacimumab, cetuximab, rituximab, daratumumab, trastuzumab and antibody-drug conjugates bretuximab-vedotin.

In a further embodiment, the invention relates to a pharmaceutical composition comprising a composition as defined herein and a pharmaceutically or veterinary acceptable excipient or carrier.

In a further embodiment, the invention relates to a kit comprising at least one inhibitor of mitochondrial transcription (IMT) as defined herein and at least one anti-cancer drug as defined herein.

In a further embodiment, the invention relates to a composition as defined herein, a pharmaceutical composition as defined herein, or a kit as defined herein for use as a medicament.

In a further embodiment, the invention relates to a composition as defined herein, a pharmaceutical composition as defined herein, or a kit as defined herein for use in a method of treating, and/or preventing cancer in a subject.

In a further embodiment, the invention relates to a composition as defined herein, a pharmaceutical composition as defined herein, or a kit as defined herein for use as defined herein, wherein the cancer is selected from the group consisting of Acute Lymphoblastic Leukemia (ALL), Acute Myeloid Leukemia (AML), Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Childhood (Brain Cancer), Atypical Teratoid/Rhabdoid Tumor, Childhood, Central Nervous System (Brain Cancer), Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors (Lung Cancer), Burkitt Lymphoma, Carcinoid Tumor (Gastrointestinal), Cardiac (Heart) Tumors (Childhood), Central Nervous System Cancer, Atypical Teratoid/Rhabdoid Tumor (Childhood) (Brain Cancer), Medulloblastoma and Other CNS Embryonal Tumors (Childhood) (Brain Cancer), Germ Cell Tumor (Childhood) (Brain Cancer), Primary CNS Lymphoma, Cervical Cancer, Childhood Cancers, Rare Cancers of Childhood, Cholangiocarcinoma, Chordoma (Childhood) (Bone Cancer), Chronic Lymphocytic Leukemia (CLL), Chronic Myelogenous Leukemia (CML), Chronic Myeloproliferative Neoplasms, Colorectal Cancer, Craniopharyngioma (Childhood) (Brain Cancer), Cutaneous T-Cell Lymphoma (Mycosis Fungoides and Sezary Syndrome), Ductal Carcinoma In Situ (DCIS), Embryonal Tumors, Medulloblastoma and Other Central Nervous System (Childhood) (Brain Cancer), Endometrial Cancer (Uterine Cancer), Ependymoma (Childhood) (Brain Cancer), Esophageal Cancer, Esthesioneuroblastoma (Head and Neck Cancer), Ewing Sarcoma (Bone Cancer), Extracranial Germ Cell Tumor (Childhood), Extragonadal Germ Cell Tumor, Eye Cancer, Intraocular Melanoma, Retinoblastoma, Fallopian Tube Cancer, Fibrous Histiocytoma of Bone (Malignant, and Osteosarcoma), Gallbladder Cancer, Gastric (Stomach) Cancer, Gastrointestinal Carcinoid Tumor, Gastrointestinal Stromal Tumors (GIST) (Soft Tissue Sarcoma), Germ Cell Tumors, Childhood Central Nervous System Germ Cell Tumors (Brain Cancer), Glioma (Brain Cancer), Glioblastoma multiforme (GBM, Brain Cancer), Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors (Childhood), Hepatocellular (Liver) Cancer, Hodgkin Lymphoma, Hypopharyngeal Cancer (Head and Neck Cancer), Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell, Small Cell, Pleuropulmonary Blastoma, and Tracheobronchial Tumor), Lymphoma, Male Breast Cancer, Malignant Fibrous Histiocytoma of Bone and Osteosarcoma, Melanoma, Intraocular (Eye)Melanoma, Merkel Cell Carcinoma (Skin Cancer), Malignant Mesothelioma, Metastatic Cancer, Melanoma Brain Metastatic Cancer, Metastatic Squamous Neck Cancer with Occult Primary (Head and Neck Cancer), Midline Tract Carcinoma With NUT Gene Changes, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Chronic Myeloproliferative Neoplasms, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer, Lip and Oral Cavity Cancer and Oropharyngeal Cancer (Head and Neck Cancer), Osteosarcoma and Undifferentiated Pleomorphic Sarcoma of Bone Treatment, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Pituitary Tumor, Plasma Cell Neoplasm/Multiple Myeloma, Pleuropulmonary Blastoma (Lung Cancer), Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Childhood (Soft Tissue Sarcoma), Salivary Gland Cancer (Head and Neck Cancer), Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Soft Tissue Sarcoma, Uterine Sarcoma, Sezary Syndrome (Lymphoma), Skin Cancer, Small Cell Lung Cancer, Small Intestine Cancer, Soft Tissue Sarcoma, Squamous Cell Carcinoma of the Skin, Metastatic Squamous Neck Cancer with Occult Primary (Head and Neck Cancer), Stomach (Gastric) Cancer, Cutaneous T-Cell Lymphoma, Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Tracheobronchial Tumors (Lung Cancer), Triple-Negative Breast Cancer, Transitional Cell Cancer of the Renal Pelvis and Ureter (Kidney (Renal Cell) Cancer), Carcinoma of Unknown Primary, Ureter and Renal Pelvis, Transitional Cell Cancer (Kidney (Renal Cell) Cancer, Urethral Cancer, Uterine Cancer, Endometrial, Uterine Sarcoma, Vaginal Cancer, Vascular Tumors (Soft Tissue Sarcoma), Vulvar Cancer, Wilms Tumor and Other Childhood Kidney Tumors.

In a further embodiment, the invention relates to a composition as defined herein, a pharmaceutical composition as defined herein, or a kit as defined herein for use in a method of treating cancer in simultaneous, alternating or subsequent combination with another cancer therapy, preferably selected from chemotherapy, immunotherapy, hormone therapy, stem cell transplantation therapy, radiation therapy or surgery.

### DETAILED DESCRIPTION OF THE INVENTION

### Definitions, Abbreviations and Acronyms

### Formula (I):

“5- or 6-membered saturated heterocycle” represents an unsubstituted or substituted saturated or partially unsaturated ring system containing 5 or 6 ring atoms and containing in addition to C ring atoms for example one to three nitrogen atoms and/or an oxygen or a sulfur atom. In a preferred embodiment, the 5- or 6-membered saturated heterocycle contains in addition to C ring atoms one N and optionally one additional heteroatom. The additional heteroatoms are preferably selected from O, N or S. Especially preferred are heterocycles with only one N as a heteroatom. Preferably, these substituted heterocycles are single or twofold substituted. The 5- or 6-membered saturated heterocycle may be substituted at the C atom(s), at the O atom(s), at the N atom(s) or at the S atom(s). Examples of 5- or 6-membered saturated heterocycle include, but are not limited to 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2,3-pyrrolin-2-yl, 2,3-pyrrolin-3-yl, 2,4-pyrrolin-2-yl, 2,4-pyrrolin-3-yl, 2,3-isoxazolin-3-yl, 3,4-isoxazolin-3-yl, 4,5-isoxazolin-3-yl, 2,3-isoxazolin-4-yl, 3,4-isoxazolin-4-yl, 4,5-isoxazolin-4-yl, 2,3-isoxazolin-5-yl, 3,4-isoxazolin-5-yl, 4,5-isoxazolin-5-yl, 2,3-isothiazolin-3-yl, 3,4-isothiazolin-3-yl, 4,5-isothiazolin-3-yl, 2,3-isothiazolin-4-yl, 3,4-isothiazolin-4-yl, 4,5-isothiazolin-4-yl, 2,3-isothiazolin-5-yl, 3,4-isothiazolin-5-yl, 4,5-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, morpholinyl, 2-piperidinyl, 3-piperidinyl, 4-piperidinyl, 1-piperazinyl, 2-piperazinyl, 1,3-dioxan-5-yl, 2-tetrahydropyranyl, 4-tetrahydropyranyl, 2-tetrahydrothienyl, 3-tetrahydropyridazinyl, 4-tetrahydropyridazinyl, 2-tetrahydropyrimidinyl, 4-tetrahydropyrimidinyl, 5-tetrahydropyrimidinyl, 2-tetrahydropyrazinyl, 1,3,5-tetrahydrotriazin-2-yl and 1,2,4-tetrahydrotriazin-3-yl, preferably piperidin-1-yl, 2-piperidinyl, 3-piperidinyl, 4-piperidinyl, 1-piperazinyl, 2-piperazinyl, 2-pyrrolidinyl, and 3-pyrrolidinyl,

The 5- or 6-membered saturated heterocycle may be each optionally and independently substituted with one or more, preferably with one of the following residues:
—C₁-C₄-alkyl;
—(CH₂)ₘ—COOR₅′ with R₅′ is —H, —C₁-C₈-alkyl, —C₂-C₄-alkyl-N-morpholine or the group

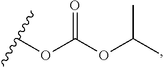

preferably —H, -methyl, -ethyl, -isopropyl, -tert-butyl, -n-heptyl, 2-morpholinoethyl or -isopropoxycarbonyloxymethyl;
—(CH₂)mCONR₆′R₇′ with R₆′ and R₇′ is independently —H, or —C₁-C₄-alkyl, preferably —H or -methyl;
—CO—(C₂-C₄-alkenyl); —CO—CH₂—Cl; —CO—CH₂—CH₃;
—NH—CO—(C₂-C₄-alkenyl); —NH—CO—CH₂—Cl; —NH—CO—CH₂—CH₃;
—F;
—CN;
—SO₃H;
—SO₂NR₈′R₉′ with R₈ and R₉ independently are —H, or —C₁-C₄-alkyl, preferably —H or -methyl;
—SONHR₁₀′ with R₁₀′ is —C₁-C₄-alkyl, preferably -methyl; or

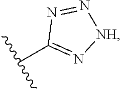 [SMILES: *c1nn[nH]n1]

and
m′=0, 1, or 2, preferably 0 or 1.

“C₁-C₄-alkyl” represents a straight-chain or branched-chain alkyl group with 1 to 4 carbon atoms. Examples of straight-chain and branched groups include, but are not limited to, methyl, ethyl, propyl, isopropyl, butyl, isobutyl, and tert.-butyl, preferably methyl and ethyl and most preferred methyl.

“C₃-C₆-cycloalkyl” represents a carbocyclic saturated ring system having 3 to 6 carbon atoms. Examples of C₃-C₆-cycloalkyl include, but are not limited cyclopropyl, cyclobutyl, cyclopentyl, or cyclohexyl, preferably cyclopentyl and cyclohexyl.

“Substitution” or “substituted” represents one or more substituents commonly known in the art, or as specifically defined herein.

“Halogen” represents fluoro, chloro, bromo or iodo, preferably represents fluoro and chloro.

“Stereoisomer(s)” as it relates to a compound of formula (I) and to its intermediate compounds represents any possible enantiomers or diastereomers of a compound of formula (I) and its salts or hydrates. In particular, the term “stereoisomer” means a single compound or a mixture of two or more compounds, wherein at least one chiral center is predominantly present in one definite isomeric form, in particular the S-enantiomer, the R-enantiomer and the racemate of a compound of formula (I). It is also possible that two or more stereogenic centers are predominantly present in one definite isomeric form of a derivative of a compound of formula (I) as defined above. In the sense of the present invention, “predominantly” has the meaning of at least 60%, preferably at least 70%, particularly preferably at least 80%, most preferably at least 90%. According to the present invention, also stereoisomers of a compound of formula (I) may be present as a salt or a hydrate.

The terms stereoisomer, salt, and hydrate may also be used in conjunction with one another. For example, a stereoisomer of a compound of formula (I) may have a salt. Combinations of these terms are considered to be within the scope of the invention.

### Formula (II):

“5- or 6-membered saturated heterocycle” represents an unsubstituted or substituted ring system containing 5 or 6 ring atoms and containing in addition to C atoms one N atom and optionally one additional heteroatom. The additional heteroatoms are preferably selected from O, N or S. Especially preferred are heterocycles with only one N as a heteroatom. Preferably, these substituted heterocycles are single or twofold substituted.

“C₁-C₄-alkyl” represents a straight-chain or branched-chain alkyl group with 1 to 4 carbon atoms. Examples of straight-chain and branched groups include, but are not limited to, methyl, ethyl, propyl, isopropyl, butyl, isobutyl, and tert.-butyl, preferably methyl and ethyl and most preferred methyl.

“C₃-C₈-cycloalkyl” represents a carbocyclic saturated ring system having 3 to 8 carbon atoms, preferably 3 to 6 carbon atoms. Examples of C₃-C₈-cycloalkyl include, but are not limited cyclopropyl, cyclobutyl, cyclopentyl, cyclohexyl, cycloheptyl or cyclooctyl, preferably cyclopentyl and cyclohexyl.

“Substitution” or “substituted” represents one or more substituents commonly known in the art, or as specifically defined herein. The substituents are preferably selected from C₁-C₄-alkyl, —COO(CH₂)_{n″}H or —COO(CH₂)_{n″}OH with n′″=0-4, —CN, and halogen. Preferred substituents are methyl, ethyl or —COOH.

“Halogen” represents fluoro, chloro, bromo or iodo, preferably fluoro and chloro.

“Stereoisomer(s)” as it relates to a compound of formula (II) and to its intermediate compounds represents any possible enantiomers or diastereomers of a compound of formula (II) and its salts or hydrates. In particular, the term “stereoisomer” means a single compound or a mixture of two or more compounds, wherein at least one chiral center is predominantly present in one definite isomeric form, in particular the S-enantiomer, the R-enantiomer and the racemate of a compound of formula (II). It is also possible that two or more stereogenic centers are predominantly present in one definite isomeric form of a derivative of a compound of formula (II) as defined above. In the sense of the present invention, “predominantly” has the meaning of at least 60%, preferably at least 70%, particularly preferably at least 80%, most preferably at least 90%. According to the present invention, also stereoisomers of a compound of formula (II) may be present as a salt or a hydrate.

The terms stereoisomer, salt, and hydrate may also be used in conjunction with one another. For example, a stereoisomer of a compound of formula (II) may have a salt. Combinations of these terms are considered to be within the scope of the invention.

### Formula (III):

“4-, 5- or 6-membered saturated heterocycle” represents an unsubstituted or substituted saturated or partially unsaturated ring system containing 4, 5 or 6 ring atoms and containing in addition to C ring atoms one to three nitrogen atoms and/or an oxygen or sulfur atom or one or two oxygen and/or sulfur atoms. In a particular preferred embodiment the “4-, 5- or 6-membered saturated heterocycle” represents an unsubstituted or substituted saturated ring system containing 4, 5 or 6 ring atoms and containing in addition to C ring atoms one to three nitrogen atoms and/or an oxygen or sulfur atom or one or two oxygen and/or sulfur atoms. In a preferred embodiment, the 4-, 5- or 6-membered saturated heterocycle contains in addition to C ring atoms one N and optionally one additional heteroatom. The additional heteroatoms are preferably selected from O, N or S. Especially preferred are heterocycles with only one N as a heteroatom. Preferably, these substituted heterocycles are single or twofold substituted. The 4-, 5- or 6-membered saturated heterocycle may be substituted at the C atom(s), at the O atom(s), at the N atom(s) or at the S atom(s). Examples of 4-, 5- or 6-membered saturated heterocycle include, but are not limited to oxetanyl, azetidinyl, 1,3-diazetinyl, thietanyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2,3-pyrrolin-2-yl, 2,3-pyrrolin-3-yl, 2,4-pyrrolin-2-yl, 2,4-pyrrolin-3-yl, 2,3-isoxazolin-3-yl, 3,4-isoxazolin-3-yl, 4,5-isoxazolin-3-yl, 2,3-isoxazolin-4-yl, 3,4-isoxazolin-4-yl, 4,5-isoxazolin-4-yl, 2,3-isoxazolin-5-yl, 3,4-isoxazolin-5-yl, 4,5-isoxazolin-5-yl, 2,3-isothiazolin-3-yl, 3,4-isothiazolin-3-yl, 4,5-isothiazolin-3-yl, 2,3-isothiazolin-4-yl, 3,4-isothiazolin-4-yl, 4,5-isothiazolin-4-yl, 2,3-isothiazolin-5-yl, 3,4-isothiazolin-5-yl, 4,5-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1-piperazinyl, 2-piperazinyl, 1,3-dioxan-5-yl, 2-tetrahydropyranyl, 4-tetrahydropyranyl, 2-tetrahydrothienyl, 3-tetrahydropyridazinyl, 4-tetrahydropyridazinyl, 2-tetrahydropyrimidinyl, 4-tetrahydropyrimidinyl, 5-tetrahydropyrimidinyl, 2-tetrahydropyrazinyl, 1,3,5-tetrahydrotriazin-2-yl and 1,2,4-tetrahydrotriazin-3-yl, preferably piperidin-1-yl, 2-piperidinyl, 3-piperidinyl, 4-piperidinyl, 1-piperazinyl, 2-piperazinyl, 2-pyrrolidinyl, and 3-pyrrolidinyl, tetrahydropyridinyl, preferably 1,2,3,6-tetrahydropyridinyl, 1,2-oxazinyl, 1,3-oxazinyl, and 1,4-oxazinyl, preferably tetrahydro-1,4-oxazinyl.

The 4-, 5- or 6-membered saturated heterocycle may be each optionally and independently substituted with one or more, preferably with one of the following residues:
—C₁-C₄-alkyl;
—C(OH)-cyclopropyl; —C(COOH)-cyclopropyl;
unsubstituted or substituted —C₃-C₆-cycloalkyl; preferably hydroxycyclopropyl or carboxycyclopropyl;
—(CH₂)ₒ′″—COOR₇′″ with
   R₇′″ is —H, —C₁-C₈-alkyl,
   preferably —H, -methyl, -ethyl, -isopropyl, or -tert-butyl, and
   o′″=0, 1 or 2; preferably 0 or 1;

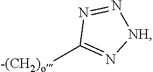

and o′″ is as defined above;
—(CH₂)ₚ′″CONR₈′″R₉′″ with
   R₈′″ and R₉′″ independently are —H, —OH, —CN, or —C₁-C₄-alkyl, preferably —H or -methyl, and
   p′″=0, 1 or 2; preferably 0 or 1;
—C(CH₃)₂—COOH;
═O or —OH;
—CO-cyclopropyl;
—CO—(C₁-C₄-alkyl), preferably —CO—CH₂—CH₃;
—CO—(CH₂)_{q}′″—NR₁₂′″R₁₃′″ with R₁₂′″ and R₁₃′″ independently are —H, —C₁-C₄-alkyl or —CN, preferably —CO—(CH₂)_{q}′″—NH₂, more preferably —CO—CH₂—NH₂, and
q″=0, 1 or 2, preferably 0 or 1;
—NH₂, —NH—CO-cyclopropyl, —NH—CO—CH₂—Cl, —NH—CO—CH₂—CH₃, —NH—CO—NH—C(CH₃)₃, —NH—SO₂CH₃, —NH—CO-phenyl, —NOH—CO—CH₃;
—F; —CN;
R₁₄′″ and R₁₅′″ forming a pyrrolidinone ring, a cyclopropanecarboxlic acid ring, an oxetane ring or a —CH₂— group;
or
—(CH₂)ᵣ′″SO₂NR₁₀′″R₁₁′″ with R₁₀′″ and R₁₁′″ independently are —H, or —C₁-C₄-alkyl, preferably —H or -methyl, preferably —CH₂SO₂NH₂ and
   r′″=0, 1 or 2, preferably 0 or 1.

“C₁-C₄-alkyl” and “C₁-C₈-alkyl” represent a straight-chain or branched-chain alkyl group with 1 to 4 or 1 to 8 carbon atoms, respectively. Examples of straight-chain and branched groups include, but are not limited to, methyl, ethyl, propyl, isopropyl, butyl, isobutyl, and tert-butyl, preferably methyl and ethyl and most preferred methyl.

“halogen-C₁-C₄-alkyl” represents a straight-chain or branched alkyl group having 1 to 4 carbon atoms (as mentioned above), it being possible for the hydrogen atoms in these groups to be partly or completely replaced by halogen atoms as mentioned above, e.g. C₁-C₂-halogenalkyl such as chloromethyl, dichloromethyl, trichloromethyl, fluoromethyl, difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2fluoroethyl, 2-chloro-2,2-difluoroethyl, 2,2-dichloro-2-fluoroethyl, 2,2,2-trichloroethyl and pentafluoroethyl;

“C₁-C₄-alkoxy” represents a straight-chain or branched-chain alkyl group with 1 to 4 or 1 to 8 carbon atoms, which are bonded to the structure via an oxygen atom (—O).

“C₁-C₄-dialkylamino” represents two straight-chain or branched alkyl groups having 1 to 4 carbon atoms (as mentioned above), which are independent of one another and are bonded to the structure via a nitrogen atom (—N:);

“C₂-C₆-alkenyl” represents a straight-chain or branched-chain hydrocarbon group comprising an olefinic bond in any desired position and 2 to 6, more preferably 2 to 4 carbon atoms. Examples of alkenyl groups include, but are not limited to, ethenyl, 1-propenyl, 2-propenyl, isopropenyl, 1-butenyl, 2-butenyl, 3-butenyl and isobutenyl. Preferred examples are 1-propenyl and 2-propenyl.

“C₂-C₆-alkynyl” represents a straight-chain or branched hydrocarbon group having 2 to 6 carbon atoms and a triple bond in any desired position, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

“C₃-C₆-cycloalkyl” represents a carbocyclic saturated ring system having 3 to 6 carbon atoms. Examples of C₃-C₆-cycloalkyl include, but are not limited cyclopropyl, cyclobutyl, cyclopentyl, or cyclohexyl, preferably cyclopentyl and cyclohexyl.

“Substitution” or “substituted” represents one or more substituents commonly known in the art, or as specifically defined herein.

“Halogen” represents fluoro, chloro, bromo or iodo, preferably represents fluoro and chloro.

“Stereoisomer(s)” as it relates to a compound of formula (III) and to its intermediate compounds represents any possible enantiomers or diastereomers of a compound of formula (III) and its salts or hydrates. In particular, the term “stereoisomer” means a single compound or a mixture of two or more compounds, wherein at least one chiral center is predominantly present in one definite isomeric form, in particular the S-enantiomer, the R-enantiomer and the racemate of a compound of formula (III). It is also possible that two or more stereogenic centers are predominantly present in one definite isomeric form of a derivative of a compound of formula (III) as defined above. In the sense of the present invention, “predominantly” has the meaning of at least 60%, preferably at least 70%, particularly preferably at least 80%, most preferably at least 90%. According to the present invention, also stereoisomers of a compound of formula (III) may be present as a salt or a hydrate.

The terms stereoisomer, salt, and hydrate may also be used in conjunction with one another. For example, a stereoisomer of a compound of formula (III) may have a salt. Combinations of these terms are considered to be within the scope of the invention.

Technical terms are used by their common sense. If a specific meaning is conveyed to certain terms, definitions of terms will be given in the following in the context of which the terms are used.

The residue definitions can be combined with one another at will, i.e. including combinations between the given preferred residues. Further, individual definitions may not apply.

### Novel Compositions

As indicated above, there is a need for compositions, which are suitable for use as a medicament. In particular, a need exists for compositions that can be used in the treatment and/or prevention of cancer.

The present invention is directed to a composition comprising at least one inhibitor of mitochondrial transcription (IMT) and at least one anti-cancer drug.

The composition according to claim 1, wherein the at least one IMT is a Mitochondrial RNA Polymerase inhibitor as determined using an assay as described herein (see Assays 1 and 2).

The IMT may be a quinolone derivative or a coumarin derivative. The IMT may also be a combination of at least two quinolone derivatives or a combination of at least two coumarin derivatives. Alternatively, the IMT may also be a combination of at least one quinolone derivative and at least one coumarin derivative.

In one embodiment, according to the present invention there is provided a composition, wherein the IMT is a compound of the general formula (I) according to WO2019/057821. The compound of the general formula (I) may be

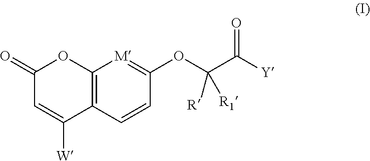

wherein
R′ is —C₁-C₄-alkyl, preferably -methyl or -ethyl, in particular -methyl;
R₁′ is —H, or -methyl, preferably —H;
M′ is CH or N;
W′ is

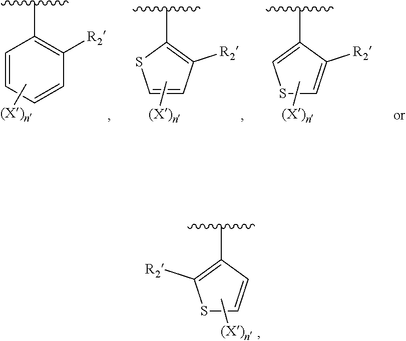

with
   R₂′ is C₁-C₄-alkyl, -halogen, —CN, preferably -methyl, -ethyl, —Cl, or —Br;
   X′ is -halogen, or —CN, preferably —Cl, —Br, or —F, in particular —F, with n′=1 or 2;
   n′=0, 1, or 2, preferably 0 or 1;
Y′ is —NR₃′R₄′ with
   R₃′ is —H, or —C₁-C₄-alkyl, preferably —H or -methyl, and
   R₄′ is —C₁-C₄-alkyl or —C₃-C₆-cycloalkyl, preferably -methyl, -ethyl, -isopropyl, or -cyclopropyl; or an unsubstituted or substituted pyridine residue; or an unsubstituted or substituted phenyl residue, preferably substituted at the para position;
Y′ is —NR₃′R₄′ with N, R₃′ and R₄′ forming an unsubstituted or substituted 5- or 6-membered saturated heterocycle; or
Y′ is —OR₁₁′, with R₁₁′ is —H or —C₁-C₄-alkyl, preferably —H, -methyl, -ethyl, or -isopropyl, or a pharmaceutically or veterinary acceptable salt, hydrate or solvate thereof.

In a preferred embodiment, the compounds of the general formula (I) are coumarin derivatives, wherein M′ is CH.

A preferred group of compounds are compounds, where Y′ is OR₁₁′, with R₁₁′ being an ethyl residue (especially compounds 40, 57, 58, 81, 82 and 113 according to Table 1), an isopropyl residue (compounds 55, 64, 92 according to Table 1) or —H (especially compounds 94, 96, 97, 99, 112 according to Table 1).

Further included are pharmaceutically or veterinary acceptable salts, hydrates or solvates of the compounds of formula (I) or its intermediate compounds disclosed herein. A pharmaceutically or veterinary acceptable salt can be an anionic counterion, e.g. an acetate, a bromide, camsylate, chloride, citrate, formate, fumarate, lactate, maleate, mesylate, nitrate, oxalate, phosphate, sulfate, tartrate, thiocyanate, or tosylate, or preferably a cationic counterion, e.g. ammonium, arginine, diethylamine, ethylenediamine, piperazine, potassium, sodium, or any other counter ion disclosed in Haynes et al. (2005). Some compounds of the invention contain one or more chiral centers due to the presence of asymmetric carbon atoms, which gives rise to stereoisomers, for example to diastereoisomers with R or S stereochemistry at each chiral center. The invention includes all such stereoisomers and diastereoisomers and mixtures thereof.

The compounds of general formula (I) or a pharmaceutically or veterinary acceptable salt, hydrate or solvate thereof, are useful as mitochondrial RNA polymerase (POLRMT) inhibitors and thereby inhibit mitochondrial DNA replication and/or mitochondrial transcription.

In the following, preferred groups of the compounds of general formula (I) of the present invention are described. The preferred groups constitute preferred embodiments of the compounds of general formula (I). Any combinations of the embodiments of the compounds of general formula (I) of the invention described herein are considered to be within the scope of the invention.

In a preferred embodiment, the invention relates to a composition, wherein the IMT is a compound of the general formula (I) as defined above, wherein
R′ is —C₁-C₄-alkyl, preferably -methyl or -ethyl, in particular -methyl;
R₁′ is —H, or -methyl, preferably —H;
M′ is —CH;
W′ is

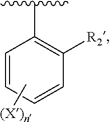

with
R₂′ is methyl, -halogen, —CN, preferably -methyl, —Cl, or —Br;
X′ is -halogen, or —CN, preferably —Cl, —Br, or —F, in particular —F, with n′=1 or 2;
n′=0, 1, or 2, preferably 0 or 1;
Y′ is —NR₃′R₄′ with
R₃′ is —H, or —C₁-C₄-alkyl, preferably —H or -methyl, and
R₄′ is —C₁-C₄-alkyl or —C₃-C₆-cycloalkyl, preferably -methyl, -ethyl, -isopropyl, or -cyclopropyl; or
   an unsubstituted or substituted pyridine residue; or
   an unsubstituted or substituted phenyl residue, preferably substituted at the para position;
Y′ is —NR₃′R₄′ with N, R₃′ and R₄′ form an unsubstituted or substituted 5- or 6-membered saturated heterocycle; or
Y′ is —OR₁₁′, with R₁₁′ is —H or —C₁-C₄-alkyl, preferably —H, -methyl, -ethyl, or -isopropyl.

This preferred group of compounds corresponds to the compounds of formula (IA)

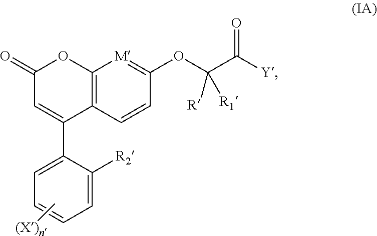

wherein R′, R₁′, R₂′, X′, n′ and Y′ are as defined in the preferred group above.

In one embodiment, the invention relates to a composition, wherein the IMT is a compound of the general formula (I) as defined above, wherein
R′ is —C₁-C₄-alkyl, preferably -methyl or -ethyl, in particular -methyl;
R₁′ is —H, or -methyl, preferably —H;
M′ is —CH;
W′ is

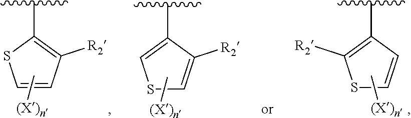

with
   R₂′ is methyl, -halogen, —CN, preferably -methyl, —Cl, or —Br;
   X′ is -halogen, or —CN, preferably —Cl, —Br, or —F, in particular —F, with n′=1 or 2;
   n′=0, 1, or 2, preferably 0 or 1;
Y′ is —NR₃′R₄′ with
R₃′ is —H, or —C₁-C₄-alkyl, preferably —H or -methyl, and
R₄′ is —C₁-C₄-alkyl or —C₃-C₆-cycloalkyl, preferably -methyl, -ethyl, -isopropyl, or -or cyclopropyl; an unsubstituted or substituted pyridine residue; or an unsubstituted or substituted phenyl residue, preferably substituted at the para position;
Y′ is —NR₃′R₄′ with N, R₃′ and R₄′ form an unsubstituted or substituted 5- or 6-membered saturated heterocycle; or
Y′ is —OR₁₁′, with R₁₁′ is —H or —C₁-C₄-alkyl, preferably —H, -methyl, -ethyl, or -isopropyl.

In one embodiment, the invention relates to a composition, wherein the IMT is a compound of the general formula (I) of the group as defined above, wherein
W′ is

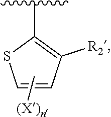

with R₂′, X′ and n′ as defined above, especially compounds 174, 177, 178, 179, 180 and 181 according to Table 1.

In one embodiment, the invention relates to a composition, wherein the IMT is a compound of the general formula (I) of the group as defined above, wherein
W′ is

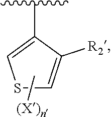

with R₂′, X′ and n′ as defined above, especially compounds 182 and 183 according to Table 1.

In one embodiment, the invention relates to a composition, wherein the IMT is a compound of the general formula (I) of the group as defined above, wherein

W′ is

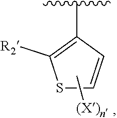

with R₂′, X′ and n′ as defined above, especially compounds 184 and 185 according to Table 1.

In another embodiment, the invention relates to a composition, wherein the IMT is a compound of the general formula (I) as defined above, wherein
Y′ is —NR₃′R₄′, with
   R₃′ is —H, or —C₁-C₄-alkyl, preferably —H or -methyl, and
   R₄′ is a pyridine residue; or a phenyl residue each optionally and independently substituted with —COOH; —COO—(C₁-C₄-alkyl);
      —(CH₂)ₚ′OH with p′=1 or 2; or —C₁-C₄-alkyl or —C₃-C₆-cycloalkyl, preferably -methyl, -ethyl, -isopropyl or -cyclopropyl.

A group of preferred compounds have an optionally substituted phenyl residue (especially compounds 130, 139, 140 and 141 according to Table 1). Another group of preferred compounds have a pyridine residue substituted with —COOH (especially compounds 139, 141, 148, 151, 152, 154, 155 and 160 according to Table 1). Another group of preferred compounds have pyridine residue substituted with —COO—(C₁-C₄-alkyl) (especially compounds 143, 146, 149, 153, 156 according to Table 1).

A specific subset of the compounds of the invention as defined above are the compounds of the general formula (I), wherein Y′ is
—NR₃′R₄′, with
R₃′ is —H, or —C₁-C₄-alkyl, preferably —H or -methyl, and
R₄′ is a pyridine residue; or a phenyl residue substituted with —(CH₂)ₚ′OH with p′=1 or 2; or —C₁-C₄-alkyl or —C₃-C₆-cycloalkyl, preferably -methyl, -ethyl, -isopropyl or -cyclopropyl.

Preferred compounds are compounds with R₃′ is H and R₄′ is a pyridine residue (especially compounds 4 and 31 according to Table 1) or a substituted phenyl residue (especially compounds 19 and 35 according to Table 1) with a 2-hydroxyethyl-substitution, i.e. with p′=2. Other preferred compounds are compounds with R₃′ is H and R₄′ is a cyclopropyl residue (especially compounds 25, 26, 61 and 90 according to Table 1). Further preferred are compounds with R₃′ is H and R₄′ is —C₁-C₄-alkyl (especially compounds 36, 38, 44, 45, 49, 51, 56, 62, 65, 68, 70, 85, 89, 91, 93, 98, 114 and 115 according to Table 1).

Another specific subset of the compounds of the invention are the compounds of the general formula (I), wherein N, R₃′ and R₄′ together form an unsubstituted or substituted piperidine, piperazine or pyrrolidine residue, each optionally and independently substituted with one or more, preferably with one of the following residues:
—C₁-C₄-alkyl;
—(CH₂)ₘ′—COOR₅′ with R₅′ is —H, —C₁-C₈-alkyl, —C₂-C₄-alkyl-N-morpholine or the group

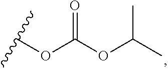 [SMILES: *OC(=O)OC(C)C]

preferably —H, -methyl, -ethyl, -isopropyl, -tert-butyl, -n-heptyl, 2-morpholinoethyl or -isopropoxycarbonyloxymethyl;
—(CH₂)ₘ′CONR₆′R₇′ with R₆′ and R₇′ is independently —H, or —C₁-C₄-alkyl, preferably —H or -methyl;
—CO—(C₂-C₄-alkenyl); —CO—CH₂—Cl; —CO—CH₂—CH₃;
—NH—CO—(C₂-C₄-alkenyl); —NH—CO—CH₂—Cl; —NH—CO—CH₂—CH₃;
—F;
—CN;
—SO₃H;
—SO₂NR₈′R₈′ with R₈′ and R₉′ independently are —H, or —C₁-C₄-alkyl, preferably —H or -methyl;
—SONHR₁₀′ with R₁₀′ is —C₁-C₄-alkyl, preferably -methyl; or

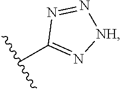 [SMILES: *c1nn[nH]n1]

and
m′=0, 1, or 2, preferably 0 or 1.

Another specific subset of the compounds of the invention are the compounds of the general formula (I), wherein N, R₃′ and R₄′ together form an unsubstituted or substituted piperidine or pyrrolidine residue, each optionally and independently substituted with one or more, preferably with one, of the following residues:
—C₁-C₄-alkyl;
—(CH₂)ₘ′—COOR₅′ with R₅′ is —H, —C₁-C₈-alkyl, —C₂-C₄-alkyl-N-morpholine or the group

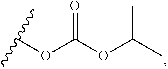 [SMILES: *OC(=O)OC(C)C]

preferably —H, -methyl, -ethyl, -isopropyl, -tert-butyl, -n-heptyl, 2-morpholinoethyl, or -isopropoxycarbonyloxymethyl; —(CH₂)ₘ′CONR₆′R₇′ with R₆′ and R₇′ is independently —H, or —C₁-C₄-alkyl, preferably —H or -methyl;
—F;
—CN;
—SO₃H;
—SO₂NR₈′R₈′ with R₈′ and R₉′ independently are —H, or —C₁-C₄-alkyl, preferably —H or -methyl;
—SONHR₁₀′ with R₁₀′ is —C₁-C₄-alkyl, preferably -methyl; or

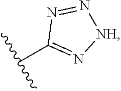 [SMILES: *c1nn[nH]n1]

and
m′=0, 1, or 2, preferably 0 or 1.

A group of preferred compounds have an unsubstituted piperidine (especially compounds 1, 2, 30 and 34 according to Table 1). Especially preferred are compounds having a substituted piperidine residue (especially compounds 3, 6, 7, 8, 9, 10, 11, 12, 14, 15, 16, 17, 18, 20, 21, 23, 24, 27, 28, 32, 33, 37, 39, 41, 42, 43, 46, 47, 48, 50, 52, 53, 54, 59, 60, 66, 67, 69, 71, 72, 73, 74, 75, 76, 78, 79, 80, 83, 84, 86, 87, 88, 95, 100, 101, 102, 103, 104, 105, 106, 107, 108, 109, 110, 111, 116, 117, 118, 119, 120, 121, 122, 123, 124, 125, 126, 127, and 128 according to Table 1).

A more preferred subgroup are compounds having a substituted piperidine residue substituted with —COOH (especially compounds 3, 8, 9, 14, 23, 27, 33, 37, 43, 46, 47, 50, 53, 54, 108, 110, 116 according to Table 1), or with —CH₂COOH (especially compounds 59, 60, 66, 72, 102, 103, 106 and 107 according to Table 1).

Another more preferred subgroup are compounds having a substituted piperidine residue substituted with —COOR₅′ or —CH₂COOR₅′ with R₅′ is 2-morpholinoethyl (especially compounds 119 and 125 according to Table 1) or with R₅′ is -isopropyl (especially compounds 117 and 123 according to Table 1), -tertbutyl (especially compounds 118 and 124 according to Table 1), n-heptyl (especially compounds 120 and 126 according to Table 1), -isopropoxycarbonyloxymethyl (especially compounds 121 and 127 according to Table 1), or with —CONHCH₃, —CH₂CONHCH₃ (especially compounds 15, 21, 122 and 128 according to Table 1) or with —CON(CH₃)₂ (especially compounds 67, 76 and 78 according to Table 1).

Another group of preferred compounds have a substituted piperidine residue substituted with —SO₂NR₈′R₉′ with R₈′ is —H and R₉′ is —H or -methyl (especially compounds 12, 18, 20, 24, 52 and 69 according to Table 1).

Another group of preferred compounds are compounds having an unsubstituted pyrrolidine residue (especially compounds 5 and 13 according to Table 1) and compounds having substituted pyrrolidine residues (especially compounds 22, 29, 63 and 77 according to Table 1).

Another specific subset of the compounds are the compounds of the general formula (I), wherein
R′ is -methyl, preferably (R)-methyl;
R₁′ is —H;
m′=0, or 1;
W′ is

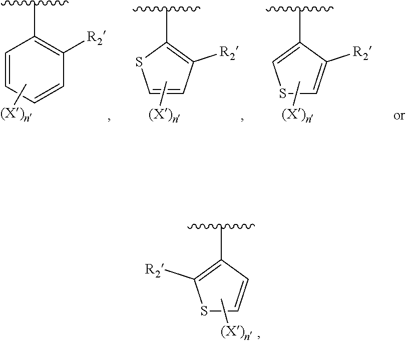

preferably

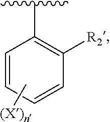

with
   R₂′ is -methyl, or —Cl;
   X′ is —F with n′=1;
Y′ is —NR₃′R₄′
   with
   R₃′ is —H, and
   R₄′ is a pyridine residue, a phenyl residue substituted at the para position, preferably substituted with —(CH₂)₂OH, or a cyclopropyl or isopropyl residue;
   or with
   N, R₃′ and R₄′ are together a piperidine residue, or a pyrrolidine residue, each optionally and independently substituted with one of the following residues: —COOH, —COOCH₃, —COOC₂H₅, —CH₂COOH, —CH₂COOCH₃, —CH₂COOCH₂CH₃, —CONH₂, —CONHCH₃, —CON(CH₃)₂, —CH₂CONHCH₃, —SO₂NH₂, —SO₂NHCH₃, or —CN.

A more preferred group of compounds are compounds having a substituted piperidine residue where R′ is methyl, R₁′ is —H, R₂′ is -methyl or —Cl, n′, m′=0 or 1, X′ is —F with n′=1 and wherein the piperidine is substituted with —COOH, —COOCH₃, —COOC₂H₅, —CH₂COOH, —CH₂COOCH₃, —CH₂COOCH₂CH₃, —CONH₂, —CONHCH₃, —CON(CH₃)₂, —SO₂NH₂, —SO₂NHCH₃, —CH₂CONHCH₃ or —CN (especially compounds 3, 6, 7, 8, 9, 12, 14, 15, 16, 17, 18, 20, 21, 23, 24, 27, 28, 32, 33, 41, 46, 47, 48, 50, 52, 53, 59, 60, 66, 67, 69, 72, 76, 78, 80, 83, 84, 86, 87, 100, 101, 102, 103, 104, 105, 106, 107, 108, 111, 122 and 128 according to Table 1). An even more preferred subgroup of this group are compounds where R′ is (R)-methyl (especially compounds 14, 23, 27, 28, 32, 100, 101, 102, 103, 104, 105, 106, 107, 108, 111, 122 and 128 according to Table 1).

A more preferred subgroup of this specific subset are compounds, wherein R′ is (R)-methyl, having a substituted piperidine residue substituted with —COOH (especially compounds 14, 23, 27 and 108 according to Table 1), or with —CH₂COOH (especially compounds 102, 103, 106 and 107 according to Table 1).

Another more preferred subgroup of this specific subset are compounds, wherein R′ is (R)-methyl, having a substituted piperidine residue substituted with —COOR₅′ or —CH₂COOR₅′, with R₅′ is 2-morpholinoethyl (especially compounds 119 and 125 according to Table 1), or with R₅ is -isopropyl (especially compounds 117 and 123 according to Table 1), -tertbutyl (compounds 118 and 124 according to Table 1), n-heptyl (especially compounds 120 and 126 according to Table 1), -isopropoxycarbonyloxymethyl (especially compounds 121 and 127 according to Table 1), or —CONHCH₃ or —CH₂CONHCH₃ (especially compounds 122 and 128 according to Table 1).

Another more preferred subgroup are compounds having a substituted pyrrolidine residue where R′ is methyl, R₁′ is —H, R₂′ is -methyl or —Cl, n′, m′=0 or 1, X′ is —F with n′=1 and wherein the pyrrolidine is substituted with —COOH, (especially compounds 22 and 29 according to Table 1) or —SO₂NH₂ (especially compounds 63 and 77 according to Table 1).

Another specific subset of compounds are compounds, wherein
R′ is (R)-methyl;
R₁′ is —H;
W′ is

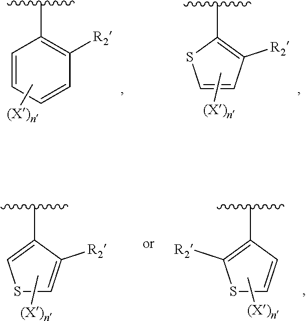

preferably

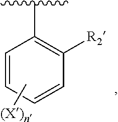

with
   R₂′ is -methyl, or —C₁, preferably —Cl;
   X′ is —F with n′=1;
Y′ is —NR₃′R₄′
   with
   N, R₃′ and R₄′ form a piperidine residue, or a pyrrolidine residue, each optionally and independently substituted with one of the following residues:
   —COOH, —CH₂COOH, —CONHCH₃, —CH₂CONHCH₃, —SO₂NH₂, —SO₂NHCH₃, or —CN, preferably —COOH, —CH₂COOH, —CONHCH₃ or —CH₂CONHCH₃, more preferably (S)—COOH, (R)—COOH, (S)—CH₂COOH or (R)—CH₂COOH, especially a piperidine residue substituted with (S)—COOH, (R)—COOH, (S)—CH₂COOH or (R)—CH₂COOH.

A more preferred group of compounds are compounds having a substituted piperidine residue where R′ is (R)-methyl, R₁′ is —H, R₂′ is -methyl or —C₁, preferably —Cl, X′ is —F, n′=1 and wherein the piperidine is substituted with —COOH, —CH₂COOH, —CONHCH₃, —CH₂CONHCH₃, —SO₂NH₂, —SO₂NHCH₃, or —CN (especially compounds 14, 106, 107, 108, 122 and 128 according to Table 1).

Another specific subset of compounds are compounds, wherein the piperidine residue or the pyrrolidine residue is substituted at the 3-position.

A more preferred group of compounds of this subset are compounds having any substituted piperidine or pyrrolidine residue as defined above at the 3-position (especially compounds 3, 6, 7, 8, 9, 10, 11, 12, 14, 15, 16, 17, 18, 20, 21, 22, 23, 24, 27, 28, 29, 32, 33, 37, 39, 41, 42, 43, 46, 47, 48, 50, 52, 53, 54, 63, 66, 67, 69, 72, 73, 75, 76, 77, 78, 79, 84, 86, 87, 88, 95, 100, 101, 102, 103, 104, 105, 106, 107, 108, 109, 110, 111, 116, 117, 118, 119, 120, 121, 122, 123, 124, 125, 126, 127, and 128 according to Table 1). More preferred within this group are compounds having a substituted piperidine residue substituted with —COOH at the 3-position (especially compounds 3, 8, 9, 14, 23, 27, 33, 37, 43, 46, 47, 50, 53, 108, 110, 116 according to Table 1), with —CH₂COOH at the 3-position (especially compounds 66, 72, 102, 103, 106 and 107 according to Table 1), with —COOR₅′ or —CH₂COOR₅′ at the 3-position, with R₅′ is 2-morpholinoethyl (especially compounds 119 and 125 according to Table 1), or with R₅′ is -isopropyl (compounds 117 and 123 according to Table 1), -tertbutyl (especially compounds 118 and 124 according to Table 1), n-heptyl (especially compounds 120 and 126 according to Table 1), -isopropoxycarbonyloxymethyl (compounds 121 and 127 according to Table 1), or with —CONHCH₃ or —CH₂CONHCH₃ at the 3-position (compounds 122 and 128 according to Table 1).

Another group of preferred compounds of this subset have a substituted piperidine residue substituted with —SO₂NR₈′R₉′ at the 3-position with R₈′ is —H and R₉′ is —H or -methyl (especially compounds 12, 18, 20, 24, 52 and 69 according to Table 1).

Another group of preferred compounds having substituted pyrrolidine residues are compounds substituted at the 3-position with —COOH (especially compounds 22, 29 according to Table 1) or with —SO₂NH₂ (especially compounds 63 and 77 according to Table 1).

A more preferred group of compounds are compounds having a substituted piperidine residue, wherein the substitution is at the 3-position, where R′ is methyl, R₁′ is —H, R₂′ is -methyl or —Cl, n′, m′=0 or 1, X′ is —F with n′=1 and wherein the piperidine is substituted with one of the following residues: —COOH, —COOCH₃, —COOC₂H₅, —CH₂COOH, —CH₂COOCH₃, —CH₂COOCH₂CH₃, —CONH₂, —CONHCH₃, —CON(CH₃)₂, —CH₂CONHCH₃, —SO₂NH₂, —SO₂NHCH₃, or —CN (especially compounds 3, 6, 7, 8, 9, 12, 14, 15, 16, 17, 18, 20, 21, 23, 24, 27, 28, 32, 33, 41, 46, 47, 48, 50, 52, 53, 66, 67, 69, 72, 76, 78, 84, 86, 87, 100, 101, 102, 103, 104, 105, 106, 107, 108, 111, 122 and 128 according to Table 1). An even more preferred subgroup of this group are compounds where R′ is (R)-methyl (especially compounds 14, 23, 27, 28, 32, 100, 101, 102, 103, 104, 105, 106, 107, 108, 111, 122 and 128 according to Table 1).

A more preferred subgroup are compounds having a substituted piperidine residue substituted with —COOH at the 3-position and wherein R′ is (R)-methyl (especially compounds 14, 23, 27 and 108 according to Table 1), or with —CH₂COOH at the 3-position and wherein R′ is (R)-methyl (especially compounds 102, 103, 106 and 107 according to Table 1).

Another more preferred subgroup are compounds, wherein R′ is (R)-methyl and having a substituted piperidine residue, wherein the substitution is at the 3-position, substituted with —COOR₅′ or —CH₂COOR₅′, with R₅′ is 2-morpholinoethyl (especially compounds 119 and 125 according to Table 1), or with R₅′ is -isopropyl (compounds 117 and 123 according to Table 1), -tertbutyl (especially compounds 118 and 124 according to Table 1), n-heptyl (especially compounds 120 and 126 according to Table 1), -isopropoxycarbonyloxymethyl (especially compounds 121 and 127 according to Table 1), or with —CONHCH₃ or —CH₂CONHCH₃ (especially compounds 122 and 128 according to Table 1).

An especially preferred group of compounds are compounds having a substituted piperidine residue where R′ is (R)-methyl, R₁′ is —H, R₂′ is -methyl, or —C₁, preferably —Cl, X′ is —F, n′=1 and wherein the piperidine is with —COOH, substituted —CH₂COOH, —CONHCH₃, —CH₂CONHCH₃, —SO₂NH₂, —SO₂NHCH₃, or —CN at the 3-position (especially compounds 14, 104, 105, 106, 107, 108, 122 and 128 according to Table 1). An especially preferred subgroup of this group are compounds with R₂′ is —Cl, X′ is —F and n′=1 (especially compounds 14, 104, 105, 106, 107, 108 122 and 128 according to Table 1).

A more preferred subgroup are compounds, wherein X′ is at the para-position, having a substituted piperidine residue substituted with —COOH at the 3-position and wherein R′ is (R)-methyl (especially compounds 14 and 108 according to Table 1), or with —CH₂COOH at the 3-position and wherein R′ is (R)-methyl (especially compounds 106 and 107 according to Table 1).

Another more preferred subgroup are compounds, wherein X is at the para-position, wherein R′ is (R)-methyl and having a substituted piperidine residue substituted with —COOR₅′ or —CH₂COOR₅′ at the 3-position, with R₅′ is 2-morpholinoethyl (especially compounds 119 and 125 according to Table 1), or with R₅′ is -isopropyl (compounds 117 and 123 according to Table 1), -tertbutyl (especially compounds 118 and 124 according to Table 1), n-heptyl (especially compounds 120 and 126 according to Table 1), -isopropoxycarbonyloxymethyl (especially compounds 121 and 127 according to Table 1), or —CONHCH₃ or —CH₂CONHCH₃ at the 3-position (especially compounds 122 and 128 according to Table 1).

An especially preferred group of compounds are compounds, wherein X′ is at the para-position, having a substituted piperidine residue where R′ is (R)-methyl, R₁′ is —H, R₂′ is -methyl, or —Cl, preferably —Cl, X′ is —F, n′=1 and wherein the piperidine is substituted with —COOH, —CH₂COOH, —CONHCH₃, —SO₂NH₂, —SO₂NHCH₃, or —CN at the 3-position (especially compounds 14, 106, 107, 108, 122 and 128 according to Table 1). An especially preferred subgroup of this group concerns compounds with R₂′ is —Cl, X′ is —F and n′=1 (especially compounds 14, 106, 107, 108, 122 and 128 according to Table 1).

Another specific subset of compounds concerns compounds selected from Table 1, or a pharmaceutically or veterinary acceptable salt, hydrate or solvate thereof.

TABLE 1
Exemplary compounds of Formula (I)

| Ex | IUPAC Name |
|---|---|
| 1 | 7-[1-methyl-2-oxo-2-(1-piperidyl)ethoxy]-4-(o-tolyl)chromen-2-one |
| 2 | 4-(2-chlorophenyl)-7-[1-methyl-2-oxo-2-(1-piperidyl)ethoxy]chromen-2-one |
| 3 | (3S)-1-[2-[4-(o-tolyl)-2-oxo-chromen-7-yl]oxypropanoyl]piperidine-3-carboxylic acid |
| 4 | 2-[4-(o-tolyl)-2-oxo-chromen-7-yl]oxy-N-(2-pyridyl)propanamide |
| 5 | 7-(1-methyl-2-oxo-2-pyrrolidin-1-yl-ethoxy)-4-(o-tolyl)chromen-2-one |
| 6 | methyl 1-[2-[4-(o-tolyl)-2-oxo-chromen-7-yl]oxypropanoyl]piperidine-3-carboxylate |
| 7 | methyl 1-[2-[4-(2-chlorophenyl)-2-oxo-chromen-7-yl]oxypropanoyl]piperidine-3- |
| | carboxylate |
| 8 | 1-[2-[4-(o-tolyl)-2-oxo-chromen-7-yl]oxypropanoyl]piperidine-3-carboxylic acid |
| 9 | 1-[2-[4-(2-chlorophenyl)-2-oxo-chromen-7-yl]oxypropanoyl]piperidine-3-carboxylic acid |
| 10 | 1-[2-[4-(o-tolyl)-2-oxo-chromen-7-yl]oxypropanoyl]piperidine-3-sulfonic acid |
| 11 | 1-[2-[4-(2-chlorophenyl)-2-oxo-chromen-7-yl]oxypropanoyl]piperidine-3-sulfonic acid |
| 12 | 1-[2-[4-(o-tolyl)-2-oxo-chromen-7-yl]oxypropanoyl]piperidine-3-sulfonamide |
| 13 | 4-(2-chlorophenyl)-7-(1-methyl-2-oxo-2-pyrrolidin-1-yl-ethoxy)chromen-2-one |
| 14 | (3S)-1-[(2R)-2-[4-(2-chloro-4-fluoro-phenyl)-2-oxo-chromen-7- |
| | yl]oxypropanoyl]piperidine-3-carboxylic acid |
| 15 | 1-[2-[4-(2-chlorophenyl)-2-oxo-chromen-7-yl]oxypropanoyl]-N-methyl-piperidine-3- |
| | carboxamide |
| 16 | ethyl (3S)-1-[2-[4-(2-chlorophenyl)-2-oxo-chromen-7-yl]oxypropanoyl]piperidine-3- |
| | carboxylate |
| 17 | ethyl (3S)-1-[2-[4-(o-tolyl)-2-oxo-chromen-7-yl]oxypropanoyl]piperidine-3-carboxylate |
| 18 | N-methyl-1-[2-[4-(o-tolyl)-2-oxo-chromen-7-yl]oxypropanoyl]piperidine-3-sulfonamide |
| 19 | N-[4-(2-hydroxyethyl)phenyl]-2-[4-(o-tolyl)-2-oxo-chromen-7-yl]oxy-propanamide |
| 20 | 1-[2-[4-(2-chlorophenyl)-2-oxo-chromen-7-yl]oxypropanoyl]-N-methyl-piperidine-3- |
| | sulfonamide |
| 21 | N-methyl-1-[2-[4-(o-tolyl)-2-oxo-chromen-7-yl]oxypropanoyl]piperidine-3-carboxamide |
| 22 | (3S)-1-[2-[4-(o-tolyl)-2-oxo-chromen-7-yl]oxypropanoyl]pyrrolidine-3-carboxylic acid |
| 23 | (3S)-1-[(2R)-2-[4-(2-chlorophenyl)-2-oxo-chromen-7-yl]oxypropanoyl]piperidine-3- |
| | carboxylic acid |
| 24 | 1-[2-[4-(2-chlorophenyl)-2-oxo-chromen-7-yl]oxypropanoyl]piperidine-3-sulfonamide |
| 25 | N-cyclopropyl-2-[4-(o-tolyl)-2-oxo-chromen-7-yl]oxy-propanamide |
| 26 | 2-[4-(2-chlorophenyl)-2-oxo-chromen-7-yl]oxy-N-cyclopropyl-propanamide |
| 27 | (3S)-1-[(2R)-2-[4-(o-tolyl)-2-oxo-chromen-7-yl]oxypropanoyl]piperidine-3-carboxylic acid |
| 28 | (3S)-1-[(2R)-2-[4-(2-chlorophenyl)-2-oxo-chromen-7-yl]oxypropanoyl]piperidine-3- |
| | carbonitrile |
| 29 | (3S)-1-[2-[4-(2-chlorophenyl)-2-oxo-chromen-7-yl]oxypropanoyl]pyrrolidine-3-carboxylic |
| | acid |
| 30 | 7-[(1R)-1-methyl-2-oxo-2-(1-piperidyl)ethoxy]-4-(o-tolyl)chromen-2-one |
| 31 | 2-[4-(2-chlorophenyl)-2-oxo-chromen-7-yl]oxy-N-(2-pyridyl)propanamide |
| 32 | (3S)-1-[(2R)-2-[4-(o-tolyl)-2-oxo-chromen-7-yl]oxypropanoyl]piperidine-3-carbonitrile |
| 33 | (3S)-1-[2-[4-(2-chlorophenyl)-2-oxo-chromen-7-yl]oxypropanoyl]piperidine-3-carboxylic |
| | acid |
| 34 | 4-(2-chlorophenyl)-7-[(1R)-1-methyl-2-oxo-2-(1-piperidyl)ethoxy]chromen-2-one |
| 35 | 2-[4-(2-chlorophenyl)-2-oxo-chromen-7-yl]oxy-N-[4-(2-hydroxyethyl)phenyl]propanamide |
| 36 | (2R)-N-isopropyl-2-[4-(o-tolyl)-2-oxo-chromen-7-yl]oxy-propanamide |
| 37 | 1-[2-[4-(2-chloro-4-fluoro-phenyl)-2-oxo-chromen-7-yl]oxypropanoyl]-3-methyl- |
| | piperidine-3-carboxylic acid |
| 38 | N,N-dimethyl-2-[4-(o-tolyl)-2-oxo-chromen-7-yl]oxy-propanamide |
| 39 | 4-(2-chlorophenyl)-7-[1-methyl-2-oxo-2-[3-(2H-tetrazol-5-yl)-1-piperidyl]ethoxy]chromen- |
| | 2-one |
| 40 | ethyl 2-[4-(2-chlorophenyl)-2-oxo-chromen-7-yl]oxypropanoate |
| 41 | 1-[2-[4-(o-tolyl)-2-oxo-chromen-7-yl]oxypropanoyl]piperidine-3-carbonitrile |
| 42 | 7-[1-methyl-2-oxo-2-[3-(2H-tetrazol-5-yl)-1-piperidyl]ethoxy]-4-(o-tolyl)chromen-2-one |
| 43 | 3-methyl-1-[2-[4-(o-tolyl)-2-oxo-chromen-7-yl]oxypropanoyl]piperidine-3-carboxylic acid |
| 44 | (2R)-N,N-dimethyl-2-[4-(o-tolyl)-2-oxo-chromen-7-yl]oxy-propanamide |
| 45 | (2R)-2-[4-(2-chlorophenyl)-2-oxo-chromen-7-yl]oxy-N,N-dimethyl-propanamide |
| 46 | (3R)-1-[2-[4-(2-chlorophenyl)-2-oxo-chromen-7-yl]oxypropanoyl]piperidine-3-carboxylic |
| | acid |
| 47 | (3S)-1-[2-[4-(2-chloro-4-fluoro-phenyl)-2-oxo-chromen-7-yl]oxypropanoyl]piperidine-3- |
| | carboxylic acid |
| 48 | 1-[2-[4-(2-chlorophenyl)-2-oxo-chromen-7-yl]oxypropanoyl]piperidine-3-carbonitrile |
| 49 | 2-[4-(2-chloro-3-fluoro-phenyl)-2-oxo-chromen-7-yl]oxy-N-isopropyl-propanamide |
| 50 | (3S)-1-[2-[4-(2-chloro-3-fluoro-phenyl)-2-oxo-chromen-7-yl]oxypropanoyl]piperidine-3- |
| | carboxylic acid |
| 51 | 2-[4-(2-chlorophenyl)-2-oxo-chromen-7-yl]oxy-N-methyl-propanamide |
| 52 | 1-[2-[4-(2-chloro-3-fluoro-phenyl)-2-oxo-chromen-7-yl]oxypropanoyl]piperidine-3- |
| | sulfonamide |
| 53 | (3R)-1-[2-[4-(o-tolyl)-2-oxo-chromen-7-yl]oxypropanoyl]piperidine-3-carboxylic acid |
| 54 | 1-[2-[4-(2-chlorophenyl)-2-oxo-chromen-7-yl]oxypropanoyl]-3-methyl-piperidine-3- |
| | carboxylic acid |
| 55 | isopropyl (2R)-2-[4-(o-tolyl)-2-oxo-chromen-7-yl]oxypropanoate |
| 56 | (2R)-2-[4-(2-chlorophenyl)-2-oxo-chromen-7-yl]oxy-N-isopropyl-propanamide |
| 57 | ethyl 2-[4-(2-chloro-3-fluoro-phenyl)-2-oxo-chromen-7-yl]oxypropanoate |
| 58 | ethyl 2-[4-(o-tolyl)-2-oxo-chromen-7-yl]oxypropanoate |
| 59 | 2-[1-[2-[4-(2-chlorophenyl)-2-oxo-chromen-7-yl]oxypropanoyl]-4-piperidyl]acetic acid |
| 60 | 2-[1-[2-[4-(o-tolyl)-2-oxo-chromen-7-yl]oxypropanoyl]-4-piperidyl]acetic acid |
| 61 | 2-[4-(2-chloro-3-fluoro-phenyl)-2-oxo-chromen-7-yl]oxy-N-cyclopropyl-propanamide |
| 62 | N-isopropyl-2-[4-(o-tolyl)-2-oxo-chromen-7-yl]oxy-propanamide |
| 63 | 1-[2-[4-(o-tolyl)-2-oxo-chromen-7-yl]oxypropanoyl]pyrrolidine-3-sulfonamide |
| 64 | isopropyl (2R)-2-[4-(2-chlorophenyl)-2-oxo-chromen-7-yl]oxypropanoate |
| 65 | 2-[4-(2-chloro-4-fluoro-phenyl)-2-oxo-chromen-7-yl]oxy-N-isopropyl-propanamide |
| 66 | 2-[1-[2-[4-(2-chlorophenyl)-2-oxo-chromen-7-yl]oxypropanoyl]-3-piperidyl]acetic acid |
| 67 | (3R)-1-[2-[4-(2-chlorophenyl)-2-oxo-chromen-7-yl]oxypropanoyl]-N,N-dimethyl- |
| | piperidine-3-carboxamide |
| 68 | 2-[4-(2-chloro-3-fluoro-phenyl)-2-oxo-chromen-7-yl]oxy-N-ethyl-propanamide |
| 69 | 1-[2-[4-(2-chloro-4-fluoro-phenyl)-2-oxo-chromen-7-yl]oxypropanoyl]piperidine-3- |
| | sulfonamide |
| 70 | 2-[4-(2-chlorophenyl)-2-oxo-chromen-7-yl]oxy-N-isopropyl-propanamide |
| 71 | 7-[2-(4,4-difluoro-1-piperidyl)-1-methyl-2-oxo-ethoxy]-4-(o-tolyl)chromen-2-one |
| 72 | 2-[1-[2-[4-(o-tolyl)-2-oxo-chromen-7-yl]oxypropanoyl]-3-piperidyl]acetic acid |
| 73 | 7-[1-methyl-2-[3-(methylsulfonimidoyl)-1-piperidyl]-2-oxo-ethoxy]-4-(o-tolyl)chromen-2- |
| | one |
| 74 | 4-(2-chlorophenyl)-7-[2-(4,4-difluoro-1-piperidyl)-1-methyl-2-oxo-ethoxy]chromen-2-one |
| 75 | ethyl 1-[2-[4-(2-chlorophenyl)-2-oxo-chromen-7-yl]oxypropanoyl]-3-methyl-piperidine-3- |
| | carboxylate |
| 76 | (3S)-1-[2-[4-(2-chlorophenyl)-2-oxo-chromen-7-yl]oxypropanoyl]-N,N-dimethyl- |
| | piperidine-3-carboxamide |
| 77 | 1-[2-[4-(2-chlorophenyl)-2-oxo-chromen-7-yl]oxypropanoyl]pyrrolidine-3-sulfonamide |
| 78 | (3R)-N,N-dimethyl-1-[2-[4-(o-tolyl)-2-oxo-chromen-7-yl]oxypropanoyl]piperidine-3- |
| | carboxamide |
| 79 | 4-(2-chlorophenyl)-7-[1-methyl-2-[3-(methylsulfonimidoyl)-1-piperidyl]-2-oxo- |
| | ethoxy]chromen-2-one |
| 80 | methyl 2-[1-[2-[4-(2-chlorophenyl)-2-oxo-chromen-7-yl]oxypropanoyl]-4-piperidyl]acetate |
| 81 | ethyl 2-[4-(2-bromophenyl)-2-oxo-chromen-7-yl]oxypropanoate |
| 82 | ethyl 2-[4-(2-chloro-4-fluoro-phenyl)-2-oxo-chromen-7-yl]oxypropanoate |
| 83 | methyl 2-[1-[2-[4-(o-tolyl)-2-oxo-chromen-7-yl]oxypropanoyl]-4-piperidyl]acetate |
| 84 | (3S)-N,N-dimethyl-1-[2-[4-(o-tolyl)-2-oxo-chromen-7-yl]oxypropanoyl]piperidine-3- |
| | carboxamide |
| 85 | N-ethyl-2-[4-(o-tolyl)-2-oxo-chromen-7-yl]oxy-propanamide |
| 86 | ethyl 2-[1-[2-[4-(2-chlorophenyl)-2-oxo-chromen-7-yl]oxypropanoyl]-3-piperidyl]acetate |
| 87 | ethyl 2-[1-[2-[4-(o-tolyl)-2-oxo-chromen-7-yl]oxypropanoyl]-3-piperidyl]acetate |
| 88 | ethyl 3-methyl-1-[2-[4-(o-tolyl)-2-oxo-chromen-7-yl]oxypropanoyl]piperidine-3- |
| | carboxylate |
| 89 | 2-[4-(2-bromophenyl)-2-oxo-chromen-7-yl]oxy-N,N-dimethyl-propanamide |
| 90 | 2-[4-(2-chloro-4-fluoro-phenyl)-2-oxo-chromen-7-yl]oxy-N-cyclopropyl-propanamide |
| 91 | 2-[4-(2-chloro-4-fluoro-phenyl)-2-oxo-chromen-7-yl]oxy-N-ethyl-propanamide |
| 92 | isopropyl 2-[4-(2-bromophenyl)-2-oxo-chromen-7-yl]oxypropanoate |
| 93 | 2-[4-(2-chlorophenyl)-2-oxo-chromen-7-yl]oxy-N,N-dimethyl-propanamide |
| 94 | 2-[4-(2-chloro-3-fluoro-phenyl)-2-oxo-chromen-7-yl]oxypropanoic acid |
| 95 | ethyl 1-[2-[4-(2-chloro-4-fluoro-phenyl)-2-oxo-chromen-7-yl]oxypropanoyl]-3-methyl- |
| | piperidine-3-carboxylate |
| 96 | 2-[4-(2-chlorophenyl)-2-oxo-chromen-7-yl]oxypropanoic acid |
| 97 | 2-[4-(o-tolyl)-2-oxo-chromen-7-yl]oxypropanoic acid |
| 98 | 2-[4-(2-chlorophenyl)-2-oxo-chromen-7-yl]oxy-N-ethyl-propanamide |
| 99 | 2-[4-(2-chloro-4-fluoro-phenyl)-2-oxo-chromen-7-yl]oxypropanoic acid |
| 100 | methyl 2-[(3S)-1-[(2R)-2-[4-(2-chlorophenyl)-2-oxo-chromen-7-yl]oxypropanoyl]-3- |
| | piperidyl]acetate |
| 101 | methyl 2-[(3R)-1-[(2R)-2-[4-(2-chlorophenyl)-2-oxo-chromen-7-yl]oxypropanoyl]-3- |
| | piperidyl]acetate |
| 102 | 2-[(3S)-1-[(2R)-2-[4-(2-chlorophenyl)-2-oxo-chromen-7-yl]oxypropanoyl]-3- |
| | piperidyl]acetic acid |
| 103 | 2-[(3R)-1-[(2R)-2-[4-(2-chlorophenyl)-2-oxo-chromen-7-yl]oxypropanoyl]-3- |
| | piperidyl]acetic acid |
| 104 | methyl 2-[(3R)-1-[(2R)-2-[4-(2-chloro-4-fluoro-phenyl)-2-oxo-chromen-7- |
| | yl]oxypropanoyl]-3-piperidyl]acetate |
| 105 | methyl 2-[(3S)-1-[(2R)-2-[4-(2-chloro-4-fluoro-phenyl)-2-oxo-chromen-7- |
| | yl]oxypropanoyl]-3-piperidyl]acetate |
| 106 | 2-[(3R)-1-[(2R)-2-[4-(2-chloro-4-fluoro-phenyl)-2-oxo-chromen-7-yl]oxypropanoyl]-3- |
| | piperidyl]acetic acid |
| 107 | 2-[(3S)-1-[(2R)-2-[4-(2-chloro-4-fluoro-phenyl)-2-oxo-chromen-7-yl]oxypropanoyl]-3- |
| | piperidyl]acetic acid |
| 108 | (3R)-1-[(2R)-2-[4-(2-chloro-4-fluoro-phenyl)-2-oxo-chromen-7- |
| | yl]oxypropanoyl]piperidine-3-carboxylic acid |
| 109 | ethyl (3S)-1-[2-[4-(2-chlorophenyl)-2-oxo-chromen-7-yl]oxybutanoyl]piperidine-3- |
| | carboxylate |
| 110 | (3S)-1-[2-[4-(2-chlorophenyl)-2-oxo-chromen-7-yl]oxybutanoyl]piperidine-3-carboxylic |
| | acid |
| 111 | ethyl (3S)-1-[(2R)-2-[4-(2-chloro-4-fluoro-phenyl)-2-oxo-chromen-7-yl]oxypropanoyl] |
| | piperidine-3-carboxylate |
| 112 | 2-[4-(2-chlorophenyl)-2-oxo-chromen-7-yl]oxy-2-methyl-propanoic acid |
| 113 | ethyl 2-[4-(2-chlorophenyl)-2-oxo-chromen-7-yl]oxy-2-methyl-propanoate |
| 114 | 2-[4-(2-chlorophenyl)-2-oxo-chromen-7-yl]oxy-N-isopropyl-2-methyl-propanamide |
| 115 | N-isopropyl-2-methyl-2-[4-(o-tolyl)-2-oxo-chromen-7-yl]oxy-propanamide |
| 116 | (3S)-1-[2-[4-(2-chlorophenyl)-2-oxo-chromen-7-yl]oxy-2-methyl-propanoyl]piperidine-3- |
| | carboxylic acid |
| 117 | isopropyl (3S)-1-[(2R)-2-[4-(2-chloro-4-fluoro-phenyl)-2-oxo-chromen-7-yl]oxypropanoyl] |
| | piperidine-3-carboxylate |
| 118 | tert-butyl (3S)-1-[(2R)-2-[4-(2-chloro-4-fluoro-phenyl)-2-oxo-chromen-7-yl]oxypropanoyl] |
| | piperidine-3-carboxylate |
| 119 | 2-morpholinoethyl (3S)-1-[(2R)-2-[4-(2-chloro-4-fluoro-phenyl)-2-oxo-chromen-7-yl] |
| | oxypropanoyl]piperidine-3-carboxylate |
| 120 | heptyl (3S)-1-[(2R)-2-[4-(2-chloro-4-fluoro-phenyl)-2-oxo-chromen-7-yl]oxypropanoyl] |
| | piperidine-3-carboxylate |
| 121 | isopropoxycarbonyloxymethyl (3S)-1-[(2R)-2-[4-(2-chloro-4-fluoro-phenyl)-2-oxo- |
| | chromen-7-yl]oxypropanoyl]piperidine-3-carboxylate |
| 122 | (3S)-N-methyl-1-[(2R)-2-[4-(2-chloro-4-fluoro-phenyl)-2-oxo-chromen-7-yl]oxypropanoyl |
| | piperidine-3-carboxamide |
| 123 | isopropyl 2-[(3R)-1-[(2R)-2-[4-(2-chloro-4-fluoro-phenyl)-2-oxo-chromen-7- |
| | yl]oxypropanoyl]-3-piperidyl]acetate |
| 124 | tert-butyl 2-[(3R)-1-[(2R)-2-[4-(2-chloro-4-fluoro-phenyl)-2-oxo-chromen-7- |
| | yl]oxypropanoyl] -3-piperidyl]acetate |
| 125 | 2-morpholinoethyl 2-[(3R)-1-[(2R)-2-[4-(2-chloro-4-fluoro-phenyl)-2-oxo-chromen-7-yl] |
| | oxypropanoyl]-3-piperidyl]acetate |
| 126 | heptyl 2-[(3R)-1-[(2R)-2-[4-(2-chloro-4-fluoro-phenyl)-2-oxo-chromen-7-yl]oxypropanoyl]- |
| | 3-piperidyl]acetate |
| 127 | isopropoxycarbonyloxymethyl 2-[(3R)-1-[(2R)-2-[4-(2-chloro-4-fluoro-phenyl)-2-oxo- |
| | chromen-7-yl]oxypropanoyl]-3-piperidyl]acetate |
| 128 | 2-[(3R)-1-[(2R)-2-[4-(2-chloro-4-fluoro-phenyl)-2-oxo-chromen-7-yl]oxypropanoyl]-3- |
| | piperidyl]-N-methyl-acetamide |
| 129 | methyl 1-[(2R)-2-[4-(2-chloro-4-fluoro-phenyl)-2-oxo-chromen-7- |
| | yl]oxypropanoyl]piperidine-4-carboxylate |
| 130 | (2R)-2-[4-(2-chloro-4-fluoro-phenyl)-2-oxo-chromen-7-yl]oxy-N-[4- |
| | (hydroxymethyl)phenyl]propanamide |
| 131 | 1-[(2R)-2-[4-(2-chloro-4-fluoro-phenyl)-2-oxo-chromen-7-yl]oxypropanoyl]piperidine-4- |
| | carboxylic acid |
| 132 | (3S)-1-[(2R)-2-[4-(2-chloro-4-fluoro-phenyl)-2-oxo-chromen-7- |
| | yl]oxypropanoyl]piperidine-3-carboxamide |
| 133 | 2-[(3R)-1-[(2R)-2-[4-(2-chloro-4-fluoro-phenyl)-2-oxo-chromen-7-yl]oxypropanoyl]-3- |
| | piperidyl]acetamide |
| 134 | (3S)-1-[(2R)-2-[4-(2-ethylphenyl)-2-oxo-chromen-7-yl]oxypropanoyl]piperidine-3- |
| | carboxylic acid |
| 135 | methyl (2S)-4-[(2R)-2-[4-(2-chloro-4-fluoro-phenyl)-2-oxo-chromen-7- |
| | yl]oxypropanoyl]piperazine-2-carboxylate |
| 136 | methyl (2R)-4-[(2R)-2-[4-(2-chloro-4-fluoro-phenyl)-2-oxo-chromen-7- |
| | yl]oxypropanoyl]piperazine-2-carboxylate |
| 137 | methyl 1-methyl-4-[rac-(2R)-2-[4-(2-chloro-4-fluoro-phenyl)-2-oxo-chromen-7- |
| | yl]oxypropanoyl]piperazine-2-carboxylate |
| 138 | (2S)-4-[(2R)-2-[4-(2-chloro-4-fluoro-phenyl)-2-oxo-chromen-7- |
| | yl]oxypropanoyl]piperazine-2-carboxylic acid |
| 139 | 3-[(2R)-2-[4-(2-chloro-4-fluoro-phenyl)-2-oxo-chromen-7- |
| | yl]oxypropanoyl]amino]benzoic acid |
| 140 | (2R)-2-[4-(2-chloro-4-fluoro-phenyl)-2-oxo-chromen-7-yl]oxy-N-[4-(2- |
| | methoxyethyl)phenyl]propanamide |
| 141 | 4-[[(2R)-2-[4-(2-chloro-4-fluoro-phenyl)-2-oxo-chromen-7- |
| | yl]oxypropanoyl]amino]benzoic acid |
| 142 | methyl 2-[(3R)-1-[(2R)-2-[4-(2-chloro-4-fluoro-phenyl)-2-oxo-chromen-7- |
| | yl]oxypropanoyl]pyrrolidin-3-yl]acetate |
| 143 | methyl 5-[[(2R)-2-[4-(2-chloro-4-fluoro-phenyl)-2-oxo-chromen-7- |
| | yl]oxypropanoyl]amino]pyridine-3-carboxylate |
| 144 | methyl 2-[(3S)-1-[(2R)-2-[4-(2-chloro-4-fluoro-phenyl)-2-oxo-chromen-7- |
| | yl]oxypropanoyl]pyrrolidin-3-yl]acetate |
| 145 | 2-[(3R)-1-[(2R)-2-[4-(2-chloro-4-fluoro-phenyl)-2-oxo-chromen-7- |
| | yl]oxypropanoyl]pyrrolidin-3-yl]acetic acid |
| 146 | methyl 2-[(2R)-2-[4-(2-chloro-4-fluoro-phenyl)-2-oxo-chromen-7- |
| | yl]oxypropanoyl]amino]pyridine-4-carboxylate |
| 147 | 2-[(3S)-1-[(2R)-2-[4-(2-chloro-4-fluoro-phenyl)-2-oxo-chromen-7- |
| | yl]oxypropanoyl]pyrrolidin-3-yl]acetic acid |
| 148 | 5-[[(2R)-2-[4-(2-chloro-4-fluoro-phenyl)-2-oxo-chromen-7- |
| | yl]oxypropanoyl]amino]pyridine-3-carboxylic acid |
| 149 | methyl 6-[[(2R)-2-[4-(2-chloro-4-fluoro-phenyl)-2-oxo-chromen-7- |
| | yl]oxypropanoylamino]pyridine-2-carboxylate |
| 150 | (2R)-4-[(2R)-2-[4-(2-chloro-4-fluoro-phenyl)-2-oxo-chromen-7- |
| | yl]oxypropanoyl]piperazine-2-carboxylic acid |
| 151 | 6-[(2R)-2-[4-(2-chloro-4-fluoro-phenyl)-2-oxo-chromen-7- |
| | yl]oxypropanoyl]amino]pyridine-2-carboxylic acid |
| 152 | 2-[[(2R)-2-[4-(2-chloro-4-fluoro-phenyl)-2-oxo-chromen-7- |
| | yl]oxypropanoyl]amino]pyridine-4-carboxylic acid |
| 153 | methyl 5-[[(2R)-2-[4-(2-chloro-4-fluoro-phenyl)-2-oxo-chromen-7- |
| | yl]oxypropanoyl]amino]pyridine-2-carboxylate |
| 154 | 5-[(2R)-2-[4-(2-chloro-4-fluoro-phenyl)-2-oxo-chromen-7- |
| | yl]oxypropanoyl]amino]pyridine-2-carboxylic acid |
| 155 | 6-[[(2R)-2-[4-(2-chloro-4-fluoro-phenyl)-2-oxo-chromen-7- |
| | yl]oxypropanoyl]amino]pyridine-3-carboxylic acid |
| 156 | methyl 4-[[(2R)-2-[4-(2-chloro-4-fluoro-phenyl)-2-oxo-chromen-7- |
| | yl]oxypropanoyl]amino]pyridine-2-carboxylate |
| 157 | methyl (3S)-1-[(2R)-2-[4-(2-chloro-4-fluoro-phenyl)-2-oxo-chromen-7- |
| | yl]oxypropanoyl]pyrrolidine-3-carboxylate |
| 158 | (3S)-1-[(2R)-2-[4-(4-fluoro-2-methyl-phenyl)-2-oxo-chromen-7- |
| | yl]oxypropanoyl]piperidine-3-carboxylic acid |
| 159 | (3S)-1-[(2R)-2-[4-(2-chloro-4-fluoro-phenyl)-2-oxo-chromen-7- |
| | yl]oxypropanoyl]pyrrolidine-3-carboxylic acid |
| 160 | 4-[(2R)-2-[4-(2-chloro-4-fluoro-phenyl)-2-oxo-chromen-7- |
| | yl]oxypropanoyl]amino]pyridine-2-carboxylic acid |
| 161 | 1-methyl-4-[rac-(2R)-2-[4-(2-chloro-4-fluoro-phenyl)-2-oxo-chromen-7- |
| | yl]oxypropanoyl]piperazine-2-carboxylic acid |
| 162 | (3S)-1-[(2R)-2-[4-(2-cyanophenyl)-2-oxo-chromen-7-yl]oxypropanoyl]piperidine-3- |
| | carboxylic acid |
| 163 | (3S)-1-[(2R)-2-[4-(2,6-dichlorophenyl)-2-oxo-chromen-7-yl]oxypropanoyl]piperidine-3- |
| | carboxylic acid |
| 164 | 4-(2-chloro-4-fluoro-phenyl)-7-[(1R)-1-methyl-2-oxo-2-(4-prop-2-enoylpiperazin-1- |
| | yl)ethoxy]chromen-2-one |
| 165 | N-[(3S)-1-[(2R)-2-[4-(2-chloro-4-fluoro-phenyl)-2-oxo-chromen-7-yl]oxypropanoyl]-3- |
| | piperidyl]prop-2-enamide |
| 166 | 7-[(1R)-2-[4-(2-chloroacetyl)piperazin-1-yl]-1-methyl-2-oxo-ethoxy]-4-(2-chloro-4-fluoro- |
| | phenyl)chromen-2-one |
| 167 | 2-chloro-N-[(3S)-1-[(2R)-2-[4-(2-chloro-4-fluoro-phenyl)-2-oxo-chromen-7- |
| | yl]oxypropanoyl]-3-piperidyl]acetamide |
| 168 | 4-(2-chloro-4-fluoro-phenyl)-7-[(1R)-1-methyl-2-oxo-2-(4-propanoylpiperazin-1- |
| | yl)ethoxy]chromen-2-one |
| 169 | N-[(3S)-1-[(2R)-2-[4-(2-chloro-4-fluoro-phenyl)-2-oxo-chromen-7-yl]oxypropanoyl]-3- |
| | piperidyl]propanamide |
| 170 | rac-(3S)-1-[2-[4-(2-chlorophenyl)-2-oxo-pyrano[2,3-b]pyridin-7- |
| | yl]oxypropanoyl]piperidine-3-carboxylic acid |
| 171 | tert-butyl rac-(3S)-1-[2-[4-(2-chlorophenyl)-2-oxo-pyrano[2,3-b]pyridin-7- |
| | yl]oxypropanoyl]piperidine-3-carboxylate |
| 172 | (3S)-1-[(2R)-2-[4-(2-chlorophenyl)-2-oxo-pyrano[2,3-b]pyridin-7- |
| | yl]oxypropanoyl]piperidine-3-carboxylic acid |
| 173 | (3S)-1-[(2R)-2-[4-(2-chloro-4-fluoro-phenyl)-2-oxo-pyrano[2,3-b]pyridin-7- |
| | yl]oxypropanoyl]piperidine-3-carboxylic acid |
| 174 | (3S)-1-[(2R)-2-[4-(3-methyl-2-thienyl)-2-oxo-chromen-7-yl]oxypropanoyl]piperidine-3- |
| | carboxylic acid |
| 175 | ethyl 2-[4-(2-fluorophenyl)-2-oxo-chromen-7-yl]oxypropanoate |
| 176 | ethyl 2-[4-(2,6-difluorophenyl)-2-oxo-chromen-7-yl]oxypropanoate |
| 177 | 7-[(1R)-1-methyl-2-oxo-2-(1-piperidyl)ethoxy]-4-(3-methyl-2-thienyl)chromen-2-one |
| 178 | (2R)-N-isopropyl-2-[4-(3-methyl-2-thienyl)-2-oxo-chromen-7-yl]oxy-propanamide |
| 179 | (2R)-N,N-dimethyl-2-[4-(3-methyl-2-thienyl)-2-oxo-chromen-7-yl]oxy-propanamide |
| 180 | ethyl 2-[(3R)-1-[(2R)-2-[4-(3-methyl-2-thienyl)-2-oxo-chromen-7-yl]oxypropanoyl]-3- |
| | piperidyl]acetate |
| 181 | 2-[(3R)-1-[(2R)-2-[4-(3-methyl-2-thienyl)-2-oxo-chromen-7-yl]oxypropanoyl]-3- |
| | piperidyl]acetic acid |
| 182 | (3S)-1-[(2R)-2-[4-(4-methyl-3-thienyl)-2-oxo-chromen-7-yl]oxypropanoyl]piperidine-3- |
| | carboxylic acid |
| 183 | 7-[(1R)-1-methyl-2-oxo-2-(1-piperidyl)ethoxy]-4-(4-methyl-3-thienyl)chromen-2-one |
| 184 | 7-[1-methyl-2-oxo-2-(1-piperidyl)ethoxy]-4-(2-methyl-3-thienyl)chromen-2-one |
| 185 | rac-(3S)-1-[2-[4-(2-methyl-3-thienyl)-2-oxo-chromen-7-yl]oxypropanoyl]piperidine-3- |
| | carboxylic acid |

In one embodiment, according to the present invention, there is provided a composition wherein the IMT is a compound of the general formula (II) according to EP3598972. A compound of the general formula (II) may be

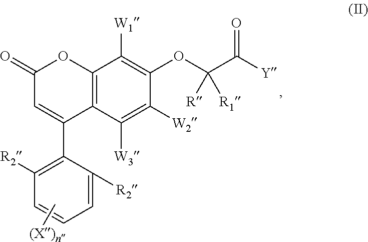

wherein
R′″ is —H or —C₁-C₄-alkyl;
R₁′″ is —H or -methyl;
R₂′″ is —H;
n′″=0, 1 or 2;
X′″ is -halogen, —C₁-C₄-alkyl, —OMe or —CN, preferably -halogen, —C₁-C₄-alkyl, or —CN, with n′″=1 or 2;
Y″ is —NR₃′″R₄′″ with
   R₃′″ is —H, or —C₁-C₄-alkyl, and
   R₄′″ is —H, —C₁-C₄-alkyl or —C₃-C₈-cycloalkyl;
      an unsubstituted or substituted pyridine residue;
      an unsubstituted or substituted pyridinylmethyl residue;
      an unsubstituted or substituted morpholinylethyl residue;
      an unsubstituted or substituted furanylmethyl residue;
      an unsubstituted or substituted phenyl residue;
      an unsubstituted or substituted benzyl residue;
      an unsubstituted or substituted phenethyl residue;
      the group

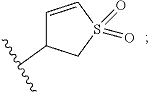 [SMILES: *C1C=CS(=O)(=O)C1]

or
the group

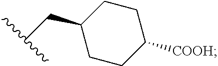 [SMILES: *C[C@H]1CC[C@H](C(=O)O)CC1]

or

Y″ is —NR₃′″R₄′″ with N, R₃′″ and R₄′″ forming an unsubstituted or substituted 5- or 6-membered saturated heterocycle, preferably an unsubstituted 5-membered saturated heterocycle, an unsubstituted or substituted 6-membered saturated heterocycle,

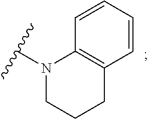 [SMILES: *N1CCCc2ccccc21]

or
Y″ is —OR₁₁′″, with R₁₁′″ is —H or —C₁-C₄-alkyl, phenyl, benzyl or 2-ethoxyethyl; and
W₁′″, W₂′″, and W₃′″ are identical or different, and are —H, -halogen, or —C₁-C₄-alkyl,
or a pharmaceutically or veterinary acceptable salt, hydrate or solvate thereof.

Further included are pharmaceutically or veterinary acceptable salts, hydrates or solvates of the compounds of formula (II) or its intermediate compounds disclosed herein. A pharmaceutically or veterinary acceptable salt can be an anionic counterion, e.g. an acetate, a bromide, camsylate, chloride, citrate, formate, fumarate, lactate, maleate, mesylate, nitrate, oxalate, phosphate, sulfate, tartrate, thiocyanate, or tosylate, or preferably a cationic counterion, e.g. ammonium, arginine, diethylamine, ethylenediamine, piperazine, potassium, sodium, or any other counter ion disclosed in Haynes et al. (2005). Some compounds of the invention contain one or more chiral centers due to the presence of asymmetric carbon atoms, which gives rise to stereoisomers, for example to diastereoisomers with R or S stereochemistry at each chiral center. The invention includes all such stereoisomers and diastereoisomers and mixtures thereof.

The compounds of general formula (II) or a pharmaceutically or veterinary acceptable salt, hydrate or solvate thereof, are useful as mitochondrial RNA polymerase (POLRMT) inhibitors and thereby inhibit mitochondrial DNA replication and/or mitochondrial transcription.

In the following, preferred groups of the compounds of general formula (II) of the present invention are described. The preferred groups constitute preferred embodiments of the compounds of general formula (II). Any combinations of the embodiments of the compounds of general formula (II) of the invention described herein are considered to be within the scope of the invention.

In one embodiment, the invention relates to a composition, wherein the IMT is a compound of formula (II) as defined above, wherein
R″ is —H or -methyl;
R₁′″ is —H; and
W₁′″, W₂′″, and W₃′″ are identical or different, and are —H or —Cl.

In another embodiment, the invention relates to a composition, wherein the IMT is a compound of formula (II) as defined above, wherein
W₁′″, W₂′″, and W₃′″ are identical and are —H.

In another embodiment, the invention relates to a composition, wherein the IMT is a compound of formula (II) as defined above, wherein
W₁′″ and W₃′″ are identical and are —H; and W₂′″ is —Cl.

In another embodiment, the invention relates to a composition, wherein the IMT is a compound of formula (II) as defined above, wherein
R′″ is -methyl; and
R₁′″ is —H.

In another embodiment, the invention relates to a composition, wherein the IMT is a compound of formula (II) as defined above, wherein
R′″ is —H and
R₁′″ is —H.

In another embodiment, the invention relates to a composition, wherein the IMT is a compound of formula (II) as defined above, wherein
n′″=1; and
X′″ is -halogen.

A preferred group of compounds are compounds of formula (II), wherein
R′″ is —H, -methyl or -ethyl;
R₁′″ is —H;
R₂′″ is —H;
n′″=0 or 1, preferably n′″=0;
X′″ is -halogen, —C₁-C₄-alkyl, —OMe or —CN, preferably -halogen, —C₁-C₄-alkyl, or —CN, with n′″=1;
Y″ is —NR₃′″R₄′″ with
   R₃′″ is —H, or —C₁-C₄-alkyl, preferably —H or -methyl, and
   R₄′″ is —H, —C₁-C₄-alkyl or —C₃-C₈-cycloalkyl;
      an unsubstituted or substituted pyridine residue;
      an unsubstituted or substituted pyridinylmethyl residue;
      an unsubstituted or substituted morpholinylethyl residue;
      an unsubstituted or substituted furanylmethyl residue;
      an unsubstituted or substituted phenyl residue;
      an unsubstituted or substituted benzyl residue;
      an unsubstituted or substituted phenethyl residue;
      the group

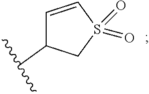 [SMILES: *C1C=CS(=O)(=O)C1]

or
the group

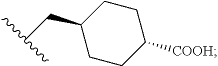 [SMILES: *C[C@H]1CC[C@H](C(=O)O)CC1]

or
Y″ is —NR₃′″R₄′″ with N, R₃′″ and R₄′″ forming an unsubstituted or substituted 5- or 6-membered saturated heterocycle, preferably an unsubstituted 5-membered saturated heterocycle, an unsubstituted or substituted 6-membered saturated heterocycle,

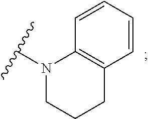 [SMILES: *N1CCCc2ccccc21]

or
Y″ is —OR₁₁′″, with R₁₁′″ is —H or —C₁-C₄-alkyl, phenyl, benzyl or 2-ethoxyethyl; and
W₁′″, W₂′″, and W₃′″ are identical or different, and are —H, -halogen, or —C₁-C₄-alkyl.

Examples for compounds of this group of compounds of Formula (II) are compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 and 99 according to Table 2.

An especially preferred group of compounds are compounds of formula (II), wherein n′″=0, i.e. compounds with an unsubstituted phenyl ring having the general formula (IIA). Thus, in one embodiment, the invention relates to compounds of the general formula (IIA)

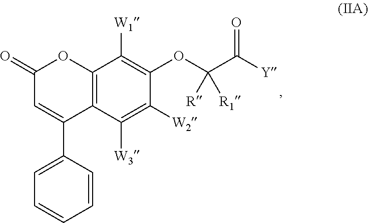

wherein
R′″ is —H or —C₁-C₄-alkyl;
R₁′″ is —H or -methyl;
Y″ is —NR₃′″R₄′″ with
   R₃′″ is —H, or —C₁-C₄-alkyl, and
   R₄′″ is —H, —C₁-C₄-alkyl or —C₃-C₈-cycloalkyl;
      an unsubstituted or substituted pyridine residue;
      an unsubstituted or substituted pyridinylmethyl residue;
      an unsubstituted or substituted morpholinylethyl residue;
      an unsubstituted or substituted furanylmethyl residue;
      an unsubstituted or substituted phenyl residue;
      an unsubstituted or substituted benzyl residue;
      an unsubstituted or substituted phenethyl residue;
      the group

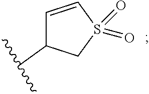 [SMILES: *C1C=CS(=O)(=O)C1]

or
the group

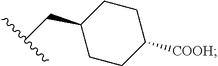 [SMILES: *C[C@H]1CC[C@H](C(=O)O)CC1]

or
Y″ is —NR₃′″R₄′″ with N, R₃′″ and R₄′″ forming an unsubstituted or substituted 5- or 6-membered saturated heterocycle,

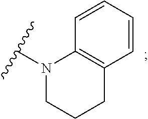 [SMILES: *N1CCCc2ccccc21]

or
Y″ is —OR₁₁′″, with R₁₁′″ is —H or —C₁-C₄-alkyl, phenyl, benzyl or 2-ethoxyethyl; and
W₁′″, W₂′″, and W₃′″ are identical or different, and are —H, -halogen, or —C₁-C₄-alkyl, or a pharmaceutically or veterinary acceptable salt, hydrate or solvate thereof.

Examples for compounds of formula (IIA) are compounds 1, 2, 3, 4, 5, 6, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 60, 61, 62, 63, 64, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 and 99 according to Table 2.

Another preferred group of compounds are compounds of formulae (II) and (IIA) as defined above, wherein
R″ is —H or -methyl;
R₁″ is —H; and
W₁″, W₂″, and W₃″ are identical or different, and are —H or —Cl.

Examples for compounds of this group of compounds of formula (II) are compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 72, 73, 74, 75, 76, 77, 78, 79, 80, 81, 82, 83, 84, 85, 86, 88, 89, 90, 92, 93, 95, 96, 98 and 99 according to Table 2.

Another preferred group of compounds of formulae (II) and (IIA) are compounds of formulae (II) and (IIA) as defined above, wherein W₁′″, W₂′″, and W₃′″ are identical and are —H. Examples for compounds of this group are compounds 1, 2, 3, 4, 5, 6, 7, 8, 9, 12, 14, 15, 16, 18, 19, 20, 21, 22, 23, 24, 25, 26, 29, 30, 31, 32, 33, 34, 35, 36, 37, 38, 39, 40, 41, 43, 44, 48, 51, 52, 53, 54, 55, 56, 60, 61, 62, 63, 64, 65, 66, 67, 68, 69, 70, 71, 72, 73, 74, 75, 76, 77, 81, 93 and 96 according to Table 2.

Another preferred group of compounds are compounds of formulae (II) and (IIA) as defined above, wherein W₁″ and W₃″ are identical and are —H; and W₂″ is —Cl. Examples for compounds of this group are compounds 10, 11, 13, 17, 27, 28, 42, 45, 46, 47, 49, 50, 57, 58, 59, 78, 79, 82, 83, 84, 85, 86, 88, 89, 90, 92, 95, 98 and 99 according to Table 2.

Another preferred group of compounds are compounds of formulae (II) and (IIA) as defined above, wherein R″ is -methyl; and R₁″ is —H. Examples for compounds of this group are compounds 1, 2, 3, 4, 5, 6, 7, 8, 9, 10, 11, 12, 13, 14, 15, 16, 17, 18, 19, 20, 21, 24, 25, 26, 27, 28, 29, 30, 31, 33, 34, 35, 36, 37, 38, 39, 41, 42, 50, 51, 52, 53, 55, 56, 58, 60, 61, 62, 63, 64, 65, 66, 67, 68, 69, 70, 72, 79, 80, 87, 89, 93, 96 and 97 according to Table 2.

Another preferred group of compounds are compounds of formulae (II) and (IIA) as defined above, wherein R″ is —H and R₁″ is —H. Examples for compounds of this group are compounds 22, 23, 40, 43, 44, 45, 46, 47, 48, 49, 54, 57, 59, 73, 74, 75, 76, 77, 78, 81, 82, 83, 84, 85, 86, 88, 90, 91, 92, 94, 95, 98 and 99 according to Table 2.

Another preferred group of compounds are compounds of formula (II) as defined above, wherein n″=1; and X″ is -halogen. Examples for compounds of this group are compounds 3, 4, 7, 8 and 41 according to Table 2.

A preferred group of compounds are compounds of formula (II), wherein
n″=0, 1 or 2,
X″ is halogen, or —CN, with n″=1 or 2;
Y″ is —NR₃″R₄″ with
   R₃″ is —H, or —C₁-C₄-alkyl, and
   R₄″ is —C₁-C₄-alkyl or —C₃-C₆-cycloalkyl; or an unsubstituted or substituted pyridine residue; or an unsubstituted or substituted phenyl residue;
Y″ is —NR₃″R₄″ with N, R₃″ and R₄″ forming an unsubstituted or substituted 5- or 6-membered saturated heterocycle; or
Y″ is —OR₁₁″, with R₁₁″ is —H or —C₁-C₄-alkyl, and
wherein R″, R₁″, R₂″, W₁″, W₂″, and W₃″ are as defined above.

Examples for compounds of this group of compounds of Formula (II) are compounds 1, 2, 3, 4, 5, 6, 7, 8, 9, 10, 11, 12, 13, 14, 15, 16, 17, 18, 19, 20, 21, 22, 23, 25, 26, 28, 30, 31, 32, 33, 34, 35, 36, 37, 38, 39, 40, 41, 43, 44, 45, 46, 47, 48, 49, 50, 51, 52, 53, 54, 55, 56, 57, 58, 60, 61, 62, 63, 64, 65, 66, 68, 69, 71, 72, 74, 76, 77, 80, 81, 82, 85, 87, 88, 91, 92, 94 and 97 according to Table 2. A preferred subgroup of these compounds are compounds, wherein n″=0, i.e. compounds 1, 2, 5, 6, 10, 11, 12, 13, 14, 15, 16, 17, 18, 19, 20, 21, 22, 23, 25, 26, 28, 30, 31, 32, 33, 34, 35, 36, 37, 38, 39, 40, 43, 44, 45, 46, 47, 48, 49, 50, 51, 52, 53, 54, 55, 56, 57, 58, 60, 61, 62, 63, 64, 66, 68, 69, 71, 72, 74, 76, 77, 80, 81, 82, 85, 87, 88, 91, 92, 94 and 97 according to Table 2. Another preferred subgroup of these compounds are compounds, wherein X″ is -halogen, preferably —C₁, —Br, or —F, or —CN, with n″=1 or 2, i.e. compounds 3, 4, 7, 8 and 41 according to Table 2.

Another preferred subgroup of these compounds are compounds of formula (II), wherein
n″=0, 1 or 2,
X″ is —Cl, —Br, or —F, or —CN
Y″ is —NR₃″R₄″ with
   R₃″ is —H, or -methyl, and
   R₄″ is -methyl, ethyl, -isopropyl or -cyclopropyl; or an unsubstituted or substituted pyridine residue; or an unsubstituted or substituted phenyl residue;
Y″ is —NR₃″R₄″ with N, R₃″ and R₄″ forming an unsubstituted or substituted 5- or 6-membered saturated heterocycle; or
Y″ is —OR₁₁″, with R₁₁″ is —H or —C₁-C₄-alkyl, and
wherein R″, R₁″, R₂″, W₁″, W₂″, and W₃″ are as defined above.

More preferred are these compounds, wherein R″ is —H and R₁″ is methyl and wherein n″=0 (especially compounds 13, 14, 21, 36, 37, 38 and 69 according to Table 2).

More preferred are these compounds, wherein the substituted phenyl residue is substituted at the para position.

More preferred are these compounds, wherein R₁₁″ is —H, -methyl, -ethyl or -isopropyl.

Another more preferred group of compounds are compounds of formula (II), wherein
R″ is —H;
R₁″ is -methyl;
wherein n″=0
Y″ is —NR₃″R₄″ with
   R₃″ is —H and
   R₄″ is an unsubstituted pyridine residue (for example compounds 6, 11, 22, 55 and 61 according to Table 2), or an unsubstituted pyridinylmethyl residue (for example compounds 59 and 99 according to Table 2), and
wherein R₂″, W₁″, W₂″, and W₃″ are as defined above.

Another more preferred group of compounds are compounds of formula (I), wherein Y″ is —NR₃″R₄″ with R₃″ is —H and R₄″ is an unsubstituted morpholinoethyl residue and wherein n″=0 (especially compounds 70, 79 and 83 according to Table 2), and wherein R″, R₁″, R₂″, W₁″, W₂″, and W₃″ are as defined above.

Another more preferred group of compounds are compounds of formula (II), wherein
R′ is —H;
R₁″ is —H or -methyl;
wherein n″=0
Y″ is —NR₃″R₄″ with
   R₃″ is —H or methyl, and
   R₄″ is an unsubstituted or substituted phenyl residue, and wherein the phenyl residue is substituted with —OCH₃, —C₂H₄OH or —C₃H₆OH (for example compounds 5, 16, 17, 18, 25, 28, 40, 57 and 72 according to Table 2), and
wherein R₂″, W₁″, W₂″, and W₃″ are as defined above.

Another more preferred group of compounds are compounds of formula (II), wherein
R′ is —H;
R₁″ is —H or -methyl;
wherein n″=0
Y″ is —NR₃″R₄″, with
   R₃″ is —H or methyl, and
   R₄″ is the group

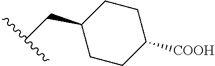 [SMILES: *C[C@H]1CC[C@H](C(=O)O)CC1]

(for example compounds 89 or 98 according to Table 2), and
wherein R₂″, W₁″, W₂″, and W₃″ are as defined above.

Another more preferred group of compounds are compounds of formula (II), wherein Y″ is —NR₃″R₄″ with N, R₃″ and R₄″ forming an unsubstituted or substituted 5- or 6-membered saturated heterocycle or

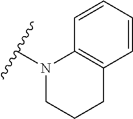 [SMILES: *N1CCCc2ccccc21]

(for example compounds 10, 13, 14, 15, 19, 20, 21, 23, 31, 35, 36, 37, 38, 39, 43, 45, 54, 60, 62, 63, 64, 68, 69, 78 and 85 according to Table 2), and wherein R″, R₁″, R₂″, n″, X″, W₁″, W₂″, and W₃″ are as defined above. A more preferred subgroup of these compounds are compounds with N, R₃″ and R₄″ forming an unsubstituted or substituted piperidine residue wherein the piperidine is substituted with —COOH, —COOCH₃, —COOC₂H₅, —CONH₂, —CONHCH₃ or —CN (for example compounds 10, 13, 14, 15, 19, 21, 23, 35, 36, 37, 38, 39, 43, 54, 60, 62, 63, 64, 68, 69, 85 according to Table 2, especially preferred are —COOH and —CH₂COOH as substituents (for example compounds 13, 14, 21, 36, 37, 54, 69 and 85 according to Table 2). Preferably these compounds have n″=0 and R″ is —H and R₁″ is -methyl (for example compounds 10, 13, 14, 15, 19, 21, 35, 36, 37, 38, 39, 60, 62, 63, 64, 68 and 69 according to Table 2). Another more preferred subgroup of these compounds are compounds with N, R₃″ and R₄″ forming a morpholino residue (for example compound 45 according to Table 2). Another more preferred subgroup of the compounds are compounds with N, R₃″ and R₄″ forming a pyrrolidine (for example compound 31 according to Table 2). Another more preferred subgroup of these compounds are compounds with N, R₃″ and R₄″ forming

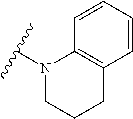 [SMILES: *N1CCCc2ccccc21]

(especially compound 78 according to Table 2).

Another more preferred group of compounds are compounds of formula (II), wherein Y″ is —OR₁₁″, with R₁₁″ is —H or —C₁-C₄-alkyl, phenyl, benzyl or 2-ethoxyethyl (for example compounds 1, 3, 4, 7, 8, 9, 24, 32, 33, 41, 44, 49, 50, 51, 52, 53, 56, 58, 65, 66, 71, 73, 74, 76, 77, 81, 82, 87, 88, 91, 92, 93, 94 and 97 according to Table 2), and wherein R″, R₁″, R₂″, n″, X″, W₁″, W₂″, and W₃″ are as defined above. A more preferred subgroup of these compounds are compounds with n″=0 and with R″ is —H and R₁″ is —H or methyl (for example compounds 1, 24, 33, 44, 49, 50, 51, 52, 53, 56, 58, 66, 73, 74, 76, 77, 81, 82, 87, 88, 91, 92, 93, 94 and 97 according to Table 2). Another more preferred subgroup of these compounds are compounds with n″=1 and with X″ is halogen, and with R″ is —H and R₁″ is —H or methyl (for example compounds 3, 4, 7, 8 and 41 according to Table 2). An even more preferred subgroup of compounds are compounds with R₁₁″ is —H (for example compounds 51, 66, 88 and 97 according to Table 2), -methyl (for example compounds 56, 58 and 91 according to Table 2), -ethyl (for example compounds 3, 4, 7, 8, 9, 32, 33, 41, 49, 50, 52, 65, 71, 81 and 87 according to Table 2), -propyl (for example compound 92 according to Table 2), -isopropyl (for example compound 53 according to Table 2), -tert-butyl (for example compounds 1, 44, 82 and 94 according to Table 2), -phenyl (for example compound 24 according to Table 2), -benzyl (for example compound 93 according to Table 2) or -2-ethoxyethylene (for example compound 73 according to Table 2).

Another preferred group of compounds are compounds of formula (II), wherein Y″ is —NR₃″R₄″, wherein R₃″=H and R₄″=H (for example compounds 67, 75, 84 according to Table 2), and wherein R″, R₁″, R₂″, n″, X″, W₁″, W₂″, and W₃″ are as defined above.

In another preferred embodiment, compounds, wherein n″=1, X″ is —OMe at the para position, Y″ is —OH, W₁″=—H or -methyl, W₂″=—H and W₃″=—H are excluded from the group of compounds of formula (II) of the invention.

It is further mentioned that some of the compounds of formula (II) may actually function as prodrugs, i.e. they are quickly converted into the active POLRMT inhibitor upon administration to the patient. Examples of potential prodrugs are especially esters (see for example compounds 1, 3, 4, 7, 8, 9, 10, 15, 23, 24, 32, 33, 35, 38, 41, 43, 44, 49, 50, 52, 53, 56, 58, 63, 64, 65, 71, 73, 74, 76, 77, 81, 82, 87, 91, 92, 93, and 94 according to Table 2).

A specific subset of compounds are compounds of formula (II) selected from Table 2, or a pharmaceutically or veterinary acceptable salt, hydrate or solvate thereof.

TABLE 2
Exemplary compounds of Formula (II)

| | IUPAC Name |
|---|---|
| 1 | tert-butyl 2-(2-oxo-4-phenyl-chromen-7-yl)oxypropanoate |
| 2 | N,N-dimethyl-2-(2-oxo-4-phenyl-chromen-7-yl)oxy-propanamide |
| 3 | ethyl 2-[4-(4-chlorophenyl)-2-oxo-chromen-7-yl]oxypropanoate |
| 4 | ethyl 2-[4-(3-chlorophenyl)-2-oxo-chromen-7-yl]oxypropanoate |
| 5 | N-[4-(3-hydroxypropyl)phenyl]-2-(2-oxo-4-phenyl-chromen-7-yl)oxy-propanamide |
| 6 | 2-(2-oxo-4-phenyl-chromen-7-yl)oxy-N-(2-pyridyl)propanamide |
| 7 | ethyl 2-[4-(3-fluorophenyl)-2-oxo-chromen-7-yl]oxypropanoate |
| 8 | ethyl 2-[4-(4-fluorophenyl)-2-oxo-chromen-7-yl]oxypropanoate |
| 9 | ethyl 2-[2-oxo-4-(p-tolyl)chromen-7-yl]oxypropanoate |
| 10 | methyl 1-[2-(6-chloro-2-oxo-4-phenyl-chromen-7-yl)oxypropanoyl]piperidine-3- |
| | carboxylate |
| 11 | 2-(6-chloro-2-oxo-4-phenyl-chromen-7-yl)oxy-N-(2-pyridyl)propanamide |
| 12 | N-isopropyl-2-(2-oxo-4-phenyl-chromen-7-yl)oxy-propanamide |
| 13 | 1-[2-(6-chloro-2-oxo-4-phenyl-chromen-7-yl)oxypropanoyl]piperidine-3-carboxylic |
| | acid |
| 14 | 1-[2-(2-oxo-4-phenyl-chromen-7-yl)oxypropanoyl]piperidine-3-carboxylic acid |
| 15 | methyl 1-[2-(2-oxo-4-phenyl-chromen-7-yl)oxypropanoyl]piperidine-3-carboxylate |
| 16 | N-[4-(2-hydroxyethyl)phenyl]-2-(2-oxo-4-phenyl-chromen-7-yl)oxy-propanamide |
| 17 | 2-(6-chloro-2-oxo-4-phenyl-chromen-7-yl)oxy-N-[4-(2- |
| | hydroxyethyl)phenyl]propanamide |
| 18 | N-[4-(2-hydroxyethyl)phenyl]-N-methyl-2-(2-oxo-4-phenyl-chromen-7-yl)oxy- |
| | propanamide |
| 19 | 1-[2-(2-oxo-4-phenyl-chromen-7-yl)oxypropanoyl]piperidine-3-carbonitrile |
| 20 | 7-[1-methyl-2-oxo-2-[3-(2H-tetrazol-5-yl)-1-piperidyl]ethoxy]-4-phenyl-chromen-2- |
| | one |
| 21 | (3S)-1-[(2R)-2-(2-oxo-4-phenyl-chromen-7-yl)oxypropanoyl]piperidine-3-carboxylic |
| | acid |
| 22 | 2-(2-oxo-4-phenyl-chromen-7-yl)oxy-N-(2-pyridyl)acetamide |
| 23 | methyl 1-[2-(2-oxo-4-phenyl-chromen-7-yl)oxyacetyl]piperidine-3-carboxylate |
| 24 | phenyl 2-(2-oxo-4-phenyl-chromen-7-yl)oxypropanoate |
| 25 | 2-(2-oxo-4-phenyl-chromen-7-yl)oxy-N-phenyl-propanamide |
| 26 | N-methyl-2-(2-oxo-4-phenyl-chromen-7-yl)oxy-propanamide |
| 27 | 2-(6-chloro-2-oxo-4-phenyl-chromen-7-yl)oxy-N-(2-hydroxy-2-phenyl- |
| | ethyl)propanamide |
| 28 | 2-(6-chloro-2-oxo-4-phenyl-chromen-7-yl)oxy-N-[4-(3- |
| | hydroxypropyl)phenyl]propanamide |
| 29 | N-(2-hydroxy-2-phenyl-ethyl)-2-(2-oxo-4-phenyl-chromen-7-yl)oxy-propanamide |
| 30 | N-ethyl-2-(2-oxo-4-phenyl-chromen-7-yl)oxy-propanamide |
| 31 | 7-(1-methyl-2-oxo-2-pyrrolidin-1-yl-ethoxy)-4-phenyl-chromen-2-one |
| 32 | ethyl 2-methyl-2-(2-oxo-4-phenyl-chromen-7-yl)oxy-propanoate |
| 33 | ethyl (2R)-2-(2-oxo-4-phenyl-chromen-7-yl)oxypropanoate |
| 34 | (2R)-N,N-dimethyl-2-(2-oxo-4-phenyl-chromen-7-yl)oxy-propanamide |
| 35 | ethyl (3S)-1-[(2R)-2-(2-oxo-4-phenyl-chromen-7-yl)oxypropanoyl]piperidine-3- |
| | carboxylate |
| 36 | (3S)-1-[2-(2-oxo-4-phenyl-chromen-7-yl)oxypropanoyl]piperidine-3-carboxylic acid |
| 37 | (3R)-1-[2-(2-oxo-4-phenyl-chromen-7-yl)oxypropanoyl]piperidine-3-carboxylic acid |
| 38 | methyl (3R)-1-[2-(2-oxo-4-phenyl-chromen-7-yl)oxypropanoyl]piperidine-3- |
| | carboxylate |
| 39 | 7-[1-methyl-2-oxo-2-(1-piperidyl)ethoxy]-4-phenyl-chromen-2-one |
| 40 | N-[4-(2-hydroxyethyl)phenyl]-N-methyl-2-(2-oxo-4-phenyl-chromen-7-yl)oxy- |
| | acetamide |
| 41 | ethyl 2-[4-(4-bromophenyl)-2-oxo-chromen-7-yl]oxypropanoate |
| 42 | 2-(6-chloro-2-oxo-4-phenyl-chromen-7-yl)oxy-N-(2-furylmethyl)propanamide |
| 43 | ethyl 1-[2-(2-oxo-4-phenyl-chromen-7-yl)oxyacetyl]piperidine-3-carboxylate |
| 44 | tert-butyl 2-(2-oxo-4-phenyl-chromen-7-yl)oxyacetate |
| 45 | 6-chloro-7-(2-morpholino-2-oxo-ethoxy)-4-phenyl-chromen-2-one |
| 46 | 2-(6-chloro-2-oxo-4-phenyl-chromen-7-yl)oxy-N-cyclopropyl-acetamide |
| 47 | 2-(6-chloro-2-oxo-4-phenyl-chromen-7-yl)oxy-N,N-diethyl-acetamide |
| 48 | N,N-diethyl-2-(2-oxo-4-phenyl-chromen-7-yl)oxy-acetamide |
| 49 | ethyl 2-(6-chloro-2-oxo-4-phenyl-chromen-7-yl)oxyacetate |
| 50 | ethyl 2-(6-chloro-2-oxo-4-phenyl-chromen-7-yl)oxypropanoate |
| 51 | 2-(2-oxo-4-phenyl-chromen-7-yl)oxypropanoic acid |
| 52 | ethyl 2-(2-oxo-4-phenyl-chromen-7-yl)oxypropanoate |
| 53 | isopropyl 2-(2-oxo-4-phenyl-chromen-7-yl)oxypropanoate |
| 54 | 1-[2-(2-oxo-4-phenyl-chromen-7-yl)oxyacetyl]piperidine-3-carboxylic acid |
| 55 | 2-(2-oxo-4-phenyl-chromen-7-yl)oxy-N-(3-pyridyl)propanamide |
| 56 | methyl 2-(2-oxo-4-phenyl-chromen-7-yl)oxypropanoate |
| 57 | 2-(6-chloro-2-oxo-4-phenyl-chromen-7-yl)oxy-N-[4-(2- |
| | hydroxyethyl)phenyl]acetamide |
| 58 | methyl 2-(6-chloro-2-oxo-4-phenyl-chromen-7-yl)oxypropanoate |
| 59 | 2-(6-chloro-2-oxo-4-phenyl-chromen-7-yl)oxy-N-(3-pyridylmethyl)acetamide |
| 60 | 1-[2-(2-oxo-4-phenyl-chromen-7-yl)oxypropanoyl]piperidine-3-carboxamide |
| 61 | 2-(2-oxo-4-phenyl-chromen-7-yl)oxy-N-(4-pyridyl)propanamide |
| 62 | N-methyl-1-[2-(2-oxo-4-phenyl-chromen-7-yl)oxypropanoyl]piperidine-3- |
| | carboxamide |
| 63 | methyl 1-[2-(2-oxo-4-phenyl-chromen-7-yl)oxypropanoyl]piperidine-4-carboxylate |
| 64 | ethyl rac-(3S)-1-[2-(2-oxo-4-phenyl-chromen-7-yl)oxypropanoyl]piperidine-3- |
| | carboxylate |
| 65 | ethyl 2-[4-(4-methoxyphenyl)-2-oxo-chromen-7-yl]oxypropanoate |
| 66 | (2R)-2-(2-oxo-4-phenyl-chromen-7-yl)oxypropanoic acid |
| 67 | 2-(2-oxo-4-phenyl-chromen-7-yl)oxypropanamide |
| 68 | rac-(3S)-N,N-dimethyl-1-[2-(2-oxo-4-phenyl-chromen-7-yl)oxypropanoyl]piperidine- |
| | 3-carboxamide |
| 69 | 1-[2-(2-oxo-4-phenyl-chromen-7-yl)oxypropanoyl]piperidine-4-carboxylic acid |
| 70 | N-(2-morpholinoethyl)-2-(2-oxo-4-phenyl-chromen-7-yl)oxy-propanamide |
| 71 | ethyl 2-(2-oxo-4-phenyl-chromen-7-yl)oxybutanoate |
| 72 | N-(4-methoxyphenyl)-2-(2-oxo-4-phenyl-chromen-7-yl)oxy-propanamide |
| 73 | 2-ethoxyethyl 2-(2-oxo-4-phenyl-chromen-7-yl)oxyacetate |
| 74 | propyl 2-(2-oxo-4-phenyl-chromen-7-yl)oxyacetate |
| 75 | 2-(2-oxo-4-phenyl-chromen-7-yl)oxyacetamide |
| 76 | butyl 2-(2-oxo-4-phenyl-chromen-7-yl)oxyacetate |
| 77 | isobutyl 2-(2-oxo-4-phenyl-chromen-7-yl)oxyacetate |
| 78 | 6-chloro-7-[2-(3,4-dihydro-2H-quinolin-1-yl)-2-oxo-ethoxy]-4-phenyl-chromen-2- |
| | one |
| 79 | 2-(6-chloro-2-oxo-4-phenyl-chromen-7-yl)oxy-N-(2-morpholinoethyl)propanamide |
| 80 | 2-(5-fluoro-2-oxo-4-phenyl-chromen-7-yl)oxy-N,N-dimethyl-propanamide |
| 81 | ethyl 2-(2-oxo-4-phenyl-chromen-7-yl)oxyacetate |
| 82 | tert-butyl 2-(6-chloro-2-oxo-4-phenyl-chromen-7-yl)oxyacetate |
| 83 | 2-(6-chloro-2-oxo-4-phenyl-chromen-7-yl)oxy-N-(2-morpholinoethyl)acetamide |
| 84 | 2-(6-chloro-2-oxo-4-phenyl-chromen-7-yl)oxyacetamide |
| 85 | 1-[2-(6-chloro-2-oxo-4-phenyl-chromen-7-yl)oxyacetyl]piperidine-3-carboxylic acid |
| 86 | 2-(6-chloro-2-oxo-4-phenyl-chromen-7-yl)oxy-N-cyclooctyl-acetamide |
| 87 | ethyl 2-(5-fluoro-2-oxo-4-phenyl-chromen-7-yl)oxypropanoate |
| 88 | 2-(6-chloro-2-oxo-4-phenyl-chromen-7-yl)oxyacetic acid |
| 89 | 4-[[2-(6-chloro-2-oxo-4-phenyl-chromen-7- |
| | yl)oxypropanoylamino]methyl]cyclohexanecarboxylic acid |
| 90 | 2-(6-chloro-2-oxo-4-phenyl-chromen-7-yl)oxy-N-(2-hydroxy-2-phenyl- |
| | ethyl)acetamide |
| 91 | methyl 2-(8-methyl-2-oxo-4-phenyl-chromen-7-yl)oxyacetate |
| 92 | propyl 2-(6-chloro-2-oxo-4-phenyl-chromen-7-yl)oxyacetate |
| 93 | benzyl 2-(2-oxo-4-phenyl-chromen-7-yl)oxypropanoate |
| 94 | tert-butyl 2-(8-methyl-2-oxo-4-phenyl-chromen-7-yl)oxyacetate |
| 95 | 2-(6-chloro-2-oxo-4-phenyl-chromen-7-yl)oxy-N-(1,1 -dioxo-2,3-dihydrothiophen-3- |
| | yl)acetamide |
| 96 | 2-[2-(2-oxo-4-phenyl-chromen-7-yl)oxypropanoylamino]-2-phenyl-acetic acid |
| 97 | 2-(2-oxo-4-phenyl-6-propyl-chromen-7-yl)oxypropanoic acid |
| 98 | 4-[[[2-(6-chloro-2-oxo-4-phenyl-chromen-7- |
| | yl)oxyacetyl]amino]methyl]cyclohexanecarboxylic acid |
| 99 | 2-(6-chloro-2-oxo-4-phenyl-chromen-7-yl)oxy-N-(2-pyridylmethyl)acetamide |

In one embodiment, according to the present invention there is provided a composition, wherein the IMT is a compound of the general formula (III) according to WO2020/188049. The compound of formula (III) may be

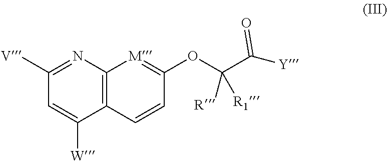

wherein
R′″ is —H or —C₁-C₄-alkyl, preferably —H, -methyl or -ethyl, in particular -methyl;
R₁′″ is —H, or -methyl, preferably —H;
M′″ is CH or N, preferably —H;
V′″ is —H, —OH, —Cl, —F, or —C₁-C₄-alkyl, preferably —H, —Cl, —F, or -methyl;
W′″ is

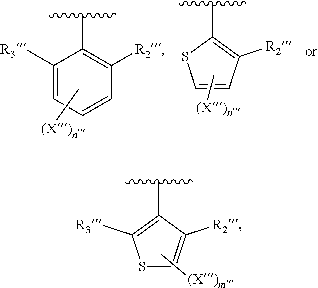

with
   R₂′″ and R₃′″ are identical or different and are —H, —C₁-C₄-alkyl, halogen-C₁-C₄-alkyl, —C₁-C₄-alkoxy, —C₁-C₄-dialkylamino, —C₂-C₆-alkenyl, —C₂-C₆-alkynyl, -halogen, —CN or —CO—NH₂;
   preferably —H, —C₁-C₄-alkyl, —CF₃, —OCH₃, —NHCH₃, —N(CH₃)₂, —F, or —Cl;
   X′″ is -halogen, or —CN, preferably —F, with n′″=1 or 2 or m″=1;
   n′″=0, 1, or 2, preferably 0 or 1;
   m′″=0 or 1;
Y′″ is —NR₄′″R₅′″ with
   R₄′″ is —H, or —C₁-C₄-alkyl, preferably —H or -methyl, and
   R₅′″ is —H, —C₁-C₄-alkyl, an unsubstituted or substituted —C₃-C₆-cycloalkyl, preferably -methyl, -ethyl, -isopropyl, or -cyclopropyl; or an unsubstituted or substituted pyridine residue; or an unsubstituted or substituted phenyl residue, preferably substituted at the para position;
Y″ is —NR₄′″R₅′″ with N, R₄′″ and R₅′″ forming an unsubstituted or substituted 4-, 5- or 6-membered saturated heterocycle, preferably an unsubstituted or substituted azetidine, an unsubstituted or substituted piperidine, an unsubstituted or substituted pyrrolidine, an unsubstituted or substituted piperazine, or an unsubstituted or substituted tetrahydropyridine residue; or
Y″ is —OR₆′″, with R₆′″ is —H or —C₁-C₄-alkyl, preferably —H, -methyl, -ethyl, -isopropyl or-tert-butyl,
   or a pharmaceutically or veterinary acceptable salt, hydrate or solvate thereof.

In a preferred embodiment, the compounds of the general formula (III) are quinoline derivatives, wherein M″ is CH.

Further included are pharmaceutically or veterinary acceptable salts, hydrates or solvates of the compounds of formula (III) or its intermediate compounds disclosed herein. A pharmaceutically or veterinary acceptable salt can be an anionic counterion, e.g. an acetate, a bromide, camsylate, chloride, citrate, formate, fumarate, lactate, maleate, mesylate, nitrate, oxalate, phosphate, sulfate, tartrate, thiocyanate, or tosylate, or preferably a cationic counterion, e.g. ammonium, arginine, diethylamine, ethylenediamine, piperazine, potassium, sodium, or any other counter ion disclosed in Haynes et al. (2005). Some compounds of the invention contain one or more chiral centers due to the presence of asymmetric carbon atoms, which gives rise to stereoisomers, for example todiastereoisomers with R or S stereochemistry at each chiral center. The invention includes all such stereoisomers and diastereoisomers and mixtures thereof.

The compounds of general formula (III) or a pharmaceutically or veterinary acceptable salt, hydrate or solvate thereof, are useful as mitochondrial RNA polymerase (POLRMT) inhibitors and thereby inhibit mitochondrial DNA replication and/or mitochondrial transcription.

In the following, preferred groups of the compounds of general formula (III) of the present invention are described. The preferred groups constitute preferred embodiments of the compounds of general formula (III). Any combinations of the embodiments of the compounds of general formula (III) of the invention described herein are considered to be within the scope of the invention.

In a preferred embodiment, the invention relates to a composition, wherein the IMT is a compound of the general formula (III) as defined above, wherein
W′″ is

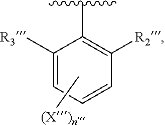

with
   R₃′″ is —H, —C₁-C₄-alkyl, —CF₃, —OCH₃, —NHCH₃, —N(CH₃)₂, acetylenyl, —F, —Cl, —Br, CN, or CONH₂;
   R₂′″ is —H, -methyl, -ethyl, isopropyl, —CF₃, —F, or —Cl; and
   X′″ is —F with n′″=1; preferably, wherein X′″ is at the para-position of the phenyl ring; or
   n′″=0.

This preferred group of compounds corresponds to the compounds of formula (IIIA)

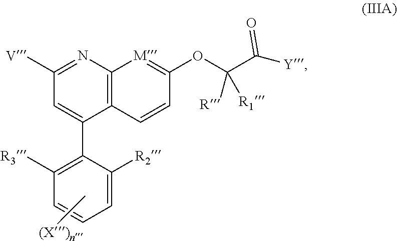

wherein R′″, R₁′″, R₂′″, R₃′″, M′″, V′″, X′″, n′″ and Y′″ are as defined in the preferred group above.

A particular preferred group of compounds are compounds of formula (IIIB)

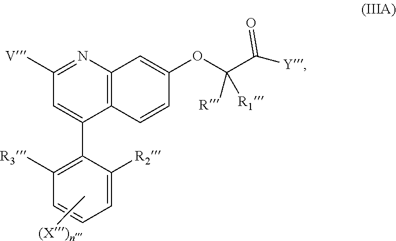

wherein R′″, R₁′″, R₂′″, R₃′″, M′″, V′″, X′″, n′″ and Y′″ are as defined in the preferred group above.

In one embodiment, the invention relates to a composition, wherein the IMT is a compound of the general formula (III) as defined above, wherein
W′″ is

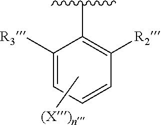

R₂′″ is —H, -methyl, -ethyl, isopropyl, —C₁, preferably -methyl or —Cl;
R₃′″ is —H, -methyl or —Cl; preferably R₂′″ is -methyl and R₃′″ is -methyl, or R₂′″ is —Cl and R₃′″ is —Cl; and
n′″=0.

In one embodiment, the invention relates to a composition, wherein the IMT is a compound of the general formula (III) as defined above, wherein
R′″ is —C₁-C₄-alkyl, preferably -methyl or -ethyl, in particular -methyl;
R₁′″ is —H, or -methyl, preferably —H;
M′″ is —CH;
V′″ is as defined above;
W′″ is

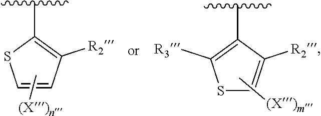

with
   R₂“ ” is —H, methyl, -halogen, —CN, preferably -methyl, —C₁, or —Br;
   R₃′″ is —H, C₁-C₄-alkyl, -halogen, —CN, preferably -methyl, -ethyl, —C₁, or —Br;
   X′″ is -halogen, or —CN, preferably —Cl, —Br, or —F, in particular —F, with n′″=1 or 2;
   n′″=0, 1, or 2, preferably 0 or 1;
   m′″=0 or 1;
Y′″ is —NR₄′″R₅′″ with
R₄′″ is —H, or —C₁-C₄-alkyl, preferably —H or -methyl, and
R₅′″ is —C₁-C₄-alkyl or —C₃-C₆-cycloalkyl, preferably -methyl, -ethyl, -isopropyl, or -cyclopropyl; or an unsubstituted or substituted pyridine residue; or an unsubstituted or substituted phenyl residue, preferably substituted at the para position;
Y′″ is —NR₄′″R₅′″ with N, R₄′″ and R₅′″ form an unsubstituted or substituted 5- or 6-membered saturated heterocycle; or
Y′″ is —OR₆′″, with R₆′″ is —H or —C₁-C₄-alkyl, preferably —H, -methyl, -ethyl, or -isopropyl.

In one embodiment, the invention relates to a composition, wherein the IMT is a compound of the general formula (III) of the group as defined above, wherein
W′″ is with

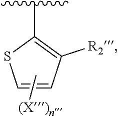

R₂′″, X′″ and n′″ as defined above, for example compound 134 according to Table 3.

In one embodiment, the invention relates to a composition, wherein the IMT is a compound of the general formula (III) of the group as defined above, wherein
W′″ is

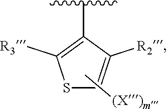

with R₃′″ is H, R₂′″, X′″ and m′″ as defined above.

In one embodiment, the invention relates to a composition, wherein the IMT is a compound of the general formula (III) of the group as defined above, wherein
W′″ is

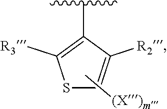

with R₂′″ is H, R₃′″, X′″ and m′″ as defined above, for example compound 133 according to Table 3.

In another embodiment, the invention relates to a composition, wherein the IMT is a compound of the general formula (III) as defined above, wherein
Y′″ is —NR₄′″R₅′″, with
   R₄′″ is —H, or —C₁-C₄-alkyl, preferably —H or -methyl, and
   R₅′″ is —H, —C₁-C₄-alkyl, unsubstituted —C₃-C₄-cycloalkyl, —C₄-cycloalkyl substituted with —COO—CH₃, preferably -methyl, -ethyl, -isopropyl, or -cyclopropyl.

A specific subset of the compounds as defined above are the compounds of the general formula (III), wherein Y′″ is —NR₄′″R₅′″, with
R₄′″ is —H, or —C₁-C₄-alkyl, preferably —H or -methyl, and
R₅′″ is an unsubstituted pyridine residue; or an unsubstituted or substituted phenyl residue, preferably unsubstituted or substituted with one substituent at the para position, wherein the substituents are selected from the group consisting of: —C₁-C₄-alkyl, —C₁-C₄-alkoxy, —(CH₂)₂—OH, —COOH, or —CO—O—(C₁-C₄-alkyl).

Another specific subset of the compounds are the compounds of the general formula (III), wherein
Y′″ is —NR₄′″R₅′″ with
   N, R₄′″ and R₅′″ forming an unsubstituted or substituted azetidine residue, an unsubstituted or substituted piperidine residue, an unsubstituted or substituted piperazine residue, an unsubstituted or substituted pyrrolidine residue, an unsubstituted or substituted morpholine residue, or an unsubstituted or substituted tetrahydropyridine residue, preferably an unsubstituted or substituted piperidine residue, each optionally and independently substituted with one or more, preferably with one of the following residues:
   —C₁-C₄-alkyl;
   —C(OH)-cyclopropyl); —C(COOH)-cyclopropyl;
   unsubstituted or substituted —C₃-C₆-cycloalkyl; preferably hydroxycyclopropyl, or carboxycyclopropyl;
   —(CH₂)ₒ′″—COOR₇′″ with
      R₇′″ is —H, —C₁-C₈-alkyl,
      preferably —H, -methyl, -ethyl, -isopropyl, or -tert-butyl, and
      o′″=0, 1 or 2; preferably 0 or 1;

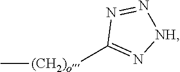

and o′″ is as defined above;
—(CH₂)ₚ′″CONR₈′″R₉′″ with
   R₈′″ and R₉′″ independently are —H, —OH, —CN or —C₁-C₄-alkyl, preferably —H or -methyl, and
   p′″=0, 1 or 2; preferably 0 or 1;
—C(CH₃)₂—COOH;
═O or —OH;
—CO-cyclopropyl;
—CO—(C₁-C₄-alkyl), preferably —CO—CH₂—CH₃;
—CO—(CH₂)_{q}′″—NR₁₂′″R₁₃′″ with R₁₂′″ and R₁₃′″ independently are —H, —C₁-C₄-alkyl or —CN, preferably —CO—(CH₂)_{q}-NH₂, more preferably —CO—CH₂—NH₂, and
   q′″=0, 1 or 2, preferably 0 or 1;
—NH₂, —NH—CO-cyclopropyl, —NH—CO—CH₂—Cl, —NH—CO—CH₂—CH₃, —NH—CO—NH—C(CH₃)₃, —NH—SO₂CH₃, —NH—CO-phenyl, —NOH—CO—CH₃;
—F; —CN;
R₁₄′″ and R₁₅′″ forming a pyrrolidinone ring, a cyclopropanecarboxlic acid ring, an oxetane ring, or a —CH₂— group; or
—(CH₂)ᵣ′″SO₂NR₁₀′″′R₁₁′″ with R₁₀′″ and R₁₁′″ independently are —H, or —C₁-C₄-alkyl, preferably —H or -methyl, preferably —CH₂SO₂NH₂ and
   r′″=0, 1 or 2, preferably 0 or 1.

Another specific subset of the compounds are the compounds of the general formula (III), wherein N, R₄′″ and R₅′″ together form an unsubstituted or substituted azetidine, piperidine, piperazine or pyrrolidine residue, each optionally and independently substituted with one or more, preferably with one of the following residues:
—C₁-C₄-alkyl, unsubstituted or substituted —C₃-C₆-cycloalkyl;
—(CH₂)ₒ′″—COOR₇′″ with
   R₇′″ is —H, —C₁-C₈-alkyl, preferably —H, -methyl, -ethyl, -isopropyl, or -tert-butyl, and
   o′″=0, 1 or 2; preferably 0 or 1;

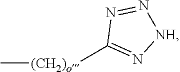

and o′″ is as defined above;
—(CH₂)ₚ′″CONR₈′″R₉′″ with
   R₈′″ and R₉′″ independently are —H, —OH, —CN, or —C₁-C₄-alkyl, preferably —H or -methyl, and
   p′″=0, 1 or 2; preferably 0 or 1;
═O or —OH;
—CO-cyclopropyl;
—CO—(C₁-C₄-alkyl), preferably —CO—CH₂—CH₃;
—CO—(CH₂)_{q}′″—NR₁₂′″R₁₃′″ with R₁₂′″ and R₁₃′″ independently are —H, —C₁-C₄-alkyl or —CN, preferably —CO—(CH₂)_{q}″—NH₂, more preferably —CO—CH₂—NH₂, and
   q′″=0, 1 or 2, preferably 0 or 1;
—NH₂, —NH—CO-cyclopropyl, —NH—CO—CH₂—Cl, —NH—CO—CH₂—CH₃, —NH—SO₂CH₃, —NH—CO-phenyl, —NOH—CO—CH₃;
—F; —CN;
R₁₄′″ and R₁₅′″ forming a pyrrolidinone ring or a cyclopropanecarboxlic acid ring; or
—(CH₂)ᵣ′″SO₂NR₁₀′″R₁₁′″ with R₁₀′″ and R₁₁′″ independently are —H, or —C₁-C₄-alkyl, preferably —H or -methyl, preferably —CH₂SO₂NH₂ and
   r′″=0, 1 or 2, preferably 0 or 1.

Another specific subset of the compounds are the compounds of the general formula (III), wherein
Y′″ is —NR₄′″R₅′″ with
   N, R₄′″ and R₅′″ forming an unsubstituted or substituted piperidine reside, an unsubstituted or substituted piperazine residue, an unsubstituted or substituted pyrrolidine residue, or an unsubstituted or substituted morpholine residue, each optionally and independently substituted with one or more, preferably with one of the following residues:
   —C₁-C₄-alkyl;
   —C(OH)cyclopropyl;
   hydroxycyclopropyl or carboxycyclopropyl;
   —(CH₂)ₒ′″—COOR₇′″ with
   R₇′″ is —H, —C₁-C₈-alkyl, preferably —H, -methyl, -ethyl, -isopropyl, or -tert-butyl, and
   o′″=0, 1 or 2;

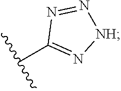 [SMILES: *c1nn[nH]n1]

—(CH₂)ₚ′″CONR₈′″R₉′″ with
R₈′″ and R₉′″ independently are —H, —OH, —CN, -methyl, or -tert-butyl and p′″=0;
—C(CH₃)₂—COOH;
═O or —OH;
—CO-cyclopropyl;
—CO—CH₂—CH₃;
—CO-tert-butyl;
—NH₂,
—CO—CH₂—NH₂;
—NH—CO—CH₂—CH₃, —NH—CO—NH—C(CH₃)₃, —NH—SO₂CH₃, —NH—CO-phenyl, —NOH—CO—CH₃;
—CN;
R₁₄′″ and R₁₅′″ forming a pyrrolidinone ring, a cyclopropanecarboxlic acid ring, an oxetane ring; or a —CH₂— group;
—SO₂NR₁₀′″R₁₁′″ with R₁₀′″ and R₁₁′″ independently are —H or -methyl; or —CH₂SO₂NH₂.

Another specific subset of the compounds are the compounds of the general formula (III), wherein N, R₄′″ and R₅′″ together form an unsubstituted or substituted piperidine or pyrrolidine residue, each optionally and independently substituted with one or more, preferably with one, of the following residues:
—(CH₂)ₒ′″—COOR₇′″ with
R₇′″ is —H, —C₁-C₈-alkyl, preferably —H, -methyl, -ethyl, -isopropyl, or -tert-butyl, and
o′″=0, 1 or 2;

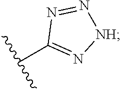 [SMILES: *c1nn[nH]n1]

—(CH₂)ₚ′″CONR₈′″R₉′″ with
R₈′″ and R₉′″ independently are —H, —OH, —CN, -methyl or -tert-butyl, and p′″=0;
═O or —OH;
—CO-cyclopropyl;
—CO—CH₂—CH₃;
—CO-tert-butyl;
—NH₂
—CO—CH₂—NH₂;
—CN;
—SO₂NR₁₀′″′R₁₁′″ with R₁₀′″ and R₁₁′″ independently are —H or -methyl; or —CH₂SO₂NH₂.

A group of preferred compounds of formula (III) have an unsubstituted piperidine, i.e. N, R₄′″ and R₅′″ together form an unsubstituted piperidine residue.

A more preferred subgroup are compounds of formula (III) having a substituted piperidine residue substituted with —COOH or with —CH₂COOH.

Another more preferred subgroup are compounds of formula (III) having a unsubstituted or substituted piperidine residue, optionally and independently substituted with one or more of the following residues: —COOH, —COOCH₃, —COOC₂H₅, —CH₂COOH, —C(CH₃)₂—COOH, —CH₂COOCH₃, —CH₂COOCH₂CH₃, —CONH₂, —CONHCH₃, —CON(CH₃)₂, —SO₂NH₂ or —CH₂SO₂NH₂.

In another embodiment, the invention relates to a composition, wherein the IMT is a compound of the general formula (III) as defined herein, wherein
V′″ is —H, —Cl, —F, or -methyl, preferably —H.

In another embodiment, the invention relates to a composition, wherein the IMT is a compound of the general formula (III) as defined herein, wherein
R′″ is -methyl, preferably —(R)-methyl; and
R₁′″ is —H.

In a preferred embodiment, R′″ is —(R)-methyl.

In another embodiment, the invention relates to a composition, wherein the IMT is a compound of the general formula (III) as defined herein, wherein X′″ is at the para-position of the phenyl ring.

Another specific subset of the compounds are the compounds of the general formula (III), wherein
R′″ is -methyl, preferably (R)-methyl;
R₁′″ is —H;
M′″ is CH;
V′″ is —H, —Cl, —F or -methyl;
W″ is

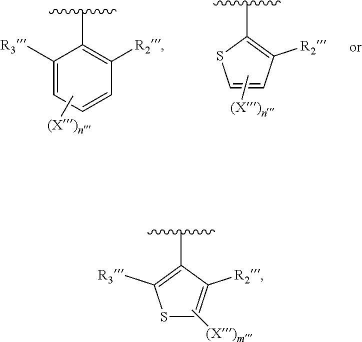

preferably

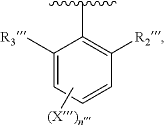

with
   R₂′″ and R₃′″ are -methyl, or —Cl with n′″=1 or 2, preferably di-methyl or di-choro with n′″=2;
   X′″ is —F with m″=1;
   n′″=0, 1, or 2
   m′″=0, or 1;
Y′″ is —NR₄′″R₅′″
   with
   R₄′″ is —H, and
   R₅′″ is a pyridine residue, a phenyl residue substituted at the para position, preferably substituted with or —(CH₂)₂OH, a cyclopropyl or isopropyl residue;
   or with
      N, R₄′″ and R₅′″ are together a piperidine residue, or a pyrrolidine residue, each optionally and independently substituted with one of the following residues: —COOH, —COOCH₃, —COOC₂H₅, —CH₂COOH, —C(CH₃)₂—COOH, CH₂COOCH₃, —CH₂COOCH₂CH₃, —CONH₂, —CONHCH₃, —CON(CH₃)₂, —SO₂NH₂ or —CH₂SO₂NH₂.

A more preferred subgroup are compounds of formula (III), wherein R′″ is (R)-methyl, having a substituted piperidine residue substituted with —COOH or with —CH₂COOH.

Another more preferred subgroup of this specific subset are compounds of formula (III),
wherein R′″ is (R)-methyl, having a substituted piperidine residue substituted with —(CH₂)ₒ′″—COOR₇′″ with —C₁-C₈-alkyl, is —H,
   preferably —H, -methyl, -ethyl, -isopropyl, or -tert-butyl, and
   o′″=0, 1 or 2; preferably 0 or 1;

Another specific subset of compounds are compounds of formula (III), wherein
R′″ is (R)-methyl;
R₁′″ is —H;
M′″ is CH;
V′″ is —H, —Cl, —F or -methyl;
W″ is

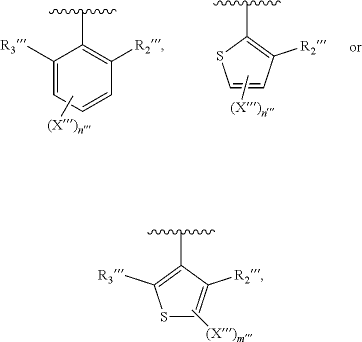

preferably

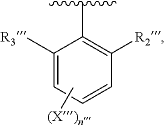

with
   R₂′″ and R₃′″ are -methyl, or —C₁, with n′″=1 or 2, preferably di-methyl or di-choro with n′″=2;
   X′″ is —F with m″=1;
   n′″=0, 1, or 2
   m′″=0, or 1;
Y′″ is —NR₄′″R₅′″
   with
   N, R₄′″ and R₅′″ form a piperidine residue, or a pyrrolidine residue, each optionally and independently substituted with one of the following residues:
   —COOH, —COOCH₃, —COOC₂H₅, —CH₂COOH, —C(CH₃)₂—COOH, —CH₂COOCH₃, —CH₂COOCH₂CH₃, —CONH₂, —CONHCH₃, —CON(CH₃)₂, —SO₂NH₂ or —CH₂SO₂NH₂,
   preferably —COOH, —CH₂COOH, or —CONHCH₃,
   more preferably (S)—COOH, (R)—COOH, (S)—CH₂COOH or (R)—CH₂COOH, especially a piperidine residue substituted with (S)—COOH, (R)—COOH, (S)—CH₂COOH or (R)—CH₂COOH.

A more preferred group of compounds are compounds of formula (III) having a substituted piperidine residue where R′″ is (R)-methyl, W′″ is

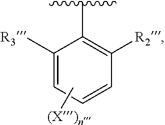

R₁′″ is —H, V′″=—H, R₂′″ and R₃′″ are independently -methyl or —Cl, preferably —Cl, X′″ is —F, n′″=1 and wherein the piperidine is substituted with —COOH, —CH₂COOH, —CONHCH₃, —CH₂CONHCH₃, —SO₂NH₂, —SO₂NHCH₃, or —CN.

Another specific subset of compounds are compounds of formula (III), wherein the piperidine residue or the pyrrolidine residue is substituted at the 3-position.

A more preferred group of compounds of this subset are compounds of formula (III) having any substituted piperidine or pyrrolidine residue as defined above at the 3-position. More preferred within this group are compounds having a substituted piperidine residue substituted with —(CH₂)ₒ′″—COOR₇′″ with R₇′″ is —H, —C₁-C₆-alkyl, preferably —H, -methyl, -ethyl, -isopropyl, or -tert-butyl, and o′″=0, 1 or 2; preferably 0 or 1; at the 3-position with —COOR₇′″ or —CH₂COOR₇′″ at the 3-position, or with R₇′″ is -isopropyl, -tert-butyl, or with —CONHCH₃ or —CH₂CONHCH₃ at the 3-position.

Another group of preferred compounds of this subset of compounds of formula (III) have a substituted piperidine residue substituted with —(CH₂)ᵣ″SO₂NR₁₀′″′R₁₁′″ with R₁₀′″ and R₁₁′″ independently are —H, or —C₁-C₄-alkyl, preferably —H or -methyl, preferably —CH₂SO₂NH₂ and r′″=0, 1 or 2, preferably 0 or 1 at the 3-position with R₁₀′″ is —H and R₁₁′″ is —H or -methyl.

A more preferred group of compounds are compounds of formula (III) having a substituted piperidine residue, wherein the substitution is at the 3-position, where R′″ is methyl, R₁′″ is —H, V′″=—H, R₂′″ and R₃′″ is -methyl or —Cl, n″, m′″=0 or 1, X′″ is —F with n′″=1 or m″=1 and wherein the piperidine is substituted with one of the following residues: —COOH, —COOCH₃, —COOC₂H₅, —CH₂COOH, —CH₂COOCH₃, —CH₂COOCH₂CH₃, —CONH₂, —CONHCH₃, —CON(CH₃)₂, —CH₂CONHCH₃, SO₂NH₂, —SO₂NHCH₃, or —CN. An even more preferred subgroup of this group are compounds where R′″ is (R)-methyl

A more preferred subgroup are compounds of formula (III) having a substituted piperidine residue substituted with —COOH at the 3-position and wherein R′″ is (R)-methyl, or with —CH₂COOH at the 3-position and wherein R′″ is (R)-methyl.

Another more preferred subgroup are compounds of formula (III), wherein R′″ is (R)-methyl and having a substituted piperidine residue, wherein the substitution is at the 3-position, substituted with —(CH₂)ₒ′″—COOR₇′″ with R₇′″ is —H, —C₁-C₆-alkyl, preferably —H, -methyl, -ethyl, -isopropyl, or -tert-butyl, and O′″=0, 1 or 2; preferably 0 or 1, preferably with R₇′″ is -isopropyl, -tert-butyl, or with —CONHCH₃ or —CH₂CONHCH₃.

An especially preferred group of compounds of formula (III) are compounds having a substituted piperidine residue where R′″ is (R)-methyl, R₁′″ is —H, V″=—H, R₂′″ and R₃′″ is -methyl, or —Cl, W′″ is

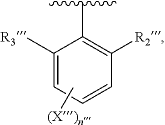

n′″=0 or 1, X′″ is —F with n′″=1 and wherein the piperidine is substituted with —COOH, —CH₂COOH, —CONHCH₃, —CH₂CONHCH₃, —SO₂NH₂, —SO₂NHCH₃, or —CN at the 3-position.

A more preferred subgroup are compounds of formula (III), wherein X′″ is at the para-position, having a substituted piperidine residue substituted with —COOH at the 3-position and wherein R′″ is (R)-methyl or with —CH₂COOH at the 3-position and wherein R′″ is (R)-methyl.

Another more preferred subgroup are compounds of formula (III), wherein X′″ is at the para-position, wherein R′″ is (R)-methyl and having a substituted piperidine residue substituted with —(CH₂)ₒ′″—COOR₇′″ with R₇′″ is —H, —C₁-C₈-alkyl, preferably —H, -methyl, -ethyl, -isopropyl, or -tert-butyl, and o′″=0, 1 or 2; preferably 0 or 1 at the 3-position, with R₇′″ is -isopropyl, -tert-butyl, n, or —CONHCH₃ or —CH₂CONHCH₃ at the 3-position.

An especially preferred group of compounds are compounds of formula (III), wherein X′″ is at the para-position, having a substituted piperidine residue where R′″ is (R)-methyl, R₁′″ is —H, W′″ is

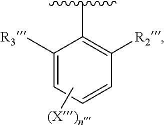

n′″=0 or 1, R₂′″ and R₃′″ are -methyl, or —C₁, preferably —Cl, X′″ is —F with n′″=1 and wherein the piperidine is substituted with —COOH, —CH₂COOH, —CONHCH₃, —SO₂NH₂, —SO₂NHCH₃, or —CN at the 3-position. An especially preferred subgroup of this group concerns compounds with R₂′″ is —Cl, X′″ is —F and n′″=1.

Another specific subset of compounds concerns compounds selected from
(3S)-1-[(2R)-2-[[4-(o-tolyl)-7-quinolyl]oxy]propanoyl]piperidine-3-carboxylic acid,
(3S)-1-[(2R)-2-[[4-(2,6-dimethylphenyl)-7-quinolyl]oxy]propanoyl]piperidine-3-carboxylic acid,
2-[(3R)-1-[(2R)-2-[[4-(2,6-dimethylphenyl)-7-quinolyl]oxy]propanoyl]-3-piperidyl]acetic acid,
ethyl (3S)-1-[(2R)-2-[[4-(2,6-dimethylphenyl)-7-quinolyl]oxy]propanoyl]piperidine-3-carboxylate,
2-[(3S)-1-[(2R)-2-[[4-(2,6-dimethylphenyl)-7-quinolyl]oxy]propanoyl]-3-piperidyl]acetic acid,
ethyl 2-[(3R)-1-[(2R)-2-[[4-(2,6-dimethylphenyl)-7-quinolyl]oxy]propanoyl]-3-piperidyl]acetate,
(3R)-1-[(2R)-2-[[4-(2-chlorophenyl)-7-quinolyl]oxy]propanoyl]piperidine-3-carboxylic acid,
ethyl 2-[(3R)-1-[(2R)-2-[[4-(o-tolyl)-7-quinolyl]oxy]propanoyl]-3-piperidyl]acetate,
(3S)-1-[(2R)-2-[[4-(2-chlorophenyl)-7-quinolyl]oxy]propanoyl]piperidine-3-carboxylic acid,
(3S)-1-[(2R)-2-[[4-(2-chloro-4-fluoro-phenyl)-7-quinolyl]oxy]propanoyl]piperidine-3-carboxylic acid,
(2R)-2-[[4-(2-chloro-4-fluoro-phenyl)-7-quinolyl]oxy]-1-[(3S)-3-(2H-tetrazol-5-yl)-1-piperidyl]propan-1-one,
(3S)-1-[(2R)-2-[[4-(2-chlorophenyl)-2-fluoro-7-quinolyl]oxy]propanoyl]piperidine-3-carboxylic acid,
2-[(3R)-1-[(2R)-2-[[4-(o-tolyl)-7-quinolyl]oxy]propanoyl]-3-piperidyl]acetic acid,
ethyl (3S)-1-[(2R)-2-[[4-(o-tolyl)-7-quinolyl]oxy]propanoyl]piperidine-3-carboxylate,
ethyl 2-[(3R)-1-[(2R)-2-[[4-(2-chloro-4-fluoro-phenyl)-7-quinolyl]oxy]propanoyl]-3-piperidyl]acetate,
2-[(3R)-1-[(2R)-2-[[4-(2-chlorophenyl)-7-quinolyl]oxy]propanoyl]-3-piperidyl]acetic acid
(3R)-1-[(2R)-2-[[4-(2-chloro-4-fluoro-phenyl)-7-quinolyl]oxy]propanoyl]piperidine-3-carboxylic acid,
2-[(3R)-1-[(2R)-2-[[4-(2-chloro-4-fluoro-phenyl)-7-quinolyl]oxy]propanoyl]-3-piperidyl]acetic acid,
(3S)-1-[(2R)-2-[[4-(2-chloro-4-fluoro-phenyl)-7-quinolyl]oxy]propanoyl]pyrrolidine-3-carboxylic acid,
(2R)-2-[[4-(2-chloro-4-fluoro-phenyl)-7-quinolyl]oxy]-1-(1-piperidyl)propan-1-one,
2-[(3R)-1-[(2R)-2-[[2-chloro-4-(o-tolyl)-7-quinolyl]oxy]propanoyl]-3-piperidyl]acetic acid,
2-[(3S)-1-[(2R)-2-[[4-(2-chloro-4-fluoro-phenyl)-7-quinolyl]oxy]propanoyl]-3-piperidyl]acetic acid,
rac-(3S)-1-[2-[[4-(2-chlorophenyl)-7-quinolyl]oxy]propanoyl]piperidine-3-carboxylic acid,
1-[(2R)-2-[[4-(2-chloro-4-fluoro-phenyl)-7-quinolyl]oxy]propanoyl]piperidine-4-carboxylic acid,
(3S)-1-[rac-(2R)-2-[[2-chloro-4-(o-tolyl)-7-quinolyl]oxy]propanoyl]piperidine-3-carboxylic acid,
3-[[(2R)-2-[[4-(2-chloro-4-fluoro-phenyl)-7-quinolyl]oxy]propanoyl]amino]benzoic acid
ethyl (3S)-1-[(2R)-2-[[4-(2-chloro-4-fluoro-phenyl)-7-quinolyl]oxy]propanoyl]piperidine-3-carboxylate,
(2R)-2-[[4-(2-chloro-4-fluoro-phenyl)-7-quinolyl]oxy]-1-(4-propanoylpiperazin-1-yl)propan-1-one,
tert-butyl (2R)-2-[[4-(2-chlorophenyl)-7-quinolyl]oxy]propanoate,
(3S)-1-[(2R)-2-[[4-(2-chloro-4-fluoro-phenyl)-7-quinolyl]oxy]propanoyl]piperidine-3-carbonitrile,
(3S)-1-[(2R)-2-[[2-methyl-4-(o-tolyl)-7-quinolyl]oxy]propanoyl]piperidine-3-carboxylic acid,
(2R)-2-[[4-(2-chloro-4-fluoro-phenyl)-7-quinolyl]oxy]-N-isopropyl-N-methyl-propanamide,
1-[rac-(2R)-2-[[4-(2-chloro-4-fluoro-phenyl)-7-quinolyl]oxy]propanoyl]piperidine-3-sulfonamide,
(3S)-1-[(2R)-2-[[2-chloro-4-(2-chlorophenyl)-7-quinolyl]oxy]propanoyl]piperidine-3-carboxylic acid,
(3S)-1-[(2R)-2-[[2-chloro-4-(4-fluoro-2-methyl-phenyl)-7-quinolyl]oxy]propanoyl]piperidine-3-carboxylic acid,
isopropyl (2R)-2-[[4-(2-chlorophenyl)-7-quinolyl]oxy]propanoate,
methyl 2-[(3R)-1-[(2R)-2-[[4-(2-chloro-4-fluoro-phenyl)-7-quinolyl]oxy]propanoyl]pyrrolidin-3-yl]acetate,
(3S)-1-[(2R)-2-[[4-(2-chloro-4-fluoro-phenyl)-7-quinolyl]oxy]propanoyl]-N-methyl-piperidine-3-carboxamide,
2-[(3S)-1-[(2R)-2-[[4-(2-chloro-4-fluoro-phenyl)-7-quinolyl]oxy]propanoyl]pyrrolidin-3-yl]acetic acid,
2-[(3R)-1-[(2R)-2-[[4-(2-chloro-4-fluoro-phenyl)-7-quinolyl]oxy]propanoyl]pyrrolidin-3-yl]acetic acid,
ethyl (3S)-1-[(2R)-2-[[4-(2-chlorophenyl)-2-fluoro-7-quinolyl]oxy]propanoyl]piperidine-3-carboxylate,
(2R)-2-[[4-(2-chloro-4-fluoro-phenyl)-7-quinolyl]oxy]-1-pyrrolidin-1-yl-propan-1-one,
(2R)-2-[[2-chloro-4-(o-tolyl)-7-quinolyl]oxy]propanoic acid,
(2R)-2-[[4-(2-chloro-4-fluoro-phenyl)-7-quinolyl]oxy]propanoic acid,
(2R)-2-[[4-(2-chloro-4-fluoro-phenyl)-7-quinolyl]oxy]-N,N-dimethyl-propanamide,
rac-(3S)-1-[2-[[4-(2-chlorophenyl)-7-quinolyl]oxy]acetyl]piperidine-3-carboxylic acid,
(2R)—N-tert-butyl-2-[[4-(2-chloro-4-fluoro-phenyl)-7-quinolyl]oxy]propanamide,
(2R)-2-[[4-(2-chlorophenyl)-7-quinolyl]oxy]-N-isopropyl-propanamide,
ethyl 2-[(3R)-1-[(2R)-2-[[2-methyl-4-(o-tolyl)-7-quinolyl]oxy]propanoyl]-3-piperidyl]acetate,
ethyl (2R)-2-[[4-(2-chlorophenyl)-7-quinolyl]oxy]propanoate,
ethyl 4-[[(2R)-2-[[4-(2-chloro-4-fluoro-phenyl)-7-quinolyl]oxy]propanoyl]amino]benzoate,
(3S)-1-[(2R)-2-[[2-chloro-4-(o-tolyl)-7-quinolyl]oxy]propanoyl]piperidine-3-carboxamide,
(2R)-2-[[4-(2-chlorophenyl)-7-quinolyl]oxy]-1-(1-piperidyl)propan-1-one,
methyl 3-[[(2R)-2-[[4-(2-chloro-4-fluoro-phenyl)-7-quinolyl]oxy]propanoyl]amino]cyclobutanecarboxylate,
(2R)-2-[[4-(2-chloro-4-fluoro-phenyl)-7-quinolyl]oxy]-1-piperazin-1-yl-propan-1-one,
2-[(3R)-1-[(2R)-2-[[2-chloro-4-(2-chloro-4-fluoro-phenyl)-7-quinolyl]oxy]propanoyl]-3-piperidyl]acetic acid,
2-[(3R)-1-[(2R)-2-[[2-chloro-4-(4-fluoro-2-methyl-phenyl)-7-quinolyl]oxy]propanoyl]-3-piperidyl]acetic acid,
ethyl 1-[(2R)-2-[[4-(2-chloro-4-fluoro-phenyl)-7-quinolyl]oxy]propanoyl]piperidine-4-carboxylate,
(3S)-1-[(2R)-2-[[4-(2-chloro-4-fluoro-phenyl)-2-methyl-7-quinolyl]oxy]propanoyl]piperidine-3-carboxylic acid,
4-[[(2R)-2-[[4-(2-chloro-4-fluoro-phenyl)-7-quinolyl]oxy]propanoyl]amino]benzoic acid,
(2R)-2-[[4-(2-chloro-4-fluoro-phenyl)-7-quinolyl]oxy]-N-isopropyl-propanamide,
(3S)-1-[2-[[5-(2-chloro-4-fluoro-phenyl)-1,8-naphthyridin-2-yl]oxy]propanoyl]piperidine-3-carboxylic acid,
4-[(2R)-2-[[4-(2-chloro-4-fluoro-phenyl)-7-quinolyl]oxy]propanoyl]piperazin-2-one,
(3S)-1-[(2R)-2-[[2-chloro-4-(2-chloro-4-fluoro-phenyl)-7-quinolyl]oxy]propanoyl]piperidine-3-carboxylic acid,
2-[(3R)-1-[(2R)-2-[[4-(2-chloro-3-fluoro-phenyl)-7-quinolyl]oxy]propanoyl]-3-piperidyl]acetic acid,
(3S)-1-[(2R)-2-[(4-phenyl-7-quinolyl)oxy]propanoyl]piperidine-3-carboxylic acid,
methyl 2-[(3S)-1-[(2R)-2-[[4-(2-chloro-4-fluoro-phenyl)-7-quinolyl]oxy]propanoyl]pyrrolidin-3-yl]acetate,
2-[(3R)-1-[(2R)-2-[[2-chloro-4-(2-chlorophenyl)-7-quinolyl]oxy]propanoyl]-3-piperidyl]-N-methyl-acetamide,
2-[(3R)-1-[(2R)-2-[[2-methyl-4-(o-tolyl)-7-quinolyl]oxy]propanoyl]-3-piperidyl]acetic acid,
(2R)-2-[[2-chloro-4-(2-chlorophenyl)-7-quinolyl]oxy]-1-[4-(cyclopropanecarbonyl)piperazin-1-yl]propan-1-one,
methyl (3R)-1-[(2R)-2-[[4-(2-chloro-4-fluoro-phenyl)-7-quinolyl]oxy]propanoyl]piperidine-3-carboxylate,
(2R)-2-[[2-chloro-4-(2-chlorophenyl)-7-quinolyl]oxy]-1-(4-propanoylpiperazin-1-yl)propan-1-one,
tert-butyl (2R)-2-[[2-chloro-4-(o-tolyl)-7-quinolyl]oxy]propanoate,
ethyl 2-[(3R)-1-[(2R)-2-[[4-(2-chloro-4-fluoro-phenyl)-2-methyl-7-quinolyl]oxy]propanoyl]-3-piperidyl]acetate,
ethyl (3S)-1-[(2R)-2-[[4-(2-chloro-4-fluoro-phenyl)-7-quinolyl]oxy]propanoyl]pyrrolidine-3-carboxylate,
(3S)-1-[(2R)-2-[[2-chloro-4-(2-chlorophenyl)-7-quinolyl]oxy]propanoyl]piperidine-3-carboxamide,
(2R)-2-[[4-(2-chloro-4-fluoro-phenyl)-7-quinolyl]oxy]-N-cyclopropyl-propanamide,
(3S)-1-[(2S)-2-[[4-(2-chlorophenyl)-7-quinolyl]oxy]propanoyl]piperidine-3-carboxylic acid,
2-[(3R)-1-[(2R)-2-[[4-(2-methyl-3-thienyl)-7-quinolyl]oxy]propanoyl]-3-piperidyl]acetic acid,
2-[rac-(3R)-1-[2-[[5-(2-chloro-4-fluoro-phenyl)-1,8-naphthyridin-2-yl]oxy]propanoyl]-3-piperidyl]acetic acid,
(3S)-1-[(2R)-2-[[2-chloro-4-(2-chloro-4-fluoro-phenyl)-7-quinolyl]oxy]propanoyl]piperidine-3-carboxamide,
isopropyl 2-[[4-(2-chlorophenyl)-7-quinolyl]oxy]acetate,
(3S)-1-[(2R)-2-[[4-(4-fluoro-2,6-dimethyl-phenyl)-7-quinolyl]oxy]propanoyl]piperidine-3-carboxylic acid,
(2R)-1-[4-(2-aminoacetyl)piperazin-1-yl]-2-[[2-chloro-4-(2-chlorophenyl)-7-quinolyl]oxy]propan-1-one,
2-[[4-(2-chlorophenyl)-7-quinolyl]oxy]-N,N-dimethyl-propanamide,
ethyl (3S)-1-[2-[[4-(2-chlorophenyl)-7-quinolyl]oxy]acetyl]piperidine-3-carboxylate,
(2R)-2-[[4-(2-chlorophenyl)-7-quinolyl]oxy]-N,N-dimethyl-propanamide,
ethyl 2-[(3S)-1-[(2R)-2-[[4-(2-chloro-4-fluoro-phenyl)-7-quinolyl]oxy]propanoyl]-3-piperidyl]acetate,
(2R)-2-[[4-(2-chlorophenyl)-2-fluoro-7-quinolyl]oxy]propanoic acid,
(2R)-2-[[2-chloro-4-(2-chloro-4-fluoro-phenyl)-7-quinolyl]oxy]propanoic acid,
ethyl 2-[(3R)-1-[(2R)-2-[[2-chloro-4-(2-chlorophenyl)-7-quinolyl]oxy]propanoyl]-3-piperidyl]acetate,
ethyl 2-[(3R)-1-[(2R)-2-[[2-chloro-4-(2-chloro-4-fluoro-phenyl)-7-quinolyl]oxy]propanoyl]-3-piperidyl]acetate,
ethyl (3S)-1-[(2R)-2-[[2-chloro-4-(4-fluoro-2-methyl-phenyl)-7-quinolyl]oxy]propanoyl]piperidine-3-carboxylate,
(2R)-2-[[4-(2-chloro-4-fluoro-phenyl)-7-quinolyl]oxy]-N-(4-pyridyl)propanamide,
(3S)-1-[(2R)-2-[[2-chloro-4-(2-chlorophenyl)-7-quinolyl]oxy]propanoyl]-N-methyl-piperidine-3-carboxamide,
methyl 3-[[(2R)-2-[[2-chloro-4-(2-chlorophenyl)-7-quinolyl]oxy]propanoyl]amino]cyclobutanecarboxylate,
2-[(3R)-1-[(2R)-2-[[2-chloro-4-(2-chloro-4-fluoro-phenyl)-7-quinolyl]oxy]propanoyl]-3-piperidyl]-N-methyl-acetamide,
(2R)-2-[[2-chloro-4-(2-chlorophenyl)-7-quinolyl]oxy]-1-(1-piperidyl)propan-1-one,
(3S)-1-[(2R)-2-[[2-chloro-4-(2-chlorophenyl)-7-quinolyl]oxy]propanoyl]-N,N-dimethyl-piperidine-3-carboxamide,
2-[(3R)-1-[(2R)-2-[[4-(2-chloro-4-fluoro-phenyl)-2-methyl-7-quinolyl]oxy]propanoyl]-3-piperidyl]acetic acid,
(3R)-1-[(2S)-2-[[4-(2-chlorophenyl)-7-quinolyl]oxy]propanoyl]piperidine-3-carboxylic acid,
(2R)-2-[[4-(2-chlorophenyl)-7-quinolyl]oxy]propanoic acid,
(2R)-2-[[2-chloro-4-(2-chlorophenyl)-7-quinolyl]oxy]-1-piperazin-1-yl-propan-1-one,
(3S)—N-methyl-1-[(2R)-2-[[2-chloro-4-(2-chloro-4-fluoro-phenyl)-7-quinolyl]oxy]propanoyl]piperidine-3-carboxamide,
2-[[4-(o-tolyl)-7-quinolyl]oxy]acetamide,
(2R)-2-[[2-chloro-4-(2-chloro-4-fluoro-phenyl)-7-quinolyl]oxy]-1-(1-piperidyl)propan-1-one,
ethyl 3-[[(2R)-2-[[4-(2-chloro-4-fluoro-phenyl)-7-quinolyl]oxy]propanoyl]amino]benzoate,
2-[[5-(2-chloro-4-fluoro-phenyl)-1,8-naphthyridin-2-yl]oxy]-N-isopropyl-propanamide,
2-[(3R)-1-[(2R)-2-[[4-(2-fluorophenyl)-7-quinolyl]oxy]propanoyl]-3-piperidyl]acetic acid,
tert-butyl (3S)-1-[(2R)-2-[[2-methyl-4-(o-tolyl)-7-quinolyl]oxy]propanoyl]piperidine-3-carboxylate,
2-[1-[(2R)-2-[[4-(2-chlorophenyl)-7-quinolyl]oxy]propanoyl]-4-piperidyl]acetic acid,
(3S)-1-[rac-(2R)-2-[[4-(2,6-dimethylphenyl)-2-methyl-7-quinolyl]oxy]propanoyl]piperidine-3-sulfonamide,
(3R)-1-[rac-(2R)-2-[[4-(2,6-dimethylphenyl)-2-methyl-7-quinolyl]oxy]propanoyl]piperidine-3-sulfonamide,
(3R)-1-[rac-(2R)-2-[[4-(2,6-dimethylphenyl)-2-methyl-7-quinolyl]oxy]propanoyl]piperidine-3-sulfonamide,
(3R)-1-[(2R)-2-[[4-(2-chloro-4-fluoro-phenyl)-2-methyl-7-quinolyl]oxy]propanoyl]piperidine-3-sulfonamide,
(3S)-1-[(2R)-2-[[4-(2-chloro-4-fluoro-phenyl)-7-quinolyl]oxy]propanoyl]piperidine-3-sulfonamide,
(3R)-1-[(2R)-2-[[4-(2-chloro-4-fluoro-phenyl)-7-quinolyl]oxy]propanoyl]piperidine-3-sulfonamide,
3-[1-[(2R)-2-[[4-(2-chlorophenyl)-7-quinolyl]oxy]propanoyl]-4-piperidyl]propanoic acid,
1-[rac-(2R)-2-[[4-(2,6-dimethylphenyl)-7-quinolyl]oxy]propanoyl]piperidine-3-sulfonamide,
2-[(3R)-1-[(2R)-2-[[4-(2,6-dichlorophenyl)-7-quinolyl]oxy]propanoyl]-3-piperidyl]acetic acid,
2-[(3R)-1-[(2R)-2-[[4-(2-ethylphenyl)-7-quinolyl]oxy]propanoyl]-3-piperidyl]acetic acid,
2-[(3R)-1-[(2R)-2-[[4-(2-isopropylphenyl)-7-quinolyl]oxy]propanoyl]-3-piperidyl]acetic acid,
[1-[rac-(2R)-2-[[4-(2-chloro-4-fluoro-phenyl)-7-quinolyl]oxy]propanoyl]-3-piperidyl]methanesulfonamide,
2-[(3R)-1-[(2R)-2-[[4-(2,6-dimethylphenyl)-2-methyl-7-quinolyl]oxy]propanoyl]-3-piperidyl]acetic acid,
(2R)-2-[[4-(2-chloro-4-fluoro-phenyl)-7-quinolyl]oxy]-N-[4-(2-hydroxyethyl)phenyl]propanamide,
(3S)-1-[(2R)-2-[[5-(2-chloro-4-fluoro-phenyl)-1,8-naphthyridin-2-yl]oxy]propanoyl]piperidine-3-carboxylic acid,
(3S)-1-[(2S)-2-[[5-(2-chloro-4-fluoro-phenyl)-1,8-naphthyridin-2-yl]oxy]propanoyl]piperidine-3-carboxylic acid,
(3S)-1-[(2S)-2-[[5-(2-chloro-4-fluoro-phenyl)-1,8-naphthyridin-2-yl]oxy]propanoyl]piperidine-3-carboxylic acid,
2-[(3R)-1-[(2R)-2-[[5-(2-chloro-4-fluoro-phenyl)-1,8-naphthyridin-2-yl]oxy]propanoyl]-3-piperidyl]acetic acid,
2-[(3R)-1-[(2R)-2-[[5-(2-chloro-4-fluoro-phenyl)-1,8-naphthyridin-2-yl]oxy]propanoyl]-3-piperidyl]acetic acid,
(3S)-1-[(2R)-2-[[4-(2,6-dichlorophenyl)-7-quinolyl]oxy]propanoyl]piperidine-3-carboxylic acid,
(2R)-2-[[4-(2-chloro-4-fluoro-phenyl)-7-quinolyl]oxy]-N-(2-pyridyl)propanamide,
(2R)-2-[[4-(2-chloro-4-fluoro-phenyl)-7-quinolyl]oxy]-N-ethyl-propanamide,
(3S)-1-[(2R)-2-[[4-(2,6-dichloro-4-fluoro-phenyl)-7-quinolyl]oxy]propanoyl]piperidine-3-carboxylic acid
2-[(3R)-1-[(2R)-2-[[4-(4-methyl-3-thienyl)-7-quinolyl]oxy]propanoyl]-3-piperidyl]acetic acid
2-[(3R)-1-[(2R)-2-[[4-(3-methyl-2-thienyl)-7-quinolyl]oxy]propanoyl]-3-piperidyl]acetic acid
2-[(3R)-1-[(2R)-2-[[4-(2-methoxyphenyl)-7-quinolyl]oxy]propanoyl]-3-piperidyl]acetic acid
2-[(3R)-1-[(2R)-2-[[4-[2-(trifluoromethyl)phenyl]-7-quinolyl]oxy]propanoyl]-3-piperidyl]acetic acid,
2-[(3R)-1-[(2R)-2-[[4-[2-(trifluoromethoxy)phenyl]-7-quinolyl]oxy]propanoyl]-3-piperidyl]acetic acid,
(3S)-1-[(2R)-2-[[4-(2,6-dimethylphenyl)-7-quinolyl]oxy]propanoyl]piperidine-3-sulfonamide,
2-[(3R)-1-[2-[[4-(2-chlorophenyl)-7-quinolyl]oxy]-2-methyl-propanoyl]-3-piperidyl]acetic acid,
2-[rac-(3R)-1-[2-[[4-(2-chlorophenyl)-7-quinolyl]oxy]butanoyl]-3-piperidyl]acetic acid,
2-[(3R)-1-[(2R)-2-[[4-(2-chloro-6-methyl-phenyl)-7-quinolyl]oxy]propanoyl]-3-piperidyl]acetic acid,
2-[(3R)-1-[(2R)-2-[[4-(2-bromophenyl)-7-quinolyl]oxy]propanoyl]-3-piperidyl]acetic acid,
2-[(3R)-1-[(2R)-2-[[4-(2-cyanophenyl)-7-quinolyl]oxy]propanoyl]-3-piperidyl]acetic acid,
2-[(3R)-1-[(2R)-2-[[4-(2-ethynylphenyl)-7-quinolyl]oxy]propanoyl]-3-piperidyl]acetic acid,
2-[(3R)-1-[(2R)-2-[[4-[2-(dimethylamino)phenyl]-7-quinolyl]oxy]propanoyl]-3-piperidyl]acetic acid,
2-[(3R)-1-[(2R)-2-[[4-(2-carbamoylphenyl)-7-quinolyl]oxy]propanoyl]-3-piperidyl]acetic acid,
2-[(3R)-1-[(2R)-2-[[4-(2,6-difluorophenyl)-7-quinolyl]oxy]propanoyl]-3-piperidyl]acetic acid,
2-[(3R)-1-[(2R)-2-[[4-(2,4-dimethyl-3-thienyl)-7-quinolyl]oxy]propanoyl]-3-piperidyl]acetic acid,
2-[(3R)-1-[(2R)-2-[[4-[2-chloro-6-(trifluoromethyl)phenyl]-7-quinolyl]oxy]propanoyl]-3-piperidyl]acetic acid,
2-[(3R)-1-[(2R)-2-[[4-(2-bromo-6-chloro-phenyl)-7-quinolyl]oxy]propanoyl]-3-piperidyl]acetic acid,
2-[(3R)-1-[(2R)-2-[[4-[2,6-bis(trifluoromethyl)phenyl]-7-quinolyl]oxy]propanoyl]-3-piperidyl]acetic acid,
2-[(3R)-1-[(2R)-2-[[4-(2-chloro-6-methoxy-phenyl)-7-quinolyl]oxy]propanoyl]-3-piperidyl]acetic acid,
2-[(3R)-1-[(2R)-2-[[4-(2,6-diisopropylphenyl)-7-quinolyl]oxy]propanoyl]-3-piperidyl]acetic acid,
(2R)-1-[(3R)-3-amino-1-piperidyl]-2-[[4-(2,6-dimethylphenyl)-7-quinolyl]oxy]propan-1-one,
(2R)-1-[(3S)-3-amino-1-piperidyl]-2-[[4-(2,6-dimethylphenyl)-7-quinolyl]oxy]propan-1-one,
N-[(3S)-1-[(2R)-2-[[4-(2,6-dimethylphenyl)-7-quinolyl]oxy]propanoyl]-3-piperidyl]propanamide,
N-tert-butyl-4-[(2R)-2-[[4-(2,6-dimethylphenyl)-7-quinolyl]oxy]propanoyl]piperazine-1-carboxamide,
(2R)-2-[[4-(2,6-dimethylphenyl)-7-quinolyl]oxy]-1-[3-(1-hydroxycyclopropyl)-1-piperidyl]propan-1-one,
8-[(2R)-2-[[4-(2,6-dimethylphenyl)-7-quinolyl]oxy]propanoyl]-2,8-diazaspiro[4.5]decan-1-one,
(2R)-2-[[4-(2,6-dimethylphenyl)-7-quinolyl]oxy]-1-(3,5-dimethylpiperazin-1-yl)propan-1-one,
N-[(3S)-1-[(2R)-2-[[4-(2,6-dimethylphenyl)-7-quinolyl]oxy]propanoyl]-3-piperidyl]-N-hydroxy-acetamide,
1-[4-[(2R)-2-[[4-(2,6-dimethylphenyl)-7-quinolyl]oxy]propanoyl]piperazin-1-yl]-2,2-dimethyl-propan-1-one,
N-[(3S)-1-[(2R)-2-[[4-(2,6-dimethylphenyl)-7-quinolyl]oxy]propanoyl]-3-piperidyl]methanesulfonamide,
N-[(3S)-1-[(2R)-2-[[4-(2,6-dimethylphenyl)-7-quinolyl]oxy]propanoyl]-3-piperidyl]benzamide,
(3S)—N-cyano-1-[(2R)-2-[[4-(2,6-dimethylphenyl)-7-quinolyl]oxy]propanoyl]piperidine-3-carboxamide,
(3S)-1-[(2R)-2-[[4-(2,6-dimethylphenyl)-7-quinolyl]oxy]propanoyl]piperidine-3-carbohydroxamic acid,
2-[(3R)-1-[(2R)-2-[[4-(2,6-dimethylphenyl)-7-quinolyl]oxy]propanoyl]-3-piperidyl]ethanehydroxamic acid,
(2R)-1-(3-aminoazetidin-1-yl)-2-[[4-(2,6-dimethylphenyl)-7-quinolyl]oxy]propan-1-one,
(2R)-2-[[4-(2,6-dimethylphenyl)-7-quinolyl]oxy]-1-[3-(1H-tetrazol-5-yl)azetidin-1-yl]propan-1-one,
3-hydroxy-1-[(2R)-2-[[4-(2,6-dimethylphenyl)-7-quinolyl]oxy]propanoyl]piperidine-3-carboxylic acid,
5-[(2R)-2-[[4-(2,6-dimethylphenyl)-7-quinolyl]oxy]propanoyl]-5-azaspiro[2.5]octane-2-carboxylic acid,
(3R)-1-[(2R)-2-[[4-(2,6-dimethylphenyl)-7-quinolyl]oxy]propanoyl]piperidine-3-sulfonamide,
(3S)-1-[(2R)-2-[[4-(2,6-dichloro-4-fluoro-phenyl)-7-quinolyl]oxy]propanoyl]-3-methyl-piperidine-3-carboxylic acid,
5-[rac-(2R)-2-[[4-(4-fluoro-2,6-dimethyl-phenyl)-7-quinolyl]oxy]propanoyl]-5-azaspiro[2.5]octane-2-carboxylic acid,
(3R)-1-[(2R)-2-[[4-(4-fluoro-2,6-dimethyl-phenyl)-7-quinolyl]oxy]propanoyl]-3-methyl-piperidine-3-carboxylic acid,
(2R)-2-[[4-(4-fluoro-2,6-dimethyl-phenyl)-7-quinolyl]oxy]-1-[(2S)-2-methyl-1-piperidyl]propan-1-one,
5-[rac-(2R)-2-[[4-(2,6-dichloro-4-fluoro-phenyl)-7-quinolyl]oxy]propanoyl]-5-azaspiro[2.5]octane-2-carboxylic acid,
(2R)-2-[[4-(4-fluoro-2,6-dimethyl-phenyl)-7-quinolyl]oxy]-1-morpholino-propan-1-one,
(2R)-2-[[4-(4-fluoro-2,6-dimethyl-phenyl)-7-quinolyl]oxy]-1-(2-oxa-7-azaspiro[3.5]nonan-7-yl)propan-1-one,
rac-(2R)-2-[[4-(4-fluoro-2,6-dimethyl-phenyl)-7-quinolyl]oxy]-1-[3-(1-hydroxycyclopropyl)-1-piperidyl]propan-1-one,
8-[(2R)-2-[[4-(4-fluoro-2,6-dimethyl-phenyl)-7-quinolyl]oxy]propanoyl]-2,8-diazaspiro[4.5]decan-1-one,
1-[1-[rac-(2R)-2-[[4-(4-fluoro-2,6-dimethyl-phenyl)-7-quinolyl]oxy]propanoyl]-3-piperidyl]cyclopropanecarboxylic acid,
2-[(3R)-1-[(2R)-2-[[4-(4-fluoro-2,6-dimethyl-phenyl)-7-quinolyl]oxy]propanoyl]-3-piperidyl]acetic acid,
(3S)-1-[(2R)-2-[[4-(4-fluoro-2,6-dimethyl-phenyl)-7-quinolyl]oxy]propanoyl]-3-methyl-piperidine-3-carboxylic acid,
N-[(3S)-1-[(2R)-2-[[4-(4-fluoro-2,6-dimethyl-phenyl)-7-quinolyl]oxy]propanoyl]-3-piperidyl]-N-hydroxy-acetamide,
2-methyl-2-[1-[rac-(2R)-2-[[4-(4-fluoro-2,6-dimethyl-phenyl)-7-quinolyl]oxy]propanoyl]-3-piperidyl]propanoic acid,
(2R)-2-[[4-(4-fluoro-2,6-dimethyl-phenyl)-7-quinolyl]oxy]-1-[(2R)-2-methyl-1-piperidyl]propan-1-one,
(3R)-1-[(2R)-2-[[4-(2,6-dichloro-4-fluoro-phenyl)-7-quinolyl]oxy]propanoyl]-3-methyl-piperidine-3-carboxylic acid,
(3S)-1-[(2R)-2-[[4-(2-chloro-4-fluoro-phenyl)-7-quinolyl]oxy]propanoyl]-3-methyl-piperidine-3-carboxylic acid,
[1-[rac-(2R)-2-[[4-(2,6-dimethylphenyl)-7-quinolyl]oxy]propanoyl]-3-piperidyl]methanesulfonamide,
rac-(2R)-1-(2,6-dimethyl-1-piperidyl)-2-[[4-(4-fluoro-2,6-dimethyl-phenyl)-7-quinolyl]oxy]propan-1-one,
2-[(3R)-1-[(2R)-2-[[4-(2,6-dichloro-4-fluoro-phenyl)-7-quinolyl]oxy]propanoyl]-3-piperidyl]acetic acid,
5-[rac-(2R)-2-[[4-(2-chloro-4-fluoro-phenyl)-7-quinolyl]oxy]propanoyl]-5-azaspiro[2.5]octane-2-carboxylic acid,
1-[1-[rac-(2R)-2-[[4-(2-chloro-4-fluoro-phenyl)-7-quinolyl]oxy]propanoyl]-3-piperidyl]cyclopropanecarboxylic acid,
2-methyl-2-[1-[rac-(2R)-2-[[4-(2,6-dichloro-4-fluoro-phenyl)-7-quinolyl]oxy]propanoyl]-3-piperidyl]propanoic acid,
1-[1-[rac-(2R)-2-[[4-(2,6-dichloro-4-fluoro-phenyl)-7-quinolyl]oxy]propanoyl]-3-piperidyl]cyclopropanecarboxylic acid,
2-methyl-2-[1-[rac-(2R)-2-[[4-(2-chloro-4-fluoro-phenyl)-7-quinolyl]oxy]propanoyl]-3-piperidyl]propanoic acid,
(3R)-1-[(2R)-2-[[4-(2-chloro-4-fluoro-phenyl)-7-quinolyl]oxy]propanoyl]-3-methyl-piperidine-3-carboxylic acid,
(2R)-2-[[4-(2,6-dichloro-4-fluoro-phenyl)-7-quinolyl]oxy]-1-[(2S)-2-methyl-1-piperidyl]propan-1-one,
rac-(2R)-2-[[4-(2-chloro-4-fluoro-phenyl)-7-quinolyl]oxy]-1-(2-methyl-1-piperidyl)propan-1-one,
(2R)-2-[[4-(2,6-dichloro-4-fluoro-phenyl)-7-quinolyl]oxy]-1-morpholino-propan-1-one,
8-[(2R)-2-[[4-(2,6-dichloro-4-fluoro-phenyl)-7-quinolyl]oxy]propanoyl]-2,8-diazaspiro[4.5]decan-1-one,
rac-(2R)-2-[[4-(2,6-dichloro-4-fluoro-phenyl)-7-quinolyl]oxy]-1-[3-(1-hydroxycyclopropyl)-1-piperidyl]propan-1-one,
(2R)-2-[[4-(2,6-dichloro-4-fluoro-phenyl)-7-quinolyl]oxy]-1-[(2R)-2-methyl-1-piperidyl]propan-1-one,
8-[(2R)-2-[[4-(2-chloro-4-fluoro-phenyl)-7-quinolyl]oxy]propanoyl]-2,8-diazaspiro[4.5]decan-1-one,
(2R)-2-[[4-(2-chloro-4-fluoro-phenyl)-7-quinolyl]oxy]-1-morpholino-propan-1-one,
(2R)-2-[[4-(2-chloro-4-fluoro-phenyl)-7-quinolyl]oxy]-1-[3-(1-hydroxycyclopropyl)-1-piperidyl]propan-1-one,
(2R)-2-[[4-(2,6-dichloro-4-fluoro-phenyl)-7-quinolyl]oxy]-1-(2-oxa-7-azaspiro[3.5]nonan-7-yl)propan-1-one,
N-hydroxy-N-[rac-(3S)-1-[rac-(2R)-2-[[4-(2-chloro-4-fluoro-phenyl)-7-quinolyl]oxy]propanoyl]-3-piperidyl]acetamide,
(2R)-2-[[4-(2-chloro-4-fluoro-phenyl)-7-quinolyl]oxy]-1-(2-oxa-7-azaspiro[3.5]nonan-7-yl)propan-1-one,
(2R)-2-[[4-(2-chloro-4-fluoro-phenyl)-7-quinolyl]oxy]-1-(2,6-dimethyl-1-piperidyl)propan-1-one,
(2R)-2-[[4-(2-chloro-4-fluoro-phenyl)-7-quinolyl]oxy]-1-(2-oxa-8-azaspiro[3.5]nonan-8-yl)propan-1-one,
(2R)-2-[[4-(2-chloro-4-fluoro-phenyl)-7-quinolyl]oxy]-1-(2-oxa-7-azaspiro[3.4]octan-7-yl)propan-1-one,
1-tert-butyl-3-[(3R)-1-[(2R)-2-[[4-(2-chloro-4-fluoro-phenyl)-7-quinolyl]oxy]propanoyl]pyrrolidin-3-yl]urea,
(2R)-2-[[4-(2-chloro-4-fluoro-phenyl)-7-quinolyl]oxy]-1-(3,3,5,5-tetramethylpiperazin-1-yl)propan-1-one,
(2R)-2-[[4-(2-chloro-4-fluoro-phenyl)-7-quinolyl]oxy]-1-(3,5-dimethylpiperazin-1-yl)propan-1-one,
(2R)-2-[[4-(2-chloro-4-fluoro-phenyl)-7-quinolyl]oxy]-1-[(1R)-2,5-diazabicyclo[2.2.1]heptan-2-yl]propan-1-one,
(2R)-2-[[4-(2-chloro-4-fluoro-phenyl)-7-quinolyl]oxy]-1-(2-oxa-6-azaspiro[3,3]heptan-6-yl)propan-1-one,
1-[(2R)-2-[[4-(2-chloro-4-fluoro-phenyl)-7-quinolyl]oxy]propanoyl]-3,6-dihydro-2H-pyridine-5-carboxylic acid,
2-[(3R)-1-[(2R)-2-[[5-(2,6-dichloro-4-fluoro-phenyl)-1,8-naphthyridin-2-yl]oxy]propanoyl]-3-piperidyl]acetic acid, and
2-[(3R)-1-[(2R)-2-[[5-(4-fluoro-2,6-dimethyl-phenyl)-1,8-naphthyridin-2-yl]oxy]propanoyl]-3-piperidyl]acetic acid,
   or a pharmaceutically or veterinary acceptable salt, hydrate or solvate thereof.

In an embodiment, the IMT is selected from the group consisting of N,N-dimethyl-2-[4-(o-tolyl)-2-oxo-chromen-7-yl]oxy-propanamide, 1-[2-[4-(2-chlorophenyl)-2-oxo-chromen-7-yl]oxypropanoyl]piperidine-3-sulfonamide, (3S)-1-[(2R)-2-[4-(2-chloro-4-fluoro-phenyl)-2-oxo-chromen-7-yl]oxypropanoyl]piperidine-3-carboxylic acid, (3S)-1-[2-[4-(2-chloro-4-fluoro-phenyl)-2-oxo-chromen-7-yl]oxypropanoyl]piperidine-3-carboxylic acid, and 2-[(3R)-1-[(2R)-2-[4-(2-chloro-4-fluoro-phenyl)-2-oxo-chromen-7-yl]oxypropanoyl]-3-piperidyl]acetic acid.

The present invention is directed to a composition comprising at least one inhibitor of mitochondrial transcription (IMT) and at least one anti-cancer drug. An anti-cancer drug is a drug that may beneficially influence the course of a cancer disease. The anti-cancer drug is preferably selected from the various “standard-of-care (SOC)” anti-cancer drugs.

In an embodiment, the anti-cancer drug is selected from the group of:
(i) a B-cell lymphocyte-2 anti-apoptotic protein (Bcl-2) inhibitor,
(ii) an inhibitor of the MEK/ERK pathway, including but not limited to a mitogen-activated protein (MAP) kinase inhibitor, MEK inhibitor or ERK inhibitor,
(iii) an inhibitor of poly-ADP Ribose-Polymerase (PARPi),
(iv) a Glucose consumption/uptake inhibitor, including but not limited to 2-deoxy glucose and derivatives and inhibitors of glucose transporters (GLUT),
(v) a dihydroorotate-dehydrogenase (DHODH) inhibitor,
(vi) a phosphatidylinositol-4,5-bisphosphate 3-kinase PIK3Cα (p110α) inhibitor, and
(vii) an immunotherapeutic agent.

Each group of anti-cancer drugs (i) to (vii) may be present in the composition according to the invention individually, or in combination.

Bcl-2 inhibitors, such as the substance venetoclax (ABT-199), are considered standard therapeutic agents in the treatment of e.g. AML patients, however, especially in the age group most affected by this clinical picture, i.e. >60 years, a relapse is observed within a few years after therapy, which is associated with the persistence of Bcl-2 inhibitor-resistant cancer stem cells.

In an embodiment of the present invention, the anti-cancer agent is a B-cell lymphocyte-2 anti-apoptotic protein (Bcl-2) inhibitor.

Therefore, in an embodiment of the present invention an IMT is used in combination with a B-cell lymphocyte-2 anti-apoptotic protein (Bcl-2) inhibitor.

In an embodiment of the present invention, the B-cell lymphocyte-2 anti-apoptotic protein (Bcl-2) inhibitor is selected from the group consisting of Venetoclax (ABT-199), Navitoclax (ABT-263) or Oblimersen (G3139).

Preferred IMTs used in combination with B-cell lymphocyte-2 anti-apoptotic protein (Bcl-2) inhibitors are N,N-dimethyl-2-[4-(o-tolyl)-2-oxo-chromen-7-yl]oxy-propanamide, 1-[2-[4-(2-chlorophenyl)-2-oxo-chromen-7-yl]oxypropanoyl]piperidine-3-sulfonamide, (3S)-1-[(2R)-2-[4-(2-chloro-4-fluoro-phenyl)-2-oxo-chromen-7-yl]oxypropanoyl]piperidine-3-carboxylic acid, (3S)-1-[2-[4-(2-chloro-4-fluoro-phenyl)-2-oxo-chromen-7-yl]oxypropanoyl]piperidine-3-carboxylic acid, and 2-[(3R)-1-[(2R)-2-[4-(2-chloro-4-fluoro-phenyl)-2-oxo-chromen-7-yl]oxypropanoyl]-3-piperidyl]acetic acid.

An IMT in combination with a B-cell lymphocyte-2 anti-apoptotic protein (Bcl-2) inhibitor may be used in the treatment and/or prevention of cancer. An IMT in combination with a B-cell lymphocyte-2 anti-apoptotic protein (Bcl-2) inhibitor is preferably used in the treatment and/or prevention of melanoma, metastatic melanoma, pancreatic cancer, hepatocellular carcinoma, lymphoma, acute myeloid leukemia, breast cancer, glioblastoma, cervical cancer, renal cancer, colorectal cancer or ovarian cancer.

Unlimited growth, as a major feature of cancer cell degeneration, is in many cases caused by dysregulation of the mitogen-activated protein kinase signaling pathway, one of the central control mechanisms of cell division. Therefore, in a number of cancers, treatments with inhibitors addressing different molecular targets of this MAPK pathway have become established. In particular, inhibitors of the MEK and ERK kinases are used in the clinic for BRAF-mutated melanoma and KRAS/BRAF-mutated colon carcinoma. The assumptions made are supported by published data suggesting a particular dependence of BRAF/MEK-resistant cancers on intact mitochondrial function (Viale et al doi:10.1038/nature13611; Zhang et al doi:10.1172/JCI82661).

Based on the assumption that deregulated and increased cell growth conditions are responsible for the need of an increased oxidative metabolism, as well as increased demand for secondary metabolites, a combination of an IMT with an inhibitor of the MEK/ERK pathway may be used. A combination of an IMT with an inhibitor of the MEK/ERK pathway shows synergistic effects for a reduction in cancer cell growth in vitro and a reduction in tumor growth in vivo to be established as beneficial in comparison to the treatment with the individual inhibitors.

Preferred IMTs used in combination with an inhibitor of the MEK/ERK pathway are N,N-dimethyl-2-[4-(o-tolyl)-2-oxo-chromen-7-yl]oxy-propanamide, 1-[2-[4-(2-chlorophenyl)-2-oxo-chromen-7-yl]oxypropanoyl]piperidine-3-sulfonamide, (3S)-1-[(2R)-2-[4-(2-chloro-4-fluoro-phenyl)-2-oxo-chromen-7-yl]oxypropanoyl]piperidine-3-carboxylic acid, (3S)-1-[2-[4-(2-chloro-4-fluoro-phenyl)-2-oxo-chromen-7-yl]oxypropanoyl]piperidine-3-carboxylic acid, and 2-[(3R)-1-[(2R)-2-[4-(2-chloro-4-fluoro-phenyl)-2-oxo-chromen-7-yl]oxypropanoyl]-3-piperidyl]acetic acid.

In an embodiment of the present invention, the inhibitor of the MEK/ERK pathway is selected from the group consisting of Vemurafenib, Dabrafenib, Ulixertinib, Encorafenib (LGX818, (S)-methyl (1-((4-(3-(5-chloro-2-fluoro-3-(methylsulfonamido)phenyl)-1-isopropyl-1H-pyrazol-4-yl)pyrimidin-2-yl)amino)propan-2-yl)carbamate), Trametinib (GSK1120212), Binimetinib (MEK162), Cobimetinib (XL518, GDC0973), Selumetinib (AZD6244), N-[(2R)-2,3-Dihydroxypropoxy]-3,4-difluoro-2-[(2-fluoro-4-iodophenyl)amino]-benzamid (PD-325901), 2-(2-chloro-4-iodophenylamino)-N-cyclopropylmethoxy-3,4-difluorobenzamide (PD-184352/CI-1040), and 3-[(2R)-2,3-Dihydroxypropyl]-6-fluor-5-[(2-fluor-4-iodphenyl)amino]-8-methylpyrido[2,3-d]pyrimidin-4,7(3H,8H)-dion (TAK-733), 2-((2-Fluoro-4-iodophenyl)amino)-N-(2-hydroxyethoxy)-1,5-dimethyl-6-oxo-1,6-dihydropyridine-3-carboxamide (AZD8330), 5-Brom-N-(2,3-dihydroxypropoxy)-3,4-difluor-2-[(2-fluor-4-iodphenyl)amino]benzamid (PD-318088).

An inhibitor of the mitochondrial transcription for the reduction of cell and tumor growth is an inhibitor of poly-ADP Ribose-Polymerase (PARPi).

In an embodiment of the present invention, the inhibitor of poly-ADP Ribose-Polymerase (PARPi) is selected from the group consisting of Olaparib, Rucaparib, Niraparib, Talazoparib, Veliparib, Pamiparib, CEP9722 (11-methoxy-2-((4-methylpiperazin-1-yl)methyl)-4,5,6,7-tetrahydro-1H-cyclopenta[a]pyrrolo[3,4-c]carbazole-1,3(2H)-dione), E7016 (10-((4-Hydroxypiperidin-1-yl)methyl)chromeno[4,3,2-de]phthalazin-3(2H)-one), Iniparib, and 3-aminobenzamide.

In an embodiment, an IMT is used in combination with an inhibitor of poly-ADP Ribose-Polymerase (PARPi).

Preferred IMTs used in combination with an inhibitor of poly-ADP Ribose-Polymerase (PARPi) are N,N-dimethyl-2-[4-(o-tolyl)-2-oxo-chromen-7-yl]oxy-propanamide, 1-[2-[4-(2-chlorophenyl)-2-oxo-chromen-7-yl]oxypropanoyl]piperidine-3-sulfonamide, (3S)-1-[(2R)-2-[4-(2-chloro-4-fluoro-phenyl)-2-oxo-chromen-7-yl]oxypropanoyl]piperidine-3-carboxylic acid, (3S)-1-[2-[4-(2-chloro-4-fluoro-phenyl)-2-oxo-chromen-7-yl]oxypropanoyl]piperidine-3-carboxylic acid, and 2-[(3R)-1-[(2R)-2-[4-(2-chloro-4-fluoro-phenyl)-2-oxo-chromen-7-yl]oxypropanoyl]-3-piperidyl]acetic acid.

An IMT in combination with an inhibitor of poly-ADP Ribose-Polymerase (PARPi) may be used in the treatment and/or prevention of cancer. An IMT in combination with an inhibitor of poly-ADP Ribose-Polymerase (PARPi) is preferably used in the treatment and/or prevention of melanoma, metastatic melanoma, pancreatic cancer, hepatocellular carcinoma, lymphoma, acute myeloid leukemia, breast cancer, glioblastoma, cervical cancer, renal cancer, colorectal cancer or ovarian cancer.

An inhibitor of the glycolysis is a Glucose consumption/uptake inhibitor, including but not limited to 2-deoxy glucose and derivatives and inhibitors of glucose transporters (GLUT).

In an embodiment of the present invention, the anti-cancer agent may be a Glucose consumption/uptake inhibitor, including but not limited to 2-deoxy glucose and derivatives and inhibitors of glucose transporters (GLUT).

Examples of Glucose consumption/uptake inhibitors are Bay-876, fifty compounds listed in Siebeneicher et al., Bioorganic & Medicinal Chemistry Letters (2016), DRB18 and WZB 117 (Shriwas et al. Cancer & Metabolism (2021) 9:14), compounds disclosed and named 8, 10 g and 15b in Liu et al., J. Med. Chem. 2020, 63, 10, 5201-5211, KL-11743 (Liu et al., Nat Cell Biol 22, 476-486 (2020)), NV-5072, NV5440, NV6297 and others disclosed in Kang et al., 2019, Cell Chemical Biology 26, 1-11, Glutor (Reckzeh et al., Cell Chem Bio. 2019; 26(9):1214-28), Chromopynones as described in Karageorgis et al., Chromopynones are pseudo natural product glucose uptake inhibitors targeting glucose transporters GLUT-1 and -3, Nature Chem 10, 1103-1111 (2018), Indomorphanes as described in Ceballos et al., Synthesis of Indomorphan Pseudo Natural Product Inhibitors of Glucose Transporters GLUT-1 and -3, Angew. Chem. Int. Ed. 58(47), 17016-17025 (2019), Ritonavir, compounds described and disclosed by Navitor Pharmaceuticals Inc. (US 2018/0127370, WO 2018/089493, WO 2018/089433), Iomet Pharma LTD (WO 2014/187922), Kadmon Corp LLC (WO 2016/210331, WO 2018/201006, WO 2020/005935) and Bayer Pharma AG (WO 2013/182612, WO 2015/078799, WO 2015/091428, WO 2016/012474, WO 2016/012481) and Lead Discovery Center GmbH and Max-Planck-Gesellschaft zur Förderung der Wissenschaften E.V. (WO2020/049124).

In an embodiment of the present invention, the Glucose consumption/uptake inhibitor is selected from 2-deoxy glucose and derivatives, and the GLUT inhibitor is BAY-876.

In an embodiment, an IMT is used in combination with a Glucose consumption/uptake inhibitor, including but not limited to 2-deoxy glucose and derivatives and inhibitors of glucose transporters (GLUT).

Preferred IMTs used in combination with Glucose consumption/uptake inhibitors are N,N-dimethyl-2-[4-(o-tolyl)-2-oxo-chromen-7-yl]oxy-propanamide, 1-[2-[4-(2-chlorophenyl)-2-oxo-chromen-7-yl]oxypropanoyl]piperidine-3-sulfonamide, (3S)-1-[(2R)-2-[4-(2-chloro-4-fluoro-phenyl)-2-oxo-chromen-7-yl]oxypropanoyl]piperidine-3-carboxylic acid, (3S)-1-[2-[4-(2-chloro-4-fluoro-phenyl)-2-oxo-chromen-7-yl]oxypropanoyl]piperidine-3-carboxylic acid, and 2-[(3R)-1-[(2R)-2-[4-(2-chloro-4-fluoro-phenyl)-2-oxo-chromen-7-yl]oxypropanoyl]-3-piperidyl]acetic acid.

An IMT in combination with inhibitor of a Glucose consumption/uptake inhibitor (GLUT) may be used in the treatment and/or prevention of cancer. An IMT in combination with a Glucose consumption/uptake inhibitor (GLUT) is preferably used in the treatment and/or prevention of melanoma, metastatic melanoma, pancreatic cancer, hepatocellular carcinoma, lymphoma, acute myeloid leukemia, breast cancer, glioblastoma, cervical cancer, renal cancer, colorectal cancer or ovarian cancer.

The mitochondrial protein dihydroorotate dehydrogenase (DHODH) catalyzes the critical step of oxidation of dihydroorotate to orotate, as part of pyrimidine biosynthesis. The pyrimidine biosynthesis is responsible for the availability of nucleotides, which are crucial in tumor growth.

In an embodiment, the anti-cancer agent may be a dihydroorotate-dehydrogenase (DHODH) inhibitor

Therefore, in an embodiment of the present invention an IMT is used in combination with a dihydroorotate-dehydrogenase (DHODH) inhibitor.

In an embodiment of the present invention, the dihydroorotate-dehydrogenase (DHODH) inhibitor is selected from the group consisting of Brequinar, Leflunomide/Teriflunomide, Enliuracil, Vidofludimus, GNF-Pf-4706, (E)-2-((2-(4-(2-chlorophenyl)thiazol-2-yl)hydrazono)methyl)benzoic acid (S312) and (E)-2-((2-(4-(2-chlorophenyl)thiazol-2-yl)-2-methylhydrazono)methyl)benzoic acid (S416).

Preferred IMTs used in combination with dihydroorotate-dehydrogenase (DHODH) inhibitors are N,N-dimethyl-2-[4-(o-tolyl)-2-oxo-chromen-7-yl]oxy-propanamide, 1-[2-[4-(2-chlorophenyl)-2-oxo-chromen-7-yl]oxypropanoyl]piperidine-3-sulfonamide, (3S)-1-[(2R)-2-[4-(2-chloro-4-fluoro-phenyl)-2-oxo-chromen-7-yl]oxypropanoyl]piperidine-3-carboxylic acid, (3S)-1-[2-[4-(2-chloro-4-fluoro-phenyl)-2-oxo-chromen-7-yl]oxypropanoyl]piperidine-3-carboxylic acid, and 2-[(3R)-1-[(2R)-2-[4-(2-chloro-4-fluoro-phenyl)-2-oxo-chromen-7-yl]oxypropanoyl]-3-piperidyl]acetic acid.

An IMT in combination with an inhibitor of DHODH may be used in the treatment and/or prevention of cancer. An IMT in combination with an inhibitor of DHODH is preferably used in the treatment and/or prevention of melanoma, metastatic melanoma, pancreatic cancer, hepatocellular carcinoma, lymphoma, acute myeloid leukemia, breast cancer, glioblastoma, cervical cancer, renal cancer, colorectal cancer or ovarian cancer.

There is a dependency of different tumor types on the capacity for metabolic adaptation, towards increased glutamine metabolism (Hao et al 2016), and a central role of the phosphatidylinositol-4,5-bisphosphate 3-kinase PIK3Cα (p110α).

In an embodiment of the present invention, the anti-cancer drug is a phosphatidylinositol-4,5-bisphosphate 3-kinase PIK3Cα (p110α) inhibitor.

In an embodiment of the present invention an IMT is used in combination with a phosphatidylinositol-4,5-bisphosphate 3-kinase PIK3Cα (p110α) inhibitor.

In an embodiment of the present invention, the phosphatidylinositol-4,5-bisphosphate 3-kinase PIK3Cα (p110α) inhibitor is selected from the group consisting of Duvelisib, Wortmannin, LY294002 (2-(4-Morpholinyl)-8-phenyl-4H-chromen-4-one), Copanlisib (BAY80-6946; 2-Amino-N-{7-methoxy-8-[3-(4-morpholinyl)propoxy]-2,3-dihydroimidazo[1,2-c]quinazolin-5-yl}-5-pyrimidinecarboxamide), AZD6482 (2-({(1R)-1-[7-Methyl-2-(4-morpholinyl)-4-oxo-4H-pyrido[1,2-a]pyrimidin-9-yl]ethyl}amino)benzoic acid), Bimiralisib (5-(4,6-dimorpholin-4-yl-1,3,5-triazin-2-yl)-4-(trifluoromethyl)pyridin-2-amine), Pictilisib (GDC0941; 2-(1H-Indazol-4-yl)-6-[[4-(methylsulfonyl)-1-piperazinyl]methyl]-4-(4-morpholinyl)thieno[3,2-d]pyrimidine), ZSTK474 (2-(Difluoromethyl)-1-[4,6-di(4-morpholinyl)-1,3,5-triazin-2-yl]-1H-benzimidazole), Omipalisib (GSK2126458; 2,4-Difluoro-N-[2-methoxy-5-[4-(4-pyridazinyl)-6-quinolinyl]-3-pyridinyl]benzenesulfonamide) or Buparlisib (BKM120; 5-[2,6-Di(4-morpholinyl)-4-pyrimidinyl]-4-(trifluoromethyl)-2-pyridinamine).

Preferred IMTs used in combination with phosphatidylinositol-4,5-bisphosphate 3-kinase PIK3Cα (p110α) inhibitors are N,N-dimethyl-2-[4-(o-tolyl)-2-oxo-chromen-7-yl]oxy-propanamide, 1-[2-[4-(2-chlorophenyl)-2-oxo-chromen-7-yl]oxypropanoyl]piperidine-3-sulfonamide, (3S)-1-[(2R)-2-[4-(2-chloro-4-fluoro-phenyl)-2-oxo-chromen-7-yl]oxypropanoyl]piperidine-3-carboxylic acid, (3S)-1-[2-[4-(2-chloro-4-fluoro-phenyl)-2-oxo-chromen-7-yl]oxypropanoyl]piperidine-3-carboxylic acid, and 2-[(3R)-1-[(2R)-2-[4-(2-chloro-4-fluoro-phenyl)-2-oxo-chromen-7-yl]oxypropanoyl]-3-piperidyl]acetic acid.

An IMT in combination with a phosphatidylinositol-4,5-bisphosphate 3-kinase PIK3Cα (p110α) inhibitors may be used in the treatment and/or prevention of cancer. An IMT in combination with phosphatidylinositol-4,5-bisphosphate 3-kinase PIK3Cα (p110α) inhibitors is preferably used in the treatment and/or prevention of melanoma, metastatic melanoma, pancreatic cancer, hepatocellular carcinoma, lymphoma, acute myeloid leukemia, breast cancer, glioblastoma, cervical cancer, renal cancer, colorectal cancer or ovarian cancer.

An immunotherapeutic agent may be defined as a substance that induces, enhances, restores or suppresses the host's immune system, or an agent that utilizes or is derived from a component of the immune system. An immunotherapeutic agent may use or modify immune mechanisms. Examples of immunotherapeutic agents are interferone gamma, axitinib (N-Methyl-2-[[3-[(E)-2-pyridin-2-ylethenyl]-1H-indazol-6-yl]sulfanyl]benzamide), lenalidomide ((3RS)-3-(4-Amino-1-oxo-1,3-dihydro-2H-isoindol-2-yl)piperidine-2,6-dione), immune check-point inhibitors pembrolizumab, cemiplimab, durvalumab, ipilimumab, nivolumab, PD-1 ligand inhibitors atezolizumab, avelumab, anti-angiogenic agents ramucirumab, bevacimumab, cetuximab, rituximab, daratumumab, trastuzumab or antibody-drug conjugates bretuximab-vedotin.

In an embodiment, an IMT, preferably an IMT as defined herein, is used in combination with an immunotherapeutic agent.

Preferred IMTs used in combination with an immunotherapeutic agent are N,N-dimethyl-2-[4-(o-tolyl)-2-oxo-chromen-7-yl]oxy-propanamide, 1-[2-[4-(2-chlorophenyl)-2-oxo-chromen-7-yl]oxypropanoyl]piperidine-3-sulfonamide, (3S)-1-[(2R)-2-[4-(2-chloro-4-fluoro-phenyl)-2-oxo-chromen-7-yl]oxypropanoyl]piperidine-3-carboxylic acid, (3S)-1-[2-[4-(2-chloro-4-fluoro-phenyl)-2-oxo-chromen-7-yl]oxypropanoyl]piperidine-3-carboxylic acid, and 2-[(3R)-1-[(2R)-2-[4-(2-chloro-4-fluoro-phenyl)-2-oxo-chromen-7-yl]oxypropanoyl]-3-piperidyl]acetic acid.

An IMT in combination with an immunotherapeutic agent may be used in the treatment and/or prevention of cancer. An IMT in combination with an immunotherapeutic agent is preferably used in the treatment and/or prevention of melanoma, metastatic melanoma, pancreatic cancer, hepatocellular carcinoma, lymphoma, acute myeloid leukemia, breast cancer, glioblastoma, cervical cancer, renal cancer, colorectal cancer or ovarian cancer.

Use of the IMT in Combination with an Ant-Cancer Drug as a Medicament

Furthermore, it has been found that the at least one IMT, such as compounds of formulae (I), (II) and (III), in combination with at least one anti-cancer drug as described herein are suitable for use as a medicament. Specifically, it has been found that the composition comprising at least one IMT and at least one anti-cancer drug as described herein can be used in the treatment of cancer.

POLRMT inhibitors previously have been described to trigger the death of AML cells allegedly through rather unspecific inhibition of mitochondrial transcription, confirming the scientific rational (Bralha et al., 2015). As described in the examples below, compositions of the invention were surprisingly and unexpectedly shown to have cytostatic and/or cytotoxic activity on a number of tumor cells and tumor models both in vitro and in vivo.

Accordingly, the composition of the invention and their pharmaceutically or veterinary acceptable salts, hydrates or solvates, exhibit valuable pharmacological properties and are therefore useful as a medicament or pharmaceutical. The medicament or pharmaceutical can be further formulated with additional pharmaceutically or veterinary acceptable carriers and/or excipients, e.g. for oral administrations in the form of tablets. Tablets may contain suitable binders, lubricants, disintegrating agents, coloring agents, flavoring agents, flow-inducing agents and/or melting agents, generally known in the art.

In one aspect, the invention relates to a pharmaceutical composition comprising a composition as defined herein and a pharmaceutically or veterinary acceptable excipient or carrier.

In a further aspect, the invention relates to a kit comprising at least one inhibitor of mitochondrial transcription (IMT) as defined herein and at least one anti-cancer drug as defined herein

Thus, in one aspect, the invention relates to a composition as defined herein, a pharmaceutical composition as defined herein, or a kit as defined herein for use as a medicament.

Compositions of the invention exhibit a marked and selective inhibitory effect on the POLRMT. This can be determined for example in the Homogeneous TR-FRET assay (see Assay 1) or the Quantitative real time-PCR assay (see Assay 2). The skilled person however may use different assays to determine the direct or indirect inhibition of POLRMT.

As mentioned above, it has been found that the compositions of the invention are useful in the treatment of cancer. There is evidence that in melanoma and especially in metastatic melanoma OXPHOS plays a major role in cancer cells and that inhibition of mitochondria in general may lead to superior treatment success. For example, it was shown that H3K4-demethylase (JARID1B) and OXPHOS dependent drug resistance play a role in metastatic melanoma (Roesch et al., 2013). Haq et al. (2013) describe that the standard of care (SoC) treatment with MEK inhibitors in melanoma leads to PGC1-a-dependent increase in OXPHOS as a drug-resistance escape route. It was also shown that the inhibition of mutated BRAF by vemurafenib increases OXPHOS dependency of BRAF mutated melanoma cells (Schöckel et al., 2015). And further, enhanced OXPHOS, glutaminolysis and β-oxidation constitute the metastatic phenotype of melanoma cells (Rodrigues et al., 2016).

For pancreatic cancer selective killing of OXPHOS-dependent Panc-1 cells has been described for treatment with arctigenin (Brecht et al., 2017). In hepatocellular carcinoma, standard of care (SoC) treatment with MEK inhibitor is leading to PGC1-a-dependent increase in OXPHOS as a drug-resistance escape route (Bhat et al., 2013, Ling et al., 2017).

For lymphoma it has been demonstrated that OXPHOS is dependent on mt-complex III inhibitor antimycin A (Dörr et al., 2013). As described above acute myeloid leukemia, POLRMT inhibitors previously have been described to trigger the death of AML cells allegedly through rather unspecific inhibition of mitochondrial transcription (Bralha et al., 2015).

Also breast cancer should be a suitable cancer indication as overexpression of progesterone receptor is present in more than 50% of all breast cancer patients, whereas progesterone is stimulating mitochondrial activity with subsequent inhibition of apoptosis (Nadji et al., 2005, Behera et al., 2009). Further, the inhibition of mTOR leads to a shift towards OXPHOS-dependence and there is a glucose-dependent effect of mTOR inhibitors in combination with metformin (Pelicano et al., 2014, Ariaans et al., 2017). Additionally, it is described that mitochondrial dysfunction caused by metformin prevents tumor growth in breast cancer (Sanchez-Alvarez et al., 2013).

For glioblastoma it is known that malignant repopulation is dependent on OXPHOS (Yeung et al., 2014). With respect to cervical cancer, POLR™ inhibitors inhibit free fatty acid oxidation (data not shown), which otherwise promote cervical cancer cell proliferation (Rodriguez-Enriquez et al., 2015). In renal cancer there is evidence that Birt-Hogg-Dubé renal tumors are associated with up-regulation of mitochondrial gene expression (Klomp et al., 2010). In colon carcinoma the rational is based on the finding that 5-fluorouracil resistant colorectal/colon cancer cells are addicted to OXPHOS to survive and enhance stem-like traits (Denise et al., 2015).

Accordingly, in another aspect, the invention relates to compositions of the invention as defined herein for use in the treatment of cancer, preferably melanoma, metastatic melanoma, pancreatic cancer, hepatocellular carcinoma, lymphoma, acute myeloid leukemia, breast cancer, glioblastoma, cervical cancer, renal cancer, colorectal cancer or ovarian cancer.

The compounds of the invention are preferably useful in a method for treating and/or preventing cancer in simultaneous, alternating or subsequent combination with another cancer therapy, preferably selected from chemotherapy, immunotherapy, hormone therapy, stem cell transplantation therapy, radiation therapy or surgery. It is likely that the cytostatic activity of the POLRMT inhibitors on tumor cells can be further enhanced by combining the treatment with the respective standard of care in order to get improved/additive treatment results. In this context simultaneous, alternating or subsequent application of the various treatments is envisaged. Any of the standard classes of cancer therapy, chemotherapy, immunotherapy, hormone therapy, stem cell transplantation therapy, radiation therapy or surgery, appears to be feasible for combination with the POLRMT inhibitors of this invention.

Thus, in another aspect, the invention relates to a composition as defined herein, a pharmaceutical composition as defined herein, or a kit as defined herein for use in a method of treating, and/or preventing cancer in a subject.

The subject according to the present invention is preferably a mammalian subject, more preferably a human subject.

In another aspect, the invention relates to a composition as defined herein, a pharmaceutical composition as defined herein, or a kit as defined herein for use in a method of treating, and/or preventing cancer in a subject, wherein the cancer is selected from the group consisting of Acute Lymphoblastic Leukemia (ALL), Acute Myeloid Leukemia (AML), Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Childhood (Brain Cancer), Atypical Teratoid/Rhabdoid Tumor, Childhood, Central Nervous System (Brain Cancer), Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors (Lung Cancer), Burkitt Lymphoma, Carcinoid Tumor (Gastrointestinal), Cardiac (Heart) Tumors (Childhood), Central Nervous System Cancer, Atypical Teratoid/Rhabdoid Tumor (Childhood) (Brain Cancer), Medulloblastoma and Other CNS Embryonal Tumors (Childhood) (Brain Cancer), Germ Cell Tumor (Childhood) (Brain Cancer), Primary CNS Lymphoma, Cervical Cancer, Childhood Cancers, Rare Cancers of Childhood, Cholangiocarcinoma, Chordoma (Childhood) (Bone Cancer), Chronic Lymphocytic Leukemia (CLL), Chronic Myelogenous Leukemia (CML), Chronic Myeloproliferative Neoplasms, Colorectal Cancer, Craniopharyngioma (Childhood) (Brain Cancer), Cutaneous T-Cell Lymphoma (Mycosis Fungoides and Sezary Syndrome), Ductal Carcinoma In Situ (DCIS), Embryonal Tumors, Medulloblastoma and Other Central Nervous System (Childhood) (Brain Cancer), Endometrial Cancer (Uterine Cancer), Ependymoma (Childhood) (Brain Cancer), Esophageal Cancer, Esthesioneuroblastoma (Head and Neck Cancer), Ewing Sarcoma (Bone Cancer), Extracranial Germ Cell Tumor (Childhood), Extragonadal Germ Cell Tumor, Eye Cancer, Intraocular Melanoma, Retinoblastoma, Fallopian Tube Cancer, Fibrous Histiocytoma of Bone (Malignant, and Osteosarcoma), Gallbladder Cancer, Gastric (Stomach) Cancer, Gastrointestinal Carcinoid Tumor, Gastrointestinal Stromal Tumors (GIST) (Soft Tissue Sarcoma), Germ Cell Tumors, Childhood Central Nervous System Germ Cell Tumors (Brain Cancer), Glioma (Brain Cancer), Glioblastoma multiforme (GBM, Brain Cancer), Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors (Childhood), Hepatocellular (Liver) Cancer, Hodgkin Lymphoma, Hypopharyngeal Cancer (Head and Neck Cancer), Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell, Small Cell, Pleuropulmonary Blastoma, and Tracheobronchial Tumor), Lymphoma, Male Breast Cancer, Malignant Fibrous Histiocytoma of Bone and Osteosarcoma, Melanoma, Intraocular (Eye)Melanoma, Merkel Cell Carcinoma (Skin Cancer), Malignant Mesothelioma, Metastatic Cancer, Melanoma Brain Metastatic Cancer, Metastatic Squamous Neck Cancer with Occult Primary (Head and Neck Cancer), Midline Tract Carcinoma With NUT Gene Changes, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Chronic Myeloproliferative Neoplasms, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer, Lip and Oral Cavity Cancer and Oropharyngeal Cancer (Head and Neck Cancer), Osteosarcoma and Undifferentiated Pleomorphic Sarcoma of Bone Treatment, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Pituitary Tumor, Plasma Cell Neoplasm/Multiple Myeloma, Pleuropulmonary Blastoma (Lung Cancer), Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Childhood (Soft Tissue Sarcoma), Salivary Gland Cancer (Head and Neck Cancer), Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Soft Tissue Sarcoma, Uterine Sarcoma, Sezary Syndrome (Lymphoma), Skin Cancer, Small Cell Lung Cancer, Small Intestine Cancer, Soft Tissue Sarcoma, Squamous Cell Carcinoma of the Skin, Metastatic Squamous Neck Cancer with Occult Primary (Head and Neck Cancer), Stomach (Gastric) Cancer, Cutaneous T-Cell Lymphoma, Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Tracheobronchial Tumors (Lung Cancer), Triple-Negative Breast Cancer, Transitional Cell Cancer of the Renal Pelvis and Ureter (Kidney (Renal Cell) Cancer), Carcinoma of Unknown Primary, Ureter and Renal Pelvis, Transitional Cell Cancer (Kidney (Renal Cell) Cancer, Urethral Cancer, Uterine Cancer, Endometrial, Uterine Sarcoma, Vaginal Cancer, Vascular Tumors (Soft Tissue Sarcoma), Vulvar Cancer, Wilms Tumor and Other Childhood Kidney Tumors

In an embodiment, the invention relates to a composition as defined herein, a pharmaceutical composition as defined herein, or a kit as defined herein for use in a method of treating, and/or preventing cancer in a subject, wherein the cancer is selected from the group consisting of melanoma, metastatic melanoma, pancreatic cancer, hepatocellular carcinoma, lymphoma, acute myeloid leukemia, breast cancer, glioblastoma, cervical cancer, renal cancer, colorectal cancer or ovarian cancer.

In an embodiment, the invention relates to a composition as defined herein, a pharmaceutical composition as defined herein, or a kit as defined herein for use in a method of treating and/or preventing cancer in a simultaneous, alternating or subsequent combination with another cancer therapy, preferably selected from chemotherapy, immunotherapy, hormone therapy, stem cell transplantation therapy, radiation therapy or surgery.

### BRIEF DESCRIPTION OF THE FIGURES

FIG. 1 shows the analysis of growth kinetics in the SRB assay format in which the ovarian carcinoma cell line A2780 was treated with the combination of a dilution series of the IMT substance (3S)-1-[2-[4-(2-chloro-4-fluoro-phenyl)-2-oxo-chromen-7-yl]oxypropanoyl]piperidine-3-carboxylic acid and the MEK inhibitor Selumetinib (AZD6244) at a constant concentration of 1 μM or 31 nM. Both tests clearly demonstrated the advantage of the combination treatment compared to the untreated or sample treated only with MEK inhibitor. These findings were further substantiated in a matrix approach, using combinations based on concentration series around the respective IC50 value of both inhibitor classes, in the CTG assay format (Table 4). In two studies, the ovarian cell line A2780 was tested for 72 hours with the IMT N,N-dimethyl-2-[4-(o-tolyl)-2-oxo-chromen-7-yl]oxy-propanamide (30 μM-15 nM) and the MEK inhibitor Selumetinib (30 μM-10 nM), or with the IMT 1-[2-[4-(2-chlorophenyl)-2-oxo-chromen-7-yl]oxypropanoyl]piperidine-3-sulfonamide (30 μM-15 nM) and the MEK inhibitor PD318088 (30 μM-10 nM).

FIG. 2 shows an in vivo xenograft study with a combination of IMT and MEKi in A2780. Time course of tumour growth (top) and body weight change (bottom). Transplant with A2780 on day 0, stratification on day six into six groups (n=8). Treatment with vehicle formulation, twice daily 25 mg/kg or 12.5 mg/kg Selumetinib, once daily 100 mg/kg (3S)-1-[(2R)-2-[4-(2-chloro-4-fluoro-phenyl)-2-oxo-chromen-7-yl]oxypropanoyl]piperidine-3-carboxylic acid, or the combination of IMT with 12.5 mg/kg MEKi or 25 mg/kg MEKi as indicated. Error bars indicate the standard deviation of the respective group. * indicates significant differences from the control group (nonparametric Whitnes-U test, p<0.05).

FIG. 3 shows a broader analysis of the apoptosis induction by the inhibitor combination used in the four AML cell lines MV4-11, MOLM-13, OCI-AML3 and OCI-AML2. This analysis was carried out using flow cytometry and the established cellular markers for necrosis, apoptosis and living cells. It was found that the IMT 2-[(3R)-1-[(2R)-2-[4-(2-chloro-4-fluoro-phenyl)-2-oxo-chromen-7-yl]oxypropanoyl]-3-piperidyl]acetic acid used in these four cell lines, unlike Venetoclax, cannot trigger apoptosis. However, the combination of both inhibitors in MV4-11 and MOLM-13 cells increases the induction of apoptosis significantly beyond the level of Venetoclax alone.

FIG. 4 shows the plasma and liver concentrations of the IMT and Bcl-2i used in combination. Combinations s of 2-[(3R)-1-[(2R)-2-[4-(2-chloro-4-fluoro-phenyl)-2-oxo-chromen-7-yl]oxypropanoyl]-3-piperidyl]acetic acid and Venetoclax were administered orally in a formulation of 0.5% methyl cellulose in water, before the respective substance concentrations (y-axis) of Venetoclax (left) or 2-[(3R)-1-[(2R)-2-[4-(2-chloro-4-fluoro-phenyl)-2-oxo-chromen-7-yl]oxypropanoyl]-3-piperidyl]acetic acid (right) after 4 hours and extraction from blood plasma or liver tissue were analysed by LC-MS. Error bars indicate the standard deviation around the mean of the multiple measurements.

FIG. 5 shows an in vivo CDX study in an MV4-11 model using the combination of IMT and Bcl-2i. A=overview of the study implementation in NBSGW mice injected with MV4-11 AML cells and treated with vehicle, IMT, Venetoclax or a combination thereof (n=10); Blood samples were taken before treatment (week 2) and at set intervals until symptoms of the disease developed; B=blood analysis for hCD45 positive cells; C=survival analysis according to Kaplan-Meier, significance calculated according to Mantle-Cox “log-rank test”; D=analysis for hCD45+ cells in bone marrow samples from the study groups at the time of termination.

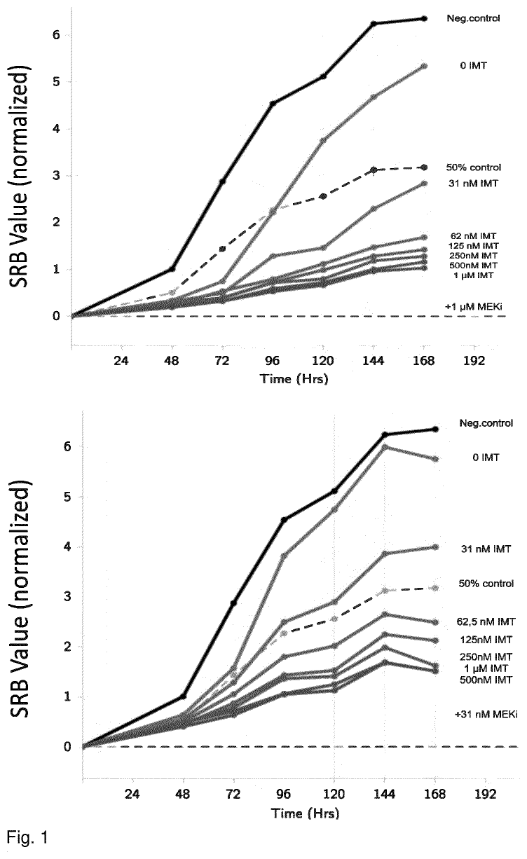

It summarizes the course of a first study in a “cell-derived xenograft (CDX)” model with the MV4-11 AML line and a dosage window of 21 days. The accumulation of AML cells (hCD45+) in peripheral blood and the general survival of the respective treatment groups were observed as the primary readout for the treatment effect. In this study, a considerable superiority of the combination of IMT and Bcl-2i was observed, which was expressed in significantly longer overall survival (+131%) compared to the untreated control group (˜ Venetoclax group). Some of the combination-treated animals showed no signs of disease by the end of the study (day 200). Since blast analyses from bone marrow samples did not differ significantly at the time of stratification of the respective treatment groups and no significant changes in body weight were measured in the combination treatment group over the course of the study, we assume that the combination of IMT and Bcl-2i is clearly advantageous.

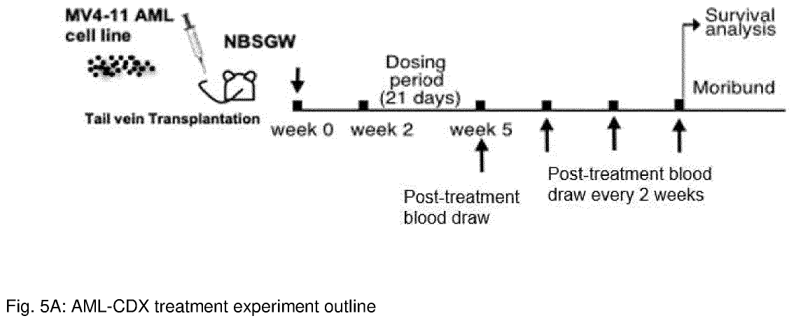

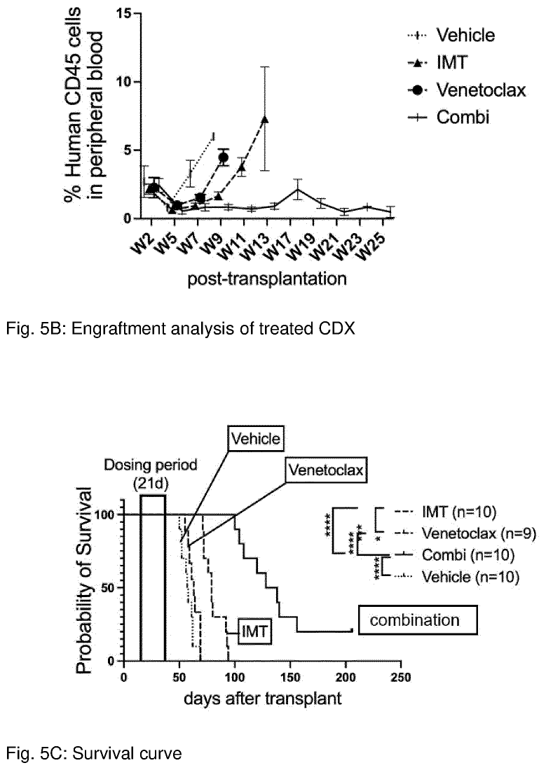

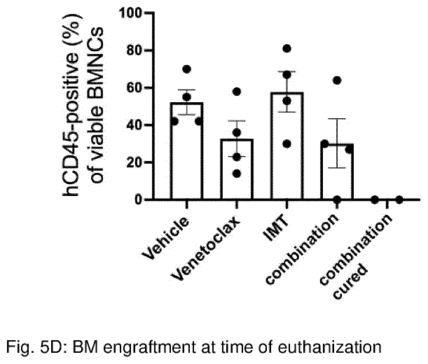

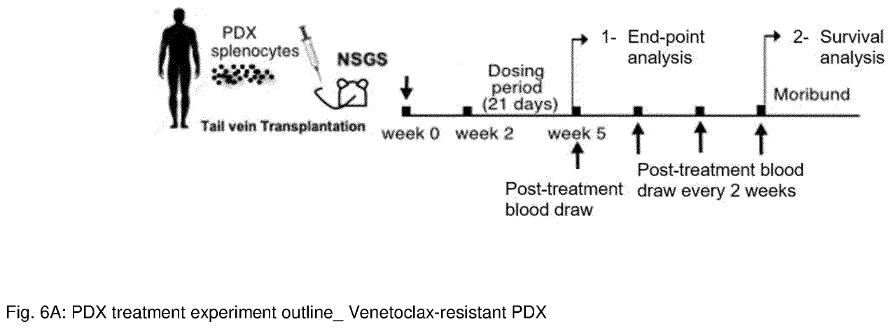

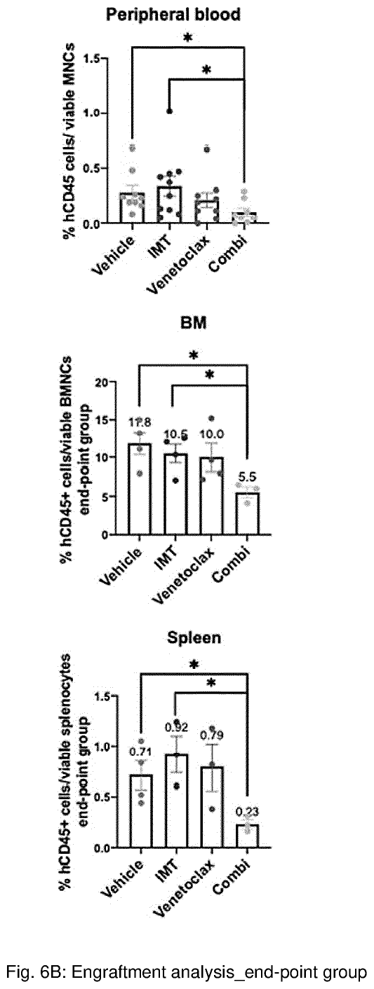

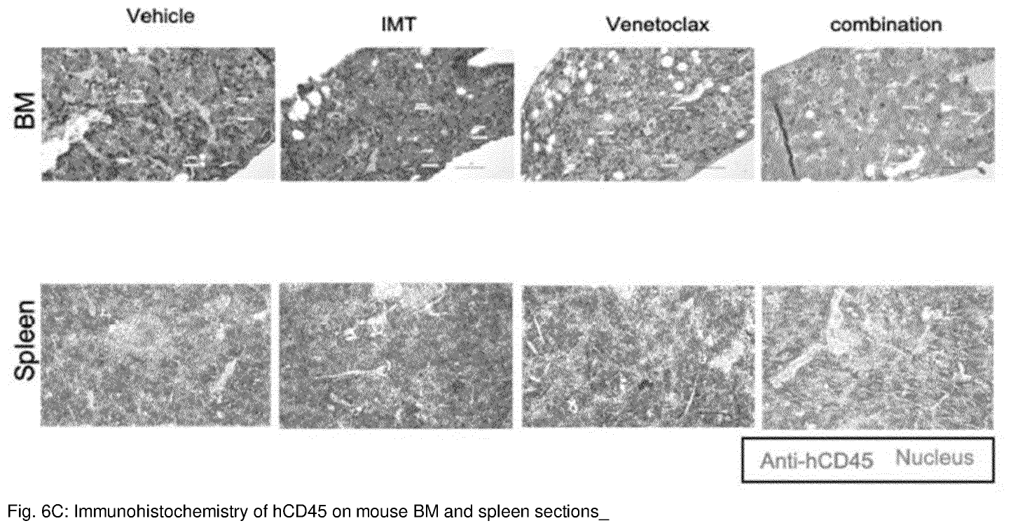

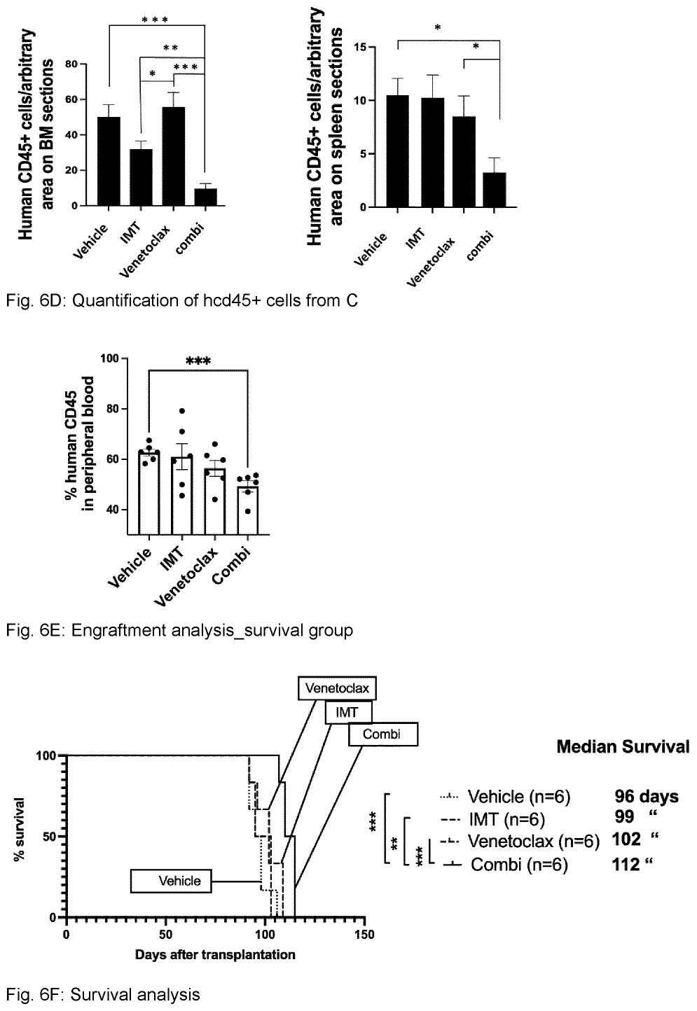

FIG. 6 shows an in vivo PDX study in a Venetoclax-resistant model using the combination of IMT and Bcl-2i. A=overview of the study implementation (corresponding to FIG. 5A) in NSGS mice injected with spleenocytes (n=12). B=flow cytometric analysis and % of live hCD45+AML cells in peripheral blood, spleen and bone marrow of the treated groups; C=immunohistological analysis of bone marrow and spleen tissue sections stained with hcd45 (brown); Quantitative analysis of the hCD45+ cells from C with three technical replicates (n=2). E=blood analysis of the survival group; F=Kaplan-Meier survival analysis (n=6 per group). FIG. 6 shows a Venetoclax-resistant PDX model in NSGS mice. The procedure was similar to that previously described for the CDX model, except for the addition of an endpoint control cohort in each treatment group. Both, flow cytometry of peripheral blood (12 B) and histological analysis of tissue sections from bone marrow and spleen (12 C) clearly show that the group treated with the combination of 2-[(3R)-1-[(2R)-2-[4-(2-chloro-4-fluoro-phenyl)-2-oxo-chromen-7-yl]oxypropanoyl]-3-piperidyl]acetic acid and Venetoclax had a significantly lower tumour load and circulating hCD45-positive AML cells than the animals treated with the individual inhibitors or control group animals. The analysis of the overall survival of the individual groups of the study (survival analysis) proves the long-lasting, synergistic effect of the treatment with the IMT/Bcl-2i combination in this disease model, despite the treatment lasting only 21 days (12 F).

### EXAMPLES

### 1. Methods of Making the Compounds Disclosed Herein

In general, the compounds of formulae (I), (II) and (III), as well as the IMTs used in the invention might be prepared by standard techniques known in the art, by known processes analogous thereto, and/or by the processes described in WO2019/057821, EP 3598972 and WO2020/188049, (hereby incorporated by reference) using starting materials which are either commercially available or producible according to conventional chemical methods. The particular processes to be utilised in the preparation of the compounds of formulae (I), (II) and (III) and the IMT depend upon the specific compound desired. Such factors as the type of substitution at various locations of the molecule and the commercial availability of the starting materials play a role in the path to be followed and in the chosen reaction conditions for the preparation of the specific compounds of formulae (I), (II) and (III) and the IMT. Those factors are readily recognised by one of ordinary skill in the art.

### 2. Assays for Determination of Inhibitors of the POLRMT

Compounds defined herein as IMT inhibitors exhibit a marked and selective inhibitory effect on the mitochondrial RNA-polymerase (POLRMT). This can be determined for example in the Homogeneous TR-FRET assay (see Assay 1) or the Quantitative real time-PCR assay (see Assay 2). The skilled person however may use different assays to determine the direct or indirect inhibition of POLRMT.

### Assay 1: Homogeneous TR-FRET Assay for HTS and Activity Determined

The TR-FRET assay was basically conducted as described in WO2016/193231A1, especially as described in example 1 (hereby incorporated by reference). With respect to the background of the mitochondrial transcription it is referred to Falkenberg et al. (2002) and Posse et al. (Posse et al., 2015). The method monitors the activity of mitochondrial RNA-polymerase via detection of the formation of its product, a 407 bp long RNA sequence. Detection of the product is facilitated by hybridization of two DNA-oligonucleotide probes to specific and adjacent sequences within the RNA product sequence. Upon annealing of the probes, two fluorophores that are coupled directly to an acceptor nucleotide probe (ATT0647, 5′) or introduced via a coupled streptavidin interacting with a biotinylated donor nucleotide probe on the other side (Europium cryptate, 3′) are brought into sufficient proximity to serve as a fluorescence-donor-acceptor pair as generally described in Walters and Namchuk (2003). Thus, a FRET signal at 665 nm is generated upon excitation at 340 nm.

Briefly, the protocol described here was applied for screening and activity determination in a low-volume 384-well microtiter plate with non-binding surface. For high-throughput application in the 1536-well microtiter plate format, volumes of the reagent mixes were adjusted, maintaining the volumetric ratio. Proteins POLRMT (NM_172551.3), TFAM (NM_009360.4) and TFB2M (NM_008249.4) were diluted from their stocks to working concentrations of 150 nM, 1.8 μM and 330 nM respectively, in a dilution buffer containing 100 mM Tris-HCl pH 8.0, 200 mM NaCl, 10% (v/v) glycerole, 2 mM glutathione (GSH), 0.5 mM EDTA and 0.1 mg/mL BSA. Protein dilutions and template DNA, comprising a pUC18 plasmid encoding the mitochondrial light strand promoter, restriction linearized proximal to the promoter 3′-end (pUC-LSP), were mixed at the twofold final assay-concentration in a reaction buffer, containing 10 mM Tris-HCl PH 7.5, 10 mM MgCl₂, 40 mM NaCl, 2 mM GSH, 0.01% (w/v) Tween-20 and 0.1 mg/mL BSA.

5 μL of this mix were dispensed, depending on the chosen microtiter plate format, using multi-channel pipettes or a Multidrop® dispenser (Thermo Fisher Scientific, Waltham MA) into the wells of a microtiter plate and incubated at room temperature (RT) for 10 min. Chemical compounds under scrutiny in the assay were applied using contact-free acoustic droplet-dispensing (Echo520® Labcyte Inc., Sunnyvale CA) from 10 mM compound stocks in 100% DMSO, to a final concentration of 10 μM or in serial dilution series of the required concentration range. Equal amounts of DMSO without any compound were added to positive control samples, followed by an incubation step at RT for 10 min.

The enzymatic reaction was started by the addition of 5 μL of a mix of dNTPs in reaction buffer to a final concentration of 500 UM each. No nucleotide mix was added to negative control samples. The content of the wells was mixed using a VarioTeleshaker™ (Thermo Fisher Scientific, Waltham MA) at 1500 rpm for 45 sec after which the microtiter plate was centrifuged at 500×g for 1 min. The samples were incubated for 2 h at RT with humidity control to avoid evaporation. The detection reagents were prepared in a buffer that was composed, such that the enzymatic reaction was terminated due to chelating of Mg-ions and increased ionic strength, containing 50 mM Tris-HCl PH 7.5, 700 mM NaCl, 20 mM EDTA, and 0.01%(w/v) Tween-20. Importantly Eu-cryptate-coupled streptavidin had to be pre-incubated with a 100-fold molar excess of a random sequence oligonucleotide for 10 min at RT in the dark to block unspecific binding of single stranded RNA to the protein. Subsequently, the blocked streptavidin(-Eu) was mixed with the DNA-probes on ice and kept away from light until use.

At the end of the enzymatic reaction time 10 μL detection reagent mix was added, such that the final concentration of fluorescent-donor probe (bio-5′-AACACATCTCT(-bio)GCCAAACCCCA-bio-3′), fluorescent-acceptor probe (ATTO647N-5′-ACAAAGAACCCTAACACCAG-3′) and streptavidin(-Eu) in each assay well was 1 nM, 3 nM, and 1 nM respectively. Assay plates were again mixed and centrifuged as above and stored at RT, protected from light for at least 2 h or until binding of the DNA probes to RNA product and binding of streptavidin(-Eu) to the biotinylated DNA probe led to the development of the maximal FRET signal. The generated signal was measured with an EnVision plate reader, including TRF light unit (Perkin Elmer, Waltham MA), using excitation at 320 nm, an integration time of 200 us and a delay time of 100 μs, prior to detection at 620 nm and 665 nm. The ratio of donor- and acceptor-fluorescence was used to assess the specific FRET signal, as a measure of the generated product content (i.e. enzymatic activity).

### Assay 2: Quantitative Real Time-PCR to Assess Cellular Activity

Quantitative real-time PCR (qRT-PCR), based on the TaqMan™ (Thermo Fisher Scientific, Waltham MA) technology, was carried out essentially as described in (Heid et al., 1996). Hela cells were plated one day before compound treatment in RPMI medium supplemented with 10% Fetal Calf Serum and 2 mM L-glutamine. Cells were incubated with dilution series of compounds or vehicle (DMSO) for 4 h, prior to harvest and extraction of the RNA using the RNeasy Mini Kit (Qiagen, Hilden D), according to the manufacturer's instructions. RNA concentrations were measured spectroscopically, using a NanoDrop-2000 (Thermo Fisher Scientific, Waltham MA) and normalized prior to cDNA synthesis, using a ‘High-Capacity cDNA Reverse Transcription Kit’ (Thermo Fisher Scientific, Waltham MA). qRT-PCR was carried out using the ‘TaqMan Fast Advance Master Mix’ (Thermo Fisher Scientific, Waltham MA) on a 7500 Fast Real-Time PCR machine (Applied Biosystems, Foster City CA)

For these measurements, three genes were used to compare the effect of the scrutinized compounds in relation to their concentration. The POLRMT-gene was used to detect potential influences on nuclear transcription. Mitochondrial transcription in vivo was monitored by measurements 7S RNA. The TBP (TATA-box binding protein) gene was employed as the control (housekeeping gene) during qRT-PCR. The short-lived mitochondrial 7S RNA, which is not post-transcriptionally stabilized, allowed us to monitor rapid changes in mitochondrial transcription activity following compound addition. Biological triplicates were analyzed using the comparative CT Method (ΔΔCt) method (Bubner and Baldwin, 2004) and reported as Rq % values (Rq=Relative quantification=2-ΔΔCt).

### Assay 3: Solforhodamine B Assay (SRB)

Solforhodamine B assay is carried out as described in Voigt W. (2005) Sulforhodamine B Assay and Chemosensitivity. (Methods in Molecular Medicine™, vol 110. Humana Press. https://doi.org/10.1385/1-59259-869-2:039).

### Assay 4: CellTiter-GLO Assay (CTG)

Cell Titer Glo reagent: Promega, Madison, USA, European Pat. No. 1131441, U.S. Pat. Nos. 7,083,911, 7,452,663 and 7,732,128.

3. Combination of IMTs with Anti-Cancer Drugs

It has been found that the composition comprising at least one IMT and at least one anti-cancer drug acts additive or synergistically to inhibit cell proliferation and can be used in the treatment of cancer. This can be determined for example in the Solforhodamine B Assay (SRB) and/or the CellTiter-GLO Assay (CTG) (see Assays 3 and 4). The skilled person however may use different assays to determine the effectivity of such described composition.

### Example 1

The general suitability of IMT substances for a combination with standard inhibitors of the MAPK signalling pathway was first determined in in vitro cell culture experiments. The change in biomass, as an indicator of cell growth, was measured with the help of the Solforhodamine B Assay (SRB) or changes in the cellular ATP content, as an indicator of metabolic activity of the cells, with the help of the CellTiter-GLO Assay (CTG).

To determine the in vitro cellular viability, cellular ATP concentrations were measured using a luminescence-based homogenous assay format, as described here. Cell lines were maintained in RPMI 1640 cell culture medium+glutamine (PAN Biotech GmbH, Aidenbach, Germany) supplemented with 10% fetal calf serum “Gold” (PAA Laboratories GmbH, Pasching, Austria) and grown in a humidified atmosphere at 37° C., 5% CO2. Optimal cell density for each cell line was determined to guarantee linearity. For viability assays with compounds, cells were then seeded at a density of 200 to 1000 per well in 25 μl in 384-well plates (Greiner Bio-One, Frickenhausen, Germany). After overnight incubation at 37° C./5% CO2, 25 nl or 75 nl compounds were added to each sample well by using BIOMEK FXP Laboratory Automation Workstation (Beckman Coulter, USA). Wells with cells and 0.1% or 0.3% DMSO in culture medium were used as positive controls, wells with cells and 10 μM staurosporine (Selleck Chemicals, Huston, USA) in culture medium were used as negative controls. Upon incubation with compounds for 72 h at 37° C./5% CO2 25 μl Cell Titer Glo reagent (Promega, Madison, USA, European Pat. No. 1131441, U.S. Pat. Nos. 7,083,911, 7,452,663 and 7,732,128) 1:2 diluted with cell culture medium) was added to each well to determine cell viability. 384well-plates were placed for 2 min on an orbital microplate shaker and incubated for further 10 min at room temperature resulting in a stabilization of light signal. Luminescence was measured by Envision Plate Reader (Perkin Elmer, USA). IC50 values were calculated with the software Excel Fit (IDBS, Guildford, UK) from 3-fold dilution series comprising 8 concentrations in duplicates.

The determination of changes in cellular biomass was based on the Solforhodamine B assay format, carried out as described in Voigt W. (2005) Sulforhodamine B Assay and Chemosensitivity. (Methods in Molecular Medicine™, vol 110. Humana Press. https://doi.org/10.1385/1-59259-869-2:039).

All in vitro cell culture experiments were carried out under near physiological glucose concentrations (5 mM) in at least two independent replicates.

First, 30 different cell lines from 14 different tissue types were tested in the SRB assay format. The cells were examined individually for changes in growth over a period of 120 hours with dilution series of both inhibitors. Untreated cells were used as a negative control group. Subsequently, a dilution series of the respective MAPK signalling pathway SOCs was treated in combination with a fixed concentration of 40 nM (3S)-1-[(2R)-2-[4-(2-chloro-4-fluoro-phenyl)-2-oxo-chromen-7-yl]oxypropanoyl]piperidine-3-carboxylic acid.

Table 3 provides an overview of the experiments with the BRAF inhibitors dabrafenib and vemurafenib, MEK inhibitors AZD8330, cobimetinib, mirdametinib (PD0325901; (R)—N-(2,3-dihydroxypropoxy)-3,4-difluoro-2-((2-fluoro-4-iodophenyl)amino)benzamide), trametinib and selumetinib, and the ERK inhibitor ulixertinib. The difference between the calculated combinatorial index (BLISS) and the observed combinatorial effect is given, as a measure of the synergism going beyond the purely additive effect. Since synergistic effects of the combination treatment could be demonstrated for a number of different cell lines from bone, intestinal, liver, lung, muscle, ovarian, cervical, prostate, skin and conjunctival tissue, as well as for blood cell lines, individual findings were exemplarily analysed more in-depth.

TABLE 3
Table 3 Combinatorial effect of the IMT (3S)-1-[(2R)-2-[4-(2-chloro-4-fluoro-phenyl)-2-oxo-chromen-7-yl]oxypropanoyl]piperidine-
3-carboxylic acid with five MEK-, two BRaf- and one ERK inhibitors in the in vitro SRB assay.

| | | Dabrafenib | Vemurafenib | | | PD | | | Ulixertinib |
|---|---|---|---|---|---|---|---|---|---|
| | | BRAF | | AZD8330 | Cobimetinib | 0325901 | Trametinib | Selumetinib | ERK |
| Origin | Cell line | inhibition | | MEK inhibition | | | | | inhibition |
| bone | MHHES1 | −0.0230 | −0.0036 | 0.0682 | −0.0055 | 0.0437 | 0.0760 | 0.0218 | 0.0992 |
| bone | U2OS | −0.0418 | 0.0282 | −0.0171 | −0.0663 | 0.0014 | −0.0358 | −0.1010 | −0.0165 |
| brain | U87MG | 0.0044 | −0.0089 | 0.0579 | 0.0347 | 0.0604 | 0.0578 | 0.0486 | 0.0859 |
| colon | COLO205 | −0.0114 | 0.0429 | 0.0127 | 0.0185 | 0.0261 | 0.0319 | 0.0142 | 0.0384 |
| colon | HCT116 | 0.0411 | −0.0013 | −0.0196 | −0.0279 | −0.0042 | −0.0027 | −0.0774 | −0.0066 |
| colon | HCT15 | 0.0551 | 0.0222 | 0.1028 | 0.0539 | 0.1249 | 0.0677 | 0.0320 | −0.0215 |
| colon | HT29 | 0.0360 | 0.0789 | 0.0488 | 0.0382 | 0.0786 | 0.0338 | 0.0236 | 0.0702 |
| colon | LOVO | −0.0384 | −0.0312 | 0.0047 | −0.0754 | −0.0229 | −0.0287 | −0.0877 | 0.0039 |
| colon | SW620 | −0.0699 | 0.0187 | −0.0037 | −0.0609 | 0.0316 | 0.0028 | −0.0547 | 0.0256 |
| connective | HT1080 | −0.1475 | 0.0000 | 0.0197 | −0.0482 | 0.0433 | 0.0288 | −0.0139 | −0.0011 |
| tissue | | | | | | | | | |
| hematological | GRANTA-519 | −0.0357 | 0.0299 | −0.0506 | −0.0008 | 0.0420 | −0.0185 | 0.0050 | 0.0393 |
| hematological | KASUMI-1 | −0.0274 | 0.0270 | −0.0326 | −0.0232 | −0.0286 | −0.0377 | −0.0114 | 0.0068 |
| hematological | MV4-11 | 0.0205 | 0.0638 | 0.0680 | 0.0051 | 0.0637 | 0.0747 | 0.0150 | 0.0079 |
| liver | HEPG2 | −0.1427 | −0.0038 | 0.0270 | −0.0133 | 0.0334 | 0.0351 | 0.0032 | 0.0953 |
| lung | A549 | −0.0892 | −0.0143 | −0.0341 | −0.0490 | −0.0245 | −0.0142 | −0.0519 | −0.0185 |
| lung | CALU6 | −0.0342 | −0.0483 | −0.0116 | 0.0286 | 0.0809 | −0.0308 | 0.0376 | 0.0100 |
| lung | IMR90 | −0.0104 | 0.0186 | 0.0338 | −0.0073 | 0.0387 | 0.0533 | −0.0291 | 0.0678 |
| lung | NClH460 | −0.0275 | 0.0565 | 0.0015 | −0.0338 | 0.0200 | 0.0180 | −0.0276 | 0.0088 |
| muscle | A673 | −0.0146 | 0.0307 | −0.0047 | 0.0185 | 0.0281 | 0.0225 | 0.0313 | 0.0876 |
| ovary | A2780 | 0.0281 | 0.0399 | 0.0118 | −0.0105 | −0.0318 | 0.0305 | −0.0046 | −0.0025 |
| ovary | SKOV3 | 0.0282 | 0.0062 | −0.0609 | 0.0499 | −0.0555 | −0.0444 | 0.0622 | 0.0284 |
| pancreas | BXPC3 | 0.0251 | 0.0084 | 0.0756 | 0.0204 | 0.0495 | 0.0889 | −0.0219 | 0.0946 |
| pancreas | MIAPACA2 | 0.0508 | 0.0241 | 0.1347 | −0.0673 | 0.0500 | 0.1602 | −0.0734 | 0.0431 |
| pancreas | PANC1 | 0.0029 | 0.0177 | 0.0157 | 0.0747 | 0.1000 | 0.0596 | 0.0867 | 0.0634 |
| placenta | JAR | 0.0431 | 0.0818 | 0.0124 | 0.0139 | 0.0256 | 0.0230 | 0.0045 | 0.0116 |
| prostate | DU145 | −0.0114 | −0.0703 | 0.0201 | 0.0164 | −0.0558 | 0.0287 | 0.0383 | 0.0554 |
| skin | A375 | 0.0337 | 0.0362 | 0.0451 | 0.0443 | 0.0418 | 0.0510 | 0.0423 | 0.0427 |
| skin | SKMEL5 | −0.0987 | 0.0351 | 0.0135 | −0.0872 | 0.0095 | 0.0072 | −0.0968 | −0.0542 |
| skin | SKMEL28 | 0.0194 | 0.0058 | 0.0300 | −0.0040 | 0.0303 | 0.0296 | 0.0096 | 0.0022 |
| cervix | HELA | −0.0022 | −0.3106 | 0.0000 | −0.0068 | −0.2549 | −0.0064 | 0.0054 | −0.0117 |

Numerical values indicate the difference between the calculated combinatorial index (BLISS) and the observed combinatorial effect, as a measure of the synergism that goes beyond the purely additive effect. Positive value=antagonistic; Negative value=synergistic.

Both series of experiments proved the combination potential of IMT and MEK inhibitors over a wide range of the inhibitor concentrations used in vitro. Furthermore, combination potential of IMT and MEK inhibitors were tested in in vivo xenograft studies in a mouse model.

TABLE 4
Combinatorial matrix of IMT and two MEKi in the A2780
cell line using the in vitro CTG assay format.
PD318088

| | 30 | 10 | 3.3 | 1.1 | 0.4 | 0.12 | 0.04 | 0 |
|---|---|---|---|---|---|---|---|---|
| 30 | 1.46 | 0.52 | 0.50 | 0.20 | 0.13 | 0.07 | 0.06 | 3 |
| 15 | 1.49 | 0.99 | 0.42 | 0.25 | 0.11 | 0.10 | 0.16 | 2 |
| 7.5 | 1.96 | 1.08 | 0.68 | 0.40 | 0.14 | 0.09 | 0.11 | 6 |
| 3.75 | 1.50 | 0.53 | 1.24 | 0.54 | 0.36 | 0.15 | 0.12 | 2 |
| 1.88 | 0.86 | 1.48 | 1.06 | 0.39 | 0.23 | 0.11 | 0.15 | 0 |
| 0.94 | 1.28 | 0.57 | 0.83 | 0.75 | 0.24 | 0.23 | 0.18 | 0 |
| 0.47 | 1.38 | 0.51 | 0.88 | 0.64 | 0.48 | 0.16 | 0.26 | 0 |
| 0.23 | 1.02 | 1.21 | 1.02 | 1.28 | 0.33 | 0.33 | 0.63 | 0 |
| 0.12 | 1.83 | 1.03 | 0.69 | 0.43 | 0.43 | 0.23 | 0.47 | 0 |
| 0.059 | 1.04 | 1.03 | 1.13 | 0.55 | 0.33 | 0.62 | 0.88 | 0 |
| 0.029 | 0.98 | 1.67 | 1.15 | 1.06 | 0.55 | 0.35 | 1.28 | |
| 0.015 | 1.48 | 1.25 | 1.38 | 0.98 | 0.35 | 1.26 | 1.34 | 49 |
| 0 | 1 | 2 | 1 | 1 | 1 | 1 | 1 | 0 |

Selumetinib

| | 30 | 10 | 3.3 | 1.1 | 0.4 | 0.12 | 0.04 | 0 |
|---|---|---|---|---|---|---|---|---|
| 30 | 1.30 | 0.60 | 0.43 | 0.16 | 0.10 | 0.04 | 0.04 | 1 |
| 15 | 1.92 | 0.79 | 0.48 | 0.32 | 0.17 | 0.13 | 0.09 | 0 |
| 7.5 | 2.48 | 1.21 | 0.55 | 0.33 | 0.16 | 0.07 | 0.10 | |
| 3.75 | 2.96 | 1.67 | 0.52 | 0.27 | 0.26 | 0.13 | 0.26 | 0 |
| 1.88 | 2.53 | 0.73 | 0.71 | 0.36 | 0.14 | 0.19 | 0.65 | 0 |
| 0.94 | 2.36 | 1.10 | 0.97 | 0.45 | 0.41 | 0.90 | 0.20 | 0 |
| 0.47 | 1.54 | 1.17 | 1.53 | 1.08 | 1.39 | 0.46 | 4.47 | |
| 0.23 | 1.92 | 1.31 | 1.50 | 0.48 | 0.42 | 0.27 | 2.06 | 0 |
| 0.117 | 1.15 | 0.92 | 1.08 | 0.35 | 1.14 | 0.47 | 0.25 | 88 |
| 0.059 | 1.35 | 1.31 | 0.67 | 0.60 | 0.20 | 0.49 | 0.23 | |
| 0.029 | 1.66 | 1.54 | 1.46 | 0.47 | 0.34 | 0.24 | 0.45 | |
| 0.015 | 0.91 | 1.05 | 0.48 | 0.84 | 0.21 | 0.72 | 0.45 | 0 |
| 0 | 1 | 1 | 1 | 1 | 1 | 1 | 1 | 0 |

TOP: A2780 cells with IMT 1-[2-[4-(2-chlorophenyl)-2-oxo-chromen-7-yl]oxypropanoyl]piperidine-3-sulfonamide (30 μM-15 nM) and the MEK inhibitor PD318088 (5-bromo-N-(2,3-dihydroxypropoxy)-3,4-difluoro-2-((2-fluoro-4-iodophenyl)amino)benzamide) (30 μM-10 nM). BOTTOM: A2780 with IMT N,N-dimethyl-2-[4-(o-tolyl)-2-oxo-chromen-7-yl]oxy-propanamide (30 μM-15 nM) and 30 μM-10 nM of the MEK inhibitor Selumetinib. Incubation time 72 h. The combinatorial indices according to Berenbaum and Cou & Talalay are provided. <1=synergism, 1-2=additivity, 2>=antagonism.

Female NMRI: nu/nu mice were injected subcutaneously with the ovarian carcinoma cell line and, after reaching a palpable tumour volume of 0.2 cm3, were randomized and divided into groups of eight animals each. In addition to a control group, which was administered only with the vehicle formulation, additional groups were given 100 mg/kg (3S)-1-[(2R)-2-[4-(2-chloro-4-fluoro-phenyl)-2-oxo-chromen-7-yl]oxypropanoyl]piperidine-3-carboxylic acid, 12.5 mg/kg AZD6244, 25 mg/kg AZD6244 or the combination of 100 mg/kg (3S)-1-[(2R)-2-[4-(2-chloro-4-fluoro-phenyl)-2-oxo-chromen-7-yl]oxypropanoyl]piperidine-3-carboxylic acid and 12.5 or 25 mg/kg AZD6244 was administered. The substance was administered orally over 21 days, twice daily in 25% PEG400, 57% HPbCD. The information published by Davies et al (Mol. Cancer Ther. 2007) was used to select the dosage and administration of AZD6244.

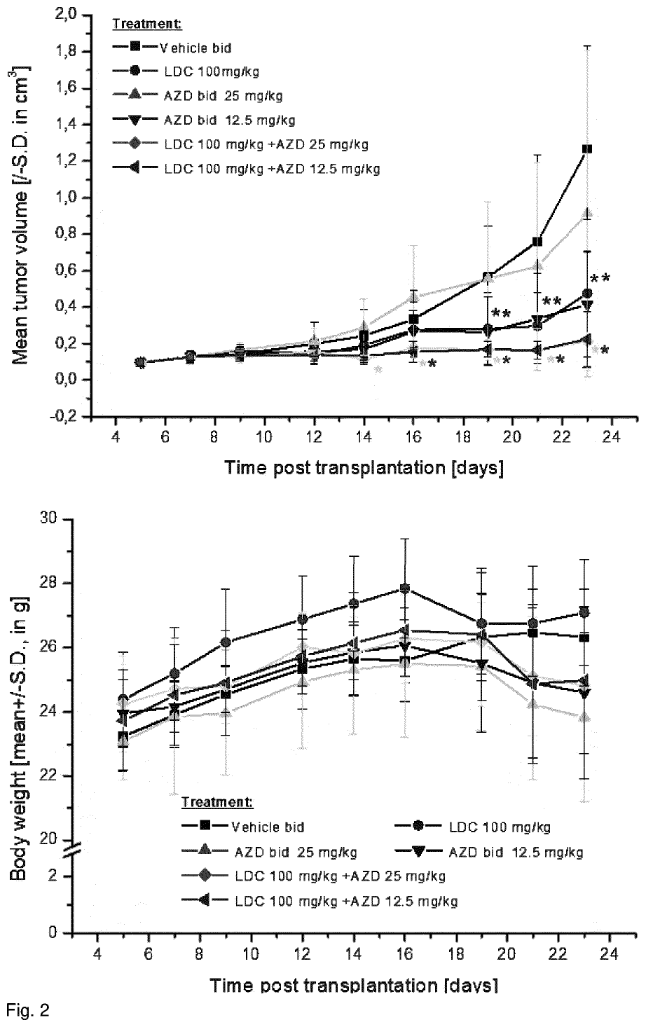

The analysis of the in vivo data generated in this way supports our claim and clearly proves the advantage of the combination treatment with IMTs compared to the single administration of the same amounts of MEK inhibitor in terms of the final tumour volume after completion of the study (FIG. 2). The combination with inhibitors of mitochondrial transcription enables the administration of lower amounts of MEK inhibitors with improved effectiveness. In addition, the combination administration is well tolerated, as the measured changes in body weight over the course of the study and the absence of acute overt effects after administration of the substance showed. This should potentially also lead to an improved of profile side effect in clinical use.

### Example 2

To establish the method described, combinations of (3S)-1-[(2R)-2-[4-(2-chloro-4-fluoro-phenyl)-2-oxo-chromen-7-yl]oxypropanoyl]piperidine-3-carboxylic acid and various PARPi were tested with regard to their effect on the cell growth of 33 tumour cell lines from different tissues, in the SRB and CTG assay format. The assay methods and protocols in cell culture and measurement followed the procedure as described in Example 1). The cell lines used came from bone, brain, intestinal, liver, lung, muscle, ovarian, pancreas, uterine, prostate, skin and cervical tissue, as well as blood cell lines and were initially tested in the SRB assay, at a fixed concentration of (3S)-1-[(2R)-2-[4-(2-chloro-4-fluoro-phenyl)-2-oxo-chromen-7-yl]oxypropanoyl]piperidine-3-carboxylic acid against concentration series of Olaparib or Ruxolitinib. Our analysis and evaluation (corresponding Example 1) shows a clear synergistic effect of (3S)-1-[(2R)-2-[4-(2-chloro-4-fluoro-phenyl)-2-oxo-chromen-7-yl]oxypropanoyl]piperidine-3-carboxylic acid for both PARP inhibitors in the majority of the cell lines and tissue types examined (Table 5). This initial study was followed by validation experiments in a matrix format and in the CTG assay, as described for Example 1) (Table 6.)

TABLE 5
Combinatorial effect of the IMT (3S)-1-[(2R)-2-[4-(2-chloro-
4-fluoro-phenyl)-2-oxo-chromen-7-yl]oxypropanoyl]piperidine-
3-carboxylic acid two PARP inhibitors in the in vitro SRB assay.

| origin | cell line | Olaparib | Ruxolitinib |
|---|---|---|---|
| bone | MHHES1 | 0.0269 | 0.0214 |
| bone | U2OS | −0.0034 | 0.0111 |
| brain | U87MG | 0.0523 | −0.0437 |
| breast | MCF7 | 0.0602 | −0.0164 |
| breast | MDAMB231 | −0.0488 | −0.0495 |
| breast | MDAMB468 | −0.0262 | −0.1189 |
| colon | COLO205 | −0.0607 | −0.0144 |
| colon | HCT116 | −0.0690 | 0.0235 |
| colon | HCT15 | −0.0472 | −0.0093 |
| colon | HT29 | 0.0068 | −0.0400 |
| colon | LOVO | −0.0819 | 0.0697 |
| colon | SW620 | 0.0198 | 0.0477 |
| hematological | GRANTA-519 | −0.0088 | −0.0280 |
| hematological | KASUMI-1 | −0.0415 | 0.0840 |
| hematological | MV4-11 | 0.0407 | 0.0451 |
| liver | HEPG2 | −0.0439 | 0.0064 |
| lung | A549 | 0.0174 | −0.0256 |
| lung | CALU6 | 0.0400 | −0.0319 |
| lung | IMR90 | 0.0084 | 0.0463 |
| lung | NCIH460 | −0.0377 | −0.0208 |
| muscle | A204 | −0.0329 | −0.0415 |
| muscle | A673 | 0.0334 | −0.0209 |
| ovary | A2780 | −0.0656 | −0.0339 |
| ovary | SKOV3 | 0.0092 | −0.0634 |
| pancreas | ASPC1 | −0.0583 | −0.0054 |
| pancreas | BXPC3 | 0.0315 | −0.0300 |
| pancreas | MIAPACA2 | 0.0529 | 0.0502 |
| pancreas | PANC1 | −0.0430 | −0.0756 |
| placenta | JAR | 0.0294 | 0.0717 |
| prostate | DU145 | 0.0557 | −0.0197 |
| skin | A375 | −0.0650 | −0.1759 |
| skin | SKMEL28 | −0.0624 | −0.0505 |
| cervix | HELA | −0.0307 | 0.0579 |

Numerical values indicate the difference between the calculated combinatorial index (BLISS) and the observed combinatorial effect, as a measure of the synergism that goes beyond the purely additive effect. Positive value=antagonistic; Negative value=synergistic.

In the concentration matrices used, 30 μM to 10 nM (3S)-1-[(2R)-2-[4-(2-chloro-4-fluoro-phenyl)-2-oxo-chromen-7-yl]oxypropanoyl]piperidine-3-carboxylic acid in combination with 30 μM to 40 nM Veliparib, Olaparib or Rucaparib in the ovary carcinoma cell line A2780 were tested. Our results from this orthogonal approach confirm the advantage of the combination of IMT and PARPi compared to treatment with just the PARPi alone for all three combinations and over a wide concentration range.

TABLE 6
Combinatorial matrix of IMT and three different
PARPi in A2780 in the in vitro CTG assay format.
A.
Olaparib

| | 30 | 10 | 3.3 | 1.1 | 0.4 | 0.1 | 0.04 | 0 |
|---|---|---|---|---|---|---|---|---|
| 30 | 0 | 1 | 4 | 12 | 18 | 19 | 22 | 33 |
| 15 | 0 | 1 | 8 | 15 | 28 | 24 | 27 | 30 |
| 7.5 | 0 | 1 | 9 | 15 | 24 | 27 | 24 | 27 |
| 3.8 | 0 | 1 | 9 | 20 | 25 | 28 | 29 | 31 |
| 1.9 | 0 | 1 | 7 | 13 | 24 | 26 | 28 | 29 |
| 0.9 | 0 | 1 | 7 | 19 | 25 | 27 | 22 | 30 |
| 0.5 | 0 | 1 | 9 | 22 | 24 | 27 | 28 | 32 |
| 0.2 | 0 | 1 | 7 | 17 | 23 | 29 | 26 | 30 |
| 0.1 | 0 | 1 | 9 | 24 | 32 | 32 | 28 | 30 |
| 0.06 | 0 | 1 | 9 | 26 | 26 | 39 | 42 | 23 |
| 0.03 | 0 | 1 | 7 | 35 | 51 | 38 | 46 | 37 |
| 0.01 | 0 | 1 | 8 | 35 | 61 | 87 | 70 | 86 |
| 0 | 0 | 1 | 14 | 27 | 46 | 85 | 99 | 100 |

Rucaparib

| | 30 | 10 | 3.3 | 1.1 | 0.4 | 0.1 | 0.04 | 0 |
|---|---|---|---|---|---|---|---|---|
| 30 | 0 | 0 | 3 | 9 | 16 | 20 | 20 | 25 |
| 15 | 0 | 0 | 4 | 8 | 12 | 18 | 20 | 25 |
| 7.5 | 0 | 1 | 3 | 9 | 12 | 18 | 18 | 26 |
| 3.8 | 0 | 0 | 2 | 15 | 16 | 18 | 25 | 25 |
| 1.9 | 0 | 1 | 4 | 8 | 17 | 17 | 23 | 23 |
| 0.94 | 0 | 0 | 4 | 14 | 8 | 12 | 24 | 25 |
| 0.47 | 0 | 0 | 3 | 12 | 20 | 20 | 24 | 29 |
| 0.23 | 0 | 0 | 6 | 12 | 16 | 30 | 29 | 28 |
| 0.12 | 0 | 0 | 3 | 14 | 18 | 31 | 29 | 34 |
| 0.06 | 0 | 0 | 3 | 17 | 27 | 32 | 25 | 30 |
| 0.03 | 0 | 0 | 6 | 18 | 32 | 39 | 41 | 33 |
| 0.01 | 0 | 0 | 4 | 19 | 46 | 45 | 66 | 68 |
| 0 | 0 | 0 | 6 | 21 | 42 | 70 | 90 | 100 |

Veliparib

| | 30 | 10 | 3.3 | 1.1 | 0.4 | 0.1 | 0.04 | 0 |
|---|---|---|---|---|---|---|---|---|
| 30 | 3 | 14 | 20 | 30 | 29 | 25 | 36 | 31 |
| 15 | 4 | 15 | 21 | 33 | 29 | 24 | 30 | 27 |
| 7.5 | 3 | 15 | 30 | 21 | 27 | 30 | 29 | 31 |
| 3.8 | 6 | 15 | 22 | 31 | 27 | 27 | 19 | 30 |
| 1.9 | 4 | 15 | 26 | 28 | 33 | 28 | 32 | 29 |
| 0.9 | 4 | 12 | 24 | 30 | 34 | 33 | 30 | 29 |
| 0.5 | 4 | 15 | 29 | 32 | 34 | 34 | 36 | 33 |
| 0.2 | 3 | 17 | 22 | 30 | 39 | 42 | 35 | 29 |
| 0.1 | 4 | 19 | 34 | 38 | 36 | 42 | 22 | 29 |
| 0.06 | 7 | 25 | 35 | 37 | 33 | 32 | 13 | 20 |
| 0.03 | 5 | 25 | 59 | 51 | 49 | 34 | 51 | 37 |
| 0.01 | 7 | 54 | 67 | 77 | 93 | 83 | 79 | 79 |
| 0 | 6 | 43 | 85 | 89 | 97 | 99 | 99 | 100 |

B.
Olaparib

| | 30 | 10 | 3.3 | 1.1 | 0.4 | 0.12 | 0.04 | 0 |
|---|---|---|---|---|---|---|---|---|
| 30 | 0.14 | 0.47 | 0.57 | 0.48 | 0.43 | 0.32 | 0.97 | 30.79 |
| 15 | 0.28 | 0.43 | 0.94 | 0.64 | 3.89 | 0.96 | 2.87 | 7.60 |
| 7.5 | 0.44 | 0.59 | 1.12 | 0.62 | 0.71 | 1.21 | 0.43 | 1.41 |
| 3.75 | 0.29 | 0.42 | 1.04 | 0.87 | 0.73 | 1.10 | 1.12 | 2.00 |
| 1.88 | 0.32 | 0.34 | 0.84 | 0.50 | 0.42 | 0.32 | 0.44 | 0.70 |
| 0.94 | 0.23 | 0.44 | 0.83 | 0.78 | 0.42 | 0.29 | 0.06 | 0.39 |
| 0.47 | 0.23 | 0.38 | 1.12 | 0.92 | 0.36 | 0.22 | 0.15 | 0.34 |
| 0.23 | 0.37 | 0.53 | 0.84 | 0.69 | 0.33 | 0.20 | 0.07 | 0.10 |
| 0.12 | 0.19 | 0.58 | 1.05 | 1.01 | 0.56 | 0.26 | 0.07 | 0.06 |
| 0.06 | 0.60 | 0.65 | 1.14 | 1.09 | 0.36 | 0.55 | 0.97 | 0.00 |
| 0.03 | 0.28 | 0.44 | 0.92 | 1.59 | 4.93 | 0.34 | 1.12 | 0.11 |
| 0.01 | 0.51 | 0.57 | 0.97 | 1.58 | 34.06 | | | |
| 0 | 0.21 | 0.50 | 1.62 | 1.14 | 0.77 | 1.31 | 4.85 | |

Rucaparib

| | 30 | 10 | 3.3 | 1.1 | 0.4 | 0.12 | 0.04 | 0 |
|---|---|---|---|---|---|---|---|---|
| 30 | 0.02 | 0.24 | 0.49 | 0.45 | 0.65 | 2.03 | 2.03 | 10.47 |
| 15 | 0.02 | 0.11 | 0.55 | 0.41 | 0.22 | 0.59 | 1.10 | 4.51 |
| 7.5 | 0.02 | 0.34 | 0.51 | 0.45 | 0.21 | 0.28 | 0.27 | 3.95 |
| 3.8 | 0.02 | 0.11 | 0.34 | 0.77 | 0.33 | 0.21 | 1.52 | 1.23 |
| 1.9 | 0.02 | 0.28 | 0.61 | 0.39 | 0.32 | 0.13 | 0.37 | 0.29 |
| 0.94 | 0.02 | 0.21 | 0.54 | 0.74 | 0.13 | 0.07 | 0.26 | 0.29 |
| 0.47 | 0.02 | 0.13 | 0.40 | 0.60 | 0.39 | 0.14 | 0.16 | 0.59 |
| 0.23 | 0.02 | 0.12 | 0.90 | 0.62 | 0.28 | 0.50 | 0.30 | 0.20 |
| 0.12 | 0.02 | 0.24 | 0.46 | 0.73 | 0.32 | 0.42 | 0.18 | 0.53 |
| 0.06 | 0.02 | 0.22 | 0.49 | 0.88 | 0.56 | 0.38 | 0.07 | 0.09 |
| 0.03 | 0.02 | 0.25 | 0.96 | 0.96 | 0.74 | 0.73 | 0.85 | 0.10 |
| 0.01 | 0.02 | 0.18 | 0.57 | 1.01 | 2.33 | 1.32 | 151.49 | |
| 0 | 0.02 | 0.15 | 0.87 | 1.13 | 0.97 | 0.96 | 1.14 | |

Combinations of one concentration (3S)-1-[(2R)-2-[4-(2-chloro-4-fluoro-phenyl)-2-oxo-chromen-7-yl]oxypropanoyl]piperidine-3-carboxylic acid (y-axis, 30 μM-15 nM) and 30 μM-10 nM of the PARP inhibitors (x-axis) Veliparib, Olaparib or Rucaparib were incubated with A2780 for 72 hours and then the cellular ATP content was determined using the CellTiter-GLO assay. A.: Measured residual activity (%) based on an untreated control group (=100%). B: The combinatorial indices are provided, based on the reference groups treated with the corresponding concentration of only one inhibitor class, according to Berenbaum and Cou & Talalay. <1=synergism, 1-2=additivity, 2>=antagonism.

### Example 3

To establish the method described, N,N-dimethyl-2-[4-(o-tolyl)-2-oxo-chromen-7-yl]oxy-propanamide, (3S)-1-[2-[4-(2-chloro-4-fluoro-phenyl)-2-oxo-chromen-7-yl]oxypropanoyl]piperidine-3-carboxylic acid, (3S)-1-[(2R)-2-[4-(2-chloro-4-fluoro-phenyl)-2-oxo-chromen-7-yl]oxypropanoyl]piperidine-3-carboxylic acid and BAY-876 were first examined with regard to their effect on the cell growth of twelve different tumour cell lines and the IC50 values were determined. This initial study was followed by validation experiments in a matrix format and in the CTG assay, as described under Example 1), in two tumour cell lines from uterine and ovarian carcinoma (HEC59 and A2780), as well as in primary human blood cells (PBMC) (Table 7).

In the concentration matrices used, 30 μM to 15 nM (3S)-1-[2-[4-(2-chloro-4-fluoro-phenyl)-2-oxo-chromen-7-yl]oxypropanoyl]piperidine-3-carboxylic acid or (3S)-1-[(2R)-2-[4-(2-chloro-4-fluoro-phenyl)-2-oxo-chromen-7-yl]oxypropanoyl]piperidine-3-carboxylic acid in combination with 30 μM to 40 nM BAY-876 were used. The results from this approach confirm the advantage of the combination of IMT and GLUTi compared to treatment with only the GLUTi BAY-876 alone for both combinations, in both tumour cell lines and over a wide concentration range. In particular, the comparison with the results from the hPBMCs suggests a positive treatment window, since a 50% growth inhibition is not achieved in these primary human blood cells even at the highest substance concentration used (30 UM).

TABLE 7
Combinatorial matrix of IMT and the GLUTi BAY-876 in three
different cell lines in the in vitro CTG assay format.
A.
HEC59

| | 30 | 10 | 3.3 | 1.111 | 0.370 | 0.123 | 0.041 | 0 |
|---|---|---|---|---|---|---|---|---|
| 10 | 1 | 2 | 2 | 2 | 6 | 5 | 11 | 47 |
| 5 | 1 | 2 | 2 | 3 | 3 | 7 | 12 | 34 |
| 2.50 | 1 | 2 | 3 | 4 | 5 | 8 | 12 | 39 |
| 1.25 | 1 | 3 | 4 | 4 | 6 | 11 | 16 | 39 |
| 0.625 | 2 | 3 | 5 | 7 | 12 | 13 | 18 | 42 |
| 0.313 | 3 | 5 | 5 | 9 | 13 | 17 | 20 | 49 |
| 0.156 | 5 | 6 | 9 | 11 | 15 | 22 | 29 | 60 |
| 0.078 | 6 | 8 | 12 | 17 | 32 | 37 | 47 | 83 |
| 0.039 | 9 | 11 | 16 | 25 | 42 | 56 | 75 | 98 |
| 0.020 | 10 | 14 | 20 | 30 | 54 | 63 | 72 | 96 |
| 0.010 | 12 | 12 | 20 | 31 | 49 | 76 | 76 | 99 |
| 0.005 | 9 | 14 | 19 | 29 | 54 | 75 | 80 | 99 |
| 0 | 8 | 11 | 18 | 32 | 58 | 59 | 74 | 100 |

hPBMCs

| | 30 | 10 | 3.33 | 1.11 | 0.37 | 0.12 | 0.04 | 0 |
|---|---|---|---|---|---|---|---|---|
| 30 | 56 | 81 | 87 | 78 | 92 | 82 | 104 | 84 |
| 15 | 54 | 62 | 78 | 80 | 88 | 81 | 80 | 88 |
| 7.50 | 53 | 65 | 73 | 93 | 98 | 87 | 101 | 90 |
| 3.75 | 63 | 85 | 84 | 107 | 77 | 99 | 100 | 104 |
| 1.88 | 59 | 70 | 90 | 92 | 94 | 102 | 86 | 94 |
| 0.94 | 68 | 77 | 80 | 100 | 79 | 96 | 84 | 100 |
| 0.47 | 62 | 90 | 88 | 87 | 92 | 96 | 90 | 102 |
| 0.234 | 90 | 91 | 76 | 92 | 90 | 106 | 85 | 94 |
| 0.117 | 63 | 80 | 85 | 112 | 95 | 99 | 88 | 98 |
| 0.059 | 64 | 97 | 78 | 95 | 107 | 94 | 84 | 98 |
| 0.029 | 84 | 85 | 81 | 76 | 91 | 98 | 110 | 99 |
| 0.015 | 75 | 79 | 89 | 95 | 88 | 10 | 79 | 97 |
| 0 | 101 | 87 | 98 | 106 | 90 | 109 | 104 | 100 |

A2780

| | 30 | 10 | 3.33 | 1.11 | 0.37 | 0.123 | 0.04 | 0 |
|---|---|---|---|---|---|---|---|---|
| 30 | 1 | 2 | 7 | 12 | 13 | 17 | 14 | 28 |
| 15 | 1 | 3 | 7 | 12 | 14 | 15 | 14 | 26 |
| 7.5 | 1 | 3 | 5 | 9 | 16 | 13 | 11 | 27 |
| 3.75 | 2 | 3 | 5 | 12 | 14 | 19 | 15 | 29 |
| 1.88 | 1 | 3 | 6 | 11 | 13 | 12 | 15 | 29 |
| 0.94 | 1 | 3 | 8 | 11 | 15 | 18 | 15 | 32 |
| 0.47 | 1 | 4 | 8 | 14 | 18 | 21 | 17 | 32 |
| 0.23 | 2 | 4 | 9 | 14 | 18 | 20 | 22 | 35 |
| 0.12 | 3 | 4 | 10 | 16 | 20 | 19 | 20 | 35 |
| 0.059 | 4 | 8 | 12 | 23 | 22 | 21 | 24 | 39 |
| 0.029 | 4 | 9 | 16 | 28 | 29 | 26 | 28 | 42 |
| 0.015 | 5 | 19 | 34 | 40 | 48 | 60 | 58 | 59 |
| 0 | 10 | 51 | 78 | 86 | 90 | 78 | 88 | 100 |

B.
HEC59:

| | 30 | 10 | 3.3 | 1.1 | 0.37 | 0.12 | 0.04 | 0 |
|---|---|---|---|---|---|---|---|---|
| 10.0 | 0.02 | 0.02 | 0.01 | 0.00 | 0.04 | 0.03 | 0.16 | 7.97 |
| 5.0 | 0.01 | 0.03 | 0.01 | 0.01 | 0.01 | 0.03 | 0.09 | 1.36 |
| 2.5 | 0.02 | 0.03 | 0.02 | 0.01 | 0.01 | 0.02 | 0.05 | 1.09 |
| 1.25 | 0.04 | 0.05 | 0.04 | 0.02 | 0.01 | 0.03 | 0.05 | 0.53 |
| 0.63 | 0.12 | 0.05 | 0.06 | 0.03 | 0.04 | 0.02 | 0.04 | 0.36 |
| 0.31 | 0.24 | 0.13 | 0.05 | 0.06 | 0.04 | 0.03 | 0.03 | 0.29 |
| 0.16 | 0.51 | 0.21 | 0.16 | 0.09 | 0.06 | 0.05 | 0.05 | 0.38 |
| 0.078 | 0.62 | 0.37 | 0.30 | 0.20 | 0.31 | 0.17 | 0.16 | 2.07 |
| 0.039 | 1.29 | 0.66 | 0.52 | 0.46 | 0.63 | 0.67 | 1.24 | 144.51 |
| 0.020 | 1.89 | 1.15 | 0.80 | 0.76 | 1.48 | 1.00 | 0.80 | 16.11 |
| 0.010 | 2.43 | 0.85 | 0.80 | 0.77 | 1.04 | 3.07 | 1.15 | 427.27 |
| 0.005 | 1.48 | 1.08 | 0.76 | 0.68 | 1.46 | 2.77 | 1.52 | 41.92 |
| 0 | 1.21 | 0.78 | 0.69 | 0.90 | 1.97 | 0.71 | 0.84 | |

hPBMCs

| | 30 | 10 | 3.3 | 1.1 | 0.37 | 0.12 | 0.04 | 0 |
|---|---|---|---|---|---|---|---|---|
| 30 | 0.17 | 0.82 | 1.44 | 0.62 | 3.39 | 0.88 | | 1.08 |
| 15 | 0.07 | 0.12 | 0.32 | 0.39 | 0.84 | 0.42 | 0.39 | 0.83 |
| 7.5 | 0.03 | 0.07 | 0.11 | 0.94 | 7.32 | 0.36 | | 0.55 |
| 3.75 | 0.03 | 0.15 | 0.14 | | 0.07 | 6.64 | | |
| 1.88 | 0.01 | 0.02 | 0.16 | 0.21 | 0.34 | | 0.08 | 0.31 |
| 0.94 | 0.01 | 0.02 | 0.02 | | 0.02 | 0.27 | 0.03 | 23.73 |
| 0.47 | 0.01 | 0.07 | 0.03 | 0.02 | 0.05 | 0.13 | 0.04 | |
| 0.23 | 0.11 | 0.07 | 0.00 | 0.03 | 0.02 | | 0.01 | 0.04 |
| 0.12 | 0.00 | 0.01 | 0.01 | | 0.03 | 0.97 | 0.01 | 0.09 |
| 0.059 | 0.00 | 0.61 | 0.00 | 0.04 | | 0.01 | 0.00 | 0.06 |
| 0.029 | 0.03 | 0.01 | 0.00 | 0.00 | 0.00 | 0.04 | | 0.04 |
| 0.015 | 0.01 | 0.00 | 0.01 | 0.03 | 0.00 | | 0.00 | 0.01 |
| 0 | | 0.02 | 0.57 | | 0.00 | | | |

A2780

| | 30 | 10 | 3.3 | 1.1 | 0.37 | 0.12 | 0.04 | 0 |
|---|---|---|---|---|---|---|---|---|
| 30 | 0.05 | 0.04 | 0.04 | 0.03 | 0.01 | 0.07 | 0.01 | 6.20 |
| 15 | 0.06 | 0.05 | 0.04 | 0.02 | 0.02 | 0.02 | 0.01 | 1.67 |
| 7.5 | 0.07 | 0.06 | 0.03 | 0.02 | 0.02 | 0.01 | 0.00 | 1.32 |
| 3.75 | 0.10 | 0.05 | 0.03 | 0.02 | 0.01 | 0.03 | 0.00 | 1.30 |
| 1.88 | 0.08 | 0.05 | 0.03 | 0.02 | 0.01 | 0.00 | 0.00 | 0.67 |
| 0.94 | 0.07 | 0.06 | 0.04 | 0.02 | 0.01 | 0.01 | 0.00 | 0.67 |
| 0.47 | 0.08 | 0.07 | 0.05 | 0.03 | 0.01 | 0.01 | 0.00 | 0.39 |
| 0.23 | 0.11 | 0.07 | 0.05 | 0.03 | 0.01 | 0.01 | 0.01 | 0.46 |
| 0.12 | 0.15 | 0.07 | 0.06 | 0.03 | 0.01 | 0.00 | 0.00 | 0.26 |
| 0.059 | 0.20 | 0.13 | 0.07 | 0.05 | 0.02 | 0.01 | 0.00 | 0.37 |
| 0.029 | 0.23 | 0.15 | 0.09 | 0.07 | 0.03 | 0.01 | 0.01 | 0.44 |
| 0.015 | 0.27 | 0.33 | 0.24 | 0.23 | 1.52 | 45.48 | 23.65 | 28.97 |
| 0 | 0.51 | 1.22 | 1.23 | 0.66 | 0.31 | 0.05 | 0.03 | |

Combinations of one concentration of IMT (μM; y-axis) and 30 μM-33 nM BAY-876 (x-axis) were incubated for 72 hours with HEC59, hPBMCs or A2780 and then the cellular ATP-content was determined using the CellTiter-GLO assay format. A: Measured residual activity (%) based on an untreated control group (=100%). B: The combinatorial indices are provided, based on the reference groups treated with the corresponding concentration of only one inhibitor class, according to Berenbaum and Cou & Talalay. <1=synergism, 1-2=additivity, 2>=antagonism.

### Example 4

To establish the method described, (3S)-1-[(2R)-2-[4-(2-chloro-4-fluoro-phenyl)-2-oxo-chromen-7-yl]oxypropanoyl]piperidine-3-carboxylic acid and teriflunomide were first examined with regard to their effect on the cell growth of the ovarian carcinoma cell line A2780 and the IC50 values were determined in the CTG assay format, as described in Example 1). These initial experiments were followed by studies in a matrix format, with combinations of both inhibitors being incubated with the A2780 cell line in concentration series from 30 μM to 10 nM ((3S)-1-[(2R)-2-[4-(2-chloro-4-fluoro-phenyl)-2-oxo-chromen-7-yl]oxypropanoyl]piperidine-3-carboxylic acid) or 30 μM to 40 nM teriflunomide) for 72 hours (Table 8).

TABLE 8
Combinatorial matrix of IMT and DHODHi in A2780 in the in vitro CTG assay format
Teriflunomide

| | | 30 | 10 | 3.3 | 1.1 | 0.4 | 0.1 | 0.04 | 0 |
|---|---|---|---|---|---|---|---|---|---|
| (3S)-1-[(2R)-2-[4-(2- | 30 | 13 | 19 | 22 | 21 | 22 | 22 | 27 | 27 |
| chloro-4-fluoro-phenyl)-2- | 15 | 13 | 22 | 18 | 22 | 25 | 21 | 23 | 28 |
| oxo-chromen-7- | 7.5 | 13 | 18 | 21 | 18 | 22 | 22 | 27 | 25 |
| yl]oxypropanoyl]piperidine- | 3.75 | 13 | 15 | 16 | 23 | 22 | 29 | 28 | 28 |
| 3-carboxylic acid (μM) | 1.88 | 13 | 19 | 23 | 24 | 22 | 24 | 31 | 26 |
| | 0.94 | 16 | 19 | 22 | 23 | 22 | 25 | 26 | 29 |
| | 0.47 | 14 | 17 | 23 | 30 | 25 | 28 | 28 | 32 |
| | 0.23 | 15 | 23 | 24 | 26 | 30 | 29 | 31 | 28 |
| | 0.12 | 17 | 23 | 26 | 28 | 32 | 30 | 31 | 32 |
| | 0.06 | 19 | 27 | 24 | 30 | 32 | 39 | 24 | 34 |
| | 0.03 | 19 | 26 | 31 | 35 | 38 | 36 | 38 | 39 |
| | 0.015 | 27 | 48 | 58 | 65 | 54 | 59 | 53 | 55 |
| | 0 | 25 | 63 | 81 | 77 | 99 | 89 | 106 | 100 |

Teriflunomide

| | | 30 | 10 | 3.3 | 1.1 | 0.4 | 0.1 | 0.04 | 0 |
|---|---|---|---|---|---|---|---|---|---|
| (3S)-1-[(2R)-2-[4-(2- | | 0.31 | 0.11 | 0.63 | 0.32 | 0.59 | 1.93 | 2.47 | 5.16 |
| chloro-4-fluoro-phenyl)-2- | 15 | 0.31 | 0.64 | 0.18 | 3.14 | 0.22 | 2.46 | 1.36 | 5.96 |
| oxo-chromen-7- | 7.5 | 0.22 | 0.13 | 0.18 | 0.04 | 0.07 | 0.18 | 0.44 | 1.32 |
| yl]oxypropanoyl]piperidine- | 3.75 | 0.22 | 0.1 | 0.29 | 0.74 | 0.03 | 0.6 | 0.76 | 0.87 |
| 3-carboxylic acid | 1.88 | 0.27 | 0.12 | 0.07 | 0.61 | 0.25 | 0.59 | 0.48 | 0.32 |
| | 0.94 | 0.31 | 0.15 | 0.07 | 0.54 | 0.07 | 0.49 | 0.78 | 0.59 |
| | 0.47 | 0.3 | 0.14 | 0.06 | 0.31 | 0.08 | 0.12 | 0.02 | 0.35 |
| | 0.234 | 0.24 | 0.27 | 0.07 | 0.07 | 0.29 | 0.9 | 0.06 | 0.43 |
| | 0.117 | 0.38 | 0.22 | 0.18 | 0.07 | 0.06 | 0.02 | 0.18 | 0.26 |
| | 0.059 | 0.47 | 0.24 | 0.34 | 0.04 | 0.24 | 1.01 | 0.35 | 0.16 |
| | 0.029 | 0.48 | 0.28 | 0.12 | 0.24 | 0.06 | 0.44 | 1.3 | 0.42 |
| | 0.015 | 0.53 | 1.66 | 0.98 | 634.38 | 6.27 | 660.99 | 239.43 | 65.66 |
| | 0 | 0.65 | 1.55 | 1.2 | 0.38 | | 0.73 | 0.19 | |

Combinations of a concentration of IMT (3S)-1-[(2R)-2-[4-(2-chloro-4-fluoro-phenyl)-2-oxo-chromen-7-yl]oxypropanoyl]piperidine-3-carboxylic acid (y-axis, 30 μM-15 nM) and 30 μM-33 nM teriflunomide (x-axis) were used for 72 hours A2780 is incubated and then the cellular ATP content is determined using the CellTiter-GLO assay. Top: Measured residual activity (%) based on an untreated control group (=100%). Bottom: The combinatorial indices are provided, based on reference groups treated with the corresponding concentration of only one inhibitor class, according to Berenbaum and Cou & Talalay. <1=synergism, 1-2=additivity, 2>=antagonism.

### Example 5

To establish the method described, combinations of (3S)-1-[(2R)-2-[4-(2-chloro-4-fluoro-phenyl)-2-oxo-chromen-7-yl]oxypropanoyl]piperidine-3-carboxylic acid and various DHODHi were tested with regard to their effect on the cell growth of 33 tumour cell lines of different tissue origin, in the SRB assay format. The assay methods and protocols in cell culture and measurement followed the procedure as described in Example 1). The cell lines used came from bone, brain, intestinal, breast, liver, lung, muscle, ovary, pancreas, uterine, prostate, skin, cervical and connective tissue, as well as blood cell lines and were tested in the SRB-Assay at a fixed concentration of (3S)-1-[(2R)-2-[4-(2-chloro-4-fluoro-phenyl)-2-oxo-chromen-7-yl]oxypropanoyl]piperidine-3-carboxylic acid against concentration series of Copanlisib (BAY80-6946), Duvelisib or GDC-0941. Our analysis and evaluation (according to Example 3) shows a clear synergistic effect of (3S)-1-[(2R)-2-[4-(2-chloro-4-fluoro-phenyl)-2-oxo-chromen-7-yl]oxypropanoyl]piperidine-3-carboxylic acid in the majority of the examined cell lines and tissue types, especially for BAY80-6946 and duvelisib (Table 9) and thus confirm the advantages of using a combination of IMT and PIK3Cα-inhibitors, compared to treatment with only the PIK3Cα inhibitors alone.

TABLE 9
Combinatorial effect of the IMT (3S)-1-[(2R)-2-[4-(2-chloro-4-
fluoro-phenyl)-2-oxo-chromen-7-yl]oxypropanoyl]piperidine-3-carboxylic
acid three inhibitors of PI3Kinase in the in vitro SRB assay.

| | | BAY | | |
|---|---|---|---|---|
| | | 80-6946 | Duvelisib | GDC-0941 |
| origin | cell line | PI3K inhibition | | |
| bone | U2OS | 0.0017 | −0.0126 | 0.0451 |
| brain | U87MG | 0.0243 | 0.0433 | 0.1078 |
| breast | MCF7 | 0.0155 | 0.0256 | 0.0454 |
| breast | MDAMB468 | 0.0183 | −0.0285 | −0.0509 |
| colon | COLO205 | 0.0531 | −0.0157 | 0.0355 |
| colon | HCT116 | −0.0195 | −0.0213 | −0.0464 |
| colon | HCT15 | 0.1444 | 0.0170 | 0.0270 |
| colon | HT29 | 0.0613 | 0.0216 | 0.1027 |
| colon | LOVO | −0.0178 | −0.0219 | −0.0539 |
| colon | SW620 | −0.0651 | −0.0623 | 0.0303 |
| connective | HT1080 | −0.0627 | −0.0565 | 0.0172 |
| tissue | | | | |
| hematological | GRANTA-519 | 0.0631 | 0.0770 | 0.0647 |
| hematological | KASUMI-1 | 0.0015 | −0.0237 | 0.0051 |
| hematological | MV4-11 | 0.0337 | 0.0611 | 0.0272 |
| liver | HEPG2 | −0.0273 | −0.0318 | −0.0006 |
| lung | A549 | 0.0159 | −0.0092 | 0.0023 |
| lung | CALU6 | 0.0374 | 0.0342 | −0.0406 |
| lung | IMR90 | 0.0281 | 0.0253 | 0.0629 |
| lung | NCIH460 | −0.0473 | −0.0651 | −0.0178 |
| muscle | A204 | −0.0098 | −0.0217 | 0.3066 |
| muscle | A673 | 0.0259 | 0.0570 | 0.0099 |
| ovary | A2780 | 0.0466 | 0.0081 | 0.0077 |
| ovary | SKOV3 | 0.0112 | 0.0398 | 0.0556 |
| pancreas | ASPC1 | 0.0488 | 0.0753 | 0.1003 |
| pancreas | BXPC3 | −0.0082 | −0.0109 | 0.0704 |
| pancreas | MIAPACA2 | 0.0574 | −0.0183 | 0.0516 |
| pancreas | PANC1 | 0.0174 | −0.0096 | −0.0128 |
| placenta | JAR | 0.0381 | 0.0624 | 0.0136 |
| prostate | DU145 | 0.0423 | 0.0362 | 0.0436 |
| skin | A375 | −0.0033 | −0.0153 | −0.0654 |
| skin | SKMEL5 | −0.0994 | −0.1083 | −0.0915 |
| skin | SKMEL28 | −0.0068 | −0.0412 | −0.0330 |
| cervix | HELA | −0.0260 | −0.0666 | −0.0531 |

Numerical values indicate the difference between the calculated combinatorial index (BLISS) and the observed combinatorial effect, as a measure of the synergism that goes beyond the purely additive effect. Positive value=antagonistic; Negative value=synergistic.

### Example 6

The general suitability of IMT substances for a combination with standard inhibitors of the Bcl-2 signalling pathway was first determined in in vitro cell culture experiments in a matrix format (Table 10). The changes in the cellular ATP content, as an indicator of the metabolic activity of the cells, were measured using the CellTiter-GLO Assay (CTG), as described in Example 1). In these tests, an additive or even synergistic effect was observed in the AML line (MV4-11) used for this combination over a wide concentration range of the inhibitors.

TABLE 10
Combinatrial matrix of IMT and Bcl-2i in MV4-11 in the in vitro CTG assay format Combinations of one concentration
of IMT 2-[(3R)-1-[(2R)-2-[4-(2-chloro-4-fluoro-phenyl)-2-oxo-chromen-7-yl]oxypropanoyl]-3-piperidyl]acetic
acid (y-axis, 60 μM-19 nM) and 3 μM-1 nM Venetoclax (x-axis) were incubated for 72 hours with MV4-11 and then
the cellular ATP content was determined using the CellTiter-GLO assay. The provided values are the combinatorial indices,
based on the reference groups treated with the corresponding concentration of only one inhibitor class, according to Loewe.

| | | Venetoclax (μM) | | | | | | | | | | | |
|---|---|---|---|---|---|---|---|---|---|---|---|---|---|
| | | 0.0001 | 0.0020 | 0.0060 | 0.01 | 0.02 | 0.04 | 0.06 | 0.1 | 0.2 | 0.4 | 1 | 2.99 |
| 2-[(3R)-1-[(2R)-2-[4-(2- | 0.019 | −22 | −13 | 12 | 2 | 0 | 1 | 1 | −1 | 0 | 1 | 0 | 0 |
| chloro-4-fluoro-phenyl)-2-oxo- | 0.059 | 12 | 18 | 29 | 25 | 12 | 7 | 5 | 3 | 2 | 1 | 1 | 1 |
| chromen-7-yl]oxypropanoyl]- | 0.199 | 13 | 20 | 33 | 28 | 16 | 8 | 6 | 3 | 2 | 1 | 1 | 1 |
| 3-piperidyl]acetic acid | 0.599 | 7 | 19 | 31 | 29 | 15 | 8 | 6 | 3 | 2 | 1 | 1 | 1 |
| (μM) | 1.99 | 9 | 15 | 32 | 29 | 16 | 8 | 6 | 3 | 2 | 1 | 1 | 1 |
| | 3.99 | 7 | 19 | 31 | 30 | 16 | 8 | 5 | 4 | 2 | 1 | 1 | 1 |
| | 5.99 | 10 | 14 | 30 | 27 | 16 | 8 | 6 | 3 | 2 | 1 | 1 | 1 |
| | 9.99 | 9 | 21 | 30 | 30 | 16 | 8 | 6 | 4 | 2 | 1 | 1 | 1 |
| | 19.99 | 14 | 17 | 31 | 30 | 17 | 8 | 6 | 3 | 2 | 1 | 1 | 1 |
| | 29.98 | 8 | 19 | 31 | 31 | 18 | 8 | 6 | 3 | 2 | 1 | 1 | 1 |
| | 39.98 | 12 | 17 | 31 | 31 | 18 | 9 | 6 | 3 | 2 | 1 | 1 | 1 |
| | 59.96 | 12 | 22 | 30 | 32 | 17 | 9 | 6 | 3 | 2 | 1 | 1 | 1 |

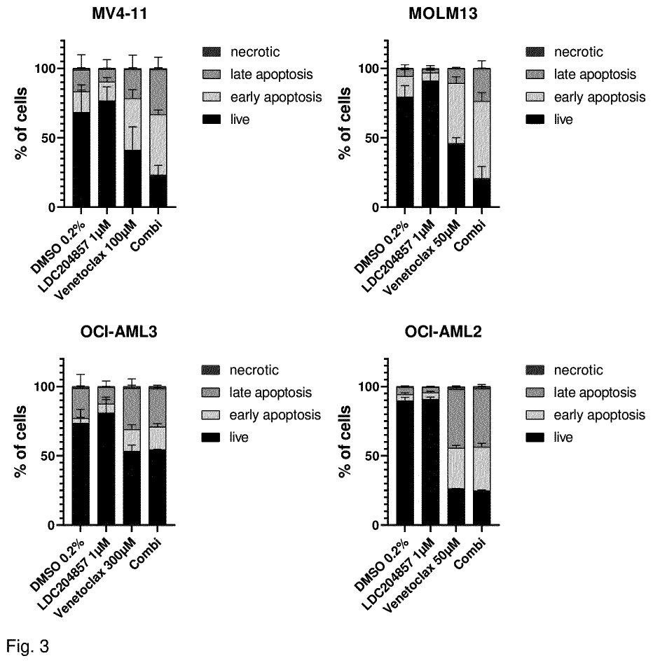

These experiments were followed by a broader analysis of the apoptosis induction by the inhibitor combination used in the four AML cell lines MV4-11, MOLM-13, OCI-AML3 and OCI-AML2 (FIG. 3). This analysis was carried out using flow cytometry and the established cellular markers for necrosis, apoptosis and living cells. It was found that the IMT 2-[(3R)-1-[(2R)-2-[4-(2-chloro-4-fluoro-phenyl)-2-oxo-chromen-7-yl]oxypropanoyl]-3-piperidyl]acetic acid used in these four cell lines, unlike Venetoclax, cannot trigger apoptosis. However, the combination of both inhibitors in MV4-11 and MOLM-13 cells increases the induction of apoptosis significantly beyond the level of Venetoclax alone.

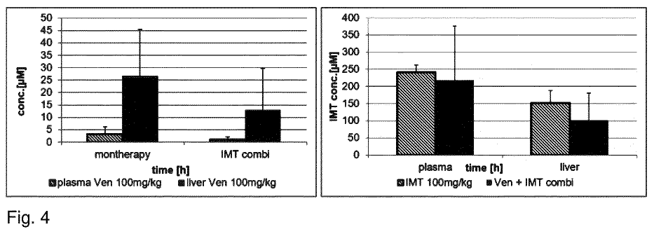

Based on these results, which demonstrated the synergistic effect of IMT and Bcl-2i in in vitro cell culture, studies were undertaken to investigate the effect of this combination in in vivo cancer models. For this, a vehicle formulation suitable for the oral administration of the combination of Venetoclax and 2-[(3R)-1-[(2R)-2-[4-(2-chloro-4-fluoro-phenyl)-2-oxo-chromen-7-yl]oxypropanoyl]-3-piperidyl]acetic acid was prepared to examine its pharmacokinetic suitability. Since the formulations based on ethanol, PEG-400 and Phosal that have been published for Venetoclax have not proven to be suitable for generating an administrable solution or suspension of IMT and Venetoclax, a simplified formulation of 0.5% methyl cellulose in water was prepared, which was tested initially in PK and toxicological studies in NBSGW mice (FIG. 4). On the one hand, these tests provided evidence that the PK parameters of the two substances used, in particular the plasma and tissue concentrations, do not significantly influence one another and that the combination is also available systemic. On the other hand, doses that could be safely used were established in this mouse model, with which the subsequent studies could be carried out.

### LITERATURE

ARIAANS, G., JALVING, M., VRIES, E. G. & JONG, S. 2017. Anti-tumor effects of everolimus and metformin are complementary and glucose-dependent in breast cancer cells. BMC Cancer, 17, 232.
ARNOLD, J. J., SMIDANSKY, E. D., MOUSTAFA, I. M. & CAMERON, C. E. 2012. Human mitochondrial RNA polymerase: structure-function, mechanism and inhibition. Biochim Biophys Acta, 1819, 948-60.
BEHERA, M. A., DAI, Q., GARDE, R., SANER, C., JUNGHEIM, E. & PRICE, T. M. 2009. Progesterone stimulates mitochondrial activity with subsequent inhibition of apoptosis in MCF-10A benign breast epithelial cells. Am J Physiol Endocrinol Metab, 297, E1089-96.
BHAT, M., SONENBERG, N. & GORES, G. J. 2013. The mTOR pathway in hepatic malignancies. Hepatology, 58, 810-8.
BRALHA, F. N., LIYANAGE, S. U., HURREN, R., WANG, X., SON, M. H., FUNG, T. A., CHINGCUANCO, F. B., TUNG, A. Y., ANDREAZZA, A. C., PSARIANOS, P., SCHIMMER, A. D., SALMENA, L. & LAPOSA, R. R. 2015. Targeting mitochondrial RNA polymerase in acute myeloid leukemia. Oncotarget, 6, 37216-28.
BRECHT, K., RIEBEL, V., COUTTET, P., PAECH, F., WOLF, A., CHIBOUT, S. D., POGNAN, F., KRAHENBUHL, S. & UTENG, M. 2017. Mechanistic insights into selective killing of OXPHOS-dependent cancer cells by arctigenin. Toxicol In Vitro, 40, 55-65.
BUBNER, B. & BALDWIN, I. T. 2004. Use of real-time PCR for determining copy number and zygosity in transgenic plants. Plant Cell Rep, 23, 263-71.
CARO, P., KISHAN, A. U., NORBERG, E., STANLEY, I. A., CHAPUY, B., FICARRO, S. B., POLAK, K., TONDERA, D., GOUNARIDES, J., YIN, H., ZHOU, F., GREEN, M. R., CHEN, L., MONTI, S., MARTO, J. A., SHIPP, M. A. & DANIAL, N. N. 2012. Metabolic signatures uncover distinct targets in molecular subsets of diffuse large B cell lymphoma. Cancer Cell, 22, 547-60.
CARROLL, S. S., TOMASSINI, J. E., BOSSERMAN, M., GETTY, K., STAHLHUT, M. W., ELDRUP, A. B., BHAT, B., HALL, D., SIMCOE, A. L., LAFEMINA, R., RUTKOWSKI, C. A., WOLANSKI, B., YANG, Z., MIGLIACCIO, G., DE FRANCESCO, R., KUO, L. C., MACCOSS, M. & OLSEN, D. B. 2003. Inhibition of hepatitis C virus RNA replication by 2′-modified nucleoside analogs. J Biol Chem, 278, 11979-84.
DENISE, C., PAOLI, P., CALVANI, M., TADDEI, M. L., GIANNONI, E., KOPETZ, S., KAZMI, S. M., PIA, M. M., PETTAZZONI, P., SACCO, E., CASELLI, A., VANONI, M., LANDRISCINA, M., CIRRI, P. & CHIARUGI, P. 2015. 5-fluorouracil resistant colon cancer cells are addicted to OXPHOS to survive and enhance stem-like traits. Oncotarget, 6, 41706-21.
DÖRR, J. R., YU, Y., MILANOVIC, M., BEUSTER, G., ZASADA, C., DABRITZ, J. H., LISEC, J., LENZE, D., GERHARDT, A., SCHLEICHER, K., KRATZAT, S., PURFURST, B., WALENTA, S., MUELLER-KLIESER, W., GRALER, M., HUMMEL, M., KELLER, U., BUCK, A. K., DORKEN, B., WILLMITZER, L., REIMANN, M., KEMPA, S., LEE, S. & SCHMITT, C. A. 2013. Synthetic lethal metabolic targeting of cellular senescence in cancer therapy. Nature, 501, 421-5.
FALKENBERG, M., GASPARI, M., RANTANEN, A., TRIFUNOVIC, A., LARSSON, N. G. & GUSTAFSSON, C. M. 2002. Mitochondrial transcription factors B1 and B2 activate transcription of human mtDNA. Nat Genet, 31, 289-94.
FULDA, S., GALLUZZI, L. & KROEMER, G. 2010. Targeting mitochondria for cancer therapy. Nat Rev Drug Discov, 9, 447-64.
GOSSELIN, F., BRITTON, R. A., DAVIES, I. W., DOLMAN, S. J., GAUVREAU, D., HOERRNER, R. S., HUGHES, G., JANEY, J., LAU, S., MOLINARO, C., NADEAU, C., O′SHEA, P. D., PALUCKI, M. & SIDLER, R. 2010. A practical synthesis of 5-lipoxygenase inhibitor MK-0633. J Org Chem, 75, 4154-60.
HANAN, E. J., VAN ABBEMA, A., BARRETT, K., BLAIR, W. S., BLANEY, J., CHANG, C., EIGENBROT, C., FLYNN, S., GIBBONS, P., HURLEY, C. A., KENNY, J. R., KULAGOWSKI, J., LEE, L., MAGNUSON, S. R., MORRIS, C., MURRAY, J., PASTOR, R. M., RAWSON, T., SIU, M., ULTSCH, M., ZHOU, A., SAMPATH, D. & LYSSIKATOS, J. P. 2012. Discovery of potent and selective pyrazolopyrimidine janus kinase 2 inhibitors. J Med Chem, 55, 10090-107.
HAQ, R., SHOAG, J., ANDREU-PEREZ, P., YOKOYAMA, S., EDELMAN, H., ROWE, G. C., FREDERICK, D. T., HURLEY, A. D., NELLORE, A., KUNG, A. L., WARGO, J. A., SONG, J. S., FISHER, D. E., ARANY, Z. & WIDLUND, H. R. 2013. Oncogenic BRAF regulates oxidative metabolism via PGC1alpha and MITF. Cancer Cell, 23, 302-15.
HAYNES, D. A., JONES, W. & MOTHERWELL, W. D. S. 2005. Occurrence of pharmaceutically acceptable anions and cations in the Cambridge Structural Database. Journal of Pharmaceutical Sciences, 94, 2111-2120.
HEID, C. A., STEVENS, J., LIVAK, K. J. & WILLIAMS, P. M. 1996. Real time quantitative PCR. Genome Res, 6, 986-94.
HSU, P. P. & SABATINI, D. M. 2008. Cancer cell metabolism: Warburg and beyond. Cell, 134, 703-7.
KLOMP, J. A., PETILLO, D., NIEMI, N. M., DYKEMA, K. J., CHEN, J., YANG, X. J., SAAF, A., ZICKERT, P., ALY, M., BERGERHEIM, U., NORDENSKJOLD, M., GAD, S., GIRAUD, S., DENOUX, Y., YONNEAU, L., MEJEAN, A., VASILIU, V., RICHARD, S., MACKEIGAN, J. P., TEH, B. T. & FURGE, K. A. 2010. Birt-Hogg-Dube renal tumors are genetically distinct from other renal neoplasias and are associated with up-regulation of mitochondrial gene expression. BMC Med Genomics, 3, 59.
LEONETTI, F., FAVIA, A., RAO, A., ALIANO, R., PALUSZCAK, A., HARTMANN, R. W. & CAROTTI, A. 2004. Design, synthesis, and 3D QSAR of novel potent and selective aromatase inhibitors. J Med Chem, 47, 6792-803.
LING, S., SONG, L., FAN, N., FENG, T., LIU, L., YANG, X., WANG, M., LI, Y., TIAN, Y., ZHAO, F., LIU, Y., HUANG, Q., HOU, Z., XU, F., SHI, L. & LI, Y. 2017. Combination of metformin and sorafenib suppresses proliferation and induces autophagy of hepatocellular carcinoma via targeting the mTOR pathway. Int J Oncol, 50, 297-309.
MITSUNOBU, O. & YAMADA, M. 1967. Preparation of Esters of Carboxylic and Phosphoric Acid via Quaternary Phosphonium Salts. Bulletin of the Chemical Society of Japan, 40, 2380-2382.
NADJI, M., GOMEZ-FERNANDEZ, C., GANJEI-AZAR, P. & MORALES, A. R. 2005. Immunohistochemistry of estrogen and progesterone receptors reconsidered: experience with 5,993 breast cancers. Am J Clin Pathol, 123, 21-7.
PELICANO, H., MARTIN, D. S., XU, R. H. & HUANG, P. 2006. Glycolysis inhibition for anticancer treatment. Oncogene, 25, 4633-46.
PELICANO, H., ZHANG, W., LIU, J., HAMMOUDI, N., DAI, J., XU, R. H., PUSZTAI, L. & HUANG, P. 2014. Mitochondrial dysfunction in some triple-negative breast cancer cell lines: role of mTOR pathway and therapeutic potential. Breast Cancer Res, 16, 434.
POSSE, V., SHAHZAD, S., FALKENBERG, M., HALLBERG, B. M. & GUSTAFSSON, C. M. 2015. TEFM is a potent stimulator of mitochondrial transcription elongation in vitro. Nucleic Acids Res, 43, 2615-24.
RODRIGUES, M. F., OBRE, E., DE MELO, F. H., SANTOS, G. C., JR., GALINA, A., JASIULIONIS, M. G., ROSSIGNOL, R., RUMJANEK, F. D. & AMOEDO, N. D. 2016. Enhanced OXPHOS, glutaminolysis and beta-oxidation constitute the metastatic phenotype of melanoma cells. Biochem J, 473, 703-15.
RODRIGUEZ-ENRIQUEZ, S., HERNANDEZ-ESQUIVEL, L., MARIN-HERNANDEZ, A., EL HAFIDI, M., GALLARDO-PEREZ, J. C., HERNANDEZ-RESENDIZ, I., RODRIGUEZ-ZAVALA, J. S., PACHECO-VELAZQUEZ, S. C. & MORENO-SANCHEZ, R. 2015. Mitochondrial free fatty acid beta-oxidation supports oxidative phosphorylation and proliferation in cancer cells. Int J Biochem Cell Biol, 65, 209-21.
ROESCH, A., VULTUR, A., BOGESKI, I., WANG, H., ZIMMERMANN, K. M., SPEICHER, D., KORBEL, C., LASCHKE, M. W., GIMOTTY, P. A., PHILIPP, S. E., KRAUSE, E., PATZOLD, S., VILLANUEVA, J., KREPLER, C., FUKUNAGA-KALABIS, M., HOTH, M., BASTIAN, B. C., VOGT, T. & HERLYN, M. 2013. Overcoming intrinsic multidrug resistance in melanoma by blocking the mitochondrial respiratory chain of slow-cycling JARID1B(high) cells. Cancer Cell, 23, 811-25.
SALEM, A. F., WHITAKER-MENEZES, D., LIN, Z., MARTINEZ-OUTSCHOORN, U. E., TANOWITZ, H. B., AL-ZOUBI, M. S., HOWELL, A., PESTELL, R. G., SOTGIA, F. & LISANTI, M. P. 2012. Two-compartment tumor metabolism: autophagy in the tumor microenvironment and oxidative mitochondrial metabolism (OXPHOS) in cancer cells. Cell Cycle, 11, 2545-56.
SANCHEZ-ALVAREZ, R., MARTINEZ-OUTSCHOORN, U. E., LAMB, R., HULIT, J., HOWELL, A., GANDARA, R., SARTINI, M., RUBIN, E., LISANTI, M. P. & SOTGIA, F. 2013. Mitochondrial dysfunction in breast cancer cells prevents tumor growth: understanding chemoprevention with metformin. Cell Cycle, 12, 172-82.
SCARPULLA, R. C. 2008. Transcriptional paradigms in mammalian mitochondrial biogenesis and function. Physiol Rev, 88, 611-38.
SCATENA, R., BOTTONI, P., PONTOGLIO, A., MASTROTOTARO, L. & GIARDINA, B. 2008. Glycolytic enzyme inhibitors in cancer treatment. Expert Opin Investig Drugs, 17, 1533-45.
SCHÖCKEL, L., GLASAUER, A., BASIT, F., BITSCHAR, K., TRUONG, H., ERDMANN, G., ALGIRE, C., HAGEBARTH, A., WILLEMS, P. H., KOPITZ, C., KOOPMAN, W. J. & HEROULT, M. 2015. Targeting mitochondrial complex I using BAY 87-2243 reduces melanoma tumor growth. Cancer Metab, 3, 11.
SIEGEL, R. L., MILLER, K. D. & JEMAL, A. 2016. Cancer statistics, 2016. CA Cancer J Clin, 66, 7-30.
TISDALE, M. J. 2002. Cachexia in cancer patients. Nat Rev Cancer, 2, 862-71.
VANDER HEIDEN, M. G., CANTLEY, L. C. & THOMPSON, C. B. 2009. Understanding the Warburg effect: the metabolic requirements of cell proliferation. Science, 324, 1029-33.
WALTERS, W. P. & NAMCHUK, M. 2003. Designing screens: how to make your hits a hit. Nat Rev Drug Discov, 2, 259-66.
WANROOIJ, S. & FALKENBERG, M. 2010. The human mitochondrial replication fork in health and disease. Biochim Biophys Acta, 1797, 1378-88.
WEINBERG, S. E. & CHANDEL, N. S. 2015. Targeting mitochondria metabolism for cancer therapy. Nat Chem Biol, 11, 9-15.
WHITAKER-MENEZES, D., MARTINEZ-OUTSCHOORN, U. E., LIN, Z., ERTEL, A., FLOMENBERG, N., WITKIEWICZ, A. K., BIRBE, R. C., HOWELL, A., PAVLIDES, S., GANDARA, R., PESTELL, R. G., SOTGIA, F., PHILP, N. J. & LISANTI, M. P. 2011. Evidence for a stromal-epithelial “lactate shuttle” in human tumors: MCT4 is a marker of oxidative stress in cancer-associated fibroblasts. Cell Cycle, 10, 1772-83.
YEUNG, K. Y., DICKINSON, A., DONOGHUE, J. F., POLEKHINA, G., WHITE, S. J., GRAMMATOPOULOS, D. K., MCKENZIE, M., JOHNS, T. G. & ST JOHN, J. C. 2014. The identification of mitochondrial DNA variants in glioblastoma multiforme. Acta Neuropathol Commun, 2, 1.
WO 2016/146583 A1
WO 2016/193231 A1
BONEKAMP, N. A., PETER, B., HILLEN, H. S., FELSER, A., BERGBREDE, T., CHOLDAS, A., HORN, M., UNGER, A., DI LUCREZIA, R., ATANASSOV, I., LI, X., KOCH, U., MENNINGER, S., BOROS, J., HABENBERGER, P., GLAVALISCO, P., CRAMER,
P., DENZEL, M. S., NUSSBAUMER, P., KLEBL, B., FALKENBERG, M., GUSTAFSSON, C. M., LARSSON, N.-G. 2020. Small-molecule inhibitors of human mitochindrial DANN transcription. Nature, 1-5.
WO2019/057821
WO2020/188049
WO2020/068979
EP02930238
WO2020/086830
BRALHA ET AL. doi: 1018632/oncotarget6129

1. A composition comprising at least one inhibitor of mitochondrial transcription (IMT) and at least one anti-cancer drug.

2. The composition according to any of the proceeding claims, wherein the at least one IMT is a compound of the general formula (I) 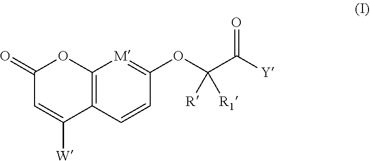
wherein
R′ is —C₁-C₄-alkyl, preferably -methyl or -ethyl, in particular -methyl;
R₁′ is —H, or -methyl, preferably —H;
M′ is CH or N;
W′ is with 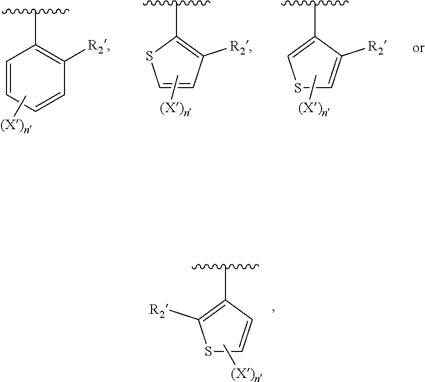
R₂′ is C₁-C₄-alkyl, -halogen, —CN, preferably -methyl, -ethyl, —C₁, or —Br;
X′ is -halogen, or —CN, preferably —Cl, —Br, or —F, in particular —F, with n′=1 or 2;
n′=0, 1, or 2, preferably 0 or 1;
Y′ is —NR₃′R₄′ with
R₃′ is —H, or —C₁-C₄-alkyl, preferably —H or -methyl, and
R₄′ is —C₁-C₄-alkyl or —C₃-C₆-cycloalkyl, preferably -methyl, -ethyl, -isopropyl, or -cyclopropyl; or
an unsubstituted or substituted pyridine residue; or
an unsubstituted or substituted phenyl residue, preferably substituted at the para position;
Y′ is —NR₃′R₄′ with N, R₃′ and R₄′ forming an unsubstituted or substituted 5- or 6-membered saturated heterocycle; or
Y′ is —OR₁₁′, with R₁₁′ is —H or —C₁-C₄-alkyl, preferably —H, -methyl, -ethyl, or -isopropyl, or a pharmaceutically or veterinary acceptable salt, hydrate or solvate thereof;
or
wherein the at least one IMT is a compound of the general formula (II) 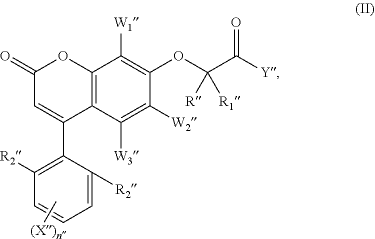
wherein
R′″ is —H or —C₁-C₄-alkyl;
R₁′ is —H or -methyl;
R₂′″ is —H;
n′″=0, 1 or 2;
X′″ is -halogen, —C₁-C₄-alkyl, —OMe or —CN, with n′″=1 or 2;
Y″ is —NR₃′″R₄′″ with
R₃′″ is —H, or —C₁-C₄-alkyl, and
R₄′″ is —H, —C₁-C₄-alkyl or —C₃-C₈-cycloalkyl;
an unsubstituted or substituted pyridine residue;
an unsubstituted or substituted pyridinylmethyl residue;
an unsubstituted or substituted morpholinylethyl residue;
an unsubstituted or substituted furanylmethyl residue;
an unsubstituted or substituted phenyl residue;
an unsubstituted or substituted benzyl residue;
an unsubstituted or substituted phenethyl residue;
the group 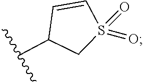 [SMILES: *C1C=CS(=O)(=O)C1]
or
the group 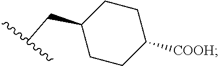 [SMILES: *C[C@H]1CC[C@H](C(=O)O)CC1]
or
Y″ is —NR₃′″R₄′″ with N, R₃′″ and R₄′″ forming an unsubstituted or substituted 5- or 6-membered saturated heterocycle, 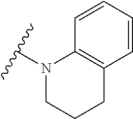 [SMILES: *N1CCCc2ccccc21]
or
Y″ is —OR₁₁′″, with R₁₁′″ is —H or —C₁-C₄-alkyl, phenyl, benzyl or 2-ethoxyethyl; and
W₁′″, W₂′″, and W₃′″ are identical or different, and are —H, -halogen, or —C₁-C₄-alkyl;
or a pharmaceutically or veterinary acceptable salt, hydrate or solvate thereof, or wherein the at least one IMT is a compound of the general formula (III) 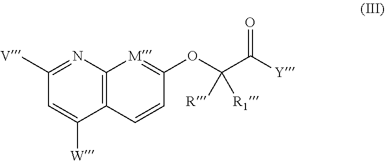
wherein
R′″ is —H, or —C₁-C₄-alkyl, preferably —H, -methyl or -ethyl; in particular methyl;
R₁′″ is —H, or -methyl, preferably —H;
M′″ is CH or N; preferably CH;
V′″ is —H, —OH, —Cl, —F, or —C₁-C₄-alkyl, preferably —H, —Cl, —F, or -methyl;
W′″ is 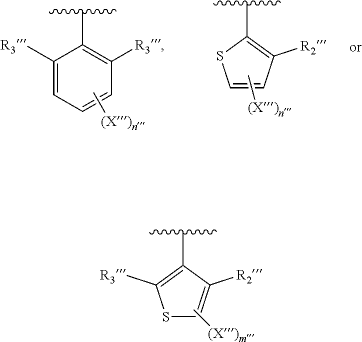
R₂′″ and R₃′″ are identical or different and are
—H, —C₁-C₄-alkyl, halogen-C₁-C₄-alkyl, —C₁-C₄-alkoxy, —C₁-C₄-dialkylamino, —C₂-C₆-alkenyl, —C₂-C₆ alkynyl, -halogen, —CN or —CO—NH₂;
preferably —H, —C₁-C₄-alkyl, —CF₃, —OCH₃, —NHCH₃, —N(CH₃)₂, —F, or —Cl;
X′″ is -halogen, or —CN, preferably —F, with n′″=1 or 2 or with m′″=1;
n′″=0, 1, or 2, preferably 0 or 1;
m′″=0 or 1, preferably 0;
Y′″ is —NR₄′″R₅′″ with
R₄′″ is —H, or —C₁-C₄-alkyl, preferably —H or -methyl, and
R₅′″ is —H, —C₁-C₄-alkyl, an unsubstituted or substituted —C₃-C₆-cycloalkyl, preferably -methyl, -ethyl, -isopropyl, or or -cyclopropyl;
an unsubstituted or substituted pyridine residue; or
an unsubstituted or substituted phenyl residue, preferably substituted at the para position; or
Y′″ is —NR₄′″R₅′″ with N, R₄′″ and R₅′″ forming an unsubstituted or substituted 4-, 5- or 6-membered saturated heterocycle, preferably an unsubstituted or substituted azetidine residue, an unsubstituted or substituted piperidine residue, an unsubstituted or substituted pyrrolidine residue, an unsubstituted or substituted piperazine residue, an unsubstituted or substituted morpholine residue, or an unsubstituted or substituted tetrahydropyridine residue; or
Y′″ is —OR₆′″, with R₆′″ is —H or —C₁-C₄-alkyl, preferably —H, -methyl, -ethyl, -isopropyl, or -tert-butyl;
or a pharmaceutically or veterinary acceptable salt, hydrate or solvate thereof.

3. The composition according to any of the proceeding claims, wherein the IMT is selected from the group consisting of:
7-[1-methyl-2-oxo-2-(1-piperidyl)ethoxy]-4-(o-tolyl)chromen-2-one, 4-(2-chlorophenyl)-7-[1-methyl-2-oxo-2-(1-piperidyl)ethoxy]chromen-2-one,
(3S)-1-[2-[4-(o-tolyl)-2-oxo-chromen-7-yl]oxypropanoyl]piperidine-3-carboxylic acid,
2-[4-(o-tolyl)-2-oxo-chromen-7-yl]oxy-N-(2-pyridyl)propanamide,
7-(1-methyl-2-oxo-2-pyrrolidin-1-yl-ethoxy)-4-(o-tolyl)chromen-2-one,
methyl 1-[2-[4-(o-tolyl)-2-oxo-chromen-7-yl]oxypropanoyl]piperidine-3-carboxylate,
methyl 1-[2-[4-(2-chlorophenyl)-2-oxo-chromen-7-yl]oxypropanoyl]piperidine-3-carboxylate,
1-[2-[4-(o-tolyl)-2-oxo-chromen-7-yl]oxypropanoyl]piperidine-3-carboxylic acid,
1-[2-[4-(2-chlorophenyl)-2-oxo-chromen-7-yl]oxypropanoyl]piperidine-3-carboxylic acid,
1-[2-[4-(o-tolyl)-2-oxo-chromen-7-yl]oxypropanoyl]piperidine-3-sulfonic acid,
1-[2-[4-(2-chlorophenyl)-2-oxo-chromen-7-yl]oxypropanoyl]piperidine-3-sulfonic acid,
1-[2-[4-(o-tolyl)-2-oxo-chromen-7-yl]oxypropanoyl]piperidine-3-sulfonamide,
4-(2-chlorophenyl)-7-(1-methyl-2-oxo-2-pyrrolidin-1-yl-ethoxy)chromen-2-one,
(3S)-1-[(2R)-2-[4-(2-chloro-4-fluoro-phenyl)-2-oxo-chromen-7-yl]oxypropanoyl]piperidine-3-carboxylic acid,
1-[2-[4-(2-chlorophenyl)-2-oxo-chromen-7-yl]oxypropanoyl]-N-methyl-piperidine-3-carboxamide,
ethyl (3S)-1-[2-[4-(2-chlorophenyl)-2-oxo-chromen-7-yl]oxypropanoyl]piperidine-3-carboxylate,
ethyl (3S)-1-[2-[4-(o-tolyl)-2-oxo-chromen-7-yl]oxypropanoyl]piperidine-3-carboxylate,
N-methyl-1-[2-[4-(o-tolyl)-2-oxo-chromen-7-yl]oxypropanoyl]piperidine-3-sulfonamide,
N-[4-(2-hydroxyethyl)phenyl]-2-[4-(o-tolyl)-2-oxo-chromen-7-yl]oxy-propanamide,
1-[2-[4-(2-chlorophenyl)-2-oxo-chromen-7-yl]oxypropanoyl]-N-methyl-piperidine-3-sulfonamide,
N-methyl-1-[2-[4-(o-tolyl)-2-oxo-chromen-7-yl]oxypropanoyl]piperidine-3-carboxamide,
(3S)-1-[2-[4-(o-tolyl)-2-oxo-chromen-7-yl]oxypropanoyl]pyrrolidine-3-carboxylic acid,
(3S)-1-[(2R)-2-[4-(2-chlorophenyl)-2-oxo-chromen-7-yl]oxypropanoyl]piperidine-3-carboxylic acid,
1-[2-[4-(2-chlorophenyl)-2-oxo-chromen-7-yl]oxypropanoyl]piperidine-3-sulfonamide,
N-cyclopropyl-2-[4-(o-tolyl)-2-oxo-chromen-7-yl]oxy-propanamide,
2-[4-(2-chlorophenyl)-2-oxo-chromen-7-yl]oxy-N-cyclopropyl-propanamide,
(3S)-1-[(2R)-2-[4-(o-tolyl)-2-oxo-chromen-7-yl]oxypropanoyl]piperidine-3-carboxylic acid,
(3S)-1-[(2R)-2-[4-(2-chlorophenyl)-2-oxo-chromen-7-yl]oxypropanoyl]piperidine-3-carbonitrile,
(3S)-1-[2-[4-(2-chlorophenyl)-2-oxo-chromen-7-yl]oxypropanoyl]pyrrolidine-3-carboxylic acid
7-[(1R)-1-methyl-2-oxo-2-(1-piperidyl)ethoxy]-4-(o-tolyl)chromen-2-one,
2-[4-(2-chlorophenyl)-2-oxo-chromen-7-yl]oxy-N-(2-pyridyl)propanamide,
(3S)-1-[(2R)-2-[4-(o-tolyl)-2-oxo-chromen-7-yl]oxypropanoyl]piperidine-3-carbonitrile,
(3S)-1-[2-[4-(2-chlorophenyl)-2-oxo-chromen-7-yl]oxypropanoyl]piperidine-3-carboxylic acid,
4-(2-chlorophenyl)-7-[(1R)-1-methyl-2-oxo-2-(1-piperidyl)ethoxy]chromen-2-one, 2-[4-(2-chlorophenyl)-2-oxo-chromen-7-yl]oxy-N-[4-(2-hydroxyethyl)phenyl]propanamide,
(2R)—N-isopropyl-2-[4-(o-tolyl)-2-oxo-chromen-7-yl]oxy-propanamide,
1-[2-[4-(2-chloro-4-fluoro-phenyl)-2-oxo-chromen-7-yl]oxypropanoyl]-3-methyl-piperidine-3-carboxylic acid,
N,N-dimethyl-2-[4-(o-tolyl)-2-oxo-chromen-7-yl]oxy-propanamide,
4-(2-chlorophenyl)-7-[1-methyl-2-oxo-2-[3-(2H-tetrazol-5-yl)-1-piperidyl]ethoxy]chromen-2-one,
ethyl 2-[4-(2-chlorophenyl)-2-oxo-chromen-7-yl]oxypropanoate,
1-[2-[4-(o-tolyl)-2-oxo-chromen-7-yl]oxypropanoyl]piperidine-3-carbonitrile,
7-[1-methyl-2-oxo-2-[3-(2H-tetrazol-5-yl)-1-piperidyl]ethoxy]-4-(o-tolyl)chromen-2-one,
3-methyl-1-[2-[4-(o-tolyl)-2-oxo-chromen-7-yl]oxypropanoyl]piperidine-3-carboxylic acid,
(2R)—N,N-dimethyl-2-[4-(o-tolyl)-2-oxo-chromen-7-yl]oxy-propanamide,
(2R)-2-[4-(2-chlorophenyl)-2-oxo-chromen-7-yl]oxy-N,N-dimethyl-propanamide,
(3R)-1-[2-[4-(2-chlorophenyl)-2-oxo-chromen-7-yl]oxypropanoyl]piperidine-3-carboxylic acid,
(3S)-1-[2-[4-(2-chloro-4-fluoro-phenyl)-2-oxo-chromen-7-yl]oxypropanoyl]piperidine-3-carboxylic acid,
1-[2-[4-(2-chlorophenyl)-2-oxo-chromen-7-yl]oxypropanoyl]piperidine-3-carbonitrile,
2-[4-(2-chloro-3-fluoro-phenyl)-2-oxo-chromen-7-yl]oxy-N-isopropyl-propanamide,
(3S)-1-[2-[4-(2-chloro-3-fluoro-phenyl)-2-oxo-chromen-7-yl]oxypropanoyl]piperidine-3-carboxylic acid,
2-[4-(2-chlorophenyl)-2-oxo-chromen-7-yl]oxy-N-methyl-propanamide,
1-[2-[4-(2-chloro-3-fluoro-phenyl)-2-oxo-chromen-7-yl]oxypropanoyl]piperidine-3-sulfonamide,
(3R)-1-[2-[4-(o-tolyl)-2-oxo-chromen-7-yl]oxypropanoyl]piperidine-3-carboxylic acid,
1-[2-[4-(2-chlorophenyl)-2-oxo-chromen-7-yl]oxypropanoyl]-3-methyl-piperidine-3-carboxylic acid,
isopropyl (2R)-2-[4-(o-tolyl)-2-oxo-chromen-7-yl]oxypropanoate,
(2R)-2-[4-(2-chlorophenyl)-2-oxo-chromen-7-yl]oxy-N-isopropyl-propanamide,
ethyl 2-[4-(2-chloro-3-fluoro-phenyl)-2-oxo-chromen-7-yl]oxypropanoate,
ethyl 2-[4-(o-tolyl)-2-oxo-chromen-7-yl]oxypropanoate,
2-[1-[2-[4-(2-chlorophenyl)-2-oxo-chromen-7-yl]oxypropanoyl]-4-piperidyl]acetic acid,
2-[1-[2-[4-(o-tolyl)-2-oxo-chromen-7-yl]oxypropanoyl]-4-piperidyl]acetic acid,
2-[4-(2-chloro-3-fluoro-phenyl)-2-oxo-chromen-7-yl]oxy-N-cyclopropyl-propanamide,
N-isopropyl-2-[4-(o-tolyl)-2-oxo-chromen-7-yl]oxy-propanamide,
1-[2-[4-(o-tolyl)-2-oxo-chromen-7-yl]oxypropanoyl]pyrrolidine-3-sulfonamide,
isopropyl (2R)-2-[4-(2-chlorophenyl)-2-oxo-chromen-7-yl]oxypropanoate,
2-[4-(2-chloro-4-fluoro-phenyl)-2-oxo-chromen-7-yl]oxy-N-isopropyl-propanamide,
2-[1-[2-[4-(2-chlorophenyl)-2-oxo-chromen-7-yl]oxypropanoyl]-3-piperidyl]acetic acid,
(3R)-1-[2-[4-(2-chlorophenyl)-2-oxo-chromen-7-yl]oxypropanoyl]-N,N-dimethyl-piperidine-3-carboxamide,
2-[4-(2-chloro-3-fluoro-phenyl)-2-oxo-chromen-7-yl]oxy-N-ethyl-propanamide,
1-[2-[4-(2-chloro-4-fluoro-phenyl)-2-oxo-chromen-7-yl]oxypropanoyl]piperidine-3-sulfonamide,
2-[4-(2-chlorophenyl)-2-oxo-chromen-7-yl]oxy-N-isopropyl-propanamide,
7-[2-(4,4-difluoro-1-piperidyl)-1-methyl-2-oxo-ethoxy]-4-(o-tolyl)chromen-2-one,
2-[1-[2-[4-(o-tolyl)-2-oxo-chromen-7-yl]oxypropanoyl]-3-piperidyl]acetic acid,
7-[1-methyl-2-[3-(methylsulfonimidoyl)-1-piperidyl]-2-oxo-ethoxy]-4-(o-tolyl)chromen-2-one,
4-(2-chlorophenyl)-7-[2-(4,4-difluoro-1-piperidyl)-1-methyl-2-oxo-ethoxy]chromen-2-one,
ethyl 1-[2-[4-(2-chlorophenyl)-2-oxo-chromen-7-yl]oxypropanoyl]-3-methyl-piperidine-3-carboxylate,
(3S)-1-[2-[4-(2-chlorophenyl)-2-oxo-chromen-7-yl]oxypropanoyl]-N,N-dimethyl-piperidine-3-carboxamide,
1-[2-[4-(2-chlorophenyl)-2-oxo-chromen-7-yl]oxypropanoyl]pyrrolidine-3-sulfonamide,
(3R)—N,N-dimethyl-1-[2-[4-(o-tolyl)-2-oxo-chromen-7-yl]oxypropanoyl]piperidine-3-carboxamide,
4-(2-chlorophenyl)-7-[1-methyl-2-[3-(methylsulfonimidoyl)-1-piperidyl]-2-oxo-ethoxy]chromen-2-one,
methyl 2-[1-[2-[4-(2-chlorophenyl)-2-oxo-chromen-7-yl]oxypropanoyl]-4-piperidyl]acetate,
ethyl 2-[4-(2-bromophenyl)-2-oxo-chromen-7-yl]oxypropanoate,
ethyl 2-[4-(2-chloro-4-fluoro-phenyl)-2-oxo-chromen-7-yl]oxypropanoate,
methyl 2-[1-[2-[4-(o-tolyl)-2-oxo-chromen-7-yl]oxypropanoyl]-4-piperidyl]acetate,
(3S)—N,N-dimethyl-1-[2-[4-(o-tolyl)-2-oxo-chromen-7-yl]oxypropanoyl]piperidine-3-carboxamide,
N-ethyl-2-[4-(o-tolyl)-2-oxo-chromen-7-yl]oxy-propanamide,
ethyl 2-[1-[2-[4-(2-chlorophenyl)-2-oxo-chromen-7-yl]oxypropanoyl]-3-piperidyl]acetate,
ethyl 2-[1-[2-[4-(o-tolyl)-2-oxo-chromen-7-yl]oxypropanoyl]-3-piperidyl]acetate,
ethyl 3-methyl-1-[2-[4-(o-tolyl)-2-oxo-chromen-7-yl]oxypropanoyl]piperidine-3-carboxylate,
2-[4-(2-bromophenyl)-2-oxo-chromen-7-yl]oxy-N,N-dimethyl-propanamide,
2-[4-(2-chloro-4-fluoro-phenyl)-2-oxo-chromen-7-yl]oxy-N-cyclopropyl-propanamide,
2-[4-(2-chloro-4-fluoro-phenyl)-2-oxo-chromen-7-yl]oxy-N-ethyl-propanamide,
isopropyl 2-[4-(2-bromophenyl)-2-oxo-chromen-7-yl]oxypropanoate,
2-[4-(2-chlorophenyl)-2-oxo-chromen-7-yl]oxy-N,N-dimethyl-propanamide,
2-[4-(2-chloro-3-fluoro-phenyl)-2-oxo-chromen-7-yl]oxypropanoic acid,
ethyl 1-[2-[4-(2-chloro-4-fluoro-phenyl)-2-oxo-chromen-7-yl]oxypropanoyl]-3-methyl-piperidine-3-carboxylate,
2-[4-(2-chlorophenyl)-2-oxo-chromen-7-yl]oxypropanoic acid,
2-[4-(o-tolyl)-2-oxo-chromen-7-yl]oxypropanoic acid,
2-[4-(2-chlorophenyl)-2-oxo-chromen-7-yl]oxy-N-ethyl-propanamide,
2-[4-(2-chloro-4-fluoro-phenyl)-2-oxo-chromen-7-yl]oxypropanoic acid,
methyl 2-[(3S)-1-[(2R)-2-[4-(2-chlorophenyl)-2-oxo-chromen-7-yl]oxypropanoyl]-3-piperidyl]acetate,
methyl 2-[(3R)-1-[(2R)-2-[4-(2-chlorophenyl)-2-oxo-chromen-7-yl]oxypropanoyl]-3-piperidyl]acetate,
2-[(3S)-1-[(2R)-2-[4-(2-chlorophenyl)-2-oxo-chromen-7-yl]oxypropanoyl]-3-piperidyl]acetic acid,
2-[(3R)-1-[(2R)-2-[4-(2-chlorophenyl)-2-oxo-chromen-7-yl]oxypropanoyl]-3-piperidyl]acetic acid,
methyl 2-[(3R)-1-[(2R)-2-[4-(2-chloro-4-fluoro-phenyl)-2-oxo-chromen-7-yl]oxypropanoyl]-3-piperidyl]acetate,
methyl 2-[(3S)-1-[(2R)-2-[4-(2-chloro-4-fluoro-phenyl)-2-oxo-chromen-7-yl]oxypropanoyl]-3-piperidyl]acetate,
2-[(3R)-1-[(2R)-2-[4-(2-chloro-4-fluoro-phenyl)-2-oxo-chromen-7-yl]oxypropanoyl]-3-piperidyl]acetic acid,
2-[(3S)-1-[(2R)-2-[4-(2-chloro-4-fluoro-phenyl)-2-oxo-chromen-7-yl]oxypropanoyl]-3-piperidyl]acetic acid,
(3R)-1-[(2R)-2-[4-(2-chloro-4-fluoro-phenyl)-2-oxo-chromen-7-yl]oxypropanoyl]piperidine-3-carboxylic acid,
ethyl (3S)-1-[2-[4-(2-chlorophenyl)-2-oxo-chromen-7-yl]oxybutanoyl]piperidine-3-carboxylate,
(3S)-1-[2-[4-(2-chlorophenyl)-2-oxo-chromen-7-yl]oxybutanoyl]piperidine-3-carboxylic acid,
ethyl (3S)-1-[(2R)-2-[4-(2-chloro-4-fluoro-phenyl)-2-oxo-chromen-7-yl]oxypropanoyl]piperidine-3-carboxylate,
2-[4-(2-chlorophenyl)-2-oxo-chromen-7-yl]oxy-2-methyl-propanoic acid,
ethyl 2-[4-(2-chlorophenyl)-2-oxo-chromen-7-yl]oxy-2-methyl-propanoate,
2-[4-(2-chlorophenyl)-2-oxo-chromen-7-yl]oxy-N-isopropyl-2-methyl-propanamide,
N-isopropyl-2-methyl-2-[4-(o-tolyl)-2-oxo-chromen-7-yl]oxy-propanamide,
(3S)-1-[2-[4-(2-chlorophenyl)-2-oxo-chromen-7-yl]oxy-2-methyl-propanoyl]piperidine-3-carboxylic acid,
isopropyl (3S)-1-[(2R)-2-[4-(2-chloro-4-fluoro-phenyl)-2-oxo-chromen-7-yl]oxypropanoyl]piperidine-3-carboxylate,
tert-butyl (3S)-1-[(2R)-2-[4-(2-chloro-4-fluoro-phenyl)-2-oxo-chromen-7-yl]oxypropanoyl]piperidine-3-carboxylate,
2-morpholinoethyl (3S)-1-[(2R)-2-[4-(2-chloro-4-fluoro-phenyl)-2-oxo-chromen-7-yl]oxypropanoyl]piperidine-3-carboxylate,
heptyl (3S)-1-[(2R)-2-[4-(2-chloro-4-fluoro-phenyl)-2-oxo-chromen-7-yl]oxypropanoyl]piperidine-3-carboxylate,
isopropoxycarbonyloxymethyl (3S)-1-[(2R)-2-[4-(2-chloro-4-fluoro-phenyl)-2-oxo-chromen-7-yl]oxypropanoyl]piperidine-3-carboxylate,
(3S)—N-methyl-1-[(2R)-2-[4-(2-chloro-4-fluoro-phenyl)-2-oxo-chromen-7-yl]oxypropanoyl piperidine-3-carboxamide,
isopropyl 2-[(3R)-1-[(2R)-2-[4-(2-chloro-4-fluoro-phenyl)-2-oxo-chromen-7-yl]oxypropanoyl]-3-piperidyl]acetate,
tert-butyl 2-[(3R)-1-[(2R)-2-[4-(2-chloro-4-fluoro-phenyl)-2-oxo-chromen-7-yl]oxypropanoyl]-3-piperidyl]acetate,
2-morpholinoethyl 2-[(3R)-1-[(2R)-2-[4-(2-chloro-4-fluoro-phenyl)-2-oxo-chromen-7-yl]oxypropanoyl]-3-piperidyl]acetate,
heptyl 2-[(3R)-1-[(2R)-2-[4-(2-chloro-4-fluoro-phenyl)-2-oxo-chromen-7-yl]oxypropanoyl]-3-piperidyl]acetate,
isopropoxycarbonyloxymethyl 2-[(3R)-1-[(2R)-2-[4-(2-chloro-4-fluoro-phenyl)-2-oxo-chromen-7-yl]oxypropanoyl]-3-piperidyl]acetate,
2-[(3R)-1-[(2R)-2-[4-(2-chloro-4-fluoro-phenyl)-2-oxo-chromen-7-yl]oxypropanoyl]-3-piperidyl]-N-methyl-acetamide,
methyl 1-[(2R)-2-[4-(2-chloro-4-fluoro-phenyl)-2-oxo-chromen-7-yl]oxypropanoyl]piperidine-4-carboxylate,
(2R)-2-[4-(2-chloro-4-fluoro-phenyl)-2-oxo-chromen-7-yl]oxy-N-[4-(hydroxymethyl)phenyl]propanamide,
1-[(2R)-2-[4-(2-chloro-4-fluoro-phenyl)-2-oxo-chromen-7-yl]oxypropanoyl]piperidine-4-carboxylic acid,
(3S)-1-[(2R)-2-[4-(2-chloro-4-fluoro-phenyl)-2-oxo-chromen-7-yl]oxypropanoyl]piperidine-3-carboxamide,
2-[(3R)-1-[(2R)-2-[4-(2-chloro-4-fluoro-phenyl)-2-oxo-chromen-7-yl]oxypropanoyl]-3-piperidyl]acetamide,
(3S)-1-[(2R)-2-[4-(2-ethylphenyl)-2-oxo-chromen-7-yl]oxypropanoyl]piperidine-3-carboxylic acid,
methyl (2S)-4-[(2R)-2-[4-(2-chloro-4-fluoro-phenyl)-2-oxo-chromen-7-yl]oxypropanoyl]piperazine-2-carboxylate,
methyl (2R)-4-[(2R)-2-[4-(2-chloro-4-fluoro-phenyl)-2-oxo-chromen-7-yl]oxypropanoyl]piperazine-2-carboxylate,
methyl 1-methyl-4-[rac-(2R)-2-[4-(2-chloro-4-fluoro-phenyl)-2-oxo-chromen-7-yl]oxypropanoyl]piperazine-2-carboxylate,
(2S)-4-[(2R)-2-[4-(2-chloro-4-fluoro-phenyl)-2-oxo-chromen-7-yl]oxypropanoyl]piperazine-2-carboxylic acid,
3-[[(2R)-2-[4-(2-chloro-4-fluoro-phenyl)-2-oxo-chromen-7-yl]oxypropanoyl]amino]benzoic acid,
(2R)-2-[4-(2-chloro-4-fluoro-phenyl)-2-oxo-chromen-7-yl]oxy-N-[4-(2-methoxyethyl)phenyl]propanamide,
4-[[(2R)-2-[4-(2-chloro-4-fluoro-phenyl)-2-oxo-chromen-7-yl]oxypropanoyl]amino]benzoic acid,
methyl 2-[(3R)-1-[(2R)-2-[4-(2-chloro-4-fluoro-phenyl)-2-oxo-chromen-7-yl]oxypropanoyl]pyrrolidin-3-yl]acetate,
methyl 5-[[(2R)-2-[4-(2-chloro-4-fluoro-phenyl)-2-oxo-chromen-7-yl]oxypropanoyl]amino]pyridine-3-carboxylate,
methyl 2-[(3S)-1-[(2R)-2-[4-(2-chloro-4-fluoro-phenyl)-2-oxo-chromen-7-yl]oxypropanoyl]pyrrolidin-3-yl]acetate,
2-[(3R)-1-[(2R)-2-[4-(2-chloro-4-fluoro-phenyl)-2-oxo-chromen-7-yl]oxypropanoyl]pyrrolidin-3-yl]acetic acid,
methyl 2-[[(2R)-2-[4-(2-chloro-4-fluoro-phenyl)-2-oxo-chromen-7-yl]oxypropanoyl]amino]pyridine-4-carboxylate,
2-[(3S)-1-[(2R)-2-[4-(2-chloro-4-fluoro-phenyl)-2-oxo-chromen-7-yl]oxypropanoyl]pyrrolidin-3-yl]acetic acid,
5-[[(2R)-2-[4-(2-chloro-4-fluoro-phenyl)-2-oxo-chromen-7-yl]oxypropanoyl]amino]pyridine-3-carboxylic acid,
methyl 6-[[(2R)-2-[4-(2-chloro-4-fluoro-phenyl)-2-oxo-chromen-7-yl]oxypropanoyl]amino]pyridine-2-carboxylate,
(2R)-4-[(2R)-2-[4-(2-chloro-4-fluoro-phenyl)-2-oxo-chromen-7-yl]oxypropanoyl]piperazine-2-carboxylic acid,
6-[[(2R)-2-[4-(2-chloro-4-fluoro-phenyl)-2-oxo-chromen-7-yl]oxypropanoyl]amino]pyridine-2-carboxylic acid,
2-[[(2R)-2-[4-(2-chloro-4-fluoro-phenyl)-2-oxo-chromen-7-yl]oxypropanoyl]amino]pyridine-4-carboxylic acid,
methyl 5-[[(2R)-2-[4-(2-chloro-4-fluoro-phenyl)-2-oxo-chromen-7-yl]oxypropanoyl]amino]pyridine-2-carboxylate,
5-[[(2R)-2-[4-(2-chloro-4-fluoro-phenyl)-2-oxo-chromen-7-yl]oxypropanoyl]amino]pyridine-2-carboxylic acid,
5-[[(2R)-2-[4-(2-chloro-4-fluoro-phenyl)-2-oxo-chromen-7-yl]oxypropanoyl]amino]pyridine-2-carboxylic acid,
6-[[(2R)-2-[4-(2-chloro-4-fluoro-phenyl)-2-oxo-chromen-7-yl]oxypropanoyl]amino]pyridine-3-carboxylic acid,
methyl 4-[[(2R)-2-[4-(2-chloro-4-fluoro-phenyl)-2-oxo-chromen-7-yl]oxypropanoyl]amino]pyridine-2-carboxylate,
methyl (3S)-1-[(2R)-2-[4-(2-chloro-4-fluoro-phenyl)-2-oxo-chromen-7-yl]oxypropanoyl]pyrrolidine-3-carboxylate,
(3S)-1-[(2R)-2-[4-(4-fluoro-2-methyl-phenyl)-2-oxo-chromen-7-yl]oxypropanoyl]piperidine-3-carboxylic acid,
(3S)-1-[(2R)-2-[4-(2-chloro-4-fluoro-phenyl)-2-oxo-chromen-7-yl]oxypropanoyl]pyrrolidine-3-carboxylic acid,
4-[[(2R)-2-[4-(2-chloro-4-fluoro-phenyl)-2-oxo-chromen-7-yl]oxypropanoyl]amino]pyridine-2-carboxylic acid,
1-methyl-4-[rac-(2R)-2-[4-(2-chloro-4-fluoro-phenyl)-2-oxo-chromen-7-yl]oxypropanoyl]piperazine-2-carboxylic acid,
(3S)-1-[(2R)-2-[4-(2-cyanophenyl)-2-oxo-chromen-7-yl]oxypropanoyl]piperidine-3-carboxylic acid,
(3S)-1-[(2R)-2-[4-(2,6-dichlorophenyl)-2-oxo-chromen-7-yl]oxypropanoyl]piperidine-3-carboxylic acid,
4-(2-chloro-4-fluoro-phenyl)-7-[(1R)-1-methyl-2-oxo-2-(4-prop-2-enoylpiperazin-1-yl)ethoxy]chromen-2-one,
N-[(3S)-1-[(2R)-2-[4-(2-chloro-4-fluoro-phenyl)-2-oxo-chromen-7-yl]oxypropanoyl]-3-piperidyl]prop-2-enamide,
7-[(1R)-2-[4-(2-chloroacetyl)piperazin-1-yl]-1-methyl-2-oxo-ethoxy]-4-(2-chloro-4-fluoro-phenyl)chromen-2-one,
2-chloro-N-[(3S)-1-[(2R)-2-[4-(2-chloro-4-fluoro-phenyl)-2-oxo-chromen-7-yl]oxypropanoyl]-3-piperidyl]acetamide,
4-(2-chloro-4-fluoro-phenyl)-7-[(1R)-1-methyl-2-oxo-2-(4-propanoylpiperazin-1-yl)ethoxy]chromen-2-one,
N-[(3S)-1-[(2R)-2-[4-(2-chloro-4-fluoro-phenyl)-2-oxo-chromen-7-yl]oxypropanoyl]-3-piperidyl]propanamide,
rac-(3S)-1-[2-[4-(2-chlorophenyl)-2-oxo-pyrano[2,3-b]pyridin-7-yl]oxypropanoyl]piperidine-3-carboxylic acid,
tert-butyl rac-(3S)-1-[2-[4-(2-chlorophenyl)-2-oxo-pyrano[2,3-b]pyridin-7-yl]oxypropanoyl]piperidine-3-carboxylate,
(3S)-1-[(2R)-2-[4-(2-chlorophenyl)-2-oxo-pyrano[2,3-b]pyridin-7-yl]oxypropanoyl]piperidine-3-carboxylic acid,
(3S)-1-[(2R)-2-[4-(2-chloro-4-fluoro-phenyl)-2-oxo-pyrano[2,3-b]pyridin-7-yl]oxypropanoyl]piperidine-3-carboxylic acid,
(3S)-1-[(2R)-2-[4-(3-methyl-2-thienyl)-2-oxo-chromen-7-yl]oxypropanoyl]piperidine-3-carboxylic acid,
ethyl 2-[4-(2-fluorophenyl)-2-oxo-chromen-7-yl]oxypropanoate,
ethyl 2-[4-(2,6-difluorophenyl)-2-oxo-chromen-7-yl]oxypropanoate,
7-[(1R)-1-methyl-2-oxo-2-(1-piperidyl)ethoxy]-4-(3-methyl-2-thienyl)chromen-2-one
(2R)—N-isopropyl-2-[4-(3-methyl-2-thienyl)-2-oxo-chromen-7-yl]oxy-propanamide
(2R)—N,N-dimethyl-2-[4-(3-methyl-2-thienyl)-2-oxo-chromen-7-yl]oxy-propanamide
ethyl 2-[(3R)-1-[(2R)-2-[4-(3-methyl-2-thienyl)-2-oxo-chromen-7-yl]oxypropanoyl]-3-piperidyl]acetate, and
2-[(3R)-1-[(2R)-2-[4-(3-methyl-2-thienyl)-2-oxo-chromen-7-yl]oxypropanoyl]-3-piperidyl]acetic acid,
(3S)-1-[(2R)-2-[4-(4-methyl-3-thienyl)-2-oxo-chromen-7-yl]oxypropanoyl]piperidine-3-carboxylic acid,
7-[(1R)-1-methyl-2-oxo-2-(1-piperidyl)ethoxy]-4-(4-methyl-3-thienyl)chromen-2-one,
7-[1-methyl-2-oxo-2-(1-piperidyl)ethoxy]-4-(2-methyl-3-thienyl)chromen-2-one,
rac-(3S)-1-[2-[4-(2-methyl-3-thienyl)-2-oxo-chromen-7-yl]oxypropanoyl]piperidine-3-carboxylic acid,
tert-butyl 2-(2-oxo-4-phenyl-chromen-7-yl)oxypropanoate
N,N-dimethyl-2-(2-oxo-4-phenyl-chromen-7-yl)oxy-propanamide
ethyl 2-[4-(4-chlorophenyl)-2-oxo-chromen-7-yl]oxypropanoate
ethyl 2-[4-(3-chlorophenyl)-2-oxo-chromen-7-yl]oxypropanoate
N-[4-(3-hydroxypropyl)phenyl]-2-(2-oxo-4-phenyl-chromen-7-yl)oxy-propanamide
2-(2-oxo-4-phenyl-chromen-7-yl)oxy-N-(2-pyridyl)propanamide
ethyl 2-[4-(3-fluorophenyl)-2-oxo-chromen-7-yl]oxypropanoate
ethyl 2-[4-(4-fluorophenyl)-2-oxo-chromen-7-yl]oxypropanoate
ethyl 2-[2-oxo-4-(p-tolyl)chromen-7-yl]oxypropanoate
methyl 1-[2-(6-chloro-2-oxo-4-phenyl-chromen-7-yl)oxypropanoyl]piperidine-3-carboxylate
2-(6-chloro-2-oxo-4-phenyl-chromen-7-yl)oxy-N-(2-pyridyl)propanamide
N-isopropyl-2-(2-oxo-4-phenyl-chromen-7-yl)oxy-propanamide 1-[2-(6-chloro-2-oxo-4-phenyl-chromen-7-yl)oxypropanoyl]piperidine-3-carboxylic acid
1-[2-(2-oxo-4-phenyl-chromen-7-yl)oxypropanoyl]piperidine-3-carboxylic acid
methyl 1-[2-(2-oxo-4-phenyl-chromen-7-yl)oxypropanoyl]piperidine-3-carboxylate
N-[4-(2-hydroxyethyl)phenyl]-2-(2-oxo-4-phenyl-chromen-7-yl)oxy-propanamide
2-(6-chloro-2-oxo-4-phenyl-chromen-7-yl)oxy-N-[4-(2-hydroxyethyl)phenyl]propanamide
N-[4-(2-hydroxyethyl)phenyl]-N-methyl-2-(2-oxo-4-phenyl-chromen-7-yl)oxy-propanamide
1-[2-(2-oxo-4-phenyl-chromen-7-yl)oxypropanoyl]piperidine-3-carbonitrile
7-[1-methyl-2-oxo-2-[3-(2H-tetrazol-5-yl)-1-piperidyl]ethoxy]-4-phenyl-chromen-2-one
(3S)-1-[(2R)-2-(2-oxo-4-phenyl-chromen-7-yl)oxypropanoyl]piperidine-3-carboxylic acid
2-(2-oxo-4-phenyl-chromen-7-yl)oxy-N-(2-pyridyl)acetamide methyl 1-[2-(2-oxo-4-phenyl-chromen-7-yl)oxyacetyl]piperidine-3-carboxylate
phenyl 2-(2-oxo-4-phenyl-chromen-7-yl)oxypropanoate
2-(2-oxo-4-phenyl-chromen-7-yl)oxy-N-phenyl-propanamide
N-methyl-2-(2-oxo-4-phenyl-chromen-7-yl)oxy-propanamide
2-(6-chloro-2-oxo-4-phenyl-chromen-7-yl)oxy-N-(2-hydroxy-2-phenyl-ethyl)propanamide
2-(6-chloro-2-oxo-4-phenyl-chromen-7-yl)oxy-N-[4-(3-hydroxypropyl)phenyl]propanamide
N-(2-hydroxy-2-phenyl-ethyl)-2-(2-oxo-4-phenyl-chromen-7-yl)oxy-propanamide
N-ethyl-2-(2-oxo-4-phenyl-chromen-7-yl)oxy-propanamide
7-(1-methyl-2-oxo-2-pyrrolidin-1-yl-ethoxy)-4-phenyl-chromen-2-one
ethyl 2-methyl-2-(2-oxo-4-phenyl-chromen-7-yl)oxy-propanoate
ethyl (2R)-2-(2-oxo-4-phenyl-chromen-7-yl)oxypropanoate
(2R)—N,N-dimethyl-2-(2-oxo-4-phenyl-chromen-7-yl)oxy-propanamide
ethyl (3S)-1-[(2R)-2-(2-oxo-4-phenyl-chromen-7-yl)oxypropanoyl]piperidine-3-carboxylate
(3S)-1-[2-(2-oxo-4-phenyl-chromen-7-yl)oxypropanoyl]piperidine-3-carboxylic acid
(3R)-1-[2-(2-oxo-4-phenyl-chromen-7-yl)oxypropanoyl]piperidine-3-carboxylic acid
methyl (3R)-1-[2-(2-oxo-4-phenyl-chromen-7-yl)oxypropanoyl]piperidine-3-carboxylate
7-[1-methyl-2-oxo-2-(1-piperidyl)ethoxy]-4-phenyl-chromen-2-one
N-[4-(2-hydroxyethyl)phenyl]-N-methyl-2-(2-oxo-4-phenyl-chromen-7-yl)oxy-acetamide
ethyl 2-[4-(4-bromophenyl)-2-oxo-chromen-7-yl]oxypropanoate
2-(6-chloro-2-oxo-4-phenyl-chromen-7-yl)oxy-N-(2-furylmethyl)propanamide
ethyl 1-[2-(2-oxo-4-phenyl-chromen-7-yl)oxyacetyl]piperidine-3-carboxylate
tert-butyl 2-(2-oxo-4-phenyl-chromen-7-yl)oxyacetate
6-chloro-7-(2-morpholino-2-oxo-ethoxy)-4-phenyl-chromen-2-one
2-(6-chloro-2-oxo-4-phenyl-chromen-7-yl)oxy-N-cyclopropyl-acetamide
2-(6-chloro-2-oxo-4-phenyl-chromen-7-yl)oxy-N,N-diethyl-acetamide
N,N-diethyl-2-(2-oxo-4-phenyl-chromen-7-yl)oxy-acetamide
ethyl 2-(6-chloro-2-oxo-4-phenyl-chromen-7-yl)oxyacetate
ethyl 2-(6-chloro-2-oxo-4-phenyl-chromen-7-yl)oxypropanoate
2-(2-oxo-4-phenyl-chromen-7-yl)oxypropanoic acid
ethyl 2-(2-oxo-4-phenyl-chromen-7-yl)oxypropanoate
isopropyl 2-(2-oxo-4-phenyl-chromen-7-yl)oxypropanoate
1-[2-(2-oxo-4-phenyl-chromen-7-yl)oxyacetyl]piperidine-3-carboxylic acid
2-(2-oxo-4-phenyl-chromen-7-yl)oxy-N-(3-pyridyl)propanamide
methyl 2-(2-oxo-4-phenyl-chromen-7-yl)oxypropanoate
2-(6-chloro-2-oxo-4-phenyl-chromen-7-yl)oxy-N-[4-(2-hydroxyethyl)phenyl]acetamide
methyl 2-(6-chloro-2-oxo-4-phenyl-chromen-7-yl)oxypropanoate
2-(6-chloro-2-oxo-4-phenyl-chromen-7-yl)oxy-N-(3-pyridylmethyl)acetamide
1-[2-(2-oxo-4-phenyl-chromen-7-yl)oxypropanoyl]piperidine-3-carboxamide
2-(2-oxo-4-phenyl-chromen-7-yl)oxy-N-(4-pyridyl)propanamide
N-methyl-1-[2-(2-oxo-4-phenyl-chromen-7-yl)oxypropanoyl]piperidine-3-carboxamide
methyl 1-[2-(2-oxo-4-phenyl-chromen-7-yl)oxypropanoyl]piperidine-4-carboxylate
ethyl rac-(3S)-1-[2-(2-oxo-4-phenyl-chromen-7-yl)oxypropanoyl]piperidine-3-carboxylate
ethyl 2-[4-(4-methoxyphenyl)-2-oxo-chromen-7-yl]oxypropanoate
(2R)-2-(2-oxo-4-phenyl-chromen-7-yl)oxypropanoic acid
2-(2-oxo-4-phenyl-chromen-7-yl)oxypropanamide
rac-(3S)—N,N-dimethyl-1-[2-(2-oxo-4-phenyl-chromen-7-yl)oxypropanoyl]piperidine-3-carboxamide
1-[2-(2-oxo-4-phenyl-chromen-7-yl)oxypropanoyl]piperidine-4-carboxylic acid
N-(2-morpholinoethyl)-2-(2-oxo-4-phenyl-chromen-7-yl)oxy-propanamide
ethyl 2-(2-oxo-4-phenyl-chromen-7-yl)oxybutanoate
N-(4-methoxyphenyl)-2-(2-oxo-4-phenyl-chromen-7-yl)oxy-propanamide
2-ethoxyethyl 2-(2-oxo-4-phenyl-chromen-7-yl)oxyacetate
propyl 2-(2-oxo-4-phenyl-chromen-7-yl)oxyacetate
2-(2-oxo-4-phenyl-chromen-7-yl)oxyacetamide
butyl 2-(2-oxo-4-phenyl-chromen-7-yl)oxyacetate
isobutyl 2-(2-oxo-4-phenyl-chromen-7-yl)oxyacetate
6-chloro-7-[2-(3,4-dihydro-2H-quinolin-1-yl)-2-oxo-ethoxy]-4-phenyl-chromen-2-one
2-(6-chloro-2-oxo-4-phenyl-chromen-7-yl)oxy-N-(2-morpholinoethyl)propanamide
2-(5-fluoro-2-oxo-4-phenyl-chromen-7-yl)oxy-N,N-dimethyl-propanamide
ethyl 2-(2-oxo-4-phenyl-chromen-7-yl)oxyacetate
tert-butyl 2-(6-chloro-2-oxo-4-phenyl-chromen-7-yl)oxyacetate
2-(6-chloro-2-oxo-4-phenyl-chromen-7-yl)oxy-N-(2-morpholinoethyl)acetamide
2-(6-chloro-2-oxo-4-phenyl-chromen-7-yl)oxyacetamide
1-[2-(6-chloro-2-oxo-4-phenyl-chromen-7-yl)oxyacetyl]piperidine-3-carboxylic acid
2-(6-chloro-2-oxo-4-phenyl-chromen-7-yl)oxy-N-cyclooctyl-acetamide
ethyl 2-(5-fluoro-2-oxo-4-phenyl-chromen-7-yl)oxypropanoate
2-(6-chloro-2-oxo-4-phenyl-chromen-7-yl)oxyacetic acid
4-[[2-(6-chloro-2-oxo-4-phenyl-chromen-7-yl)oxypropanoylamino]methyl]cyclohexanecarboxylic acid
2-(6-chloro-2-oxo-4-phenyl-chromen-7-yl)oxy-N-(2-hydroxy-2-phenyl-ethyl)acetamide
methyl 2-(8-methyl-2-oxo-4-phenyl-chromen-7-yl)oxyacetate
propyl 2-(6-chloro-2-oxo-4-phenyl-chromen-7-yl)oxyacetate
benzyl 2-(2-oxo-4-phenyl-chromen-7-yl)oxypropanoate
tert-butyl 2-(8-methyl-2-oxo-4-phenyl-chromen-7-yl)oxyacetate
2-(6-chloro-2-oxo-4-phenyl-chromen-7-yl)oxy-N-(1,1-dioxo-2,3-dihydrothiophen-3-yl)acetamide
2-[2-(2-oxo-4-phenyl-chromen-7-yl)oxypropanoylamino]-2-phenyl-acetic acid
2-(2-oxo-4-phenyl-6-propyl-chromen-7-yl)oxypropanoic acid
4-[[[2-(6-chloro-2-oxo-4-phenyl-chromen-7-yl)oxyacetyl]amino]methyl]cyclohexanecarboxylic acid
2-(6-chloro-2-oxo-4-phenyl-chromen-7-yl)oxy-N-(2-pyridylmethyl)acetamide,
7-[1-methyl-2-oxo-2-(1-piperidyl)ethoxy]-4-(o-tolyl)chromen-2-one, 4-(2-chlorophenyl)-7-[1-methyl-2-oxo-2-(1-piperidyl)ethoxy]chromen-2-one,
(3S)-1-[2-[4-(o-tolyl)-2-oxo-chromen-7-yl]oxypropanoyl]piperidine-3-carboxylic acid,
2-[4-(o-tolyl)-2-oxo-chromen-7-yl]oxy-N-(2-pyridyl)propanamide,
7-(1-methyl-2-oxo-2-pyrrolidin-1-yl-ethoxy)-4-(o-tolyl)chromen-2-one,
methyl 1-[2-[4-(o-tolyl)-2-oxo-chromen-7-yl]oxypropanoyl]piperidine-3-carboxylate,
methyl 1-[2-[4-(2-chlorophenyl)-2-oxo-chromen-7-yl]oxypropanoyl]piperidine-3-carboxylate,
1-[2-[4-(o-tolyl)-2-oxo-chromen-7-yl]oxypropanoyl]piperidine-3-carboxylic acid,
1-[2-[4-(2-chlorophenyl)-2-oxo-chromen-7-yl]oxypropanoyl]piperidine-3-carboxylic acid,
1-[2-[4-(o-tolyl)-2-oxo-chromen-7-yl]oxypropanoyl]piperidine-3-sulfonic acid,
1-[2-[4-(2-chlorophenyl)-2-oxo-chromen-7-yl]oxypropanoyl]piperidine-3-sulfonic acid,
1-[2-[4-(o-tolyl)-2-oxo-chromen-7-yl]oxypropanoyl]piperidine-3-sulfonamide,
4-(2-chlorophenyl)-7-(1-methyl-2-oxo-2-pyrrolidin-1-yl-ethoxy)chromen-2-one,
(3S)-1-[(2R)-2-[4-(2-chloro-4-fluoro-phenyl)-2-oxo-chromen-7-yl]oxypropanoyl]piperidine-3-carboxylic acid,
1-[2-[4-(2-chlorophenyl)-2-oxo-chromen-7-yl]oxypropanoyl]-N-methyl-piperidine-3-carboxamide,
ethyl (3S)-1-[2-[4-(2-chlorophenyl)-2-oxo-chromen-7-yl]oxypropanoyl]piperidine-3-carboxylate,
ethyl (3S)-1-[2-[4-(o-tolyl)-2-oxo-chromen-7-yl]oxypropanoyl]piperidine-3-carboxylate,
N-methyl-1-[2-[4-(o-tolyl)-2-oxo-chromen-7-yl]oxypropanoyl]piperidine-3-sulfonamide,
N-[4-(2-hydroxyethyl)phenyl]-2-[4-(o-tolyl)-2-oxo-chromen-7-yl]oxy-propanamide,
1-[2-[4-(2-chlorophenyl)-2-oxo-chromen-7-yl]oxypropanoyl]-N-methyl-piperidine-3-sulfonamide,
N-methyl-1-[2-[4-(o-tolyl)-2-oxo-chromen-7-yl]oxypropanoyl]piperidine-3-carboxamide,
(3S)-1-[2-[4-(o-tolyl)-2-oxo-chromen-7-yl]oxypropanoyl]pyrrolidine-3-carboxylic acid,
(3S)-1-[(2R)-2-[4-(2-chlorophenyl)-2-oxo-chromen-7-yl]oxypropanoyl]piperidine-3-carboxylic acid,
1-[2-[4-(2-chlorophenyl)-2-oxo-chromen-7-yl]oxypropanoyl]piperidine-3-sulfonamide,
N-cyclopropyl-2-[4-(o-tolyl)-2-oxo-chromen-7-yl]oxy-propanamide,
2-[4-(2-chlorophenyl)-2-oxo-chromen-7-yl]oxy-N-cyclopropyl-propanamide,
(3S)-1-[(2R)-2-[4-(o-tolyl)-2-oxo-chromen-7-yl]oxypropanoyl]piperidine-3-carboxylic acid,
(3S)-1-[(2R)-2-[4-(2-chlorophenyl)-2-oxo-chromen-7-yl]oxypropanoyl]piperidine-3-carbonitrile,
(3S)-1-[2-[4-(2-chlorophenyl)-2-oxo-chromen-7-yl]oxypropanoyl]pyrrolidine-3-carboxylic acid
7-[(1R)-1-methyl-2-oxo-2-(1-piperidyl)ethoxy]-4-(o-tolyl)chromen-2-one,
2-[4-(2-chlorophenyl)-2-oxo-chromen-7-yl]oxy-N-(2-pyridyl)propanamide,
(3S)-1-[(2R)-2-[4-(o-tolyl)-2-oxo-chromen-7-yl]oxypropanoyl]piperidine-3-carbonitrile,
(3S)-1-[2-[4-(2-chlorophenyl)-2-oxo-chromen-7-yl]oxypropanoyl]piperidine-3-carboxylic acid,
4-(2-chlorophenyl)-7-[(1R)-1-methyl-2-oxo-2-(1-piperidyl)ethoxy]chromen-2-one,
2-[4-(2-chlorophenyl)-2-oxo-chromen-7-yl]oxy-N-[4-(2-hydroxyethyl)phenyl]propanamide,
(2R)—N-isopropyl-2-[4-(o-tolyl)-2-oxo-chromen-7-yl]oxy-propanamide,
1-[2-[4-(2-chloro-4-fluoro-phenyl)-2-oxo-chromen-7-yl]oxypropanoyl]-3-methyl-piperidine-3-carboxylic acid,
N,N-dimethyl-2-[4-(o-tolyl)-2-oxo-chromen-7-yl]oxy-propanamide,
4-(2-chlorophenyl)-7-[1-methyl-2-oxo-2-[3-(2H-tetrazol-5-yl)-1-piperidyl]ethoxy]chromen-2-one,
ethyl 2-[4-(2-chlorophenyl)-2-oxo-chromen-7-yl]oxypropanoate,
1-[2-[4-(o-tolyl)-2-oxo-chromen-7-yl]oxypropanoyl]piperidine-3-carbonitrile,
7-[1-methyl-2-oxo-2-[3-(2H-tetrazol-5-yl)-1-piperidyl]ethoxy]-4-(o-tolyl)chromen-2-one,
3-methyl-1-[2-[4-(o-tolyl)-2-oxo-chromen-7-yl]oxypropanoyl]piperidine-3-carboxylic acid,
(2R)—N,N-dimethyl-2-[4-(o-tolyl)-2-oxo-chromen-7-yl]oxy-propanamide,
(2R)-2-[4-(2-chlorophenyl)-2-oxo-chromen-7-yl]oxy-N,N-dimethyl-propanamide,
(3R)-1-[2-[4-(2-chlorophenyl)-2-oxo-chromen-7-yl]oxypropanoyl]piperidine-3-carboxylic acid,
(3S)-1-[2-[4-(2-chloro-4-fluoro-phenyl)-2-oxo-chromen-7-yl]oxypropanoyl]piperidine-3-carboxylic acid,
1-[2-[4-(2-chlorophenyl)-2-oxo-chromen-7-yl]oxypropanoyl]piperidine-3-carbonitrile,
2-[4-(2-chloro-3-fluoro-phenyl)-2-oxo-chromen-7-yl]oxy-N-isopropyl-propanamide,
(3S)-1-[2-[4-(2-chloro-3-fluoro-phenyl)-2-oxo-chromen-7-yl]oxypropanoyl]piperidine-3-carboxylic acid,
2-[4-(2-chlorophenyl)-2-oxo-chromen-7-yl]oxy-N-methyl-propanamide,
1-[2-[4-(2-chloro-3-fluoro-phenyl)-2-oxo-chromen-7-yl]oxypropanoyl]piperidine-3-sulfonamide,
(3R)-1-[2-[4-(o-tolyl)-2-oxo-chromen-7-yl]oxypropanoyl]piperidine-3-carboxylic acid,
1-[2-[4-(2-chlorophenyl)-2-oxo-chromen-7-yl]oxypropanoyl]-3-methyl-piperidine-3-carboxylic acid,
isopropyl (2R)-2-[4-(o-tolyl)-2-oxo-chromen-7-yl]oxypropanoate,
(2R)-2-[4-(2-chlorophenyl)-2-oxo-chromen-7-yl]oxy-N-isopropyl-propanamide,
ethyl 2-[4-(2-chloro-3-fluoro-phenyl)-2-oxo-chromen-7-yl]oxypropanoate,
ethyl 2-[4-(o-tolyl)-2-oxo-chromen-7-yl]oxypropanoate,
2-[1-[2-[4-(2-chlorophenyl)-2-oxo-chromen-7-yl]oxypropanoyl]-4-piperidyl]acetic acid,
2-[1-[2-[4-(o-tolyl)-2-oxo-chromen-7-yl]oxypropanoyl]-4-piperidyl]acetic acid,
2-[4-(2-chloro-3-fluoro-phenyl)-2-oxo-chromen-7-yl]oxy-N-cyclopropyl-propanamide,
N-isopropyl-2-[4-(o-tolyl)-2-oxo-chromen-7-yl]oxy-propanamide,
1-[2-[4-(o-tolyl)-2-oxo-chromen-7-yl]oxypropanoyl]pyrrolidine-3-sulfonamide,
isopropyl (2R)-2-[4-(2-chlorophenyl)-2-oxo-chromen-7-yl]oxypropanoate,
2-[4-(2-chloro-4-fluoro-phenyl)-2-oxo-chromen-7-yl]oxy-N-isopropyl-propanamide,
2-[1-[2-[4-(2-chlorophenyl)-2-oxo-chromen-7-yl]oxypropanoyl]-3-piperidyl]acetic acid,
(3R)-1-[2-[4-(2-chlorophenyl)-2-oxo-chromen-7-yl]oxypropanoyl]-N,N-dimethyl-piperidine-3-carboxamide,
2-[4-(2-chloro-3-fluoro-phenyl)-2-oxo-chromen-7-yl]oxy-N-ethyl-propanamide,
1-[2-[4-(2-chloro-4-fluoro-phenyl)-2-oxo-chromen-7-yl]oxypropanoyl]piperidine-3-sulfonamide,
2-[4-(2-chlorophenyl)-2-oxo-chromen-7-yl]oxy-N-isopropyl-propanamide,
7-[2-(4,4-difluoro-1-piperidyl)-1-methyl-2-oxo-ethoxy]-4-(o-tolyl)chromen-2-one,
2-[1-[2-[4-(o-tolyl)-2-oxo-chromen-7-yl]oxypropanoyl]-3-piperidyl]acetic acid,
7-[1-methyl-2-[3-(methylsulfonimidoyl)-1-piperidyl]-2-oxo-ethoxy]-4-(o-tolyl)chromen-2-one,
4-(2-chlorophenyl)-7-[2-(4,4-difluoro-1-piperidyl)-1-methyl-2-oxo-ethoxy]chromen-2-one,
ethyl 1-[2-[4-(2-chlorophenyl)-2-oxo-chromen-7-yl]oxypropanoyl]-3-methyl-piperidine-3-carboxylate,
(3S)-1-[2-[4-(2-chlorophenyl)-2-oxo-chromen-7-yl]oxypropanoyl]-N,N-dimethyl-piperidine-3-carboxamide,
1-[2-[4-(2-chlorophenyl)-2-oxo-chromen-7-yl]oxypropanoyl]pyrrolidine-3-sulfonamide,
(3R)—N,N-dimethyl-1-[2-[4-(o-tolyl)-2-oxo-chromen-7-yl]oxypropanoyl]piperidine-3-carboxamide,
4-(2-chlorophenyl)-7-[1-methyl-2-[3-(methylsulfonimidoyl)-1-piperidyl]-2-oxo-ethoxy]chromen-2-one,
methyl 2-[1-[2-[4-(2-chlorophenyl)-2-oxo-chromen-7-yl]oxypropanoyl]-4-piperidyl]acetate,
ethyl 2-[4-(2-bromophenyl)-2-oxo-chromen-7-yl]oxypropanoate,
ethyl 2-[4-(2-chloro-4-fluoro-phenyl)-2-oxo-chromen-7-yl]oxypropanoate,
methyl 2-[1-[2-[4-(o-tolyl)-2-oxo-chromen-7-yl]oxypropanoyl]-4-piperidyl]acetate,
(3S)—N,N-dimethyl-1-[2-[4-(o-tolyl)-2-oxo-chromen-7-yl]oxypropanoyl]piperidine-3-carboxamide,
N-ethyl-2-[4-(o-tolyl)-2-oxo-chromen-7-yl]oxy-propanamide,
ethyl 2-[1-[2-[4-(2-chlorophenyl)-2-oxo-chromen-7-yl]oxypropanoyl]-3-piperidyl]acetate,
ethyl 2-[1-[2-[4-(o-tolyl)-2-oxo-chromen-7-yl]oxypropanoyl]-3-piperidyl]acetate,
ethyl 3-methyl-1-[2-[4-(o-tolyl)-2-oxo-chromen-7-yl]oxypropanoyl]piperidine-3-carboxylate,
2-[4-(2-bromophenyl)-2-oxo-chromen-7-yl]oxy-N,N-dimethyl-propanamide,
2-[4-(2-chloro-4-fluoro-phenyl)-2-oxo-chromen-7-yl]oxy-N-cyclopropyl-propanamide,
2-[4-(2-chloro-4-fluoro-phenyl)-2-oxo-chromen-7-yl]oxy-N-ethyl-propanamide,
isopropyl 2-[4-(2-bromophenyl)-2-oxo-chromen-7-yl]oxypropanoate,
2-[4-(2-chlorophenyl)-2-oxo-chromen-7-yl]oxy-N,N-dimethyl-propanamide,
2-[4-(2-chloro-3-fluoro-phenyl)-2-oxo-chromen-7-yl]oxypropanoic acid,
ethyl 1-[2-[4-(2-chloro-4-fluoro-phenyl)-2-oxo-chromen-7-yl]oxypropanoyl]-3-methyl-piperidine-3-carboxylate,
2-[4-(2-chlorophenyl)-2-oxo-chromen-7-yl]oxypropanoic acid,
2-[4-(o-tolyl)-2-oxo-chromen-7-yl]oxypropanoic acid,
2-[4-(2-chlorophenyl)-2-oxo-chromen-7-yl]oxy-N-ethyl-propanamide,
2-[4-(2-chloro-4-fluoro-phenyl)-2-oxo-chromen-7-yl]oxypropanoic acid,
methyl 2-[(3S)-1-[(2R)-2-[4-(2-chlorophenyl)-2-oxo-chromen-7-yl]oxypropanoyl]-3-piperidyl]acetate,
methyl 2-[(3R)-1-[(2R)-2-[4-(2-chlorophenyl)-2-oxo-chromen-7-yl]oxypropanoyl]-3-piperidyl]acetate,
2-[(3S)-1-[(2R)-2-[4-(2-chlorophenyl)-2-oxo-chromen-7-yl]oxypropanoyl]-3-piperidyl]acetic acid,
2-[(3R)-1-[(2R)-2-[4-(2-chlorophenyl)-2-oxo-chromen-7-yl]oxypropanoyl]-3-piperidyl]acetic acid,
methyl 2-[(3R)-1-[(2R)-2-[4-(2-chloro-4-fluoro-phenyl)-2-oxo-chromen-7-yl]oxypropanoyl]-3-piperidyl]acetate,
methyl 2-[(3S)-1-[(2R)-2-[4-(2-chloro-4-fluoro-phenyl)-2-oxo-chromen-7-yl]oxypropanoyl]-3-piperidyl]acetate,
2-[(3R)-1-[(2R)-2-[4-(2-chloro-4-fluoro-phenyl)-2-oxo-chromen-7-yl]oxypropanoyl]-3-piperidyl]acetic acid,
2-[(3S)-1-[(2R)-2-[4-(2-chloro-4-fluoro-phenyl)-2-oxo-chromen-7-yl]oxypropanoyl]-3-piperidyl]acetic acid,
(3R)-1-[(2R)-2-[4-(2-chloro-4-fluoro-phenyl)-2-oxo-chromen-7-yl]oxypropanoyl]piperidine-3-carboxylic acid,
ethyl (3S)-1-[2-[4-(2-chlorophenyl)-2-oxo-chromen-7-yl]oxybutanoyl]piperidine-3-carboxylate,
(3S)-1-[2-[4-(2-chlorophenyl)-2-oxo-chromen-7-yl]oxybutanoyl]piperidine-3-carboxylic acid,
ethyl (3S)-1-[(2R)-2-[4-(2-chloro-4-fluoro-phenyl)-2-oxo-chromen-7-yl]oxypropanoyl]piperidine-3-carboxylate,
2-[4-(2-chlorophenyl)-2-oxo-chromen-7-yl]oxy-2-methyl-propanoic acid,
ethyl 2-[4-(2-chlorophenyl)-2-oxo-chromen-7-yl]oxy-2-methyl-propanoate,
2-[4-(2-chlorophenyl)-2-oxo-chromen-7-yl]oxy-N-isopropyl-2-methyl-propanamide,
N-isopropyl-2-methyl-2-[4-(o-tolyl)-2-oxo-chromen-7-yl]oxy-propanamide,
(3S)-1-[2-[4-(2-chlorophenyl)-2-oxo-chromen-7-yl]oxy-2-methyl-propanoyl]piperidine-3-carboxylic acid,
isopropyl (3S)-1-[(2R)-2-[4-(2-chloro-4-fluoro-phenyl)-2-oxo-chromen-7-yl]oxypropanoyl]piperidine-3-carboxylate,
tert-butyl (3S)-1-[(2R)-2-[4-(2-chloro-4-fluoro-phenyl)-2-oxo-chromen-7-yl]oxypropanoyl]piperidine-3-carboxylate,
2-morpholinoethyl (3S)-1-[(2R)-2-[4-(2-chloro-4-fluoro-phenyl)-2-oxo-chromen-7-yl]oxypropanoyl]piperidine-3-carboxylate,
heptyl (3S)-1-[(2R)-2-[4-(2-chloro-4-fluoro-phenyl)-2-oxo-chromen-7-yl]oxypropanoyl]piperidine-3-carboxylate,
isopropoxycarbonyloxymethyl (3S)-1-[(2R)-2-[4-(2-chloro-4-fluoro-phenyl)-2-oxo-chromen-7-yl]oxypropanoyl]piperidine-3-carboxylate,
(3S)—N-methyl-1-[(2R)-2-[4-(2-chloro-4-fluoro-phenyl)-2-oxo-chromen-7-yl]oxypropanoyl piperidine-3-carboxamide,
isopropyl 2-[(3R)-1-[(2R)-2-[4-(2-chloro-4-fluoro-phenyl)-2-oxo-chromen-7-yl]oxypropanoyl]-3-piperidyl]acetate,
tert-butyl 2-[(3R)-1-[(2R)-2-[4-(2-chloro-4-fluoro-phenyl)-2-oxo-chromen-7-yl]oxypropanoyl]-3-piperidyl]acetate,
2-morpholinoethyl 2-[(3R)-1-[(2R)-2-[4-(2-chloro-4-fluoro-phenyl)-2-oxo-chromen-7-yl]oxypropanoyl]-3-piperidyl]acetate,
heptyl 2-[(3R)-1-[(2R)-2-[4-(2-chloro-4-fluoro-phenyl)-2-oxo-chromen-7-yl]oxypropanoyl]-3-piperidyl]acetate,
isopropoxycarbonyloxymethyl 2-[(3R)-1-[(2R)-2-[4-(2-chloro-4-fluoro-phenyl)-2-oxo-chromen-7-yl]oxypropanoyl]-3-piperidyl]acetate,
2-[(3R)-1-[(2R)-2-[4-(2-chloro-4-fluoro-phenyl)-2-oxo-chromen-7-yl]oxypropanoyl]-3-piperidyl]-N-methyl-acetamide,
methyl 1-[(2R)-2-[4-(2-chloro-4-fluoro-phenyl)-2-oxo-chromen-7-yl]oxypropanoyl]piperidine-4-carboxylate,
(2R)-2-[4-(2-chloro-4-fluoro-phenyl)-2-oxo-chromen-7-yl]oxy-N-[4-(hydroxymethyl)phenyl]propanamide,
1-[(2R)-2-[4-(2-chloro-4-fluoro-phenyl)-2-oxo-chromen-7-yl]oxypropanoyl]piperidine-4-carboxylic acid,
(3S)-1-[(2R)-2-[4-(2-chloro-4-fluoro-phenyl)-2-oxo-chromen-7-yl]oxypropanoyl]piperidine-3-carboxamide,
2-[(3R)-1-[(2R)-2-[4-(2-chloro-4-fluoro-phenyl)-2-oxo-chromen-7-yl]oxypropanoyl]-3-piperidyl]acetamide,
(3S)-1-[(2R)-2-[4-(2-ethylphenyl)-2-oxo-chromen-7-yl]oxypropanoyl]piperidine-3-carboxylic acid,
methyl (2S)-4-[(2R)-2-[4-(2-chloro-4-fluoro-phenyl)-2-oxo-chromen-7-yl]oxypropanoyl]piperazine-2-carboxylate,
methyl (2R)-4-[(2R)-2-[4-(2-chloro-4-fluoro-phenyl)-2-oxo-chromen-7-yl]oxypropanoyl]piperazine-2-carboxylate,
methyl 1-methyl-4-[rac-(2R)-2-[4-(2-chloro-4-fluoro-phenyl)-2-oxo-chromen-7-yl]oxypropanoyl]piperazine-2-carboxylate,
(2S)-4-[(2R)-2-[4-(2-chloro-4-fluoro-phenyl)-2-oxo-chromen-7-yl]oxypropanoyl]piperazine-2-carboxylic acid,
3-[[(2R)-2-[4-(2-chloro-4-fluoro-phenyl)-2-oxo-chromen-7-yl]oxypropanoyl]amino]benzoic acid,
(2R)-2-[4-(2-chloro-4-fluoro-phenyl)-2-oxo-chromen-7-yl]oxy-N-[4-(2-methoxyethyl)phenyl]propanamide,
4-[[(2R)-2-[4-(2-chloro-4-fluoro-phenyl)-2-oxo-chromen-7-yl]oxypropanoyl]amino]benzoic acid,
methyl 2-[(3R)-1-[(2R)-2-[4-(2-chloro-4-fluoro-phenyl)-2-oxo-chromen-7-yl]oxypropanoyl]pyrrolidin-3-yl]acetate,
methyl 5-[[(2R)-2-[4-(2-chloro-4-fluoro-phenyl)-2-oxo-chromen-7-yl]oxypropanoyl]amino]pyridine-3-carboxylate,
methyl 2-[(3S)-1-[(2R)-2-[4-(2-chloro-4-fluoro-phenyl)-2-oxo-chromen-7-yl]oxypropanoyl]pyrrolidin-3-yl]acetate,
2-[(3R)-1-[(2R)-2-[4-(2-chloro-4-fluoro-phenyl)-2-oxo-chromen-7-yl]oxypropanoyl]pyrrolidin-3-yl]acetic acid,
methyl 2-[[(2R)-2-[4-(2-chloro-4-fluoro-phenyl)-2-oxo-chromen-7-yl]oxypropanoyl]amino]pyridine-4-carboxylate,
2-[(3S)-1-[(2R)-2-[4-(2-chloro-4-fluoro-phenyl)-2-oxo-chromen-7-yl]oxypropanoyl]pyrrolidin-3-yl]acetic acid,
5-[[(2R)-2-[4-(2-chloro-4-fluoro-phenyl)-2-oxo-chromen-7-yl]oxypropanoyl]amino]pyridine-3-carboxylic acid,
methyl 6-[[(2R)-2-[4-(2-chloro-4-fluoro-phenyl)-2-oxo-chromen-7-yl]oxypropanoyl]amino]pyridine-2-carboxylate,
(2R)-4-[(2R)-2-[4-(2-chloro-4-fluoro-phenyl)-2-oxo-chromen-7-yl]oxypropanoyl]piperazine-2-carboxylic acid,
6-[[(2R)-2-[4-(2-chloro-4-fluoro-phenyl)-2-oxo-chromen-7-yl]oxypropanoyl]amino]pyridine-2-carboxylic acid,
2-[[(2R)-2-[4-(2-chloro-4-fluoro-phenyl)-2-oxo-chromen-7-yl]oxypropanoyl]amino]pyridine-4-carboxylic acid,
methyl 5-[[(2R)-2-[4-(2-chloro-4-fluoro-phenyl)-2-oxo-chromen-7-yl]oxypropanoyl]amino]pyridine-2-carboxylate,
5-[[(2R)-2-[4-(2-chloro-4-fluoro-phenyl)-2-oxo-chromen-7-yl]oxypropanoyl]amino]pyridine-2-carboxylic acid,
5-[[(2R)-2-[4-(2-chloro-4-fluoro-phenyl)-2-oxo-chromen-7-yl]oxypropanoyl]amino]pyridine-2-carboxylic acid,
6-[[(2R)-2-[4-(2-chloro-4-fluoro-phenyl)-2-oxo-chromen-7-yl]oxypropanoyl]amino]pyridine-3-carboxylic acid,
methyl 4-[[(2R)-2-[4-(2-chloro-4-fluoro-phenyl)-2-oxo-chromen-7-yl]oxypropanoyl]amino]pyridine-2-carboxylate,
methyl (3S)-1-[(2R)-2-[4-(2-chloro-4-fluoro-phenyl)-2-oxo-chromen-7-yl]oxypropanoyl]pyrrolidine-3-carboxylate,
(3S)-1-[(2R)-2-[4-(4-fluoro-2-methyl-phenyl)-2-oxo-chromen-7-yl]oxypropanoyl]piperidine-3-carboxylic acid,
(3S)-1-[(2R)-2-[4-(2-chloro-4-fluoro-phenyl)-2-oxo-chromen-7-yl]oxypropanoyl]pyrrolidine-3-carboxylic acid,
4-[[(2R)-2-[4-(2-chloro-4-fluoro-phenyl)-2-oxo-chromen-7-yl]oxypropanoyl]amino]pyridine-2-carboxylic acid,
1-methyl-4-[rac-(2R)-2-[4-(2-chloro-4-fluoro-phenyl)-2-oxo-chromen-7-yl]oxypropanoyl]piperazine-2-carboxylic acid,
(3S)-1-[(2R)-2-[4-(2-cyanophenyl)-2-oxo-chromen-7-yl]oxypropanoyl]piperidine-3-carboxylic acid,
(3S)-1-[(2R)-2-[4-(2,6-dichlorophenyl)-2-oxo-chromen-7-yl]oxypropanoyl]piperidine-3-carboxylic acid,
4-(2-chloro-4-fluoro-phenyl)-7-[(1R)-1-methyl-2-oxo-2-(4-prop-2-enoylpiperazin-1-yl)ethoxy]chromen-2-one,
N-[(3S)-1-[(2R)-2-[4-(2-chloro-4-fluoro-phenyl)-2-oxo-chromen-7-yl]oxypropanoyl]-3-piperidyl]prop-2-enamide,
7-[(1R)-2-[4-(2-chloroacetyl)piperazin-1-yl]-1-methyl-2-oxo-ethoxy]-4-(2-chloro-4-fluoro-phenyl)chromen-2-one,
2-chloro-N-[(3S)-1-[(2R)-2-[4-(2-chloro-4-fluoro-phenyl)-2-oxo-chromen-7-yl]oxypropanoyl]-3-piperidyl]acetamide,
4-(2-chloro-4-fluoro-phenyl)-7-[(1R)-1-methyl-2-oxo-2-(4-propanoylpiperazin-1-yl)ethoxy]chromen-2-one,
N-[(3S)-1-[(2R)-2-[4-(2-chloro-4-fluoro-phenyl)-2-oxo-chromen-7-yl]oxypropanoyl]-3-piperidyl]propanamide,
rac-(3S)-1-[2-[4-(2-chlorophenyl)-2-oxo-pyrano[2,3-b]pyridin-7-yl]oxypropanoyl]piperidine-3-carboxylic acid,
tert-butyl rac-(3S)-1-[2-[4-(2-chlorophenyl)-2-oxo-pyrano[2,3-b]pyridin-7-yl]oxypropanoyl]piperidine-3-carboxylate,
(3S)-1-[(2R)-2-[4-(2-chlorophenyl)-2-oxo-pyrano[2,3-b]pyridin-7-yl]oxypropanoyl]piperidine-3-carboxylic acid,
(3S)-1-[(2R)-2-[4-(2-chloro-4-fluoro-phenyl)-2-oxo-pyrano[2,3-b]pyridin-7-yl]oxypropanoyl]piperidine-3-carboxylic acid,
(3S)-1-[(2R)-2-[4-(3-methyl-2-thienyl)-2-oxo-chromen-7-yl]oxypropanoyl]piperidine-3-carboxylic acid,
ethyl 2-[4-(2-fluorophenyl)-2-oxo-chromen-7-yl]oxypropanoate,
ethyl 2-[4-(2,6-difluorophenyl)-2-oxo-chromen-7-yl]oxypropanoate,
7-[(1R)-1-methyl-2-oxo-2-(1-piperidyl)ethoxy]-4-(3-methyl-2-thienyl)chromen-2-one
(2R)—N-isopropyl-2-[4-(3-methyl-2-thienyl)-2-oxo-chromen-7-yl]oxy-propanamide
(2R)—N,N-dimethyl-2-[4-(3-methyl-2-thienyl)-2-oxo-chromen-7-yl]oxy-propanamide
ethyl 2-[(3R)-1-[(2R)-2-[4-(3-methyl-2-thienyl)-2-oxo-chromen-7-yl]oxypropanoyl]-3-piperidyl]acetate,
2-[(3R)-1-[(2R)-2-[4-(3-methyl-2-thienyl)-2-oxo-chromen-7-yl]oxypropanoyl]-3-piperidyl]acetic acid,
(3S)-1-[(2R)-2-[4-(4-methyl-3-thienyl)-2-oxo-chromen-7-yl]oxypropanoyl]piperidine-3-carboxylic acid,
7-[(1R)-1-methyl-2-oxo-2-(1-piperidyl)ethoxy]-4-(4-methyl-3-thienyl)chromen-2-one,
7-[1-methyl-2-oxo-2-(1-piperidyl)ethoxy]-4-(2-methyl-3-thienyl)chromen-2-one,
rac-(3S)-1-[2-[4-(2-methyl-3-thienyl)-2-oxo-chromen-7-yl]oxypropanoyl]piperidine-3-carboxylic acid
7-[1-methyl-2-oxo-2-(1-piperidyl)ethoxy]-4-(o-tolyl)chromen-2-one, 4-(2-chlorophenyl)-7-[1-methyl-2-oxo-2-(1-piperidyl)ethoxy]chromen-2-one,
(3S)-1-[2-[4-(o-tolyl)-2-oxo-chromen-7-yl]oxypropanoyl]piperidine-3-carboxylic acid,
2-[4-(o-tolyl)-2-oxo-chromen-7-yl]oxy-N-(2-pyridyl)propanamide,
7-(1-methyl-2-oxo-2-pyrrolidin-1-yl-ethoxy)-4-(o-tolyl)chromen-2-one,
methyl 1-[2-[4-(o-tolyl)-2-oxo-chromen-7-yl]oxypropanoyl]piperidine-3-carboxylate,
methyl 1-[2-[4-(2-chlorophenyl)-2-oxo-chromen-7-yl]oxypropanoyl]piperidine-3-carboxylate,
1-[2-[4-(o-tolyl)-2-oxo-chromen-7-yl]oxypropanoyl]piperidine-3-carboxylic acid,
1-[2-[4-(2-chlorophenyl)-2-oxo-chromen-7-yl]oxypropanoyl]piperidine-3-carboxylic acid,
1-[2-[4-(o-tolyl)-2-oxo-chromen-7-yl]oxypropanoyl]piperidine-3-sulfonic acid,
1-[2-[4-(2-chlorophenyl)-2-oxo-chromen-7-yl]oxypropanoyl]piperidine-3-sulfonic acid,
1-[2-[4-(o-tolyl)-2-oxo-chromen-7-yl]oxypropanoyl]piperidine-3-sulfonamide,
4-(2-chlorophenyl)-7-(1-methyl-2-oxo-2-pyrrolidin-1-yl-ethoxy)chromen-2-one,
(3S)-1-[(2R)-2-[4-(2-chloro-4-fluoro-phenyl)-2-oxo-chromen-7-yl]oxypropanoyl]piperidine-3-carboxylic acid,
1-[2-[4-(2-chlorophenyl)-2-oxo-chromen-7-yl]oxypropanoyl]-N-methyl-piperidine-3-carboxamide,
ethyl (3S)-1-[2-[4-(2-chlorophenyl)-2-oxo-chromen-7-yl]oxypropanoyl]piperidine-3-carboxylate,
ethyl (3S)-1-[2-[4-(o-tolyl)-2-oxo-chromen-7-yl]oxypropanoyl]piperidine-3-carboxylate,
N-methyl-1-[2-[4-(o-tolyl)-2-oxo-chromen-7-yl]oxypropanoyl]piperidine-3-sulfonamide,
N-[4-(2-hydroxyethyl)phenyl]-2-[4-(o-tolyl)-2-oxo-chromen-7-yl]oxy-propanamide,
1-[2-[4-(2-chlorophenyl)-2-oxo-chromen-7-yl]oxypropanoyl]-N-methyl-piperidine-3-sulfonamide,
N-methyl-1-[2-[4-(o-tolyl)-2-oxo-chromen-7-yl]oxypropanoyl]piperidine-3-carboxamide,
(3S)-1-[2-[4-(o-tolyl)-2-oxo-chromen-7-yl]oxypropanoyl]pyrrolidine-3-carboxylic acid,
(3S)-1-[(2R)-2-[4-(2-chlorophenyl)-2-oxo-chromen-7-yl]oxypropanoyl]piperidine-3-carboxylic acid,
1-[2-[4-(2-chlorophenyl)-2-oxo-chromen-7-yl]oxypropanoyl]piperidine-3-sulfonamide,
N-cyclopropyl-2-[4-(o-tolyl)-2-oxo-chromen-7-yl]oxy-propanamide,
2-[4-(2-chlorophenyl)-2-oxo-chromen-7-yl]oxy-N-cyclopropyl-propanamide,
(3S)-1-[(2R)-2-[4-(o-tolyl)-2-oxo-chromen-7-yl]oxypropanoyl]piperidine-3-carboxylic acid,
(3S)-1-[(2R)-2-[4-(2-chlorophenyl)-2-oxo-chromen-7-yl]oxypropanoyl]piperidine-3-carbonitrile,
(3S)-1-[2-[4-(2-chlorophenyl)-2-oxo-chromen-7-yl]oxypropanoyl]pyrrolidine-3-carboxylic acid,
7-[(1R)-1-methyl-2-oxo-2-(1-piperidyl)ethoxy]-4-(o-tolyl)chromen-2-one,
2-[4-(2-chlorophenyl)-2-oxo-chromen-7-yl]oxy-N-(2-pyridyl)propanamide,
(3S)-1-[(2R)-2-[4-(o-tolyl)-2-oxo-chromen-7-yl]oxypropanoyl]piperidine-3-carbonitrile,
(3S)-1-[2-[4-(2-chlorophenyl)-2-oxo-chromen-7-yl]oxypropanoyl]piperidine-3-carboxylic acid,
4-(2-chlorophenyl)-7-[(1R)-1-methyl-2-oxo-2-(1-piperidyl)ethoxy]chromen-2-one,
2-[4-(2-chlorophenyl)-2-oxo-chromen-7-yl]oxy-N-[4-(2-hydroxyethyl)phenyl]propanamide,
(2R)—N-isopropyl-2-[4-(o-tolyl)-2-oxo-chromen-7-yl]oxy-propanamide,
1-[2-[4-(2-chloro-4-fluoro-phenyl)-2-oxo-chromen-7-yl]oxypropanoyl]-3-methyl-piperidine-3-carboxylic acid,
N,N-dimethyl-2-[4-(o-tolyl)-2-oxo-chromen-7-yl]oxy-propanamide,
4-(2-chlorophenyl)-7-[1-methyl-2-oxo-2-[3-(2H-tetrazol-5-yl)-1-piperidyl]ethoxy]chromen-2-one,
ethyl 2-[4-(2-chlorophenyl)-2-oxo-chromen-7-yl]oxypropanoate,
1-[2-[4-(o-tolyl)-2-oxo-chromen-7-yl]oxypropanoyl]piperidine-3-carbonitrile,
7-[1-methyl-2-oxo-2-[3-(2H-tetrazol-5-yl)-1-piperidyl]ethoxy]-4-(o-tolyl)chromen-2-one,
3-methyl-1-[2-[4-(o-tolyl)-2-oxo-chromen-7-yl]oxypropanoyl]piperidine-3-carboxylic acid,
(2R)—N,N-dimethyl-2-[4-(o-tolyl)-2-oxo-chromen-7-yl]oxy-propanamide,
(2R)-2-[4-(2-chlorophenyl)-2-oxo-chromen-7-yl]oxy-N,N-dimethyl-propanamide,
(3R)-1-[2-[4-(2-chlorophenyl)-2-oxo-chromen-7-yl]oxypropanoyl]piperidine-3-carboxylic acid,
(3S)-1-[2-[4-(2-chloro-4-fluoro-phenyl)-2-oxo-chromen-7-yl]oxypropanoyl]piperidine-3-carboxylic acid,
1-[2-[4-(2-chlorophenyl)-2-oxo-chromen-7-yl]oxypropanoyl]piperidine-3-carbonitrile,
2-[4-(2-chloro-3-fluoro-phenyl)-2-oxo-chromen-7-yl]oxy-N-isopropyl-propanamide,
(3S)-1-[2-[4-(2-chloro-3-fluoro-phenyl)-2-oxo-chromen-7-yl]oxypropanoyl]piperidine-3-carboxylic acid,
2-[4-(2-chlorophenyl)-2-oxo-chromen-7-yl]oxy-N-methyl-propanamide,
1-[2-[4-(2-chloro-3-fluoro-phenyl)-2-oxo-chromen-7-yl]oxypropanoyl]piperidine-3-sulfonamide,
(3R)-1-[2-[4-(o-tolyl)-2-oxo-chromen-7-yl]oxypropanoyl]piperidine-3-carboxylic acid,
1-[2-[4-(2-chlorophenyl)-2-oxo-chromen-7-yl]oxypropanoyl]-3-methyl-piperidine-3-carboxylic acid,
isopropyl (2R)-2-[4-(o-tolyl)-2-oxo-chromen-7-yl]oxypropanoate,
(2R)-2-[4-(2-chlorophenyl)-2-oxo-chromen-7-yl]oxy-N-isopropyl-propanamide,
ethyl 2-[4-(2-chloro-3-fluoro-phenyl)-2-oxo-chromen-7-yl]oxypropanoate,
ethyl 2-[4-(o-tolyl)-2-oxo-chromen-7-yl]oxypropanoate,
2-[1-[2-[4-(2-chlorophenyl)-2-oxo-chromen-7-yl]oxypropanoyl]-4-piperidyl]acetic acid,
2-[1-[2-[4-(o-tolyl)-2-oxo-chromen-7-yl]oxypropanoyl]-4-piperidyl]acetic acid,
2-[4-(2-chloro-3-fluoro-phenyl)-2-oxo-chromen-7-yl]oxy-N-cyclopropyl-propanamide,
N-isopropyl-2-[4-(o-tolyl)-2-oxo-chromen-7-yl]oxy-propanamide,
1-[2-[4-(o-tolyl)-2-oxo-chromen-7-yl]oxypropanoyl]pyrrolidine-3-sulfonamide,
isopropyl (2R)-2-[4-(2-chlorophenyl)-2-oxo-chromen-7-yl]oxypropanoate,
2-[4-(2-chloro-4-fluoro-phenyl)-2-oxo-chromen-7-yl]oxy-N-isopropyl-propanamide,
2-[1-[2-[4-(2-chlorophenyl)-2-oxo-chromen-7-yl]oxypropanoyl]-3-piperidyl]acetic acid,
(3R)-1-[2-[4-(2-chlorophenyl)-2-oxo-chromen-7-yl]oxypropanoyl]-N,N-dimethyl-piperidine-3-carboxamide,
2-[4-(2-chloro-3-fluoro-phenyl)-2-oxo-chromen-7-yl]oxy-N-ethyl-propanamide,
1-[2-[4-(2-chloro-4-fluoro-phenyl)-2-oxo-chromen-7-yl]oxypropanoyl]piperidine-3-sulfonamide,
2-[4-(2-chlorophenyl)-2-oxo-chromen-7-yl]oxy-N-isopropyl-propanamide,
7-[2-(4,4-difluoro-1-piperidyl)-1-methyl-2-oxo-ethoxy]-4-(o-tolyl)chromen-2-one,
2-[1-[2-[4-(o-tolyl)-2-oxo-chromen-7-yl]oxypropanoyl]-3-piperidyl]acetic acid,
7-[1-methyl-2-[3-(methylsulfonimidoyl)-1-piperidyl]-2-oxo-ethoxy]-4-(o-tolyl)chromen-2-one,
4-(2-chlorophenyl)-7-[2-(4,4-difluoro-1-piperidyl)-1-methyl-2-oxo-ethoxy]chromen-2-one,
ethyl 1-[2-[4-(2-chlorophenyl)-2-oxo-chromen-7-yl]oxypropanoyl]-3-methyl-piperidine-3-carboxylate,
(3S)-1-[2-[4-(2-chlorophenyl)-2-oxo-chromen-7-yl]oxypropanoyl]-N,N-dimethyl-piperidine-3-carboxamide,
1-[2-[4-(2-chlorophenyl)-2-oxo-chromen-7-yl]oxypropanoyl]pyrrolidine-3-sulfonamide,
(3R)—N,N-dimethyl-1-[2-[4-(o-tolyl)-2-oxo-chromen-7-yl]oxypropanoyl]piperidine-3-carboxamide,
4-(2-chlorophenyl)-7-[1-methyl-2-[3-(methylsulfonimidoyl)-1-piperidyl]-2-oxo-ethoxy]chromen-2-one,
methyl 2-[1-[2-[4-(2-chlorophenyl)-2-oxo-chromen-7-yl]oxypropanoyl]-4-piperidyl]acetate,
ethyl 2-[4-(2-bromophenyl)-2-oxo-chromen-7-yl]oxypropanoate,
ethyl 2-[4-(2-chloro-4-fluoro-phenyl)-2-oxo-chromen-7-yl]oxypropanoate,
methyl 2-[1-[2-[4-(o-tolyl)-2-oxo-chromen-7-yl]oxypropanoyl]-4-piperidyl]acetate,
(3S)—N,N-dimethyl-1-[2-[4-(o-tolyl)-2-oxo-chromen-7-yl]oxypropanoyl]piperidine-3-carboxamide,
N-ethyl-2-[4-(o-tolyl)-2-oxo-chromen-7-yl]oxy-propanamide,
ethyl 2-[1-[2-[4-(2-chlorophenyl)-2-oxo-chromen-7-yl]oxypropanoyl]-3-piperidyl]acetate,
ethyl 2-[1-[2-[4-(o-tolyl)-2-oxo-chromen-7-yl]oxypropanoyl]-3-piperidyl]acetate,
ethyl 3-methyl-1-[2-[4-(o-tolyl)-2-oxo-chromen-7-yl]oxypropanoyl]piperidine-3-carboxylate,
2-[4-(2-bromophenyl)-2-oxo-chromen-7-yl]oxy-N,N-dimethyl-propanamide,
2-[4-(2-chloro-4-fluoro-phenyl)-2-oxo-chromen-7-yl]oxy-N-cyclopropyl-propanamide,
2-[4-(2-chloro-4-fluoro-phenyl)-2-oxo-chromen-7-yl]oxy-N-ethyl-propanamide,
isopropyl 2-[4-(2-bromophenyl)-2-oxo-chromen-7-yl]oxypropanoate,
2-[4-(2-chlorophenyl)-2-oxo-chromen-7-yl]oxy-N,N-dimethyl-propanamide,
2-[4-(2-chloro-3-fluoro-phenyl)-2-oxo-chromen-7-yl]oxypropanoic acid,
ethyl 1-[2-[4-(2-chloro-4-fluoro-phenyl)-2-oxo-chromen-7-yl]oxypropanoyl]-3-methyl-piperidine-3-carboxylate,
2-[4-(2-chlorophenyl)-2-oxo-chromen-7-yl]oxypropanoic acid,
2-[4-(o-tolyl)-2-oxo-chromen-7-yl]oxypropanoic acid,
2-[4-(2-chlorophenyl)-2-oxo-chromen-7-yl]oxy-N-ethyl-propanamide,
2-[4-(2-chloro-4-fluoro-phenyl)-2-oxo-chromen-7-yl]oxypropanoic acid,
methyl 2-[(3S)-1-[(2R)-2-[4-(2-chlorophenyl)-2-oxo-chromen-7-yl]oxypropanoyl]-3-piperidyl]acetate,
methyl 2-[(3R)-1-[(2R)-2-[4-(2-chlorophenyl)-2-oxo-chromen-7-yl]oxypropanoyl]-3-piperidyl]acetate,
2-[(3S)-1-[(2R)-2-[4-(2-chlorophenyl)-2-oxo-chromen-7-yl]oxypropanoyl]-3-piperidyl]acetic acid,
2-[(3R)-1-[(2R)-2-[4-(2-chlorophenyl)-2-oxo-chromen-7-yl]oxypropanoyl]-3-piperidyl]acetic acid,
methyl 2-[(3R)-1-[(2R)-2-[4-(2-chloro-4-fluoro-phenyl)-2-oxo-chromen-7-yl]oxypropanoyl]-3-piperidyl]acetate,
methyl 2-[(3S)-1-[(2R)-2-[4-(2-chloro-4-fluoro-phenyl)-2-oxo-chromen-7-yl]oxypropanoyl]-3-piperidyl]acetate,
2-[(3R)-1-[(2R)-2-[4-(2-chloro-4-fluoro-phenyl)-2-oxo-chromen-7-yl]oxypropanoyl]-3-piperidyl]acetic acid,
2-[(3S)-1-[(2R)-2-[4-(2-chloro-4-fluoro-phenyl)-2-oxo-chromen-7-yl]oxypropanoyl]-3-piperidyl]acetic acid,
(3R)-1-[(2R)-2-[4-(2-chloro-4-fluoro-phenyl)-2-oxo-chromen-7-yl]oxypropanoyl]piperidine-3-carboxylic acid,
ethyl (3S)-1-[2-[4-(2-chlorophenyl)-2-oxo-chromen-7-yl]oxybutanoyl]piperidine-3-carboxylate,
(3S)-1-[2-[4-(2-chlorophenyl)-2-oxo-chromen-7-yl]oxybutanoyl]piperidine-3-carboxylic acid,
ethyl (3S)-1-[(2R)-2-[4-(2-chloro-4-fluoro-phenyl)-2-oxo-chromen-7-yl]oxypropanoyl]piperidine-3-carboxylate,
2-[4-(2-chlorophenyl)-2-oxo-chromen-7-yl]oxy-2-methyl-propanoic acid,
ethyl 2-[4-(2-chlorophenyl)-2-oxo-chromen-7-yl]oxy-2-methyl-propanoate,
2-[4-(2-chlorophenyl)-2-oxo-chromen-7-yl]oxy-N-isopropyl-2-methyl-propanamide,
N-isopropyl-2-methyl-2-[4-(o-tolyl)-2-oxo-chromen-7-yl]oxy-propanamide,
(3S)-1-[2-[4-(2-chlorophenyl)-2-oxo-chromen-7-yl]oxy-2-methyl-propanoyl]piperidine-3-carboxylic acid,
isopropyl (3S)-1-[(2R)-2-[4-(2-chloro-4-fluoro-phenyl)-2-oxo-chromen-7-yl]oxypropanoyl]piperidine-3-carboxylate,
tert-butyl (3S)-1-[(2R)-2-[4-(2-chloro-4-fluoro-phenyl)-2-oxo-chromen-7-yl]oxypropanoyl]piperidine-3-carboxylate,
2-morpholinoethyl (3S)-1-[(2R)-2-[4-(2-chloro-4-fluoro-phenyl)-2-oxo-chromen-7-yl]oxypropanoyl]piperidine-3-carboxylate,
heptyl (3S)-1-[(2R)-2-[4-(2-chloro-4-fluoro-phenyl)-2-oxo-chromen-7-yl]oxypropanoyl]piperidine-3-carboxylate,
isopropoxycarbonyloxymethyl (3S)-1-[(2R)-2-[4-(2-chloro-4-fluoro-phenyl)-2-oxo-chromen-7-yl]oxypropanoyl]piperidine-3-carboxylate,
(3S)—N-methyl-1-[(2R)-2-[4-(2-chloro-4-fluoro-phenyl)-2-oxo-chromen-7-yl]oxypropanoyl piperidine-3-carboxamide,
isopropyl 2-[(3R)-1-[(2R)-2-[4-(2-chloro-4-fluoro-phenyl)-2-oxo-chromen-7-yl]oxypropanoyl]-3-piperidyl]acetate,
tert-butyl 2-[(3R)-1-[(2R)-2-[4-(2-chloro-4-fluoro-phenyl)-2-oxo-chromen-7-yl]oxypropanoyl]-3-piperidyl]acetate,
2-morpholinoethyl 2-[(3R)-1-[(2R)-2-[4-(2-chloro-4-fluoro-phenyl)-2-oxo-chromen-7-yl]oxypropanoyl]-3-piperidyl]acetate,
heptyl 2-[(3R)-1-[(2R)-2-[4-(2-chloro-4-fluoro-phenyl)-2-oxo-chromen-7-yl]oxypropanoyl]-3-piperidyl]acetate,
isopropoxycarbonyloxymethyl 2-[(3R)-1-[(2R)-2-[4-(2-chloro-4-fluoro-phenyl)-2-oxo-chromen-7-yl]oxypropanoyl]-3-piperidyl]acetate, and
2-[(3R)-1-[(2R)-2-[4-(2-chloro-4-fluoro-phenyl)-2-oxo-chromen-7-yl]oxypropanoyl]-3-piperidyl]-N-methyl-acetamide,
(3S)-1-[(2R)-2-[[4-(o-tolyl)-7-quinolyl]oxy]propanoyl]piperidine-3-carboxylic acid,
(3S)-1-[(2R)-2-[[4-(2,6-dimethylphenyl)-7-quinolyl]oxy]propanoyl]piperidine-3-carboxylic acid,
2-[(3R)-1-[(2R)-2-[[4-(2,6-dimethylphenyl)-7-quinolyl]oxy]propanoyl]-3-piperidyl]acetic acid,
ethyl (3S)-1-[(2R)-2-[[4-(2,6-dimethylphenyl)-7-quinolyl]oxy]propanoyl]piperidine-3-carboxylate,
2-[(3S)-1-[(2R)-2-[[4-(2,6-dimethylphenyl)-7-quinolyl]oxy]propanoyl]-3-piperidyl]acetic acid,
ethyl 2-[(3R)-1-[(2R)-2-[[4-(2,6-dimethylphenyl)-7-quinolyl]oxy]propanoyl]-3-piperidyl]acetate,
(3R)-1-[(2R)-2-[[4-(2-chlorophenyl)-7-quinolyl]oxy]propanoyl]piperidine-3-carboxylic acid,
ethyl 2-[(3R)-1-[(2R)-2-[[4-(o-tolyl)-7-quinolyl]oxy]propanoyl]-3-piperidyl]acetate,
(3S)-1-[(2R)-2-[[4-(2-chlorophenyl)-7-quinolyl]oxy]propanoyl]piperidine-3-carboxylic acid,
(3S)-1-[(2R)-2-[[4-(2-chloro-4-fluoro-phenyl)-7-quinolyl]oxy]propanoyl]piperidine-3-carboxylic acid,
(2R)-2-[[4-(2-chloro-4-fluoro-phenyl)-7-quinolyl]oxy]-1-[(3S)-3-(2H-tetrazol-5-yl)-1-piperidyl]propan-1-one,
(3S)-1-[(2R)-2-[[4-(2-chlorophenyl)-2-fluoro-7-quinolyl]oxy]propanoyl]piperidine-3-carboxylic acid,
2-[(3R)-1-[(2R)-2-[[4-(o-tolyl)-7-quinolyl]oxy]propanoyl]-3-piperidyl]acetic acid,
ethyl (3S)-1-[(2R)-2-[[4-(o-tolyl)-7-quinolyl]oxy]propanoyl]piperidine-3-carboxylate,
ethyl 2-[(3R)-1-[(2R)-2-[[4-(2-chloro-4-fluoro-phenyl)-7-quinolyl]oxy]propanoyl]-3-piperidyl]acetate,
2-[(3R)-1-[(2R)-2-[[4-(2-chlorophenyl)-7-quinolyl]oxy]propanoyl]-3-piperidyl]acetic acid
(3R)-1-[(2R)-2-[[4-(2-chloro-4-fluoro-phenyl)-7-quinolyl]oxy]propanoyl]piperidine-3-carboxylic acid,
2-[(3R)-1-[(2R)-2-[[4-(2-chloro-4-fluoro-phenyl)-7-quinolyl]oxy]propanoyl]-3-piperidyl]acetic acid,
(3S)-1-[(2R)-2-[[4-(2-chloro-4-fluoro-phenyl)-7-quinolyl]oxy]propanoyl]pyrrolidine-3-carboxylic acid,
(2R)-2-[[4-(2-chloro-4-fluoro-phenyl)-7-quinolyl]oxy]-1-(1-piperidyl)propan-1-one,
2-[(3R)-1-[(2R)-2-[[2-chloro-4-(o-tolyl)-7-quinolyl]oxy]propanoyl]-3-piperidyl]acetic acid,
2-[(3S)-1-[(2R)-2-[[4-(2-chloro-4-fluoro-phenyl)-7-quinolyl]oxy]propanoyl]-3-piperidyl]acetic acid,
rac-(3S)-1-[[2-[4-(2-chlorophenyl)-7-quinolyl]oxy]propanoyl]piperidine-3-carboxylic acid,
1-[(2R)-2-[[4-(2-chloro-4-fluoro-phenyl)-7-quinolyl]oxy]propanoyl]piperidine-4-carboxylic acid,
(3S)-1-[rac-(2R)-2-[[2-chloro-4-(o-tolyl)-7-quinolyl]oxy]propanoyl]piperidine-3-carboxylic acid,
3-[[(2R)-2-[[4-(2-chloro-4-fluoro-phenyl)-7-quinolyl]oxy]propanoyl]amino]benzoic acid ethyl (3S)-1-[(2R)-2-[[4-(2-chloro-4-fluoro-phenyl)-7-quinolyl]oxy]propanoyl]piperidine-3-carboxylate,
(2R)-2-[[4-(2-chloro-4-fluoro-phenyl)-7-quinolyl]oxy]-1-(4-propanoylpiperazin-1-yl)propan-1-one,
tert-butyl (2R)-2-[[4-(2-chlorophenyl)-7-quinolyl]oxy]propanoate,
(3S)-1-[(2R)-2-[[4-(2-chloro-4-fluoro-phenyl)-7-quinolyl]oxy]propanoyl]piperidine-3-carbonitrile,
(3S)-1-[(2R)-2-[[2-methyl-4-(o-tolyl)-7-quinolyl]oxy]propanoyl]piperidine-3-carboxylic acid,
(2R)-2-[[4-(2-chloro-4-fluoro-phenyl)-7-quinolyl]oxy]-N-isopropyl-N-methyl-propanamide,
1-[rac-(2R)-2-[[4-(2-chloro-4-fluoro-phenyl)-7-quinolyl]oxy]propanoyl]piperidine-3-sulfonamide,
(3S)-1-[(2R)-2-[[2-chloro-4-(2-chlorophenyl)-7-quinolyl]oxy]propanoyl]piperidine-3-carboxylic acid,
(3S)-1-[(2R)-2-[[2-chloro-4-(4-fluoro-2-methyl-phenyl)-7-quinolyl]oxy]propanoyl]piperidine-3-carboxylic acid,
isopropyl (2R)-2-[[4-(2-chlorophenyl)-7-quinolyl]oxy]propanoate,
methyl 2-[(3R)-1-[(2R)-2-[[4-(2-chloro-4-fluoro-phenyl)-7-quinolyl]oxy]propanoyl]pyrrolidin-3-yl]acetate,
(3S)-1-[(2R)-2-[[4-(2-chloro-4-fluoro-phenyl)-7-quinolyl]oxy]propanoyl]-N-methyl-piperidine-3-carboxamide,
2-[(3S)-1-[(2R)-2-[[4-(2-chloro-4-fluoro-phenyl)-7-quinolyl]oxy]propanoyl]pyrrolidin-3-yl]acetic acid,
2-[(3R)-1-[(2R)-2-[[4-(2-chloro-4-fluoro-phenyl)-7-quinolyl]oxy]propanoyl]pyrrolidin-3-yl]acetic acid,
ethyl (3S)-1-[(2R)-2-[[4-(2-chlorophenyl)-2-fluoro-7-quinolyl]oxy]propanoyl]piperidine-3-carboxylate,
(2R)-2-[[4-(2-chloro-4-fluoro-phenyl)-7-quinolyl]oxy]-1-pyrrolidin-1-yl-propan-1-one,
(2R)-2-[[2-chloro-4-(o-tolyl)-7-quinolyl]oxy]propanoic acid,
(2R)-2-[[4-(2-chloro-4-fluoro-phenyl)-7-quinolyl]oxy]propanoic acid,
(2R)-2-[[4-(2-chloro-4-fluoro-phenyl)-7-quinolyl]oxy]-N,N-dimethyl-propanamide,
rac-(3S)-1-[2-[[4-(2-chlorophenyl)-7-quinolyl]oxy]acetyl]piperidine-3-carboxylic acid,
(2R)—N-tert-butyl-2-[[4-(2-chloro-4-fluoro-phenyl)-7-quinolyl]oxy]propanamide,
(2R)-2-[[4-(2-chlorophenyl)-7-quinolyl]oxy]-N-isopropyl-propanamide,
ethyl 2-[(3R)-1-[(2R)-2-[[2-methyl-4-(o-tolyl)-7-quinolyl]oxy]propanoyl]-3-piperidyl]acetate,
ethyl (2R)-2-[[4-(2-chlorophenyl)-7-quinolyl]oxy]propanoate,
ethyl 4-[[(2R)-2-[[4-(2-chloro-4-fluoro-phenyl)-7-quinolyl]oxy]propanoyl]amino]benzoate,
(3S)-1-[(2R)-2-[[2-chloro-4-(o-tolyl)-7-quinolyl]oxy]propanoyl]piperidine-3-carboxamide,
(2R)-2-[[4-(2-chlorophenyl)-7-quinolyl]oxy]-1-(1-piperidyl)propan-1-one,
methyl 3-[[(2R)-2-[[4-(2-chloro-4-fluoro-phenyl)-7-quinolyl]oxy]propanoyl]amino]cyclobutanecarboxylate,
(2R)-2-[[4-(2-chloro-4-fluoro-phenyl)-7-quinolyl]oxy]-1-piperazin-1-yl-propan-1-one,
2-[(3R)-1-[(2R)-2-[[2-chloro-4-(2-chloro-4-fluoro-phenyl)-7-quinolyl]oxy]propanoyl]-3-piperidyl]acetic acid,
2-[(3R)-1-[(2R)-2-[[2-chloro-4-(4-fluoro-2-methyl-phenyl)-7-quinolyl]oxy]propanoyl]-3-piperidyl]acetic acid,
ethyl 1-[(2R)-2-[[4-(2-chloro-4-fluoro-phenyl)-7-quinolyl]oxy]propanoyl]piperidine-4-carboxylate,
(3S)-1-[(2R)-2-[[4-(2-chloro-4-fluoro-phenyl)-2-methyl-7-quinolyl]oxy]propanoyl]piperidine-3-carboxylic acid,
4-[[(2R)-2-[[4-(2-chloro-4-fluoro-phenyl)-7-quinolyl]oxy]propanoyl]amino]benzoic acid,
(2R)-2-[[4-(2-chloro-4-fluoro-phenyl)-7-quinolyl]oxy]-N-isopropyl-propanamide,
(3S)-1-[2-[[5-(2-chloro-4-fluoro-phenyl)-1,8-naphthyridin-2-yl]oxy]propanoyl]piperidine-3-carboxylic acid,
4-[(2R)-2-[[4-(2-chloro-4-fluoro-phenyl)-7-quinolyl]oxy]propanoyl]piperazin-2-one,
(3S)-1-[(2R)-2-[[2-chloro-4-(2-chloro-4-fluoro-phenyl)-7-quinolyl]oxy]propanoyl]piperidine-3-carboxylic acid,
2-[(3R)-1-[(2R)-2-[[4-(2-chloro-3-fluoro-phenyl)-7-quinolyl]oxy]propanoyl]-3-piperidyl]acetic acid,
(3S)-1-[(2R)-2-[(4-phenyl-7-quinolyl)oxy]propanoyl]piperidine-3-carboxylic acid,
methyl 2-[(3S)-1-[(2R)-2-[[4-(2-chloro-4-fluoro-phenyl)-7-quinolyl]oxy]propanoyl]pyrrolidin-3-yl]acetate,
2-[(3R)-1-[(2R)-2-[[2-chloro-4-(2-chlorophenyl)-7-quinolyl]oxy]propanoyl]-3-piperidyl]-N-methyl-acetamide,
2-[(3R)-1-[(2R)-2-[[2-methyl-4-(o-tolyl)-7-quinolyl]oxy]propanoyl]-3-piperidyl]acetic acid,
(2R)-2-[[2-chloro-4-(2-chlorophenyl)-7-quinolyl]oxy]-1-[4-(cyclopropanecarbonyl)piperazin-1-yl]propan-1-one,
methyl (3R)-1-[(2R)-2-[[4-(2-chloro-4-fluoro-phenyl)-7-quinolyl]oxy]propanoyl]piperidine-3-carboxylate,
(2R)-2-[[2-chloro-4-(2-chlorophenyl)-7-quinolyl]oxy]-1-(4-propanoylpiperazin-1-yl)propan-1-one,
tert-butyl (2R)-2-[[2-chloro-4-(o-tolyl)-7-quinolyl]oxy]propanoate,
ethyl 2-[(3R)-1-[(2R)-2-[[4-(2-chloro-4-fluoro-phenyl)-2-methyl-7-quinolyl]oxy]propanoyl]-3-piperidyl]acetate,
ethyl (3S)-1-[(2R)-2-[[4-(2-chloro-4-fluoro-phenyl)-7-quinolyl]oxy]propanoyl]pyrrolidine-3-carboxylate,
(3S)-1-[(2R)-2-[[2-chloro-4-(2-chlorophenyl)-7-quinolyl]oxy]propanoyl]piperidine-3-carboxamide,
(2R)-2-[[4-(2-chloro-4-fluoro-phenyl)-7-quinolyl]oxy]-N-cyclopropyl-propanamide,
(3S)-1-[(2S)-2-[[4-(2-chlorophenyl)-7-quinolyl]oxy]propanoyl]piperidine-3-carboxylic acid,
2-[(3R)-1-[(2R)-2-[[4-(2-methyl-3-thienyl)-7-quinolyl]oxy]propanoyl]-3-piperidyl]acetic acid,
2-[rac-(3R)-1-[2-[[5-(2-chloro-4-fluoro-phenyl)-1,8-naphthyridin-2-yl]oxy]propanoyl]-3-piperidyl]acetic acid,
(3S)-1-[(2R)-2-[[2-chloro-4-(2-chloro-4-fluoro-phenyl)-7-quinolyl]oxy]propanoyl]piperidine-3-carboxamide,
isopropyl 2-[[4-(2-chlorophenyl)-7-quinolyl]oxy]acetate,
(3S)-1-[(2R)-2-[[4-(4-fluoro-2,6-dimethyl-phenyl)-7-quinolyl]oxy]propanoyl]piperidine-3-carboxylic acid,
(2R)-1-[4-(2-aminoacetyl)piperazin-1-yl]-2-[[2-chloro-4-(2-chlorophenyl)-7-quinolyl]oxy]propan-1-one,
2-[[4-(2-chlorophenyl)-7-quinolyl]oxy]-N,N-dimethyl-propanamide,
ethyl (3S)-1-[2-[[4-(2-chlorophenyl)-7-quinolyl]oxy]acetyl]piperidine-3-carboxylate,
(2R)-2-[[4-(2-chlorophenyl)-7-quinolyl]oxy]-N,N-dimethyl-propanamide,
ethyl 2-[(3S)-1-[(2R)-2-[[4-(2-chloro-4-fluoro-phenyl)-7-quinolyl]oxy]propanoyl]-3-piperidyl]acetate,
(2R)-2-[[4-(2-chlorophenyl)-2-fluoro-7-quinolyl]oxy]propanoic acid,
(2R)-2-[[2-chloro-4-(2-chloro-4-fluoro-phenyl)-7-quinolyl]oxy]propanoic acid,
ethyl 2-[(3R)-1-[(2R)-2-[[2-chloro-4-(2-chlorophenyl)-7-quinolyl]oxy]propanoyl]-3-piperidyl]acetate,
ethyl 2-[(3R)-1-[(2R)-2-[[2-chloro-4-(2-chloro-4-fluoro-phenyl)-7-quinolyl]oxy]propanoyl]-3-piperidyl]acetate,
ethyl (3S)-1-[(2R)-2-[[2-chloro-4-(4-fluoro-2-methyl-phenyl)-7-quinolyl]oxy]propanoyl]piperidine-3-carboxylate,
(2R)-2-[[4-(2-chloro-4-fluoro-phenyl)-7-quinolyl]oxy]-N-(4-pyridyl)propanamide,
(3S)-1-[(2R)-2-[[2-chloro-4-(2-chlorophenyl)-7-quinolyl]oxy]propanoyl]-N-methyl-piperidine-3-carboxamide,
methyl 3-[[(2R)-2-[[2-chloro-4-(2-chlorophenyl)-7-quinolyl]oxy]propanoyl]amino]cyclobutanecarboxylate,
2-[(3R)-1-[(2R)-2-[[2-chloro-4-(2-chloro-4-fluoro-phenyl)-7-quinolyl]oxy]propanoyl]-3-piperidyl]-N-methyl-acetamide,
(2R)-2-[[2-chloro-4-(2-chlorophenyl)-7-quinolyl]oxy]-1-(1-piperidyl)propan-1-one,
(3S)-1-[(2R)-2-[[2-chloro-4-(2-chlorophenyl)-7-quinolyl]oxy]propanoyl]-N,N-dimethyl-piperidine-3-carboxamide,
2-[(3R)-1-[(2R)-2-[[4-(2-chloro-4-fluoro-phenyl)-2-methyl-7-quinolyl]oxy]propanoyl]-3-piperidyl]acetic acid,
(3R)-1-[(2S)-2-[[4-(2-chlorophenyl)-7-quinolyl]oxy]propanoyl]piperidine-3-carboxylic acid,
(2R)-2-[[4-(2-chlorophenyl)-7-quinolyl]oxy]propanoic acid,
(2R)-2-[[2-chloro-4-(2-chlorophenyl)-7-quinolyl]oxy]-1-piperazin-1-yl-propan-1-one,
(3S)—N-methyl-1-[(2R)-2-[[2-chloro-4-(2-chloro-4-fluoro-phenyl)-7-quinolyl]oxy]propanoyl]piperidine-3-carboxamide,
2-[[4-(o-tolyl)-7-quinolyl]oxy]acetamide,
(2R)-2-[[2-chloro-4-(2-chloro-4-fluoro-phenyl)-7-quinolyl]oxy]-1-(1-piperidyl)propan-1-one,
ethyl 3-[[(2R)-2-[[4-(2-chloro-4-fluoro-phenyl)-7-quinolyl]oxy]propanoyl]amino]benzoate,
2-[[5-(2-chloro-4-fluoro-phenyl)-1,8-naphthyridin-2-yl]oxy]-N-isopropyl-propanamide,
2-[(3R)-1-[(2R)-2-[[4-(2-fluorophenyl)-7-quinolyl]oxy]propanoyl]-3-piperidyl]acetic acid,
tert-butyl (3S)-1-[(2R)-2-[[2-methyl-4-(o-tolyl)-7-quinolyl]oxy]propanoyl]piperidine-3-carboxylate,
2-[1-[(2R)-2-[[4-(2-chlorophenyl)-7-quinolyl]oxy]propanoyl]-4-piperidyl]acetic acid,
(3S)-1-[rac-(2R)-2-[[4-(2,6-dimethylphenyl)-2-methyl-7-quinolyl]oxy]propanoyl]piperidine-3-sulfonamide,
(3R)-1-[rac-(2R)-2-[[4-(2,6-dimethylphenyl)-2-methyl-7-quinolyl]oxy]propanoyl]piperidine-3-sulfonamide,
(3R)-1-[rac-(2R)-2-[[4-(2,6-dimethylphenyl)-2-methyl-7-quinolyl]oxy]propanoyl]piperidine-3-sulfonamide,
(3R)-1-[(2R)-2-[[4-(2-chloro-4-fluoro-phenyl)-2-methyl-7-quinolyl]oxy]propanoyl]piperidine-3-sulfonamide,
(3S)-1-[(2R)-2-[[4-(2-chloro-4-fluoro-phenyl)-7-quinolyl]oxy]propanoyl]piperidine-3-sulfonamide,
(3R)-1-[(2R)-2-[[4-(2-chloro-4-fluoro-phenyl)-7-quinolyl]oxy]propanoyl]piperidine-3-sulfonamide,
3-[1-[(2R)-2-[[4-(2-chlorophenyl)-7-quinolyl]oxy]propanoyl]-4-piperidyl]propanoic acid,
1-[rac-(2R)-2-[[4-(2,6-dimethylphenyl)-7-quinolyl]oxy]propanoyl]piperidine-3-sulfonamide,
2-[(3R)-1-[(2R)-2-[[4-(2,6-dichlorophenyl)-7-quinolyl]oxy]propanoyl]-3-piperidyl]acetic acid,
2-[(3R)-1-[(2R)-2-[[4-(2-ethylphenyl)-7-quinolyl]oxy]propanoyl]-3-piperidyl]acetic acid,
2-[(3R)-1-[(2R)-2-[[4-(2-isopropylphenyl)-7-quinolyl]oxy]propanoyl]-3-piperidyl]acetic acid,
[1-[rac-(2R)-2-[[4-(2-chloro-4-fluoro-phenyl)-7-quinolyl]oxy]propanoyl]-3-piperidyl]methanesulfonamide,
2-[(3R)-1-[(2R)-2-[[4-(2,6-dimethylphenyl)-2-methyl-7-quinolyl]oxy]propanoyl]-3-piperidyl]acetic acid,
(2R)-2-[[4-(2-chloro-4-fluoro-phenyl)-7-quinolyl]oxy]-N-[4-(2-hydroxyethyl)phenyl]propanamide,
(3S)-1-[(2R)-2-[[5-(2-chloro-4-fluoro-phenyl)-1,8-naphthyridin-2-yl]oxy]propanoyl]piperidine-3-carboxylic acid,
(3S)-1-[(2S)-2-[[5-(2-chloro-4-fluoro-phenyl)-1,8-naphthyridin-2-yl]oxy]propanoyl]piperidine-3-carboxylic acid,
(3S)-1-[(2S)-2-[[5-(2-chloro-4-fluoro-phenyl)-1,8-naphthyridin-2-yl]oxy]propanoyl]piperidine-3-carboxylic acid,
2-[(3R)-1-[(2R)-2-[[5-(2-chloro-4-fluoro-phenyl)-1,8-naphthyridin-2-yl]oxy]propanoyl]-3-piperidyl]acetic acid,
2-[(3R)-1-[(2R)-2-[[5-(2-chloro-4-fluoro-phenyl)-1,8-naphthyridin-2-yl]oxy]propanoyl]-3-piperidyl]acetic acid,
(3S)-1-[(2R)-2-[[4-(2,6-dichlorophenyl)-7-quinolyl]oxy]propanoyl]piperidine-3-carboxylic acid,
(2R)-2-[[4-(2-chloro-4-fluoro-phenyl)-7-quinolyl]oxy]-N-(2-pyridyl)propanamide,
(2R)-2-[[4-(2-chloro-4-fluoro-phenyl)-7-quinolyl]oxy]-N-ethyl-propanamide,
(3S)-1-[(2R)-2-[[4-(2,6-dichloro-4-fluoro-phenyl)-7-quinolyl]oxy]propanoyl]piperidine-3-carboxylic acid,
2-[(3R)-1-[(2R)-2-[[4-(4-methyl-3-thienyl)-7-quinolyl]oxy]propanoyl]-3-piperidyl]acetic acid,
2-[(3R)-1-[(2R)-2-[[4-(3-methyl-2-thienyl)-7-quinolyl]oxy]propanoyl]-3-piperidyl]acetic acid,
2-[(3R)-1-[(2R)-2-[[4-(2-methoxyphenyl)-7-quinolyl]oxy]propanoyl]-3-piperidyl]acetic acid,
2-[(3R)-1-[(2R)-2-[[4-[2-(trifluoromethyl)phenyl]-7-quinolyl]oxy]propanoyl]-3-piperidyl]acetic acid,
2-[(3R)-1-[(2R)-2-[[4-[2-(trifluoromethoxy)phenyl]-7-quinolyl]oxy]propanoyl]-3-piperidyl]acetic acid,
(3S)-1-[(2R)-2-[[4-(2,6-dimethylphenyl)-7-quinolyl]oxy]propanoyl]piperidine-3-sulfonamide,
2-[(3R)-1-[2-[[4-(2-chlorophenyl)-7-quinolyl]oxy]-2-methyl-propanoyl]-3-piperidyl]acetic acid,
2-[rac-(3R)-1-[2-[[4-(2-chlorophenyl)-7-quinolyl]oxy]butanoyl]-3-piperidyl]acetic acid,
2-[(3R)-1-[(2R)-2-[[4-(2-chloro-6-methyl-phenyl)-7-quinolyl]oxy]propanoyl]-3-piperidyl]acetic acid,
2-[(3R)-1-[(2R)-2-[[4-(2-bromophenyl)-7-quinolyl]oxy]propanoyl]-3-piperidyl]acetic acid,
2-[(3R)-1-[(2R)-2-[[4-(2-cyanophenyl)-7-quinolyl]oxy]propanoyl]-3-piperidyl]acetic acid,
2-[(3R)-1-[(2R)-2-[[4-(2-ethynylphenyl)-7-quinolyl]oxy]propanoyl]-3-piperidyl]acetic acid,
2-[(3R)-1-[(2R)-2-[[4-[2-(dimethylamino)phenyl]-7-quinolyl]oxy]propanoyl]-3-piperidyl]acetic acid,
2-[(3R)-1-[(2R)-2-[[4-(2-carbamoylphenyl)-7-quinolyl]oxy]propanoyl]-3-piperidyl]acetic acid,
2-[(3R)-1-[(2R)-2-[[4-(2,6-difluorophenyl)-7-quinolyl]oxy]propanoyl]-3-piperidyl]acetic acid,
2-[(3R)-1-[(2R)-2-[[4-(2,4-dimethyl-3-thienyl)-7-quinolyl]oxy]propanoyl]-3-piperidyl]acetic acid,
2-[(3R)-1-[(2R)-2-[[4-[2-chloro-6-(trifluoromethyl)phenyl]-7-quinolyl]oxy]propanoyl]-3-piperidyl]acetic acid,
2-[(3R)-1-[(2R)-2-[[4-(2-bromo-6-chloro-phenyl)-7-quinolyl]oxy]propanoyl]-3-piperidyl]acetic acid,
2-[(3R)-1-[(2R)-2-[[4-[2,6-bis(trifluoromethyl)phenyl]-7-quinolyl]oxy]propanoyl]-3-piperidyl]acetic acid,
2-[(3R)-1-[(2R)-2-[[4-(2-chloro-6-methoxy-phenyl)-7-quinolyl]oxy]propanoyl]-3-piperidyl]acetic acid,
2-[(3R)-1-[(2R)-2-[[4-(2,6-diisopropylphenyl)-7-quinolyl]oxy]propanoyl]-3-piperidyl]acetic acid,
(2R)-1-[(3R)-3-amino-1-piperidyl]-2-[[4-(2,6-dimethylphenyl)-7-quinolyl]oxy]propan-1-one,
(2R)-1-[(3S)-3-amino-1-piperidyl]-2-[[4-(2,6-dimethylphenyl)-7-quinolyl]oxy]propan-1-one,
N-[(3S)-1-[(2R)-2-[[4-(2,6-dimethylphenyl)-7-quinolyl]oxy]propanoyl]-3-piperidyl]propanamide,
N-tert-butyl-4-[(2R)-2-[[4-(2,6-dimethylphenyl)-7-quinolyl]oxy]propanoyl]piperazine-1-carboxamide,
(2R)-2-[[4-(2,6-dimethylphenyl)-7-quinolyl]oxy]-1-[3-(1-hydroxycyclopropyl)-1-piperidyl]propan-1-one,
8-[(2R)-2-[[4-(2,6-dimethylphenyl)-7-quinolyl]oxy]propanoyl]-2,8-diazaspiro[4.5]decan-1-one,
(2R)-2-[[4-(2,6-dimethylphenyl)-7-quinolyl]oxy]-1-(3,5-dimethylpiperazin-1-yl)propan-1-one,
N-[(3S)-1-[(2R)-2-[[4-(2,6-dimethylphenyl)-7-quinolyl]oxy]propanoyl]-3-piperidyl]-N-hydroxy-acetamide,
1-[4-[(2R)-2-[[4-(2,6-dimethylphenyl)-7-quinolyl]oxy]propanoyl]piperazin-1-yl]-2,2-dimethyl-propan-1-one,
N-[(3S)-1-[(2R)-2-[[4-(2,6-dimethylphenyl)-7-quinolyl]oxy]propanoyl]-3-piperidyl]methanesulfonamide,
N-[(3S)-1-[(2R)-2-[[4-(2,6-dimethylphenyl)-7-quinolyl]oxy]propanoyl]-3-piperidyl]benzamide,
(3S)—N-cyano-1-[(2R)-2-[[4-(2,6-dimethylphenyl)-7-quinolyl]oxy]propanoyl]piperidine-3-carboxamide,
(3S)-1-[(2R)-2-[[4-(2,6-dimethylphenyl)-7-quinolyl]oxy]propanoyl]piperidine-3-carbohydroxamic acid,
2-[(3R)-1-[(2R)-2-[[4-(2,6-dimethylphenyl)-7-quinolyl]oxy]propanoyl]-3-piperidyl]ethanehydroxamic acid,
(2R)-1-(3-aminoazetidin-1-yl)-2-[[4-(2,6-dimethylphenyl)-7-quinolyl]oxy]propan-1-one,
(2R)-2-[[4-(2,6-dimethylphenyl)-7-quinolyl]oxy]-1-[3-(1H-tetrazol-5-yl)azetidin-1-yl]propan-1-one,
3-hydroxy-1-[(2R)-2-[[4-(2,6-dimethylphenyl)-7-quinolyl]oxy]propanoyl]piperidine-3-carboxylic acid,
5-[(2R)-2-[[4-(2,6-dimethylphenyl)-7-quinolyl]oxy]propanoyl]-5-azaspiro[2.5]octane-2-carboxylic acid,
(3R)-1-[(2R)-2-[[4-(2,6-dimethylphenyl)-7-quinolyl]oxy]propanoyl]piperidine-3-sulfonamide,
(3S)-1-[(2R)-2-[[4-(2,6-dichloro-4-fluoro-phenyl)-7-quinolyl]oxy]propanoyl]-3-methyl-piperidine-3-carboxylic acid,
5-[rac-(2R)-2-[[4-(4-fluoro-2,6-dimethyl-phenyl)-7-quinolyl]oxy]propanoyl]-5-azaspiro[2.5]octane-2-carboxylic acid,
(3R)-1-[(2R)-2-[[4-(4-fluoro-2,6-dimethyl-phenyl)-7-quinolyl]oxy]propanoyl]-3-methyl-piperidine-3-carboxylic acid,
(2R)-2-[[4-(4-fluoro-2,6-dimethyl-phenyl)-7-quinolyl]oxy]-1-[(2S)-2-methyl-1-piperidyl]propan-1-one,
5-[rac-(2R)-2-[[4-(2,6-dichloro-4-fluoro-phenyl)-7-quinolyl]oxy]propanoyl]-5-azaspiro[2.5]octane-2-carboxylic acid,
(2R)-2-[[4-(4-fluoro-2,6-dimethyl-phenyl)-7-quinolyl]oxy]-1-morpholino-propan-1-one,
(2R)-2-[[4-(4-fluoro-2,6-dimethyl-phenyl)-7-quinolyl]oxy]-1-(2-oxa-7-azaspiro[3.5]nonan-7-yl)propan-1-one,
rac-(2R)-2-[[4-(4-fluoro-2,6-dimethyl-phenyl)-7-quinolyl]oxy]-1-[3-(1-hydroxycyclopropyl)-1-piperidyl]propan-1-one,
8-[(2R)-2-[[4-(4-fluoro-2,6-dimethyl-phenyl)-7-quinolyl]oxy]propanoyl]-2,8-diazaspiro[4.5]decan-1-one,
1-[1-[rac-(2R)-2-[[4-(4-fluoro-2,6-dimethyl-phenyl)-7-quinolyl]oxy]propanoyl]-3-piperidyl]cyclopropanecarboxylic acid,
2-[(3R)-1-[(2R)-2-[[4-(4-fluoro-2,6-dimethyl-phenyl)-7-quinolyl]oxy]propanoyl]-3-piperidyl]acetic acid,
(3S)-1-[(2R)-2-[[4-(4-fluoro-2,6-dimethyl-phenyl)-7-quinolyl]oxy]propanoyl]-3-methyl-piperidine-3-carboxylic acid,
N-[(3S)-1-[(2R)-2-[[4-(4-fluoro-2,6-dimethyl-phenyl)-7-quinolyl]oxy]propanoyl]-3-piperidyl]-N-hydroxy-acetamide,
2-methyl-2-[1-[rac-(2R)-2-[[4-(4-fluoro-2,6-dimethyl-phenyl)-7-quinolyl]oxy]propanoyl]-3-piperidyl]propanoic acid,
(2R)-2-[[4-(4-fluoro-2,6-dimethyl-phenyl)-7-quinolyl]oxy]-1-[(2R)-2-methyl-1-piperidyl]propan-1-one,
(3R)-1-[(2R)-2-[[4-(2,6-dichloro-4-fluoro-phenyl)-7-quinolyl]oxy]propanoyl]-3-methyl-piperidine-3-carboxylic acid,
(3S)-1-[(2R)-2-[[4-(2-chloro-4-fluoro-phenyl)-7-quinolyl]oxy]propanoyl]-3-methyl-piperidine-3-carboxylic acid,
[1-[rac-(2R)-2-[[4-(2,6-dimethylphenyl)-7-quinolyl]oxy]propanoyl]-3-piperidyl]methanesulfonamide,
rac-(2R)-1-(2,6-dimethyl-1-piperidyl)-2-[[4-(4-fluoro-2,6-dimethyl-phenyl)-7-quinolyl]oxy]propan-1-one,
2-[(3R)-1-[(2R)-2-[[4-(2,6-dichloro-4-fluoro-phenyl)-7-quinolyl]oxy]propanoyl]-3-piperidyl]acetic acid,
5-[rac-(2R)-2-[[4-(2-chloro-4-fluoro-phenyl)-7-quinolyl]oxy]propanoyl]-5-azaspiro[2.5]octane-2-carboxylic acid,
1-[1-[rac-(2R)-2-[[4-(2-chloro-4-fluoro-phenyl)-7-quinolyl]oxy]propanoyl]-3-piperidyl]cyclopropanecarboxylic acid,
2-methyl-2-[1-[rac-(2R)-2-[[4-(2,6-dichloro-4-fluoro-phenyl)-7-quinolyl]oxy]propanoyl]-3-piperidyl]propanoic acid,
1-[1-[rac-(2R)-2-[[4-(2,6-dichloro-4-fluoro-phenyl)-7-quinolyl]oxy]propanoyl]-3-piperidyl]cyclopropanecarboxylic acid,
2-methyl-2-[1-[rac-(2R)-2-[[4-(2-chloro-4-fluoro-phenyl)-7-quinolyl]oxy]propanoyl]-3-piperidyl]propanoic acid,
(3R)-1-[(2R)-2-[[4-(2-chloro-4-fluoro-phenyl)-7-quinolyl]oxy]propanoyl]-3-methyl-piperidine-3-carboxylic acid,
(2R)-2-[[4-(2,6-dichloro-4-fluoro-phenyl)-7-quinolyl]oxy]-1-[(2S)-2-methyl-1-piperidyl]propan-1-one,
rac-(2R)-2-[[4-(2-chloro-4-fluoro-phenyl)-7-quinolyl]oxy]-1-(2-methyl-1-piperidyl)propan-1-one,
(2R)-2-[[4-(2,6-dichloro-4-fluoro-phenyl)-7-quinolyl]oxy]-1-morpholino-propan-1-one,
8-[(2R)-2-[[4-(2,6-dichloro-4-fluoro-phenyl)-7-quinolyl]oxy]propanoyl]-2,8-diazaspiro[4.5]decan-1-one,
rac-(2R)-2-[[4-(2,6-dichloro-4-fluoro-phenyl)-7-quinolyl]oxy]-1-[3-(1-hydroxycyclopropyl)-1-piperidyl]propan-1-one,
(2R)-2-[[4-(2,6-dichloro-4-fluoro-phenyl)-7-quinolyl]oxy]-1-[(2R)-2-methyl-1-piperidyl]propan-1-one,
8-[(2R)-2-[[4-(2-chloro-4-fluoro-phenyl)-7-quinolyl]oxy]propanoyl]-2,8-diazaspiro[4.5]decan-1-one,
(2R)-2-[[4-(2-chloro-4-fluoro-phenyl)-7-quinolyl]oxy]-1-morpholino-propan-1-one,
(2R)-2-[[4-(2-chloro-4-fluoro-phenyl)-7-quinolyl]oxy]-1-[3-(1-hydroxycyclopropyl)-1-piperidyl]propan-1-one,
(2R)-2-[[4-(2,6-dichloro-4-fluoro-phenyl)-7-quinolyl]oxy]-1-(2-oxa-7-azaspiro[3.5]nonan-7-yl)propan-1-one,
N-hydroxy-N-[rac-(3S)-1-[rac-(2R)-2-[[4-(2-chloro-4-fluoro-phenyl)-7-quinolyl]oxy]propanoyl]-3-piperidyl]acetamide,
(2R)-2-[[4-(2-chloro-4-fluoro-phenyl)-7-quinolyl]oxy]-1-(2-oxa-7-azaspiro[3.5]nonan-7-yl)propan-1-one,
(2R)-2-[[4-(2-chloro-4-fluoro-phenyl)-7-quinolyl]oxy]-1-(2,6-dimethyl-1-piperidyl)propan-1-one,
(2R)-2-[[4-(2-chloro-4-fluoro-phenyl)-7-quinolyl]oxy]-1-(2-oxa-8-azaspiro[3.5]nonan-8-yl)propan-1-one,
(2R)-2-[[4-(2-chloro-4-fluoro-phenyl)-7-quinolyl]oxy]-1-(2-oxa-7-azaspiro[3.4]octan-7-yl)propan-1-one,
1-tert-butyl-3-[(3R)-1-[(2R)-2-[[4-(2-chloro-4-fluoro-phenyl)-7-quinolyl]oxy]propanoyl]pyrrolidin-3-yl]urea,
(2R)-2-[[4-(2-chloro-4-fluoro-phenyl)-7-quinolyl]oxy]-1-(3,3,5,5-tetramethylpiperazin-1-yl)propan-1-one,
(2R)-2-[[4-(2-chloro-4-fluoro-phenyl)-7-quinolyl]oxy]-1-(3,5-dimethylpiperazin-1-yl)propan-1-one,
(2R)-2-[[4-(2-chloro-4-fluoro-phenyl)-7-quinolyl]oxy]-1-[(1R)-2,5-diazabicyclo[2.2.1]heptan-2-yl]propan-1-one,
(2R)-2-[[4-(2-chloro-4-fluoro-phenyl)-7-quinolyl]oxy]-1-(2-oxa-6-azaspiro[3.3]heptan-6-yl)propan-1-one,
1-[(2R)-2-[[4-(2-chloro-4-fluoro-phenyl)-7-quinolyl]oxy]propanoyl]-3,6-dihydro-2H-pyridine-5-carboxylic acid,
2-[(3R)-1-[(2R)-2-[[5-(2,6-dichloro-4-fluoro-phenyl)-1,8-naphthyridin-2-yl]oxy]propanoyl]-3-piperidyl]acetic acid, and
2-[(3R)-1-[(2R)-2-[[5-(4-fluoro-2,6-dimethyl-phenyl)-1,8-naphthyridin-2-yl]oxy]propanoyl]-3-piperidyl]acetic acid,
or a pharmaceutically or veterinary acceptable salt, hydrate or solvate thereof.

4. The composition according to any of the preceding claims, wherein the at least one anti-cancer drug is selected from the group of (i) a B-cell lymphocyte-2 anti-apoptotic protein (Bcl-2) inhibitor, (ii) an inhibitor of the MEK/ERK pathway, including but not limited to a mitogen-activated protein (MAP) kinase inhibitor, MEK inhibitor or ERK inhibitor, (iii) an inhibitor of poly-ADP Ribose-Polymerase (PARPi), (iv) a Glucose consumption/uptake inhibitor, including but not limited to 2-deoxy glucose and derivatives and inhibitors of glucose transporters (GLUT), (v) a dihydroorotate-dehydrogenase (DHODH) inhibitor, (vi) a phosphatidylinositol-4,5-bisphosphate 3-kinase PIK3Cα (p110α), and (vii) an immunotherapeutic agent.

5. The composition according to claim 4, wherein the B-cell lymphocyte-2 anti-apoptotic protein (Bcl-2) inhibitor (i) is selected from the group consisting of Venetoclax (ABT-199), Navitoclax (ABT-263) and Oblimersen (G3139).

6. The composition according to claim 4, wherein the inhibitor of the MEK/ERK pathway (ii) is selected from the group consisting of Vemurafenib, Dabrafenib, Ulixertinib, Encorafenib (LGX818, (S)-methyl (1-((4-(3-(5-chloro-2-fluoro-3-(methylsulfonamido)phenyl)-1-isopropyl-1H-pyrazol-4-yl)pyrimidin-2-yl)amino)propan-2-yl)carbamate), Trametinib (GSK1120212), Binimetinib (MEK162), Cobimetinib (XL518, GDC0973), Selumetinib (AZD6244), N-[(2R)-2,3-Dihydroxypropoxy]-3,4-difluoro-2-[(2-fluoro-4-iodophenyl)amino]-benzamid (PD-325901), 2-(2-chloro-4-iodophenylamino)-N-cyclopropylmethoxy-3,4-difluorobenzamide (PD-184352/CI-1040), 3-[(2R)-2,3-Dihydroxypropyl]-6-fluor-5-[(2-fluor-4-iodphenyl)amino]-8-methylpyrido[2,3-d]pyrimidin-4,7(3H,8H)-dion (TAK-733), 2-((2-Fluoro-4-iodophenyl)amino)-N-(2-hydroxyethoxy)-1,5-dimethyl-6-oxo-1,6-dihydropyridine-3-carboxamide (AZD8330), and 5-Brom-N-(2,3-dihydroxypropoxy)-3,4-difluor-2-[(2-fluor-4-iodphenyl)amino]benzamid (PD-318088).

7. The composition according to claim 4, wherein the inhibitor of poly-ADP Ribose-Polymerase (PARPi) (iii) is selected from the group consisting of Olaparib, Rucaparib, Niraparib, Talazoparib, Veliparib, Pamiparib, CEP9722(11-methoxy-2-((4-methylpiperazin-1-yl)methyl)-4,5,6,7-tetrahydro-1H-cyclopenta[a]pyrrolo[3,4-c]carbazole-1,3(2H)-dione), E7016 (10-((4-Hydroxypiperidin-1-yl)methyl)chromeno[4,3,2-de]phthalazin-3(2H)-one), Iniparib, and 3-aminobenzamide.

8. The composition according to claim 4, wherein the Glucose consumption/uptake inhibitor (iv) is selected from 2-deoxy glucose and derivatives, and the GLUT inhibitor is BAY-876.

9. The composition according to claim 4, wherein the dihydroorotate-dehydrogenase (DHODH) inhibitor (v) is selected from the group consisting of Brequinar, Leflunomide/Teriflunomide, Enliuracil, Vidofludimus, GNF-Pf-4706, (E)-2-((2-(4-(2-chlorophenyl)thiazol-2-yl)hydrazono)methyl)benzoic acid (S312) and (E)-2-((2-(4-(2-chlorophenyl)thiazol-2-yl)-2-methylhydrazono)methyl)benzoic acid (S416).

10. The composition according to claim 4, wherein the phosphatidylinositol-4,5-bisphosphate 3-kinase PIK3Cα (p110α) inhibitor (vi) is selected from the group consisting of Duvelisib, Wortmannin, LY294002 (2-(4-Morpholinyl)-8-phenyl-4H-chromen-4-one), Copanlisib (BAY80-6946; 2-Amino-N-{7-methoxy-8-[3-(4-morpholinyl)propoxy]-2,3-dihydroimidazo[1,2-c]quinazolin-5-yl}-5-pyrimidinecarboxamide), AZD6482 (2-({(1R)-1-[7-Methyl-2-(4-morpholinyl)-4-oxo-4H-pyrido[1,2-a]pyrimidin-9-yl]ethyl}amino)benzoic acid), Bimiralisib (5-(4,6-dimorpholin-4-yl-1,3,5-triazin-2-yl)-4-(trifluoromethyl)pyridin-2-amine), Pictilisib (GDC0941; 2-(1H-Indazol-4-yl)-6-[[4-(methylsulfonyl)-1-piperazinyl]methyl]-4-(4-morpholinyl)thieno[3,2-d]pyrimidine), ZSTK474 (2-(Difluoromethyl)-1-[4,6-di(4-morpholinyl)-1,3,5-triazin-2-yl]-1H-benzimidazole), Omipalisib (GSK2126458; 2,4-Difluoro-N-[2-methoxy-5-[4-(4-pyridazinyl)-6-quinolinyl]-3-pyridinyl]benzenesulfonamide) and Buparlisib (BKM120; 5-[2,6-Di(4-morpholinyl)-4-pyrimidinyl]-4-(trifluoromethyl)-2-pyridinamine).

11. The composition according to claim 4, wherein the immunotherapeutic agent (vii) is selected from the group consisting of immune-stimulating agents interferone gamma, axitinib (N-Methyl-2-[[3-[(E)-2-pyridin-2-ylethenyl]-1H-indazol-6-yl]sulfanyl]benzamide), lenalidomide ((3RS)-3-(4-Amino-1-oxo-1,3-dihydro-2H-isoindol-2-yl)piperidine-2,6-dione), immune check-point inhibitors pembrolizumab, cemiplimab, durvalumab, ipilimumab, nivolumab, PD-1 ligand inhibitors atezolizumab, avelumab, anti-angiogenic agents ramucirumab, bevacimumab, cetuximab, rituximab, daratumumab, trastuzumab and antibody-drug conjugates bretuximab-vedotin.

12. A pharmaceutical composition comprising a composition as defined in any of the preceding claims and a pharmaceutically or veterinary acceptable excipient or carrier.

13. A kit comprising at least one inhibitor of mitochondrial transcription (IMT) as defined in any of the preceding claims and at least one anti-cancer drug as defined in any of the preceding claims.

14. A composition as defined in any of claims 1 to 11, a pharmaceutical composition as defined in claim 12, or a kit as defined in claim 13 for use as a medicament.

15. A composition as defined in any of claims 1 to 11, a pharmaceutical composition as defined in claim 12, or a kit as defined in claim 13 for use in a method of treating, and/or preventing cancer in a subject.

16. The composition as defined in any of claims 1 to 11, the pharmaceutical composition as defined in claim 12, or the kit as defined in claim 13 for use according to claim 14 or 15, wherein the cancer is selected from the group consisting of Acute Lymphoblastic Leukemia (ALL), Acute Myeloid Leukemia (AML), Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Childhood (Brain Cancer), Atypical Teratoid/Rhabdoid Tumor, Childhood, Central Nervous System (Brain Cancer), Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors (Lung Cancer), Burkitt Lymphoma, Carcinoid Tumor (Gastrointestinal), Cardiac (Heart) Tumors (Childhood), Central Nervous System Cancer, Atypical Teratoid/Rhabdoid Tumor (Childhood) (Brain Cancer), Medulloblastoma and Other CNS Embryonal Tumors (Childhood) (Brain Cancer), Germ Cell Tumor (Childhood) (Brain Cancer), Primary CNS Lymphoma, Cervical Cancer, Childhood Cancers, Rare Cancers of Childhood, Cholangiocarcinoma, Chordoma (Childhood) (Bone Cancer), Chronic Lymphocytic Leukemia (CLL), Chronic Myelogenous Leukemia (CML), Chronic Myeloproliferative Neoplasms, Colorectal Cancer, Craniopharyngioma (Childhood) (Brain Cancer), Cutaneous T-Cell Lymphoma (Mycosis Fungoides and Sezary Syndrome), Ductal Carcinoma In Situ (DCIS), Embryonal Tumors, Medulloblastoma and Other Central Nervous System (Childhood) (Brain Cancer), Endometrial Cancer (Uterine Cancer), Ependymoma (Childhood) (Brain Cancer), Esophageal Cancer, Esthesioneuroblastoma (Head and Neck Cancer), Ewing Sarcoma (Bone Cancer), Extracranial Germ Cell Tumor (Childhood), Extragonadal Germ Cell Tumor, Eye Cancer, Intraocular Melanoma, Retinoblastoma, Fallopian Tube Cancer, Fibrous Histiocytoma of Bone (Malignant, and Osteosarcoma), Gallbladder Cancer, Gastric (Stomach) Cancer, Gastrointestinal Carcinoid Tumor, Gastrointestinal Stromal Tumors (GIST) (Soft Tissue Sarcoma), Germ Cell Tumors, Childhood Central Nervous System Germ Cell Tumors (Brain Cancer), Glioma (Brain Cancer), Glioblastoma multiforme (GBM, Brain Cancer), Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors (Childhood), Hepatocellular (Liver) Cancer, Hodgkin Lymphoma, Hypopharyngeal Cancer (Head and Neck Cancer), Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell, Small Cell, Pleuropulmonary Blastoma, and Tracheobronchial Tumor), Lymphoma, Male Breast Cancer, Malignant Fibrous Histiocytoma of Bone and Osteosarcoma, Melanoma, Intraocular (Eye)Melanoma, Merkel Cell Carcinoma (Skin Cancer), Malignant Mesothelioma, Metastatic Cancer, Melanoma Brain Metastatic Cancer, Metastatic Squamous Neck Cancer with Occult Primary (Head and Neck Cancer), Midline Tract Carcinoma With NUT Gene Changes, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Chronic Myeloproliferative Neoplasms, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer, Lip and Oral Cavity Cancer and Oropharyngeal Cancer (Head and Neck Cancer), Osteosarcoma and Undifferentiated Pleomorphic Sarcoma of Bone Treatment, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Pituitary Tumor, Plasma Cell Neoplasm/Multiple Myeloma, Pleuropulmonary Blastoma (Lung Cancer), Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Childhood (Soft Tissue Sarcoma), Salivary Gland Cancer (Head and Neck Cancer), Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Soft Tissue Sarcoma, Uterine Sarcoma, Sezary Syndrome (Lymphoma), Skin Cancer, Small Cell Lung Cancer, Small Intestine Cancer, Soft Tissue Sarcoma, Squamous Cell Carcinoma of the Skin, Metastatic Squamous Neck Cancer with Occult Primary (Head and Neck Cancer), Stomach (Gastric) Cancer, Cutaneous T-Cell Lymphoma, Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Tracheobronchial Tumors (Lung Cancer), Triple-Negative Breast Cancer, Transitional Cell Cancer of the Renal Pelvis and Ureter (Kidney (Renal Cell) Cancer), Carcinoma of Unknown Primary, Ureter and Renal Pelvis, Transitional Cell Cancer (Kidney (Renal Cell) Cancer, Urethral Cancer, Uterine Cancer, Endometrial, Uterine Sarcoma, Vaginal Cancer, Vascular Tumors (Soft Tissue Sarcoma), Vulvar Cancer, Wilms Tumor and Other Childhood Kidney Tumors.

17. A composition as defined in any of claims 1 to 11, a pharmaceutical composition as defined in claim 12, or a kit as defined in claim 13 for use in a method of treating cancer in simultaneous, alternating or subsequent combination with another cancer therapy, preferably selected from chemotherapy, immunotherapy, hormone therapy, stem cell transplantation therapy, radiation therapy and surgery.
